(19) United States
(12) Patent Application Publication
(10) Pub. No.: US 2021/0061861 A1
(43) Pub. Date: Mar. 4, 2021

(54) D-PEPTIDIC COMPOUNDS FOR VEGF

(71) Applicants: Reflexion Pharmaceuticals, Inc., Incline Village, NV (US); The Governing Council of the University of Toronto, Toronto (CA)

(72) Inventors: Paul Marinec, Incline Village, NV (US); Kyle Landgraf, Incline Village, NV (US); Dana Ault-Riche, Incline Village, NV (US); Kurt Deshayes, San Francisco, CA (US); Maruti Uppalapati, Saskatoon (CA); Sachdev S. Sidhu, Toronto (CA)

(21) Appl. No.: 16/826,050

(22) Filed: Mar. 20, 2020

Related U.S. Application Data
(60) Provisional application No. 62/822,241, filed on Mar. 22, 2019.
(60) Provisional application No. 62/865,469, filed on Jun. 24, 2019.

(51) Int. Cl. C07K 14/00; A61K 49/00

(52) U.S. Cl. CPC C07K 14/001; A61K 38/00; A61K 49/0043; A61K 49/0056

(57) ABSTRACT

D-peptidic compounds that specifically bind to VEGF are provided. Also provided are multivalent D-peptidic compounds that include two or more of the domains connected via linking components. The multivalent (e.g., bivalent, trivalent, tetravalent, etc.) compounds can include multiple distinct domains that specifically bind to different binding sites on a target protein to provide for high affinity binding to, and potent activity against, the VEGF target protein. D-peptidic GA and Z domains that find use in the multivalent compounds are also provided, which polypeptides have specificity-determining motifs (SDM) for specific binding to VEGF (e.g., VEGF-A). Since the target protein is homodimeric (e.g., VEGF-A), the D-peptidic compounds may be similarly dimeric, and include a dimer of multivalent (e.g., bivalent) D-peptidic compounds. Also provided are methods for treating a disease or condition associated with VEGF or angiogenesis in a subject such as age-related macular degeneration (AMD) or cancer.

### CROSS-REFERENCE TO RELATED APPLICATIONS

This application claims the benefit of U.S. Provisional Patent Application No. 62/822,241, filed Mar. 22, 2019, and U.S. Provisional Patent Application No. 62/865,469, filed Jun. 24, 2019, which applications are incorporated herein by reference in their entirety.

### INTRODUCTION

Vascular endothelial cell growth factor (VEGF-A), is a key regulator of both normal and abnormal or pathological angiogenesis. In addition to being an angiogenic factor in angiogenesis and vasculogenesis, VEGF is a pleiotropic growth factor that exhibits multiple biological effects in other physiological processes, such as endothelial cell survival, vessel permeability and vasodilation, monocyte chemotaxis and calcium influx. Angiogenesis is an important cellular event in which vascular endothelial cells proliferate to form new vessels from an existing vascular network. Angiogenesis is implicated in the pathogenesis of a variety of disorders, such as tumors, proliferative retinopathies, age-related macular degeneration (AMD), rheumatoid arthritis (RA), and psoriasis. Angiogenesis is essential for the growth of most primary tumors and their subsequent metastasis in a variety of cancers.

The concentration of VEGF-A in eye fluids is correlated to the presence of active proliferation of blood vessels in patients with diabetic and other ischemia-related retinopathies. Furthermore, VEGF is localized in choroidal neovascular membranes in patients affected by AMD. Wet AMD is preceded by dry AMD, a condition characterized by the development of yellow-white deposits under the retina, along with variable thinning and dysfunction of the retinal tissue, although lacking any abnormal new blood vessel growth. Dry AMD converts to wet AMD when new and abnormal blood vessels invade the retina. This abnormal new blood vessel growth is called choroidal neovascularization (CNV). Anti-VEGF-A drugs find use in the treatment of wet AMD.

VEGF-A targeted therapies find use in the treatment of a variety of cancers. However, in some cases, patients eventually develop resistance to such therapy. Combination therapies that target VEGF-A and one more additional cancer targets are currently of interest, e.g., Programmed cell death protein 1 (PD-1) or Programmed death-ligand 1 (PD-L1). For example, a combination therapy targeting VEGF-A and PD-L1 using bevacizumab and atezolizumab showed a reduced risk of disease progression or death in patients with PD-L1 positive metastatic renal cell carcinoma.

The ability to manipulate the interactions of proteins such as VEGF-A is of interest for both basic biological research and for the development of therapeutics and diagnostics. Protein ligands can form large binding surfaces with multiple contacts to a target molecule that leads to binding events with high specificity and affinity. For example, antibodies are a class of protein that has yielded specific and tight binding ligands for various target proteins. In addition, Mandal et al. (“Chemical synthesis and X-ray structure of a heterochiral {D-protein antagonist plus VEGF} protein complex by racemic crystallography”, Proc. Natl. Acad. Sci. USA 109, 14779-14784 (2012)) and Uppalapati et al. (“A potent D-protein antagonist of VEGF-A is nonimmunogenic, metabolically stable and longer-circulating in vivo”, ACS Chem Biol (2016)) describe a D-protein antagonist of VEGF-A. Because of the diversity of target molecules of interest and the binding properties of protein ligands, the preparation of binding proteins with useful functions is of interest.

### SUMMARY

D-peptidic compounds that specifically bind to vascular endothelial cell growth factor (VEGF) are provided. The subject compounds can include a VEGF-A binding GA domain. The subject compounds can include a VEGF-A binding Z domain motif. Also provided are multivalent compounds that include two or more of the subject D-peptidic domains connected via linking components. The multivalent (e.g., bivalent, trivalent, tetravalent, etc.) D-peptidic compounds can include multiple distinct domains that specifically bind to different binding sites on a target protein to provide for high affinity binding to, and potent activity against, the VEGF target protein. D-peptidic GA and Z domains that find use in the multivalent compounds are also provided, which polypeptides have specificity-determining motifs (SDM) for specific binding to VEGF (e.g., VEGF-A). Since the target protein is homodimeric (e.g., VEGF-A), the D-peptidic compounds may be similarly dimeric, and include a dimer of multivalent (e.g., bivalent) D-peptidic compounds. The subject D-peptidic compounds find use in a variety of applications in which specific binding to VEGF-A target is desired. Methods for using the compounds are provided, including methods for treating a disease or condition associated with VEGF in a subject or associated with angiogenesis in a subject such as methods for treating a subject for age-related macular degeneration (AMD) or cancer.

### BRIEF DESCRIPTION OF THE DRAWINGS

FIG. 1 shows a view of the X-ray crystal structure of exemplary compound 1.1.1(c21a) (white stick representation) in complex with VEGF-A (space filling representation). The binding site residues of VEGF-A are depicted in pink. VEGF-A (8-109) binding site residues are indicated in bold:

(SEQ ID NO: 88)
GQNHHEVVKFMDVYQRSYCHPIETLVDIFQEYPDEIEYIFKPSCVPLMRC
GGCCNDEGLECVPTEESNITMQIMRIKPHQGQHIGEMSFLQHNKCECRPK
KD.

FIG. 2 shows an overlay of the X-ray crystal structure of exemplary compound 1.1.1(c21a) (white stick representation) in complex with VEGF-A (space filling representation) overlaid with the structure of the D-protein antagonist described by Mandal et al. (Proc. Natl. Acad. Sci. USA 109, 14779-14784 (2012)) (magenta stick representation). The binding site residues of VEGF-A are depicted in pink. The structure shows that compound 1.1.1(c21a) binds at the same antagonist site as the compound of Mandal et al.

FIG. 3A-3B show a side by side comparison of the three-helix bundle structures of a L-protein GA domain and an exemplary D-peptidic compound that specifically binds VEGF-A. FIG. 3A shows one view of an X-ray crystal structure of a L-protein GA domain (Protein Data Bank structure 1tf0) and a schematic indicating the arrangement of Helices 1-3. FIG. 3B shows a similar view of the X-ray crystal structure of compound 1.1.1(c21a) in complex with VEGF-A (not shown in this view) and a schematic indicating the arrangement of Helices 1-3.

FIG. 4 shows a view of the X-ray crystal structure of compound 1.1.1(c21a) in complex with VEGF-A (not shown in this view). Helix 1 (201), Helix 2 (202) and Helix 3 (203) are alpha-helix regions of the D-peptidic compound corresponding to those of the native GA domain. 206 is a phenylalanine residue at position 31 (f31). 205, 207 and 210 are histidine residues at positions 27 (h27), 34 (h34) and 40 (h40), respectively. 209 is a tyrosine residue at position 37 (y37). 204 and 208 are Helix 2-terminating proline residues located at positions 26 (p26) and 36 (p36), respectively.

FIG. 5 depicts the binding interface between an exemplary D-peptidic compound (1.1.1 c21a); stick representation) and VEGF-A (space filing representation) taken from the X-ray crystal structure of the complex. Residue f31 (206) of the compound projects into a binding pocket of VEGF-A at the binding interface of the complex. Histidine residues at positions 27 (205), 34 (207) and 40 (210) make additional contacts with the VEGF-A at the binding interface. The sidechain of residue y37 (209) projects towards the VEGF-A surface but does not make close contacts.

FIG. 6A-6D depicts a structural model for the subject compounds based on a three-helix bundle structure. FIG. 6A shows a schematic of the arrangement of three helices in a native GA domain. FIG. 6B shows a schematic of the arrangement of the three helices in a D-peptidic GA domain motif. FIG. 6C shows Degrado's structural model of antiparallel three-stranded helices based on hydrophobic packing of heptad repeat units; seven residue motifs (abcdefg)n that form helical segments having characteristic residues at particular positions of the motif. FIG. 6D shows the adaptation of Degrado's heptad repeat model to the D-peptidic three-helix domain motif

FIG. 7A-7B depicts the three-helix bundle structural model for the subject D-peptidic compounds. FIG. 7A depicts a first arrangement of Helices 1-3 as found in a GA domain motif. FIG. 7B shows the structural model for the three helix bundle of the subject compounds.

FIG. 8A-8C depict a structural model for the subject compounds based on a two-helix complex structure. FIG. 8A depicts a first arrangement of Helices A-B in side view and top view consistent with that found in a GA domain motif, where N and C denote the N-terminal and C-terminal of the peptidic compound. FIG. 8B shows the structural heptad repeat model for the two helix complex of the subject compounds including a g-g face which contacts the VEGF-A. FIG. 8C depicts a variant motif including selected VEGF-A contacting residues located in the solvent exposed c and g positions (in blue) of the two helix complex heptad repeat model (see FIG. 8B) defined by Helix A and Helix B, where h* is histidine or an analog thereof, is phenylalanine or an analog thereof and u is a non-polar amino acid residue. In FIG. 8C, the “_” indicate positions of the underlying scaffold domain and the dashed lines indicate locations of possible interhelix contacts or linkages of residues.

FIG. 9A-9C depicts a structural model for the subject compounds that relates compound sequence to the three-helix bundle structure. FIG. 9A shows a three dimensional representation of a portion of the heptad repeat model for an exemplary compound. Selected residues of compound 1.1.1 (c21a) are assigned to the positions of the heptad repeat unit model, consistent with the X-ray crystal structure of the compound in complex with VEGF-A. The VEGF-A binding face of the compound defined by Helix 2 and Helix 3 corresponds to the g-g face of the heptad repeat model.

FIG. 9B shows a view of the X-ray crystal structure of compound 1.1.1 (c21a) with a and d residues of the heptad register shown in red, which pack in the core of the three helix bundle structure. FIG. 9C shows a linear alignment of the sequence with the heptad repeat model of the tertiary structure (H1=Helix 1; H2=Helix 2; H3=Helix 3) with core residues indicated in red and selected VEGF-A contacting residues indicated in blue. It is understood that that structural model depicted in FIG. 9A can be extended to show all of the residues in each of Helices 1-3 based on the register shown in FIG. 9C. For simplicity, only a portion of the structure is depicted.

FIG. 10A-10B provide further depictions of specific and general heptad repeat models of the subject compounds. FIG. 10A shows an alignment of the sequence of exemplary compound 1.1.1 (c21a) with the heptad repeat model of the tertiary structure where hydrophobic contacts of core residues between the helices of the three-helix bundle are depicted with arrows. FIG. 10B depicts a variant motif including selected VEGF-A contacting residues located in the solvent exposed c and g positions of the g-g face (see FIGS. 7B and 8A) defined by Helix 2 and Helix 3, where h* is histidine or an analog thereof, is phenylalanine or an analog thereof and u is a non-polar amino acid residue. In FIG. 10B, the “_” indicate positions of an underlying scaffold domain and the dashed lines indicate possible hydrophobic contacts of core residues between the helices of the three-helix bundle.

FIG. 11 shows an expanded stick view of a portion of the X-ray crystal structure of an exemplary D-peptidic compound (1.1.1 (c21a)) taken from of the binding complex with VEGF-A (not shown). The fragment corresponds to a part of the Helix 2-Linker 2-Helix 3 region spanning positions 26-45. 202 indicates Helix 2, 203 indicates Helix 3 which are joined by Linker 2. Hydrophobic residues at positions 32, 35, 41 and 44 are included in Helix 2-Helix 3 intramolecular contacts.

FIG. 12 shows an expanded ribbon view of a portion of the X-ray crystal structure of L-protein GA domain (1tf0). The view corresponds to a part of the Helix 2 to Helix 3 region spanning positions 31-44. 102 and 103 are alpha-helix regions of the native GA domain structure corresponding to Helix 2 (202) and Helix 3 (203) regions, respectively. Linker 2 is a linking region. Residues at positions 32, 35, 41 and 44 are shown which are part of the intramolecular hydrophobic contacts between Helix 2-Helix 3, similar with those shown in FIG. 12.

FIG. 13 shows structural depictions and underlying sequence (SEQ ID NO:2) of the scaffolded library SCF32 based on the GA domain of protein G (e.g., Protein Data Bank (PDB) structure 1tf0) including sequence positions (bold) randomized for mirror image phage display screening against VEGF-A.

FIG. 14 shows an alignment of a selection of GA scaffold domains of interest (SEQ ID NO: 6-21) and a GA domain consensus sequence (SEQ ID NO: 1) (FIG. 1 of Johansson et al. (“Structure, Specificity, and Mode of Interaction for Bacterial Albumin-binding Modules”, J. Biol. Chem., Vol. 277, No. 10, pp. 8114-8120, 2002) which can be adapted for use as scaffold domains in the subject compounds.

FIG. 15 shows an alignment of a GA scaffold domain (SEQ ID NO: 2) and exemplary VEGF-A binding compounds: 1 (SEQ ID NO: 106), 1.1 (SEQ ID NO: 22), 1.1.1 (SEQ ID NO: 23) and 1.1.1 (c21a) (SEQ ID NO: 24).

FIG. 16 shows melting and refolding curves for exemplary compound 1.1.1. The melting temperature was determined to be approximately 50° C.

FIG. 17 shows a view of the X-ray crystal structure of the dimeric complex between an exemplary D-peptidic compound (1.1.1 (c21 a); stick representation) and VEGF-A (space filing representation).

FIG. 18A-18B depict the design of an exemplary compound ((−)-TIDQW) having a truncated N-terminal relative to compound 1.1.1 (c21a). FIG. 18A shows an expanded view of the X-ray crystal structure of the complex between exemplary D-peptidic compound (1.1.1 (c21a); stick representation) and VEGF-A (space filing representation), which indicates that the N-terminal residues of Helix 1 which do not make contacts with Helix 2 or Helix 3. In some cases, select N-terminal residues can be truncated from Helix 1 without significant loss of stability or binding affinity. FIG. 18B shows a side by side comparison of structures of the truncated (−) TIDQW versus non-truncated (+)-TIDQW compound 1.1.1(c21a).

FIG. 19A-19C show a series of positions in the compound where affinity maturation is performed or optional point mutations are incorporated. FIGS. 19A and 19B depict a view of compound 1.1.1(c21a) either isolated (FIG. 19A) or in complex with VEGF-A (FIG. 19B) taken from the X-ray crystal structure. FIG. 19C shows the sequence of compound 1.1.1(c21a) (SEQ ID NO: 24) and notes mutations of interest.

FIG. 20 shows an expanded view of the X-ray crystal structure of compound 1.1.1(c21a) (stick representation) in complex with VEGF-A (space filling representation) with the phenylalanine (f) residue at position 31 shown in yellow projecting into a binding pocket of VEGF-A at the binding interface of the complex.

FIG. 21 shows an expanded view of the f31 residue sidechain projecting into a binding pocket of the VEGF-A binding interface where selected distances between the phenyl ring and adjacent residues of VEGF-A are shown in angstroms. Analysis of the complex structure indicates various phenylalanine analogs are tolerated at position 31, e.g., an analog including a substituent at the 3, 4 and/or 5 positions of the phenyl ring that can occupy the available space (4.6 to 5.3 angstrom) of the binding pocket of VEGF-A.

FIG. 22 shows an expanded view of the X-ray crystal structure of compound 1.1.1(c21a) (stick representation) in complex with VEGF-A (space filling representation) with selected Helix 2 contacts shown. 205 and 207 are histidine residues at positions 27 and 34, respectively. The structure shows a weak hydrogen bond (approx. 4.6 angstrom) between a nitrogen atom of histidine 34 (h34; 207) and adjacent Asp90 of VEGF-A. 209 is the tyrosine residue of the compound at position 37 that projects towards the VEGF-A surface. Analysis of the complex structure indicates various histidine analogs are tolerated at positions 27 and 34, e.g., an analog including a substituted or unsubstituted aryl or heterocyclic ring that can occupy the available space on the surface of VEGF-A and/or make a stronger hydrogen bond (e.g., of <4.6 angstrom in length) to adjacent residues of VEGF-A .

FIG. 23 shows an expanded view of the X-ray crystal structure of compound 1.1.1(c21a) (stick representation) in complex with VEGF-A (space filling representation) with selected Helix 3 contacts shown. The structure shows a medium strength hydrogen bond (2.9 angstrom) between a nitrogen atom of histidine 40 (h40; 210) and adjacent residue Tyr48 of VEGF-A. Analysis of the complex structure indicates various histidine analogs are tolerated at position 40, including analogs that can occupy the available space and retain or strengthen the hydrogen bond to VEGF-A.

FIG. 24 shows an expanded view of the X-ray crystal structure of compound 1.1.1(c21a) (pink and green stick representation) in complex with VEGF-A (cyan ribbon) focusing on the tyrosine (y) residue at position 37 (209) of Linker 2. The distances between the y37 oxygen and oxygen or nitrogen atoms of proximate resides on the VEGF-A surface are shown, e.g., 6.5 and 7.2 angstrom, which indicate that various tyrosine analogs are tolerated at position 37, e.g., an analog including an substituted or unsubstituted, alkyl-aryl or alkyl-heteroaryl extended sidechain group that can make closer contacts (e.g., hydrophobic contacts and/or a hydrogen bond) with adjacent residues of VEGF-A.

FIG. 25 shows an expanded view of the X-ray crystal structure of compound 1.1.1(c21a) (stick representation) in complex with VEGF-A (space filling representation) focusing on the histidine residue (h) at position 27 (205). Analysis of the structure indicates that a variety of aromatic residues or histidine analogs can be utilized at position 27 to contact the same pocket on the surface of VEGF-A and, in some cases, to increase desirable hydrophobic contacts. Also shown is a glutamic acid residue at positions 25 (e25, 211) of the [Linker 1] region, which makes contact with VEGF-A, including a hydrogen bond (2.5 angstroms) to a main chain carbonyl group of the peptidic backbone of VEGF-A.

FIG. 26 shows a sequence logo of selected positions of all the clones identified during a phage display mirror image screening for D-VEGF-A binder, where the sequence logo is aligned in comparison to corresponding residues of the Compound 1 sequence and native GA domain (GA-wt).

FIG. 27A-27B, show a comparison of the structures of a L-protein GA domain (FIG. 27A) and D-compound 1.1.1(c21a) (FIG. 27B) indicating the angle of alignment between Helices 2 and 3 is increased in the VEGF-A binding compound.

FIG. 28A-28B show two depictions of the X ray crystal structure of D-peptidic compound 11055 bound to VEGF-A homodimer. FIG. 28A shows D-peptidic compound 11055 binds to VEGF-A primarily via binding contacts of helix 2 (H2) of the variant GA domain of compound 11055. FIG. 28B shows the structure of FIG. 28A, where the D-peptidic compound 11055 is represented with a space filling model, overlaid with the structure of VEGFR2 (Domains 2 and 3) bound to VEGF-A. The overlay shows that D-peptidic compound 11055 blocks binding of domain 2 (D2) of VEGFR2 to VEGF-A.

FIG. 29A-29B show depictions of the structure (FIG. 29A) and sequence (FIG. 29B) of an affinity maturation library designed to screen for and identify residues at particular positions that stabilize the variant GA domain fold of compound 11055. A total of 7 residues were selected for mutation at the packing interface between helix 1 (H1) and the loop connecting helix 2 (H2) and helix 3 (H3).

FIG. 30A-30C show results of screening for high affinity VEGF-A binding compounds which compounds include a consensus sequence logo having cysteine residues at positions 7 and 38 (FIG. 30A) and selected variant sequences of interest (FIG. 30B) (SEQ ID NOs: 108-113) with their binding affinities for VEGF-A versus parent compound 11055. FIG. 30C shows an expanded view of the structure of the parent compound 11055 (FIG. 29A) with identified variant amino acid residue positions 17c and v38c shown in yellow to be proximate to each other (betaC to betaC interhelix distance of 5.9 angstroms) such that inclusion of 17c and v38c variations would provide for formation of stabilizing disulfide bond between those residues.

FIG. 31A-31B shows graphs of data that demonstrate the activity of VEGF-A D-peptides. FIG. 31A shows VEGF-A antagonistic activity of select compounds in a VEGFR1 binding ELISA. FIG. 31B shows inhibition of cell proliferation in response to VEGF signaling by select compounds versus an bevacizumab control.

FIG. 32A-32B show depictions of the structure (FIG. 32A) and sequence (FIG. 32B) of a phage display library based on a parent Z-domain scaffold. Ten positions (X) were selected within helix 1 to helix 2 of the Z domain for randomization using kunkel mutagenesis with trinucleotide codons representing all the amino acids except cysteine (FIG. 32B).

FIG. 33A-33B show the results of mirror image phage display screening for binding to VEGF-A using a Z domain phage display library. FIG. 33A shows a consensus sequence logo that provide for binding to VEGF-A. FIG. 33B shows selected variant Z domain sequences of interest (SEQ ID NOs: 114-118) with their binding affinities for native L-VEGF-A. NB refers to non-binding.

FIG. 34 shows a surface plasmon resonance (SPR) sensorgram showing additive binding of compounds 978336 and 11055, indicating that compound 978336 (a variant Z domain compound) binds to a binding site on VEGF-A that is non-overlapping and independent of the binding site of compound 11055 (variant GA domain compound).

FIG. 35A-35G show three depictions of the X ray crystal structure of D-peptidic compound 978336 bound to VEGF-A homodimer. FIG. 35A shows two monomeric D-peptidic compounds 978336 bound to their binding sites of VEGF-A. FIG. 35B shows the structure of FIG. 35A, where the D-peptidic compound 978336 are represented with a space filling model, overlaid with the structure of VEGFR2 (Domains 2 and 3) bound to VEGF-A. The overlay shows that D-peptidic compound 978336 blocks binding of domain 3 (D3) of VEGFR2 to VEGF-A. FIG. 35C shows the structure of 978336 in isolation looking at the VEGF-A binding face of the compound with the variant amino acid residues selected from the Z domain library shown in red. FIG. 35D shows an expanded view of the protein to protein contacts (top panel) and the binding site on VEGF-A (bottom panel) of compound 978336 (SEQ ID NO: 117) including the configuration of variant amino acids in contact with the binding site (top panel). FIG. 35E-35G illustrate the affinity maturation studies of exemplary VEGF-A binding compound 978336 (SEQ ID NO: 117), a consensus sequence (SEQ ID NO: 158) identified (FIG. 35F) and the sequence of an exemplary compound 980181 (SEQ ID NO: 119).

FIG. 36A-36B illustrate the structure based-design of an exemplary bivalent compound conjugate, including compounds 11055 and 978336 conjugated via N-terminal cysteine residues using a bis-maleimide PEG8 linker (FIG. 36A). FIG. 36B illustrates the sequence of bivalent compound 979111 including a N-terminal to N-terminal linkage via conjugation with a bismaleimide PEG8 bifunctional which exhibited a binding affinity of 1.7 nM for L-VEGF-A as measured by SPR.

FIG. 37A-37B show depictions of the structure (FIG. 37A) and sequence (FIG. 37B) of a phage display library (SEQ ID NO: 159) based on a parent GA domain scaffold (SEQ ID NO: 2). Eleven positions (X) were selected within helix 2 to helix 3 of the GA domain scaffold for randomization using kunkel mutagenesis with trinucleotide codons representing all amino acids except cysteine.

FIG. 38A-38E illustrate the design, synthesis and sequence of exemplary dimeric bivalent (i.e., tetradomain-containing) compounds 980870 and 980871. FIG. 38A shows a depiction of the X ray crystal structures of exemplary compounds 11055 and 978336 bound to VEGF-A and the design of linkers for producing an exemplary dimeric, bivalent VEGF-A binding compound. Residue k19 of compound 11055 and residue k7 of compound 978336 can be connected through their sidechain amino groups via a linker, e.g., of approximately 23 angstroms or more in length. FIG. 38B shows a synthetic scheme for use in preparing linked tetradomain compounds 980870 and 980871. D-Pra is a D-propargylglycine residue linked to the amine sidechain of k7 of compound 980181 via a —NH-PEG2-CO—linker. An azido-CH₂CONH-PEG2/3-CO— group is linked to the amine sidechain of k19 of compound 979110 and subsequently conjugated to the propargyl group using click chemistry to form an interdomain linker. FIG. 38C shows depictions of the sequences of exemplary tetradomain compounds prepared via the scheme of FIG. 38B. FIG. 38D is a schematic diagram of an exemplary bivalent compound including a linker L¹ between residue x¹⁹ of the GA domain and residue x⁷ of the Z domain. FIG. 38E is a schematic diagram of an exemplary dimeric bivalent compound including a second linker L² between the C-terminal residues of the GA and Z domains.

FIG. 39A-39B show graphs of the results of assays measuring in vitro (FIG. 39A) and cell based (FIG. 39B) antagonist activity against VEGF-A of exemplary dimeric bivalent (i.e., tetradomain-containing) compounds compared to monovalent domains 979110 and 980181 and bevacizumab.

FIG. 40A-40C show activity data for D-protein VEGF-A antagonists developed using mirror-image phage display. (FIG. 40A) Phage titration ELISA of GA-domain and Z-domain hits against the D-VEGF-A target showing titratable binding. (FIG. 40B) Phage competition ELISA using the synthetic L-enantiomer corresponding to the GA-domain hit as a soluble competitor to displace phage binding to D-VEGF-A. (FIG. 40C) Titrations of synthetic D-proteins RFX-11055 and RFX-978336 in a VEGF-A blocking ELISA showing antagonistic activity relative to bevacizumab.

FIG. 41A-41F shows structures of the D-proteins RFX-11055 and RFX-978336 in complex with VEGF-A. (FIGS. 41A and 41B) Overview of RFX-11055 (purple) and RFX-978336 (blue) bound to distinct non-overlapping epitopes at distal ends of a VEGF-A homodimer (grey). (FIGS. 41C and 41D) Interfacial D-amino acid side chains contacting VEGF-A depicted for RFX-11055 and RFX-978336 with selected library residues (orange) and original scaffold residues (blue) within helix 2 and 3 for RFX-11055 and helix 1 and 2 for RFX-978336. VEGF-A is shown with electrostatic surface potential to highlight positive (blue), negative (red) and neutral hydrophobic (white) contact sites. (FIG. 41E) Previously reported crystal structure of VEGF-A (grey) in complex with VEGFR-1 receptor (light orange). Ig domains 2 and 3 (D2 and D3) of VEGFR-1 are isolated to highlight molecular interactions in receptor engagement of VEGF-A (PDB code: 5T89) (24). (FIG. 41F) Overlay of RFX-11055 and RFX-978336 on the VEGF-A/VEGFR-1 complex to demonstrate direct competition with D2 and D3 as the mechanism for VEGF-A blockade.

FIG. 42A-42C illustrate structure-guided affinity maturation of RFX-11055 and RFX-978336. (FIG. 42A) Structure of RFX-11055 (purple) bound to VEGF-A (grey) showing seven residues (orange) targeted for affinity maturation libraries to stabilize packing between helix 1 and the helix 2-3 binding interface. (FIG. 42B) Structure of RFX-978336 (blue) bound to VEGF-A (grey) showing the helix 1-2 binding interface and the four residues selected for soft-randomization libraries. (FIG. 42C) Titrations of affinity matured D-proteins RFX-979110 and RFX-980181 in the VEGF blocking ELISA showing antagonistic activity relative to bevacizumab.

FIG. 43A-43B show in vitro activity of the D-protein heterodimeric VEGF-A antagonist. (FIG. 43A) Titrations of the affinity matured D-protein RFX-979110 and the high-affinity heterodimer RFX-980869 in the VEGF-A blocking ELISA, compared with bevacizumab and a VEGFR1-Fc soluble decoy receptor. (FIG. 43B) Cell activity assay showing that RFX-980869 potently blocks VEGF-A signaling through VEGFR2, with potency comparable to bevacizumab.

FIG. 44A-44B show in vivo activity of D-protein RFX-980869 in a rabbit eye model of wet AMD. (FIG. 44A) Representative fluorescein angiography (FA) images depicting the extent of VEGF-A165-induced vascular leakage at Day 5 and Day 26 post administration of respective drugs (control=no drug treatment). (FIG. 44B) Plots of individual FA scores at Day 5 and Day 26. Scoring is as follows: 0=major vessels straight with some tortuosity of small vessels and no dilation, 1=increased tortuosity of major vessels and some dilation, 2=leakage between major vessels and significant dilation, 3=leakage between major and minor vessels and minor vessels still visible, 4=leakage between major and minor vessels and minor vessels poorly visible or not visible. N=5 rabbits per group (10 eyes). All data is plotted as mean±SEM. (****p<0.0001, Mann-whitney test)

FIG. 45A-45D show tumor growth inhibition activity of RFX-980869 and lack of immunogenicity. (FIG. 45A) MC38 tumor growth curves in C57BL6 mice showing dose-dependent efficacy of both RFX-980869 and nivolumab. N=6 mice per group. (FIG. 45B) Day 15 tumor volumes (*p<0.05, Mann-whitney test) (FIG. 45C) Anti-drug-antibodies from MC38 tumor study measured in Day 22 serum samples using an ELISA for antigen-specific serum IgG. (FIG. 45D) Anti-drug-antibody titers measured from Day 42 serum after subcutaneous immunization of corresponding drugs in BALB/c mice. N=5 mice per group. All data is plotted as mean±SEM.

FIG. 46A-46C show phage display libraries and sequences of D-proteins. (FIG. 46A) GA-domain scaffold sequence and library used for panning Underlined residues in GA library were hard-randomized with NNK codons for full amino acid diversity. Underlined residues in the AM library were hard randomized using NNC codon for 15 amino acid diversity including cysteine. Lowercase amino acids for RFX-11055 and RFX-979110 denote D-amino acids. Sequences from top to bottom: (SEQ ID NO: 2; SEQ ID NO: 108; SEQ ID NO: 108; SEQ ID NO: 108; SEQ ID NO: 113) (FIG. 46B) Z-domain scaffold sequence and library used for panning Underlined residues in GA library were hard randomized for full amino acid diversity using trinucleotide codons for each amino acid, with the exception of cysteine. Underlined residues in the AM library were soft-randomized using codons to incorporate 30% mutation rate at each amino acid. Lowercase amino acids for RFX-978336 and RFX-980181 denote D-amino acids. Sequences from top to bottom: SEQ ID NO: 163; SEQ ID NO: 117; SEQ ID NO: 117; SEQ ID NO: 117; SEQ ID NO: 119). (FIG. 46C) Full D-amino acid sequence for heterodimeric antagonist 980869.

FIG. 47 shows SPR sensorgrams of kinetic binding parameters measured for D-proteins and bevacizumab.

FIG. 48 shows SPR-based epitope mapping of RFX-978336 and RFX-11055. In the first association step, 5 μM of RFX-978336 is used to saturate VEGF-A on the chip surface. In the second association step, 1 μM of RFX-11055 is included with 5 μM of RFX-978336 and exhibits additive binding to VEGF-A indicating the site for RFX-11055 is not blocked by RFX-978336. Both D-proteins display complete dissociation from VEGF-A.

FIG. 49A-49B illustrate structural characterization of the VEGF-A/VEGFR-1 contacts. (FIG. 49A) Previous structure solved for VEGF-A (grey) in complex with VEGFR-1 (light orange) depicting the epitope on VEGF-A contacted by D2 and D3 Ig-domains of VEGFR-1 colored by element (white carbon, red oxygen, blue nitrogen, and yellow sulfur) (PDB ID: 5T89, 24). (FIG. 49B) Open book representation of (FIG. 49A) with the D2 and D3 domains rotated 180 degrees away from VEGF-A and electrostatic surface potential shown for both molecules. The D2 and D3 binding sites are encircled highlighting the predominant non-polar hydrophobic nature of the D2 interaction and polar hydrophilic nature of the D3 interaction.

FIG. 50A-50B illustrate the design and synthesis of the heterodimeric D-protein RFX-980869. (FIG. 50A) Structural overlay of RFX-11055 (purple) and RFX-978336 (blue) bound to VEGF-A (grey) showing the Lysine residues (K19 on RFX-11055 and K7 on RFX-978336) in sphere representation with distance measurements for proposed PEG linkages. (FIG. 50B) Synthesis scheme for creating the D-protein heterodimer, RFX-980869, using solid phase peptide synthesis with peptide and PEG moieties equipped with ‘Click’ chemistry functional groups.

FIG. 51 shows a table with a summary of SPR-derived kinetic binding parameters for D-proteins and bevacizumab.

FIG. 52 shows a table with a summary of IC50 values for D-proteins and bevacizumab blocking VEGF-A121 binding to VEGFR1-Fc in non-equilibrium ELISA.

FIG. 53 shows a table with data collection and refinement statistics for VEGF/D-protein complexes.

FIG. 54 shows a table with a summary of IC50 values for D-proteins and bevacizumab blocking VEGF-121A binding to VEGFR1-Fc in an equilibrium binding ELISA and VEGF-A signaling inhibition in a cell signaling assay.

FIG. 55 shows a sequence logo of selected positions of all the clones identified during a phage display mirror image screening for D-peptidic Z domain VEGF-A binder, where the sequence logo is aligned in comparison to corresponding residues of the native Z domain (Z-wt).

### DETAILED DESCRIPTION

### Multivalent D-Peptidic Binding Compounds

As summarized above, aspects of this disclosure include multivalent D-peptidic compounds that specifically bind with high affinity to VEGF. This disclosure provides a class of multivalent compounds that is capable of specifically binding to a VEGF target protein at two or more distinct binding sites on the target protein. The term “multivalent” refers to interactions between a compound and a target protein that can occur at two or more separate and distinct sites of a target protein molecule. The multivalent D-peptidic compounds are capable of multiple binding interactions that can occur cooperatively to provide for high affinity binders to target proteins and potent biological effects on the function of the target protein. The term “multimeric” refers to a compound that includes two (i.e., dimeric), three (i.e., trimeric) or more monomeric peptidic units (e.g., domains) When the multimeric compound is homologous each peptidic unit can have the same binding property, i.e. each monomeric unit is capable of binding to the same binding site(s) on a VEGF target protein molecule. Such multimeric compounds can find use in binding target proteins that occur naturally as homodimers or are capable of multimerization. A dimeric compound can bind simultaneously to the two identical binding sites on the two molecules of the VEGF target protein homodimer. In some instances, depending on the target protein, the multivalent D-peptidic compounds of this disclosure can be multimerized, e.g., a dimeric bivalent D-peptidic compound can include a dimer of two bivalent D-peptidic compounds. In certain cases, the multimeric compound is heterologous and each peptidic unit (e.g., domain or bivalent unit) specifically binds a different target site or protein.

The multivalent peptidic compound includes at least two peptidic domains where each domain has a specificity determining motif composed of variant amino acids configured to provide a interface of specific protein-protein interactions at a binding site. When multiple peptidic domains are linked together they can simultaneously contact the target protein and provide multiple interfaces at multiple binding sites. The multiple protein-protein binding interactions can occur cooperatively via an avidity effect to provide for significantly higher effective affinities than is possible to achieve for any one D-peptidic domain alone. The present disclosure discloses use of mirror image phage display screening using scaffolded small protein domain libraries to produce multiple peptidic domains binding multiple target binding sites, and that such domains can be successfully linked to produce high affinity binders exhibiting a strong avidity effect. The multimeric compounds demonstrated by the inventors have affinity comparable to or better than corresponding antibody agents and provide for effective biological activity against VEGF target protein in vivo.

In general, the VEGF target protein is a naturally occurring L-protein and the compound is a D-peptidic compound. It is understood that for any of the D-peptidic compounds described herein, a L-peptidic version of the compound is also included in the present disclosure, which specifically binds to a D-VEGF target protein. The subject peptidic compounds were identified in part by using methods of mirror image screening of a variety of scaffolded domain phage display libraries for binding to a synthetic D-VEGF target protein.

D-peptidic compounds can provide a number of desirable properties for therapeutic applications in comparison to a corresponding L-polypeptide, such as proteolytic stability, substantially reduced immunogenicity and long in vivo half life. The D-peptidic compounds of this disclosure are generally significantly smaller in size by comparison to an antibody agent for VEGF. In some cases, the smaller size and properties of the subject compounds provide for routes of administration, tissue distribution and tissue penetration, and dosage regimens that are superior to antibody-based therapeutics.

This disclosure provides a multivalent D-peptidic compound including at least first and second D-peptidic domains. The first and second D-peptidic domains can specifically bind to distinct non-overlapping binding sites of the target protein and can be linked to each other via a linking component (e.g., as described herein). The linking component can be configured to allow for simultaneous or sequential binding to the target protein. By “sequential binding” it is meant that binding of the first D-peptidic domain to the target can increases the likelihood binding by the second D-peptidic domain will occur, even if binding does not occur simultaneously.

The first and second D-peptidic domains can be heterologous to each other, i.e., the domains are of different domain types. For example, the first D-peptidic domain may be a variant GA domain and the second D-peptidic domain may be a variant Z domain, or vice versa. Mirror image phage display screening of VEGF using two different scaffolded domain libraries provides variant domain binders that are directed towards two different binding sites on the VEGF.

When the multivalent D-peptidic compound includes only two such domains it can be termed bivalent. Trivalent, tetravalent and higher multivalencies are also possible. Trivalent D-peptidic compounds can include three D-peptidic domains connected via two linking components in a linear fashion, or via a single trivalent linking component. Trivalent D-peptidic compounds can include two of the same D-peptidic compounds connected via a disulfide linkage between two cysteine residues on each D-peptidic compound and a linking component between one of the disulfide linked D-peptidic compounds and a third D-peptidic compound. Tetravalent and higher multivalent compounds can similarly be linked, either in a linear fashion via bivalent linking components, or in a branched configuration via one or more multivalent or branched linking components.

### Linking Components

The term “linking component” is meant to cover multivalent moieties capable of establishing covalent links between two or more D-peptidic domains of the subject compounds. Sometimes, the linking component is bivalent. Alternatively, the linking component is trivalent or dendritic. A linking component may be installed during synthesis of D-peptidic domain polypeptides, or post-synthesis, e.g., via conjugation of two or more D-peptidic domains that are already folded. A linking component may be installed in a subject compound via conjugation of two D-peptidic domains using a bifunctional linker. A linking component may also be designed such that it may be incorporated during synthesis of the D-peptidic domain polypeptides, e.g., where the linking component is itself peptidic and is prepared via solid phase peptide synthesis (SPPS) of a sequence of amino acid residues. In addition, chemoselective functional groups and/or linkers may be installed during polypeptide synthesis to provide for facile conjugation of a D-peptidic domain after SPPS.

Any convenient linking groups or linkers can be adapted for use as a linking component in the subject multivalent compounds. Linking groups and linker units of interest include, but are not limited to, amino acid residue(s), polypeptide, PEG units, (PEG)n linker (e.g., where n is 2-50, such as 2-40, 2-30, 2-20 or 2-10), terminal-modified PEG (e.g., —NH(CH₂)ₘO[(CH₂)₂O](CH₂)ₚCO—, or —NH(CH₂)ₘO.[(CH₂)₂O]ₙ(CH₂)ₘNH—, or —CO(CH₂)ₚO[(CH₂)₂O]ₙ(CH₂)ₚCO— linking groups where m is 2-6, p is 1-6 and n is 1-50, such as 1-20, 1-12 or 1-6), C1-C6alkyl or substituted C1-C6alkyl linkers, C2-C12alkyl or substituted C2-C12alkyl linkers, succinyl (e.g., —COCH₂CH₂CO—) units, diaminoethylene units (e.g., —NRCH₂CH₂NR— wherein R is H, alkyl or substituted alkyl), —CO(CH₂)ₘCO—, —NR(CH₂)ₚNR—, —CO(CH₂)ₘNR—, —CO(CH₂)ₘO—, —CO(CH₂)ₘS— (wherein m is 1 to 6, p is 2-6 and each R is independently H, C(1-6)alkyl or substituted C(1-6)alkyl), and combinations thereof, e.g., connected via linking functional groups such as amide (e.g., —CONH— or —CONR— where R is C1-C6alkyl), sulfonamide, carbamate, carbonyl (—CO—), ether, thioether, ester, thioester, amino (—NH—) and the like. The linking component can be peptidic, e.g., a linker including a sequence of amino acid residues. The linking component can be a linker of formula -(L¹)ₐ-(L²)_{b}-(L³)_{c}-(L⁴)_{d}-(L⁵)ₑ-, where L¹ to L⁵ are each independently a linker unit, and a, b, c, d and e are each independently 0 or 1, wherein the sum of a, b, c, d and e is 1 to 5. Other linkers are also possible, as shown in the multimeric compounds described herein.

The linking component can include a terminal-modified PEG linker that is connected to the D-peptidic compounds using any convenient linking chemistry. PEG is polyethylene glycol. The term “terminal-modified PEG” refers to polyethylene glycol of any convenient length where one or both of the terminals are modified to include a chemoselective functional group suitable for conjugation, e.g., to another linking group moiety or to the terminal or sidechain of a peptidic compound. The Examples section describes use of several exemplary terminal-modified PEG bifunctional linkers having terminal maleimide functional groups for conjugating chemoselectively to a thiol group, such as a cysteine residue installed in the sequence of a D-peptidic domain. The D-peptidic compounds can be modified at the N- and/or C-terminals of the GA domain motifs to include one or more additional amino acid residues that can provide for a particular linkage or linking chemistry to connect to a multivalent linking group group, such as a cysteine or a lysine.

Chemoselective reactive functional groups that may be utilized in linking the subject peptidic compounds via a linking group, include, but are not limited to: an amino group (e.g., a N-terminal amino or a lysine sidechain group), an azido group, an alkynyl group, a phosphine group, a thiol (e.g., a cysteine residue), a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

Any convenient multivalent linker may be utilized in the subject multimers. By multivalent is meant that the linker includes two or more terminal or sidechain groups suitable for attachment to components of the subject compounds, e.g., peptidic domains, as described herein. In some cases, the multivalent linker is bivalent or trivalent. In some instances, the multivalent linker is a dendrimer scaffold. Any convenient dendrimer scaffold may be adapted for use in the subject multimers. The dendrimer scaffold is a branched molecule that includes at least one branching point and two or more terminals suitable for connecting to the N-terminal or C-terminal of a domain via optional linkers. The dendrimer scaffold may be selected to provide a desired spatial arrangement of two or more domains. In some cases, the spatial arrangement of the two or more domains is selected to provide for a desired binding affinity and avidity for the VEGF target protein.

In some cases, the multivalent linker group is derived from/includes a chemoselective reactive functional group that is capable of conjugating to a compatible function group on a second peptidic domain. In certain cases, the multivalent linker group is a specific binding moiety (e.g., biotin or a peptide tag) that is capable of specifically binding to a multivalent binding moiety (e.g., a streptavidin or an antibody). In certain cases, the multivalent linker group is a specific binding moiety that is capable of forming a homodimer or a heterodimer directly with a second specific binding moiety of a second compound. As such, in some cases, where the compound includes a molecule of interest that includes a multivalent linker group, the compound may be part of a multimer. Alternatively, the compound may be a monomer that is capable of being multimerized either directly with one or more other compounds, or indirectly via binding to a multivalent binding moiety.

### Exemplary Multivalent D-Peptidic Compounds

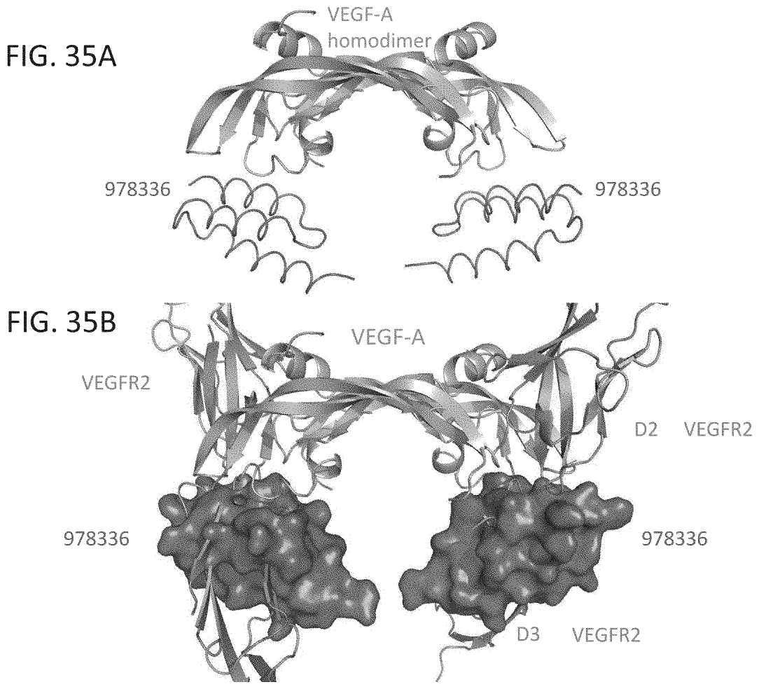

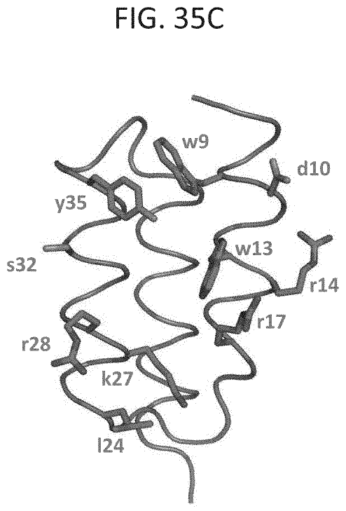

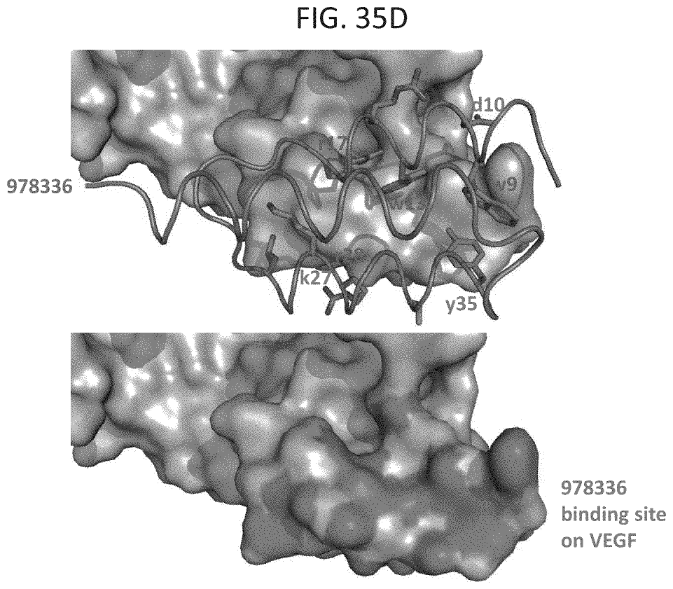

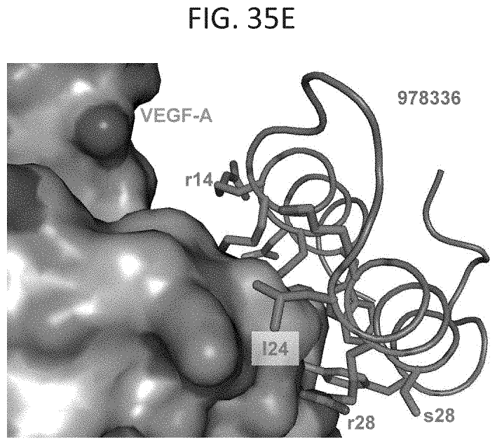

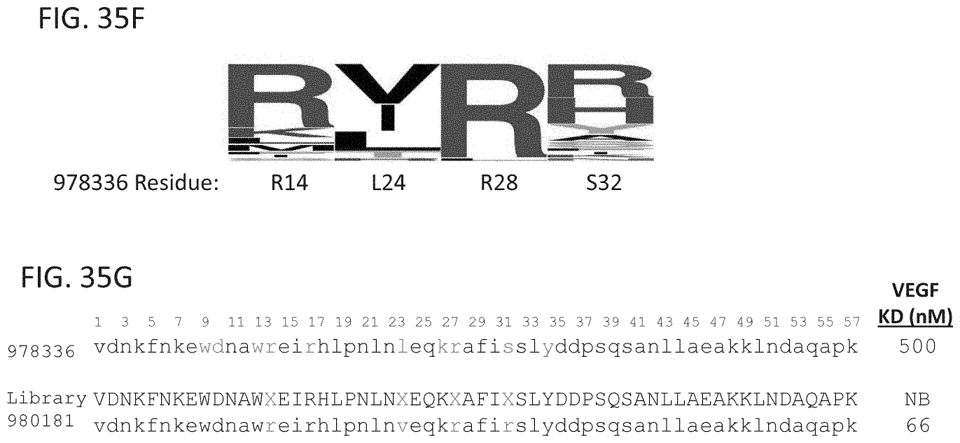

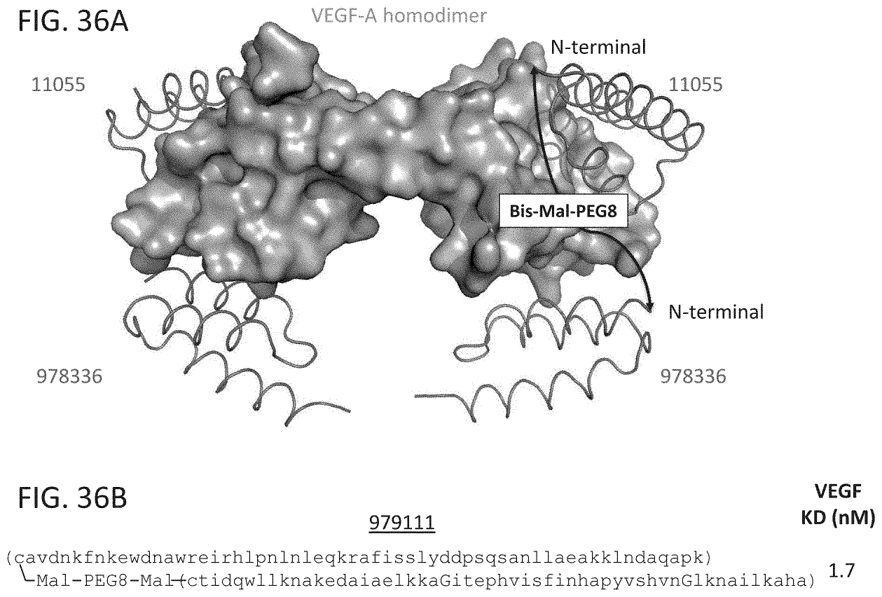

This disclosure provides multivalent compounds that bind VEGF-A. The multivalent VEGF-A binding compound can be bivalent and include two distinct variant domains connected via a linking component (e.g., as described herein). Exemplary single D-peptidic domains that specifically bind VEGF-A are disclosed herein that bind to one of two different binding sites on the target protein. FIG. 36A shows the crystal structures of two such single domains simulataneously bound to target VEGF-A. VEGF-A specific variant GA domain polypeptides are described herein that bind at a first binding site of VEGF-A. In some cases, the first binding site is defined by the amino acid sidechains F43, M44, Y47, Y51, N88, D89, L92, I72, K74, M107, I109, Q115 and I117 of VEGF-A. In some cases, VEGF-A specific polypeptide is a locked variant GA domain (e.g., as described herein). Any of the subject VEGF-A specific D-peptidic variant GA domain polypeptides can be connected via a linking component to a second D-peptidic domain that specifically binds to a second and distinct binding site of the target VEGF-A. In some case, the second binding site is defined by the amino acid sidechains E90, F62, D67, 169, E70, K110, P111, H112 and Q113 of VEGF-A. See FIG. 36A showing exemplary Z domain polypeptide binding at a site distinct from the exemplary GA domain polypeptide, compound 11055. At least one or both of the target binding sites should partially overlap the VEGFR2 binding site on the VEGF-A target protein in order to provide antagonist activity. See e.g., FIG. 35B.

D-peptidic variant GA domain polypeptides which can be linked to a D-peptidic variant Z domain polypeptide in order to provide a VEGF-A binding bivalent compound include, but are not limited to, compounds 11055, 979102 and 979107-979110, and variants thereof (e.g., as described herein).

D-peptidic variant Z domain polypeptides which can be linked to a D-peptidic variant GA domain polypeptide in order to provide a VEGF-A binding bivalent compound include, but are not limited to, compounds 978333 to 978337,980181, 980174-980180, and 981188-981190, and variants thereof (e.g., as described herein).

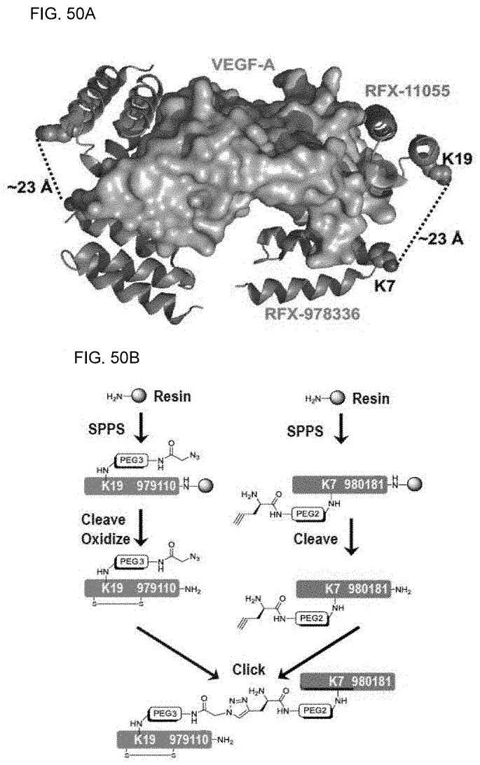

In FIG. 36A a schematic of one possible linking component is shown connecting the N-terminals of the two D-peptidic domains. In some cases, the N-terminal to N-terminal linker is a (PEG)n bifunctional linker, wherein n is 2-20, such as 4-20 or 8-20 (e.g., n is 5, 6, 7, 8, 9, 10, 11 or 12). Any convenient chemoselective functional groups may be incorporated in the the D-peptidic domains being linked in order to provide for conjugation. The interdomain linkages can be achieved post peptide synthesis using compatible chemoselective functional groups (e.g., as described herein). Linking components can also be incorporated into the D-peptidic polypeptide of the subject multivalent compounds during solid phase peptide synthesis (SPPS). See e.g., FIG. 50B.

In some cases, the N-terminal to N-terminal linker can be installed by extending the polypeptide sequence of the domains to incorporate a cysteine residues that provide for conjugation to a maleimide comprising homobifunctional PEG linker. For example, both compounds 11055 and 978336 were chemically synthesized with additional N-terminal cysteine residues, which were conjugated with a bis-maleimide PEG8 linker using conventional methods to provide for an N-terminal to N-terminal linkage (FIG. 36A). For example, Table 5 provides details of an exemplary bivalent compound that binds VEGF-A with high affinity, compound 979111. FIG. 50A shows a view of the crytal structures of D-peptidic domains 11055 and 978336 bound to VEGF-A, and a location for an alternative interdomain linker, i.e. from k19 of variant GA domain to k7 of variant Z domain, that could be utilized to prepare a bivalent compound from a variety of variant GA domain and Z domain polypeptides that bind VEGF-A.

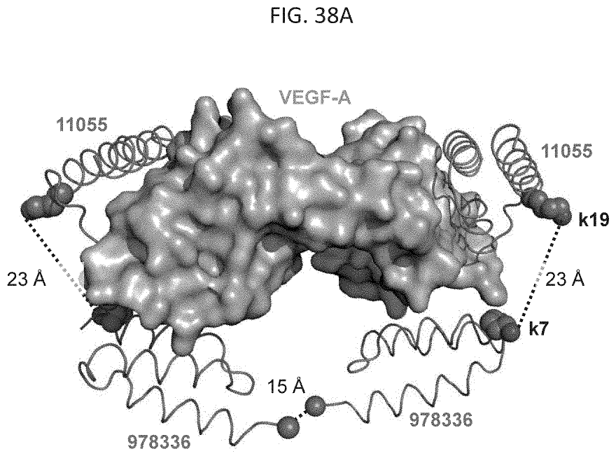

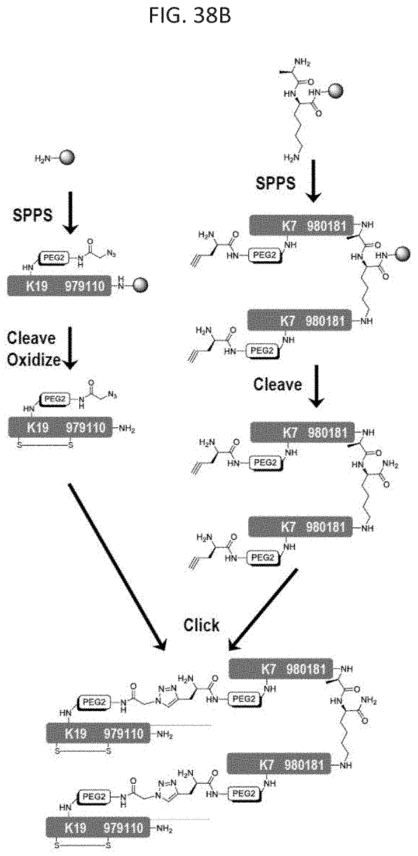

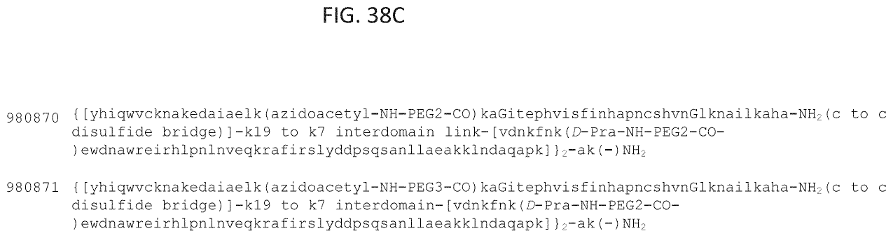

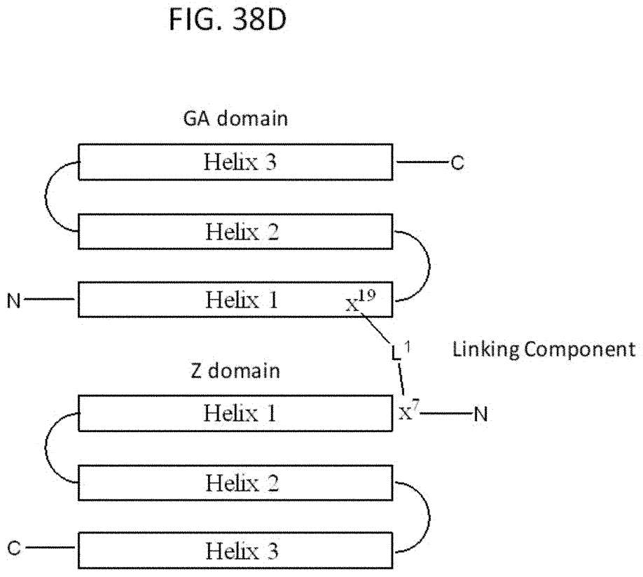

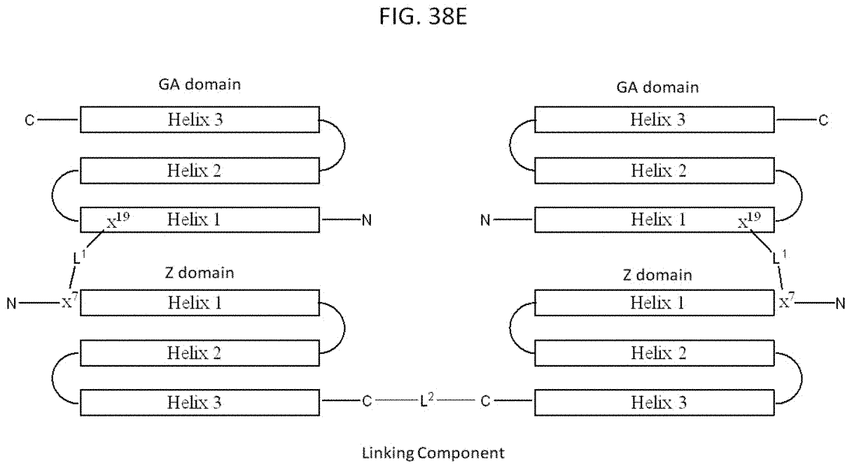

FIG. 38D shows a general structure an exemplary bivalent compound including a linker L¹ between residue x¹⁹ of the GA domain and residue x⁷ of the Z domain. Any exemplary D-peptidic GA domain (e.g., as described herein) and D-peptidic Z domain (e.g., as described herein) can be configured with a linking component L¹ as shown in FIG. 38D. In some embodiments, x¹⁹ and x⁷ residues are each independently lysine and ornithine, and the linker has one of the following structures:

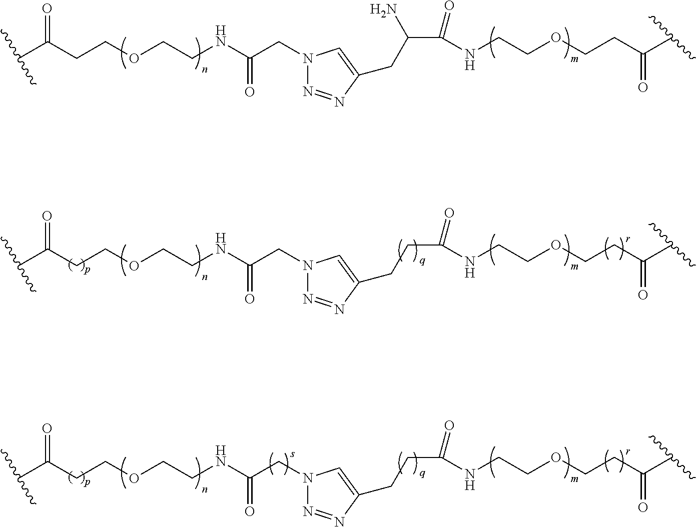

where n and m are independently 1-12, such as 1-6; and p, q and r are each independently 0-3, such as 0 or 1; and s is 1-6, such as 1-3. In some cases of L¹, n+m is 2-6, such as 3, 4 or 5. In some cases of L¹, n and m are each 2. In some cases of L¹, n and m are each 3. In some cases of L¹, p, q and r are each 1. In some cases of L¹, p is 0. In some cases of L¹, q is 0. In some cases of L¹, r is 0. In some cases of L¹, s is 2. In some cases of L¹, s is 3.

FIG. 38E is a schematic diagram of an exemplary dimeric bivalent compound including a second linker L² between the C-terminal residues of the GA and Z domains. FIG.38B shows an exemplary linker L² that was used to link the C-terminal residues of the Z domains of 2 bivalent compounds, and that was capable of being installed during SPPS. The C-terminal to C-terminal linker can include one or more amino acid residues, and one or more linking units (e.g., as described herein). including at least one residue that provides for branching (e.g., lysine), and coupling of amino acids, e.g., to amino sidechain and alpha-amino groups. The C-terminal to C-terminal linker can include one or more amino acid residues, and one or more linking units (e.g., as described herein). In some cases, one or more residues can be installed at the C-terminal of the domain during SPPS that provide for covalent linking whereby the protein domains are capable of simultaneously binding to the VEGF target.

### Exemplary Multimeric Multivalent D-Peptidic Compounds

Aspects of this disclosure include multimeric (e.g., dimeric, trimeric or tetrameric, etc) D-peptidic compounds that include any two or more of the subject variant domain polypeptides and/or bivalent compounds described herein. A multimer of the present disclosure can refer to a compound having two or more homologous domains or two or more homologous bivalent compounds. As such, a dimer of a bivalent compound can include two molecules of any one of the bivalent compounds described herein, connected via a linking component. The target molecule VEGF-A can be a homodimer, and a homologous dimeric compound can provide for binding to analogous sites on each VEGF-A target monomer. For example, FIG. 36A shows an overlay of the crystal structures of two molecules of domain 11055 and two molecules of domain 978336 bound to VEGF-A dimer. Exemplary sites for incorporating chemical linkages to connect the four domains is indicated. Exemplary linking components are elaborated in FIGS. 38B and 38C. In some cases, dimerization of the bivalent compound (11055+978336) is achieved using a peptidic linker between the C-terminals of the domains. For example, Table 5 and FIG. 38C show the sequences and configuration of exemplary VEGF-A binding dimeric bivalent compounds 980870 and 980871, which demonstrates any convenient linking groups may be linked to the C-terminal of a polypeptide domain to introduce a dimerizing linking component, either during SPPS (see FIG. 38B) or post SPPS (e.g., as described herein).

### Peptidic Domains

Any convenient peptidic domains can be utilized in the subject compounds. A variety of small protein domains are utilized in phage display screening that can be adapted for use in methods of mirror image screening against target proteins as described herein. A small peptidic domain of interest can consist of a single chain polypeptide sequence of 25 to 80 amino acid residues, such as 30 to 70 residues, 40 to 70 residues, 40 to 60 residues, 45 to 60 residues, 50 to 60 residues, or 52 to 58 residues. The peptidic domain can have a molecular weight (MW) of 1 to 20 kilodaltons (kDa), such as 2 to 15 kDa, 2 to 10 kDa, 2 to 8 kDa, 3 to 8 kDa or 4 to 6 kDa.

The peptidic domain can be a three helix bundle domain. A three helix bundle domain has a structure consisting of two parallel helices and one anti-parallel helix joined by loop regions. Three helix bundle domains of interest include, but are not limited to, GA domains, Z domains and albumin-binding domains (ABD) domains.

Based on the present disclosure, it is understood that several of the amino acid residues of the peptidic domain motif which are not located at the target binding surface of the structure can be modified without having a significant detrimental effect on three dimensional structure or the target binding activity of the resulting modified compound. As such, several amino acids modifications/mutations can be incorporated into the subject compounds as needed in order to impart a desirable property on the compound, including but not limited to, increased water solubility, ease of chemical synthesis, cost of synthesis, conjugation site, interhelix linkage site, stability, isoelectric point (pI), aggregation resistance and/or reduced non-specific binding. The positions of the mutations may be selected so as to avoid or minimize any disruption to the specificity determining motif (SDM) or the underlying three dimensional structure of the target binding domain motif that provides for specific binding to the target protein. For example, mutation of solvent exposed positions on the opposite side of the domain structure from the binding surface can be made to introduce desirable variant amino acid residues, e.g., to increase solubility or provide a desirable protein pI, or incorporate a conjugation or linkage site. In some cases, based on the three dimensional structure of the target binding domain motif, the positions of mutations can be selected to provide for increased stability (e.g., via introduction of variant amino acid(s) into the core packing residues of the structure) or increased binding affinity (e.g., via introduction of variant amino acid(s) in the SDM). In some instances, the compound includes two or more, such as 3 or more, 4 or more, 5 or more, 6 or more, 7 or more, 8 or more, 9 or more, or 10 or more surface mutations at positions that are not part of the binding surface to the VEGF target protein.

### VEGF-Binding Z Domain

This disclosure provides D-peptidic Z domains that specifically bind VEGF. The Z domain can include a VEGF specificity-determining motif (SDM) defined by 5 or more variant amino acid residues (e.g., 5, 6, 7, 8, 9 or 10 variant amino acid residues) located at positions 9, 10, 13, 14, 17, 24, 27, 28, 32 and/or 35 of a Z domain. It is understood that a variety of underlying Z domain scaffolds or peptidic framework sequences can be utilized to provide the characteristic three dimensional structure of the Z domain.

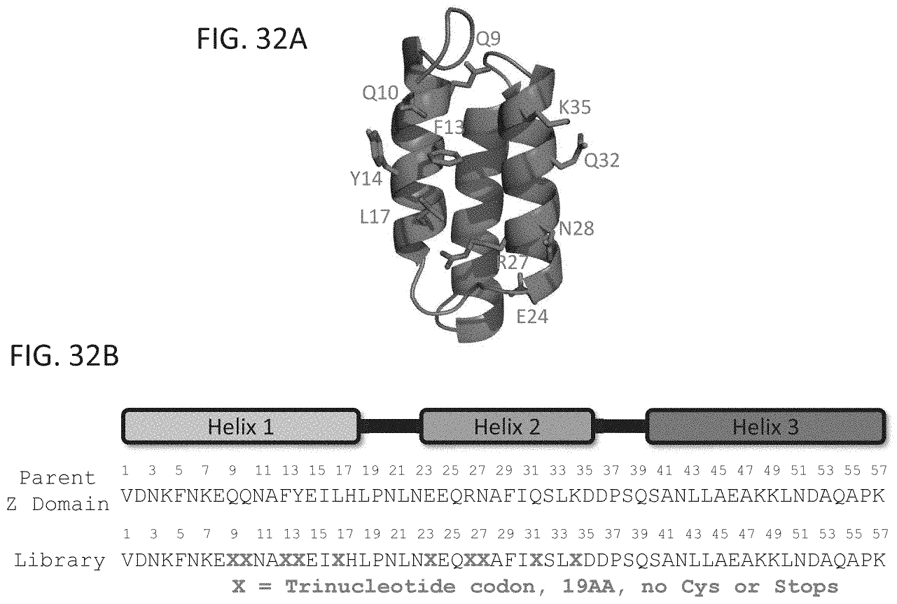

The term “Z domain” refers to a peptidic domain having a three-helix bundle tertiary structure that is related to the immunoglobulin G binding domain of protein A. In the Protein Data Bank (PDB), structure 2spz provides an exemplary Z domain structure. See also, FIG. 32A and FIG. 32B which include depictions of a native Z domain structure and one exemplary sequence of an unmodified native Z domain. The term “Z domain scaffold” refers to an underlying Z domain sequence which provides a characteristic 3-helix bundle structure and can be adapted for use in the subject compounds. A “variant Z domain” is a Z domain including variant amino acids at select positions of the three-helix bundle tertiary structure that provide for specific binding to a target protein. A Z domain motif can be generally described by the formula:

[Helix 3]-[Linker 1]-[Helix 2]-[Linker 2]-[Helix 1]

wherein [Linker 1] and [Linker 2] are independently peptidic linking sequences of between 1 and 10 residues and [Helix 1], [Helix 2] and [Helix 3] are as described above for the GA domain.

Z domains of interest include, but are not limited to, those described by Nygren (“Alternative binding proteins: Affibody binding proteins developed from a small three-helix bundle scaffold”, FEBS Journal 275 (2008) 2668-2676), US20160200772, U.S. Pat. No. 9,469,670 and a 33-residue minimized Z-domain of protein A described by Tjhung et al. (Front. Microbiol., 28 Apr. 2015), the disclosures of which are herein incorporated by reference in their entirety.

For purposes of describing some exemplary VEGF-A specific Z domains of this disclosure, a numbered 57 residue scaffold sequence of FIG. 36B is utilized. In some embodiments, the D-peptidic Z domain is a three-helix bundle of the structural formula: [Helix 1^{(#8-18)}]-[Linker 1^{(#19-24)}]-[Helix 2^{(#25-36)}]-[Linker 2^{(#37-40)}]-[Helix 3^{(#41-54)}] wherein: # denotes reference positions of amino acid residues comprised in the D-peptidic GA domain. It is understood that the helixes 1-3 can be defined to include one or more additional residues extended at a terminal of the helix, and that residue located at such a terminal can have a partial helical configuration, and/or be at the beginning of a turn or loop region. In some cases, Helix 1 of the Z domain can further include one or more additional amino acid residues at the N-terminal, e.g., helical residues at position 7, and optionally position 6. In some cases, Helix 1 of the Z domain can further include an amino acid residue at position 7. In some cases, the Z domain includes residues N-terminal to position 8 that can provide for desirable properties such as, Helix 1 stabilization, stabilization of the three helix bundle, additional VEGF binding contacts, Helix 1 extension, and linking to a second domain or moiety of interest (e.g., as described herein). In some cases, the Z domain includes residues C-terminal to position 54 that can provide for desirable properties such as, Helix 3 stabilization, stabilization of the three helix bundle, additional VEGF binding contacts, Helix 3 extension, and linking to a second domain or moiety of interest (e.g., as described herein).

D-peptidic Z domain compounds can specifically bind VEGF-A at a binding site defined by the amino acid sidechains E90, F62, D67, I69, E70, K110, P111, H112 and Q113 of VEGF.

Exemplary VEGF-A binding D-peptidic Z domains include those described in Table 4 and by the sequences of compounds 978333 to 978337 and 980181 (SEQ ID NOs: 114-119), 980174-980180 and 981188-981190 (SEQ ID NOs: 120-129). In view of the structures and sequence variants described in the present disclosure, it is understood that a number of amino acid substitutions may be made to the sequences of the exemplary compounds while retaining specific binding to VEGF-A. By selecting positions of the variant Z domain where variability is tolerated without adversely affecting the three dimensional architecture of the Z domain, a number of amino acid substitutions may be incorporated.

As such, this disclosure includes a sequence of 978333 to 978337 and 980181 (SEQ ID NOs: 114-119), 980174-980180, and 981188-981190 (SEQ ID NOs: 120-129) having 1-10 amino acid substitutions (e.g., 1-8, 1-6 or 1-5 substitutions, such as 1, 2, 3, 4 or 5 amino acid substitutions). The 1-10 amino acid substitutions can be substitutions for amino acids based on physical properties of the amino acid sidechains, e.g., according to Table 6. Sometimes, an amino acid of a sequence of 978333 to 978337 and 980181 (SEQ ID NOs: 114-119), 980174-980180 and 981188-981190 (SEQ ID NOs: 120-129) is substituted with a similar amino acid according to Table 6. In some cases, the substitution is for a conservative amino acid substitution or a highly conservative amino acid substitution according to Table 6.

This disclosure includes VEGF-A binding D-peptidic Z domains described by a sequence having 80% or more sequence identity with a sequence of 978333 to 978337 and 980181 (SEQ ID NOs: 114-119), 980174-980180, and 981188-981190 (SEQ ID NOs: 120-129) such as 85% or more, 87% or more, 89% or more, 91% or more, 93% or more, 94% or more, 96% or more, 98% or more sequence identity.

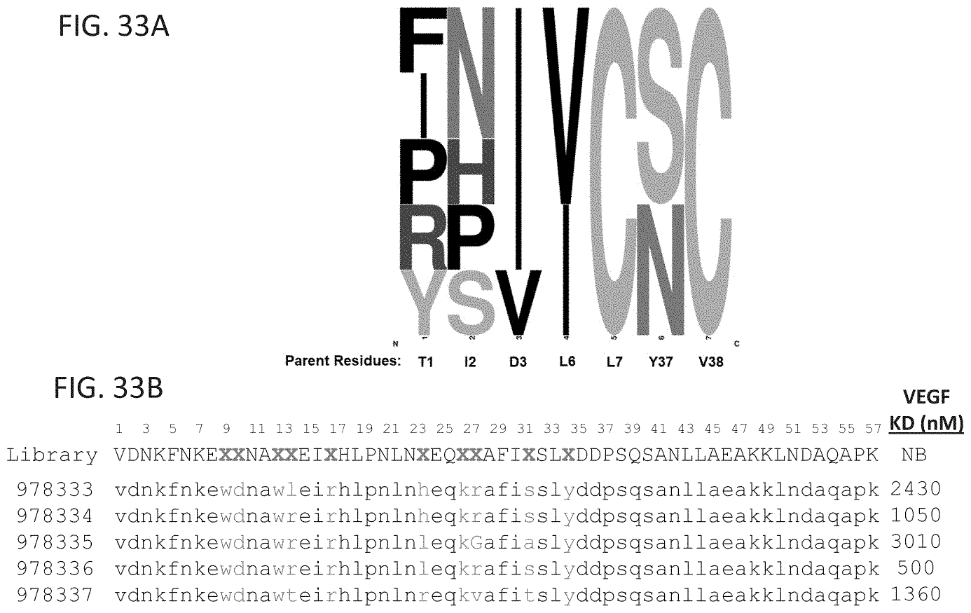

The VEGF-A binding D-peptidic Z domains can have amino acid residues at positions 9, 10, 13, 14, 17, 24, 27, 28, 32 and 35 of a Z domain scaffold that are defined by the specificity-determining motif (SDM) depicted in FIG. 33A and/or FIG. 35F. In some cases, the specificity-determining motif (SDM) is defined by the following sequence motif:

(SEQ ID NO: 160)
w⁹d¹⁰--w¹³x¹⁴--r¹⁷------x²⁴--k²⁷x²⁸---x³²--y³⁵

wherein: x¹⁴, x²⁴, x²⁸ and x³² are each independently any amino acid residue. In certain cases of the SDM: x¹⁴ is selected from l, r and t; x²⁴ is selected from h, i. l, r and v; x²⁸ is selected from G, r and v; and x³² is selected from a, r, h, s and t. In certain cases, the specificity-determining motifs (SDM) is:

(SEQ ID NO: 161)
w⁹d¹⁰--w¹³r¹⁴--r¹⁷------l²⁴--k²⁷r²⁸---s³²--y³⁵;
or
(SEQ ID NO: 162)
w⁹d¹⁰--w¹³r¹⁴--r¹⁷------v²⁴--k²⁷r²⁸---r³²--y³⁵.

In some embodiments, the D-peptidic compound that specifically binds VEGF comprises a D-peptidic Z domain comprising a VEGF specificity-determining motif (SDM) defined by the following amino acid residues:

(SEQ ID NO: 160)
w⁹d¹⁰--w¹³x¹⁴--r¹⁷------x²⁴--k²⁷x²⁸---x³²--y³⁵

wherein:
x¹⁴ is selected from l, r and t;
x²⁴ is selected from h, i, l, r and v;
x²⁸ is selected from G, r and v;
x³² is selected from a, r, h, s and t; and
x³⁵ is selected from k or y.

In some embodiments of the VEGF SDM, x¹⁴ is 1. In some embodiments of the VEGF SDM, x¹⁴ is r. In some embodiments of the VEGF SDM, x¹⁴ is t.

In some embodiments of the VEGF SDM, x²⁴ is h. In some embodiments of the VEGF SDM, x²⁴ is i. In some embodiments of the VEGF SDM, x²⁴ is 1. In some embodiments of the VEGF SDM, x²⁴ is r. In some embodiments of the VEGF SDM, x²⁴ is v.

In some embodiments of the VEGF SDM, x²⁸ is G. In some embodiments of the VEGF SDM, x²⁸ is r. In some embodiments of the VEGF SDM, x²⁸ is v.

In some embodiments of the VEGF SDM, x³² is a. In some embodiments of the VEGF SDM, x³² is r. In some embodiments of the VEGF SDM, x³² is h. In some embodiments of the VEGF SDM, x³² is s. In some embodiments of the VEGF SDM, x³² is t.

In some embodiments of the VEGF SDM, x³⁵ is k. In some embodiments of the VEGF SDM, x³⁵ is y.

In some embodiments, the VEGF SDM is defined by the following residues:

(SEQ ID NO: 161)
w⁹d¹⁰--w¹³r¹⁴--r¹⁷------l²⁴--k²⁷r²⁸---s³²--y³⁵
or
(SEQ ID NO: 162)
w⁹d¹⁰--w¹³r¹⁴--r¹⁷------v²⁴--k²⁷r²⁸---r³²--y³⁵.

In some embodiments of the GA domain, the SDM residues are comprised in a peptidic framework sequence comprising peptidic framework residues defined by the following amino acid residues: --n¹¹a--e¹⁵i-h¹⁸lpnln-e²⁵q--a²⁹fi-s³³l-.

In some embodiments, the GA domain comprises a SDM-containing sequence having 80% or more (e.g., 85% or more, 90% or more, or 95% or more) identity to the amino acid sequence:

(SEQ ID NO: 133)
w⁹d¹⁰naw¹³x¹⁴eir¹⁷hlpnlnx²⁴eqk²⁷x²⁸afix³²sly³⁵

wherein:

x¹⁴ is selected from l, r and t;

x²⁴ is selected from h, i, l, r and v;

x²⁸ is selected from G, r and v;

x³² is selected from a, r, h, s and t; and

x³⁵ is selected from k or y.

In some embodiments of the SDM-containing sequence, x¹⁴ is 1. In some embodiments of the SDM-containing sequence, x¹⁴ is r. In some embodiments of the SDM-containing sequence, x¹⁴ is t.

In some embodiments of the SDM-containing sequence, x²⁴ is h. In some embodiments of the SDM-containing sequence, x²⁴ is i. In some embodiments of the SDM-containing sequence, x²⁴ is 1. In some embodiments of the SDM-containing sequence, x²⁴ is r. In some embodiments of the SDM-containing sequence, x²⁴ is v.

In some embodiments of the SDM-containing sequence, x²⁸ is G. In some embodiments of the SDM-containing sequence, x²⁸ is r. In some embodiments of the SDM-containing sequence, x²⁸ is v.

In some embodiments of the SDM-containing sequence, x³² is a. In some embodiments of the SDM-containing sequence, x³² is r. In some embodiments of the SDM-containing sequence, x³² is h. In some embodiments of the SDM-containing sequence, x³² is s. In some embodiments of the SDM-containing sequence, x³² is t. p In some embodiments of the SDM-containing sequence, x³⁵ is k. In some embodiments of the SDM-containing sequence, x³⁵ is y.

In some embodiments of the compound, Helix 3^{(#41-54)} of the Z domain comprises a peptidic framework sequence s⁴¹ anllaeakklnda⁵⁴ (SEQ ID NO: 134).

In some embodiments the D-peptidic Z domain comprises a C-terminal peptidic framework sequence: d³⁶dpsqsanllaeakklndaqapl⁵⁸ (SEQ ID NO: 135).

In some embodiments the D-peptidic Z domain comprises a N-terminal peptidic framework sequence: v¹dnkfnke⁸ (SEQ ID NO: 136).

VEGF-binding GA domain

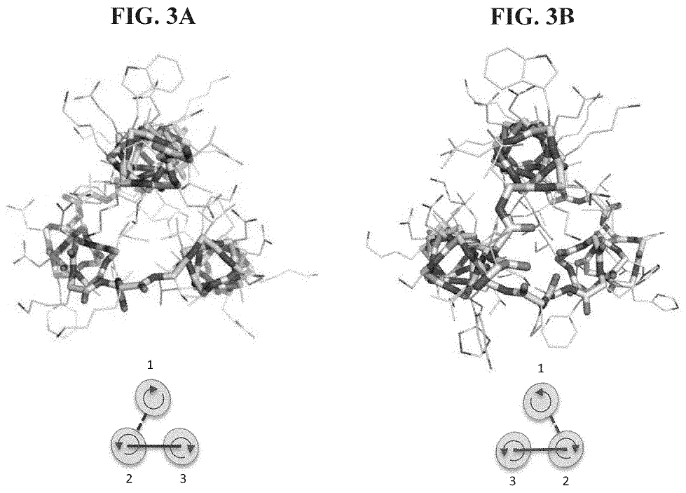

The term “GA domain” and “GA domain motif” refer to a peptidic domain having a three-helix bundle tertiary structure that is related to the albumin binding domain of protein G. In the Protein Data Bank (PDB) structure 1tf0 provides an exemplary GA domain structure. FIGS. 3, 7A-7B, 10A and FIG. 10B include depictions of a native GA domain structure and one exemplary sequence of an unmodified native GA domain. The term “GA domain scaffold” refers to an underlying peptidic framework sequence which provides a characteristic 3-helix bundle structure and can be adapted for use in the subject compounds. In some cases, the GA domain scaffold or peptidic framework sequence has a consensus sequence as defined in Table 3. Table 3 provides a list of exemplary GA domain scaffold sequences which can be adapted for use in the subject compounds. The terms “variant GA domain”, “VEGF-binding GA domain” and “GA domain that binds VEGF” are used interchangeably and refer to a GA domain that includes variant amino acids at select positions of the three-helix bundle tertiary structure which together provide for specific binding to the VEGF target protein.

A GA domain can be described by the structural formula:

[Helix 1]-[Linker 1]-[Helix 2]-[Linker 2]-[Helix 3]

where [Helix 1], [Helix 2] and [Helix 3] are helical regions of a characteristic three-helix bundle linked via peptidic linkers [Linker 1] and [Linker 2]. In the three-helix bundle, [Helix 1], [Helix 2] and [Helix 3] are linked peptidic regions wherein [Helix 2] is configured substantially anti-parallel to a two-helix complex of parallel alpha helices [Helix 1] and [Helix 3]. [Linker 1] and [Linker 3] can each independently include a sequence of 1 to 10 amino acid residues. In some cases, [Linker 1] is longer in length than [Linker 3]. The GA domain can be a peptidic sequence of between 30 and 90 residues, such as between 30 and 80 residues, between 40 and 70 residues, between 45 and 60 residues, between 45 and 60 residues, or between 45 and 55 residues. In certain instances, the GA domain motif is a peptidic sequence of between 35 and 55 residues, such as between 40 and 55 residues, or between 45 and 55 residues. In certain embodiments, the GA domain motif is a peptidic sequence of 45, 46, 47, 48, 49, 50, 51, 52 or 53 residues.

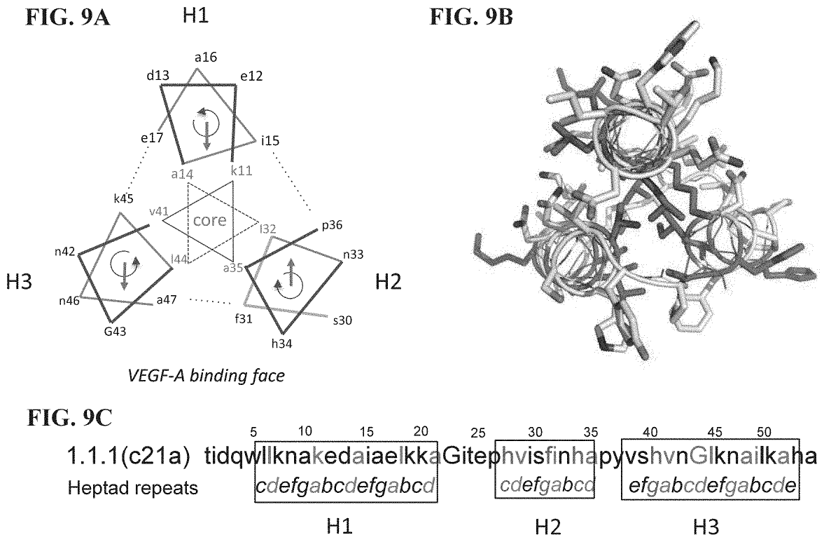

In some embodiments, the D-peptidic GA domain is a three-helix bundle of the structural formula:

[Helix 1^{(#6-21)}]-[Linker 1^{(#22-26)}]-[Helix 2^{(#27-35)}]-[Linker 2^{(#36-37)}]-[Helix 3^{(#38-51)}]

wherein: # denotes reference positions of amino acid residues comprised in the D-peptidic GA domain, e.g., according to the numbering scheme shown in FIG. 9C.

GA domains of interest include those described by Jonsson et al. (Engineering of a femtomolar affinity binding protein to human serum albumin, Protein Engineering, Design & Selection, 21(8), 2008, 515-527), the disclosure of which is herein incorporated by reference in its entirety, and which includes a GA domain and phage display library having a scaffold sequence (G148-GA3) with library mutations at positions 25, 27, 31, 34, 36, 37, 39, 40, 43, 44 and 47 of the scaffold. Other GA domains of interest include but are not limited to those described in U.S. Pat. Nos. 6,534,628 and 6,740,734, the disclosures of which are herein incorporated by reference in their entirety.

The variant GA domains of this disclosure can have a specificity-determining motif (SDM) that includes 5 or more variant amino acid residues at positions selected from 25, 27, 30, 31, 34, 36, 37, 39, 40 and 42-48. In some instances, the specificity-determining motif (SDM) further includes a variant amino acid at position 28 of a GA domain.

### Locked GA Domain

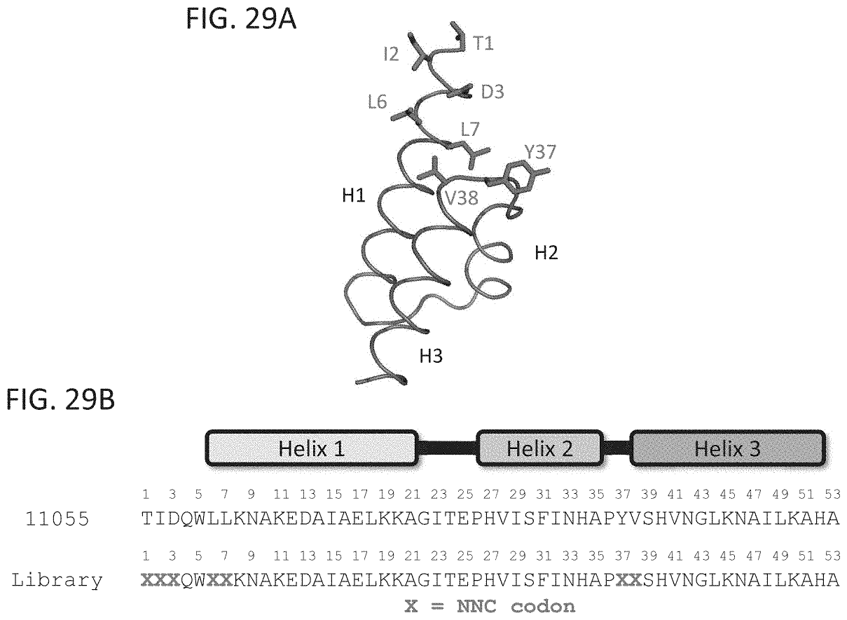

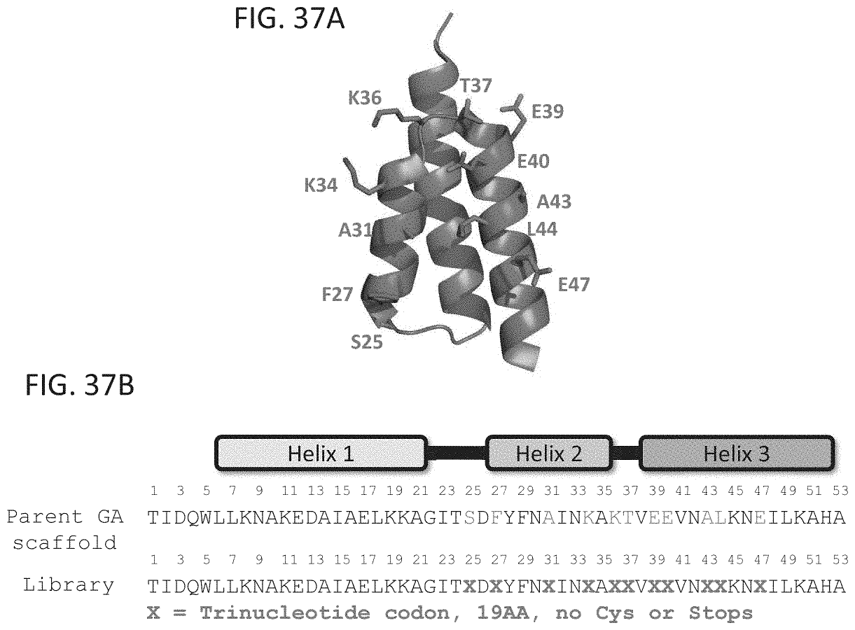

This disclosure includes variant GA domain compounds having an interhelix linker or bridge between adjacent residues of helix 1 and helix 3. The term “locked variant GA domain” and “locked GA domain” refers to a variant GA domain that includes a structure stabilizing linker between any two helices of GA domain. Sometimes, the linked adjacent residues are located at the ends of the helices 1 and 3. FIGS. 29A and 37A show structures of a GA scaffold domain that illustrates the configuration of helices 1-3 in the three-helix bundle. The interhelix linker can be located between amino acid residues at positions 7 (helix 1) and 38 (helix 3) of the domain which are proximal to each other in the three dimensional structure of the domain. Positions 7 and 38 can be considered to be core facing residues located at the ends of helices that are capable of making stabilizing contacts with the hydrophobic core of the structure. The interhelix linker can have a backbone of 3 to 7 atoms in length as measured between the alpha-carbons of the linked amino acid residues. For example a disulfide linkage between two cysteine residues provides a backbone of 4 atoms in length (—CH₂—S—S—CH₂—) between the alpha-carbons of the two cysteine amino acid residues.

A variety of compatible natural and non-naturally occurring amino acid residues can be incorporated at positions 7 and 38 of a GA domain and which are able to be conjugated to each other to provide for the interhelix linker. Compatible residues include, but are not limited to, aspartate or glutamate linked to serine or cysteine via ester or thioester linkage, aspartate or glutamate linked to ornithine or lysine via an amide linkage. As such, the interhelix linker can include one or more groups selected from C₍₁₋₆₎alkyl, substituted C₍₁₋₆₎alkyl, —(CHR)ₙ—CONH—(CHR)ₘ—, and —(CHR)ₙ—S—S—(CHR)ₘ—, wherein each R is independently H, C₍₁₋₆₎alkyl or substituted C₍₁₋₆₎alkyl and n+m=2, 3, 4 or 5. Any convenient non-naturally occurring residues can be utilized to incorporate compatible chemoselective tags at the amino acid residue sidechains of positions 7 and 38, e.g., click chemistry tags such as azide and alkyne tags, which can be conjugated to each other post polypeptide synthesis.

Incorporation of an intradomain linker can provide an increase in stability and/or binding affinity for VEGF target protein. In some cases, the binding affinity (K_{D}) of the D-peptidic compound for VEGF is 3-fold or more stronger (i.e., a 3-fold lower K_{D}) than a control polypeptide lacking the intradomain linker, such as 5-fold or more stronger, 10-fold or more stronger, 30 fold or more stronger, or even stronger. Exemplary locked variant GA domain compounds that specifically bind VEGF-A are described below in greater detail.

A variant GA domain polypeptide can include a N-terminal region from position 1 to about position 6 that can be considered non-overlapping with Helix 2 and Helix 3 because this region is not directly involved in contacts with the adjacent helix 2-loop-helix 3 region of the folded three helix bundle structure (see e.g., FIG. 32A). In the subject D-peptidic compounds, a N-terminal region from positions 1-5 of the GA domain can be optionally retained in the sequence and optimized to provide for a desirable property, such as increased water solubility, stability or affinity. It is understood that the N-terminal region of the variant D-peptidic compounds can be substituted, modified or truncated without significantly adversely affecting the activity of the compound. The N-terminal region can be modified to provide for conjugation or linkage to a molecule of interest (e.g., as described herein), or to another D-peptidic domain or multivalent compound (e.g., as described herein). In some cases, the N-terminal residues have a helical propensity that provides for an extended helical structure of Helix 1. Alternatively, the N-terminal region can incorporate helix capping residues that stabilize the N-terminus of helix 1. In some cases, a variant GA domain compound is truncated at the N-terminus by removal of 1, 2, 3, 4 or 5 residues (i.e., truncation of positions 1-5) relative to a parent GA domain structure as shown in FIG. 32A. In such cases, the numbering scheme of the subject compounds is retained as shown in FIG. 32B. Similarly, one, two or three C-terminal residues at the terminus of helix 3 may be truncated without adversely affecting the stability and target binding capability of the three helix bundle structure.

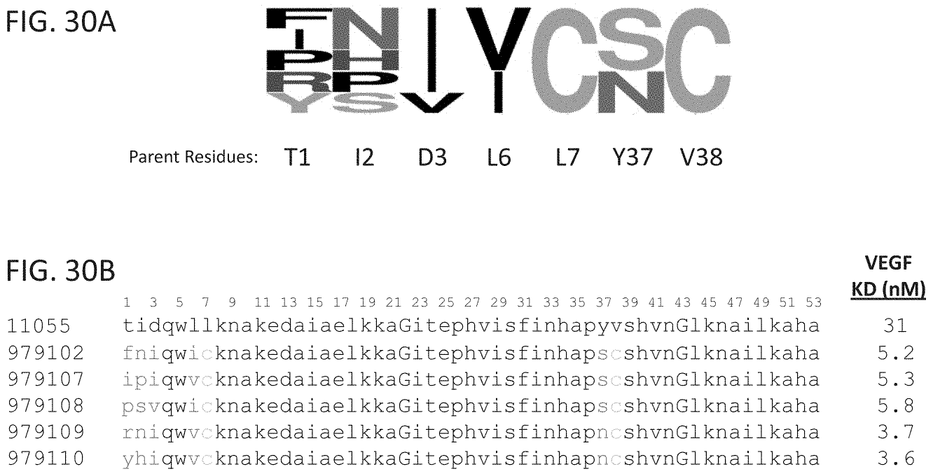

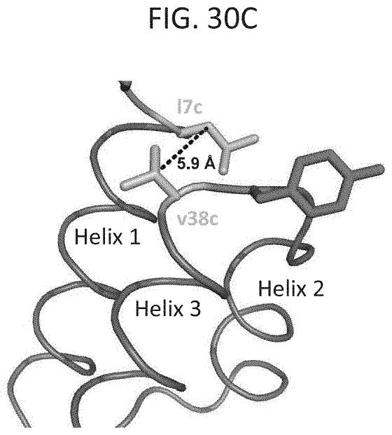

FIG. 29A-29B shows the design of an exemplary affinity maturation library focused at positions 1-3, 6, 7 and 37-38 of a variant GA domain compound. FIG. 30A-30B shows the results of the screening and variant GA domain compounds having a c7-c38 disulfide bridge and an improved binding affinity for VEGF-A. A variety of variant amino acid residues are tolerated at positions 1-3 of the N-terminal region of the compounds.

In some embodiments, a D-peptidic GA domain includes one or more (e.g., both) of the following segments (I)-(II):

(I)
(SEQ ID NO: 142)
x¹x²x³qwx⁶x⁷
(II)
x³⁷x³⁸

wherein:

x¹ to x³ are independently selected from any D-amino acid residue;

x⁶ is selected from i and v;

x³⁷ is selected from s and n; and

x⁷ and x³⁸ are amino acid residues connected via an intradomain/interhelix linker having a backbone of 3 to 7 atoms in length as measured between the alpha-carbons of amino acid residues x⁷ and x³⁸. In some embodiments of formula (I), x¹ to x³ are independently selected from f, h, i, p, r, y, n, s and v. In some embodiments of formula (I), x⁶ is v. In some embodiments of formula (II), X³⁷ is n.

The intradomain/interhelix linker can be composed of a disulfide bridge or linkage between sidechains of the x⁷ and x³⁸ amino acid residues. Any convenient natural or non-naturally occurring thiol containing amino acids can be utilized to provide the intradomain linker Amino acid residues x⁷ and x³⁸ that can be connected via a disulfide linkage include: cysteine⁷-cysteine³⁸ disulfide; homocysteine⁷-cysteine³⁸ disulfide; cysteine⁷-homocysteine³⁸ disulfide; and homocysteine⁷-homocysteine³⁸ disulfide. Alternatively, the intradomain/interhelix linker can include an amide bond linkage between sidechains of the x⁷ and x³⁸ amino acid residues. Any convenient natural or non-naturally occurring amine and carboxylic acid containing amino acids can be utilized to provide the intradomain linker Amino acid residues x⁷ and x³⁸ that can be connected via an amide linkage include: Asp7-Dap38, Asp7-Dab38, Asp7-0rn38, Glu7-Dap38, Glu7-Dap38 and Glu7-0rn38, where Dap is α,β-diaminopropionic acid, Dab is α,γ-diaminobutyric acid and Orn is ornithine. The pairs of x⁷ and x³⁸ residues can be D-amino acid residues. Any convenient chemoselective functional groups and conjugates thereof may be utilized to achieve an intradomain/interhelix linkage, including but not limited to, azide-alkyne, thiol-maleimide, thiol-haloacetyl, thiol-vinyl sulfone, ester, thioester, amide, ether and thioether.

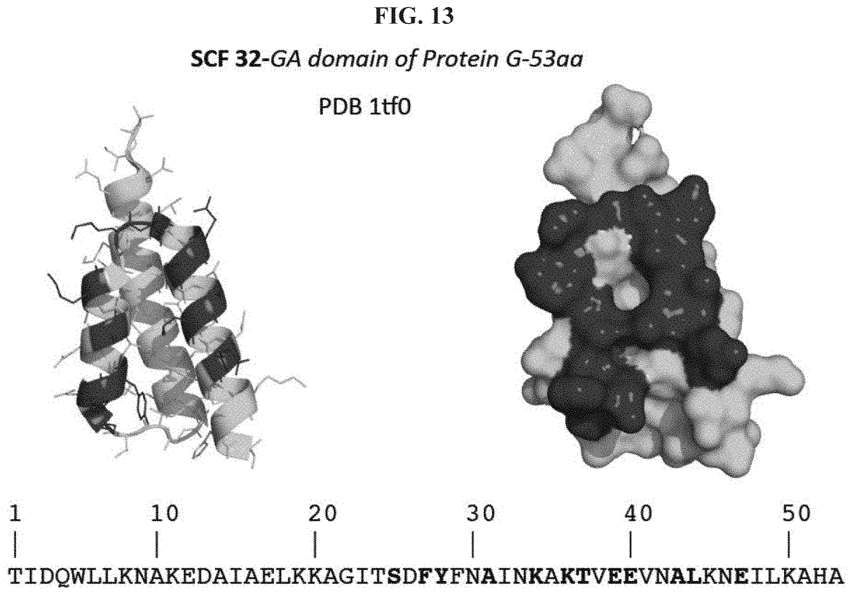

FIG. 13 shows a depiction of a GA domain library including an underlying 53 residue scaffold sequence (SEQ ID NO: 2) and mutation positions in bold at positions 25, 27, 28, 31, 34, 36, 37, 39, 40, 43, 44 and 47 of the scaffold which define one of the phage display libraries used in the screening. Selected hit compounds derived from screening of the scaffold domain libraries were identified. The subject compounds include underlying scaffold domain which presents a VEGF-A binding face that makes contact with the target protein and provides for specific binding to VEGF-A. The selected compounds from the selected GA domain library hits were subjected to additional affinity maturation and point mutation studies (e.g., as described herein) to assess variant amino acids at several additional positions of the GA domain motif e.g., positions 26, 29 and 30. An X-ray crystal structure of an exemplary D-peptidic compound having a GA domain scaffold in complex with VEGF-A is described herein which provides a structural model for the subject VEGF-A binding compounds.

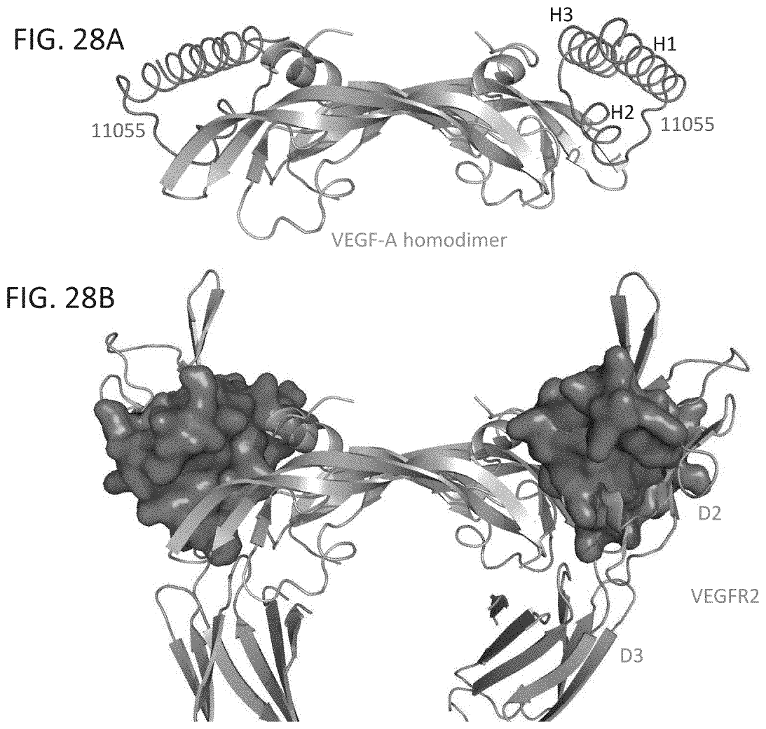

The D-peptidic variant GA domain compound can specifically bind to VEGF-A at a binding site defined by the amino acid sidechains F43, M44, Y47, Y51, N88, D89, L92, I72, K74, M107, 1109, Q115 and I117 of VEGF-A (see FIG. 28A-28B).

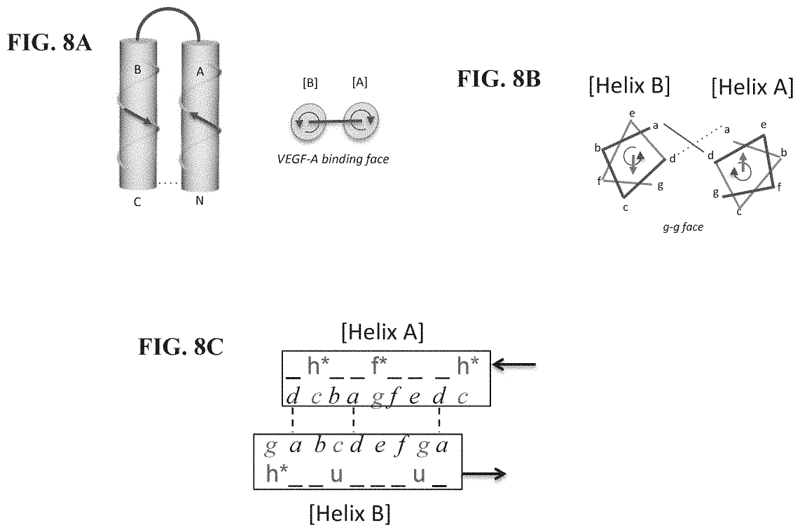

In some cases, a VEGF-A binding motif includes at least two antiparallel helical regions [Helix A] and [Helix B] that are in contact with each other and together define a VEGF-A binding face. That portion of a VEGF-A binding motif that includes the antiparallel complex of [Helix A] and [Helix B] can be referred to as a “two-helix complex” structure. FIG. 8A-8B, depict a heptad repeat structural model for the two-helix complex structure. In some instances, VEGF-A contacting residues of interest can be located at surface mutation or boundary mutation positions of the two helix complex, such as c or g positions of a heptad repeat. FIG. 8C shows one exemplary arrangement of VEGF-A contacting residues on the g-g face of the two-helix complex structure. The VEGF-A binding face can include 4 or more residues, such as 5 or more, 6 or more, 7 or more, 8 or more, 9 or more, or 10 or more VEGF-A contacting residues, where the residues include residues of both of [Helx A] and [Helix B]. In certain cases, the VEGF-A contacting residues are independently selected from non-polar, aromatic, heterocyclic and carbocyclic residues (e.g., as described herein). The two helices of the two-helix complex can be connected via any convenient linkages that preserve the substantially antiparallel configuration of [Helix A] and [Helix B]. In some cases, [Helix A] and [Helix B] are linked via a C (Helix A) to N (Helix B) peptidic linker. In some cases, [Helix A] and [Helix B] are linked via a C (Helix A) to N (Helix B) peptidic linker. FIG. 8A, depicts a possible terminal linkage (solid blue line) for the two-helix complex structure.

The two-helix complex can be further stabilized by any convenient methods, including but not limited to, incorporation of residues that provide for desirable helix-helix packing interactions or hydrophilicity at solvent exposed positions, incorporation of interhelix linkages, incorporation of intrahelix linkages, incorporation of a constrained turns or linker that connects the helices, and linkage to a third peptidic region capable of stabilizing contacts with both [Helix A] and [Helix B]. FIG. 8B-8C, depict various interhelix sidechain to sidechain linkages (e.g., dotted lines) which can be installed between any two convenient residues. Similarly, stabilizing intrahelix sidechain to sidechain or sidechain to terminal linkages can be installed to provide a desired stability to the structure of the compound. Interhelix and intrahelix linkages of interest that find use in the subject compounds include, but are not limited to, Cys-Cys disulfide linkages, stapled peptide linkages, and non-native crosslinks, such as those linkages prepared by ring-closing metathesis and those linkages described by Douse et al. (ACS Chem Biol. 2014 Oct. 17; 9(10):2204-9).

In some embodiments, the two-helix complex can be stabilized by a third helix (Helix C) which contacts both [Helix A] and [Helix B] at the opposite side of the VEGF-A binding face of the compound and which together define a three-helix bundle. As used herein, the terms “three-helix bundle” and “three-helix bundle motif” are used interchangeably to refer to a three-helix bundle that is a small protein tertiary structure including three substantially parallel or antiparallel alpha helices. The three helices are based on a linear sequence of linked helical regions arranged in a parallel-antiparallel-parallel configuration in the three-helix bundle structure.

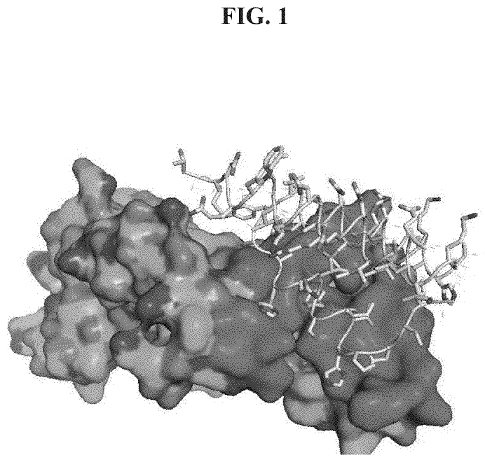

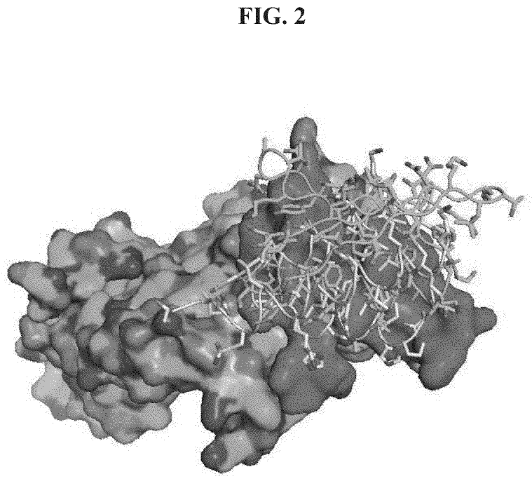

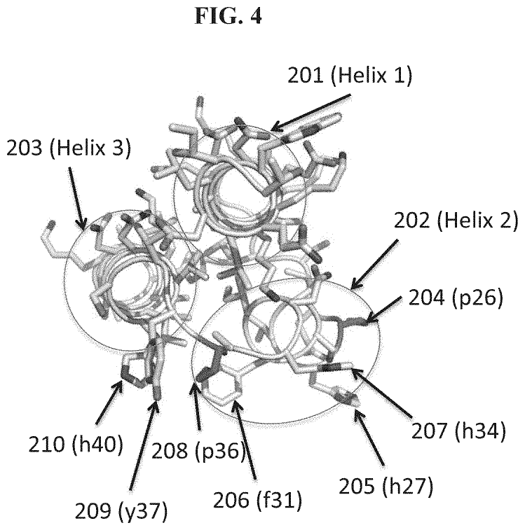

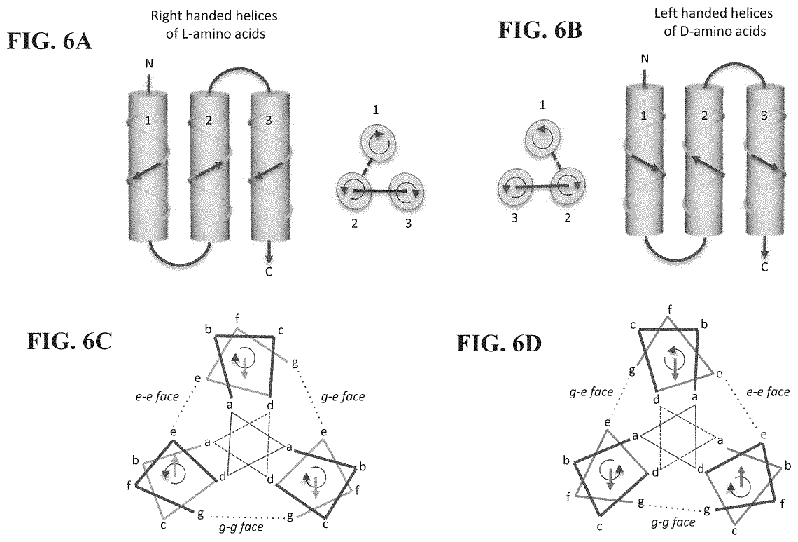

DeGrado et al. (Analysis and design of three-stranded coiled coils and three-helix bundles”, Folding & Design 1998, 3: R29-R40) provides a model for the assembly of three-stranded coiled coils and three-helix bundles, the disclosure of which is herein incorporated by reference in its entirety. Three-helix bundles can be single stranded structures with loops connecting helices that have regular contacts with each other in a non-polar core. The three helices of the structure can show an approximate seven-residue repeat motif, designated by the letters in italics a-g, i.e., (abcdefg)ₙ. The heptad designations a, c, d, e, f and g do not correspond to the single letter codes for particular amino acids, but rather to positions in the heptad sequence. Non-polar residues can occur at positions a and d of the heptad including sidechain groups packing into the center of the structure to provide hydrophobic stabilization. The non-polar a and d residues can pack into layers. In some cases, charged sidechains can occur at the interfacial e and g positions, where the non-polar portions of their sidechains can shield the hydrophobic core and the polar portions can engage in electrostatic or hydrogen bonding interactions. In some cases, solvent exposed positions b and c can be occupied by polar residues. In some instances, position f is highly solvent exposed and can be occupied by polar or charged residues. FIG. 6D shows the D-peptidic heptad repeat model of a three helix bundle showing two parallel helices and one anti-parallel helix. In some cases, the residues at the g-g face formed by the combined surface of helices 2 and 3 are modified to include VEGF-A contacting residues which are configured to interact with the surface of VEGF-A and provide specific binding. It is understood that a two helix complex version of the structural model depicted in FIG. 6D is possible, which is shown in FIG. 8B. Any convenient stabilizing elements can be utilized in the subject compounds (e.g., as described herein) to maintain the desired arrangement of two helices and presentation of VEGF-A binding residues which provides for specific binding to VEGF-A. The subject compounds can have a VEGF-A binding GA domain motif having a three-helix bundle tertiary structure into which variant amino acid residues are incorporated to provide a binding surface capable of specifically binding to VEGF-A. FIGS. 1-2 depict the binding interface between an exemplary peptidic compound and VEGF-A. FIG. 3A and FIG. 3B show a side by side comparison of the three-helix bundle X ray crystal structures of a L-protein GA domain and an exemplary D-peptidic compound. Comparison of FIG. 3A and FIG. 3B indicates that the peptidic compound can retain the basic three-helix bundle structural motif of the parent GA domain. In certain cases, the alpha-helical structure of the compound is substantially the same as the native GA scaffold domain. The modifying variant amino acids can include helix terminating residues at the terminals of the Helix 2 region that are not present in the GA scaffold domain. The variant amino acids of the Helix 2 region can also include three or more VEGF-A contacting residues, e.g., aromatic amino acid residues. FIG. 4 depicts helix-terminating proline residues at positions 26 and 36 (p26; 204 and p36; 208), and VEGF-A contacting phenylalanine at position 31 (f31; 206) and histidine residues at positions 27 and 34 (h27; 205 and h34; 207) of the Helix 2 region of an exemplary VEGF-A binding compound.

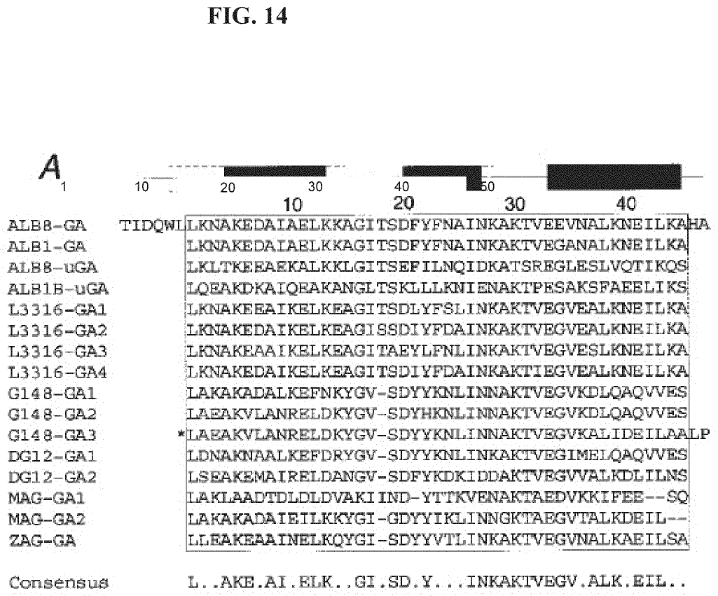

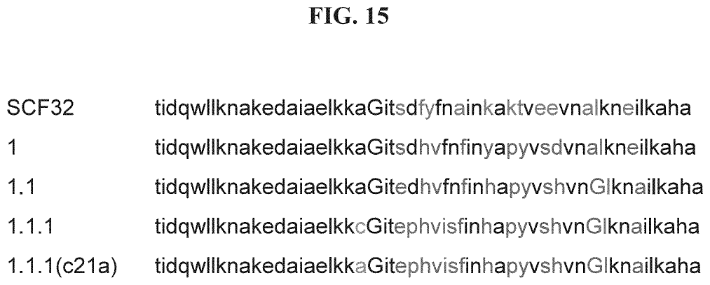

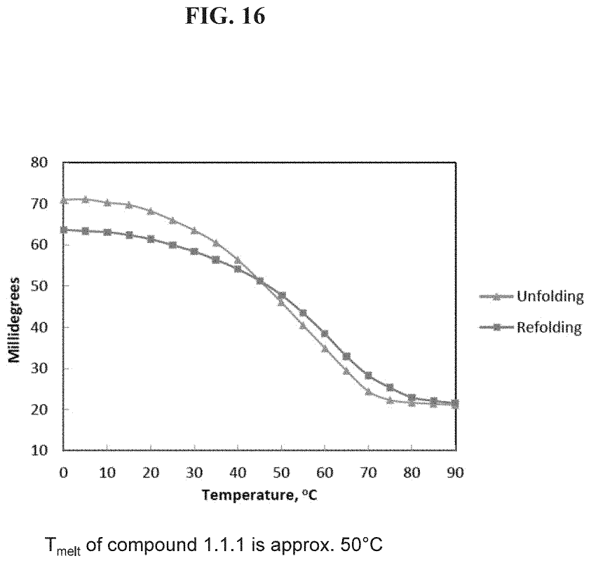

In certain embodiments of the compounds described herein, a numbering scheme is utilized for convenience and simplicity to refer to particular positions in the structure and/or sequence of the compounds, e.g., positions at which particular variant amino acid residues of interest are incorporated into a GA scaffold domain. This numbering scheme is based on that utilized for the 53 residue GA scaffold domain depicted in FIG. 13. It is understood that any convenient alignment methods can be used to compare a particular embodiment of the subject compounds to the reference numbering scheme of FIG. 15 in order to assign a numbered location to an amino acid residue of interest, e.g., a location in a motif or a structural model as described herein. FIG. 14 shows an exemplary alignment of a variety of GA scaffold domain sequences of interest, any one of which could serve as an underlying parent sequence for a subject compound. FIG. 14 also references the sequences to the numbering scheme of FIG. 13. It is further understood that the numbering scheme of 1-53 in FIG. 13 is not meant to be limiting in terms of determining the total number of amino acid residues or length of a linear compound sequence or in terms of defining each and every residue of a particular compound.

In some cases, the subject compounds include one or more variations relative to a numbered parent sequence, such as, a N-terminal truncation (e.g., from position 1), a C-terminal truncation (e.g., from position 53), a deletion (e.g., of a single residue position at any convenient location of the parent sequence), an insertion (e.g., of 1, 2, 3 or more contiguous residues between two particular numbered positions of a parent sequence). In certain cases, such variations which are incorporated into the subject compounds substantially preserve the three dimensional structure of the three-helix bundle that provides for specific binding to the target. The subject compounds can further include variant amino acids at one or more positions of the parent structure or sequence, such as 2 or more, 3 or more, 4 or more, 5 or more, 6 or more, 7 or more, 8 or more, 9 or more, 10 or more, 11 or more, 12 or more, 13 or more, 14 or more or 15 or more positions, e.g., as described in the following embodiments.

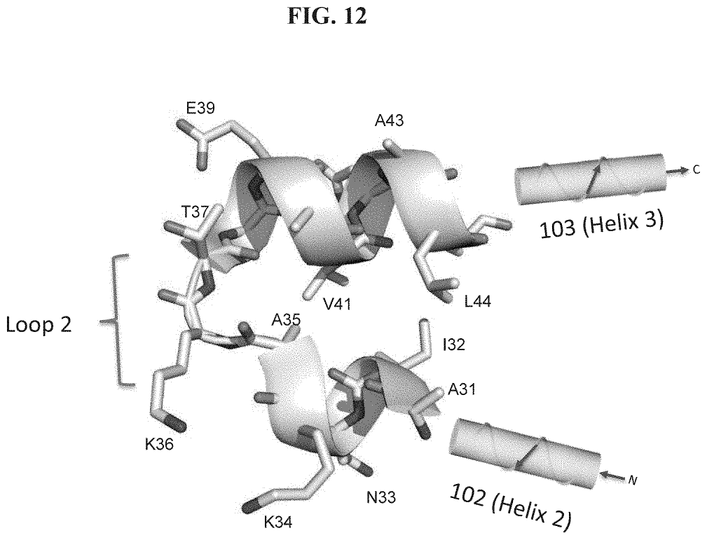

As described herein, the subject compounds can have a three-helix bundle structure where particular solvent exposed variant amino acids located at particular positions of [Helix 2] and [Helix 3] can form contacts with the VEGF-A. In some cases, additional contacts can occur at particular residues of [Linker 2] and/or [Linker 1]. FIG. 1 depicts the binding interface between an exemplary peptidic compound and VEGF-A as taken from an X-ray crystal structure of the complex. In certain cases, variant amino acids located at additional positions of [Helix 2], [Helix 3], [Linker 2] and/or [Linker 1] provide a desirable stabilization of the modified three helix bundle structure. For example, in FIG. 4, exemplary [Helix 2] terminating residues are shown (e.g., proline residues 204 and 208) that can, in some cases, impart a desirable increased stabilization to [Helix 2]. In certain instances, the hydrophobic core of the modified three helix bundle is defined by substantially the same amino acid residues as those of a parent GA scaffold domain. For example, FIG. 11 shows an expanded view of part of the [Helix 2]-[Linker 2]-[Helix 3] structure of an exemplary D-peptidic compound including adjacent hydrophobic residues i32 (isoleucine, position 32) and a35 (alanine, position 35) of [Helix 2] and adjacent hydrophobic residues v41 (valine, position 41) and 144 (leucine, position 44) of [Helix 3] which provide desirable intramolecular hydrophobic contacts. In FIG. 12 is shown an expanded view of a similar region of a native L-peptidic GA domain, where analogous residues 132 (isoleucine, position 32), A35 (alanine, position 35), V41 (valine, position 41) and L44 (leucine, position 44) provide for similar desirable intramolecular hydrophobic contacts that are characteristic of the GA scaffold domain three helix bundle structure.

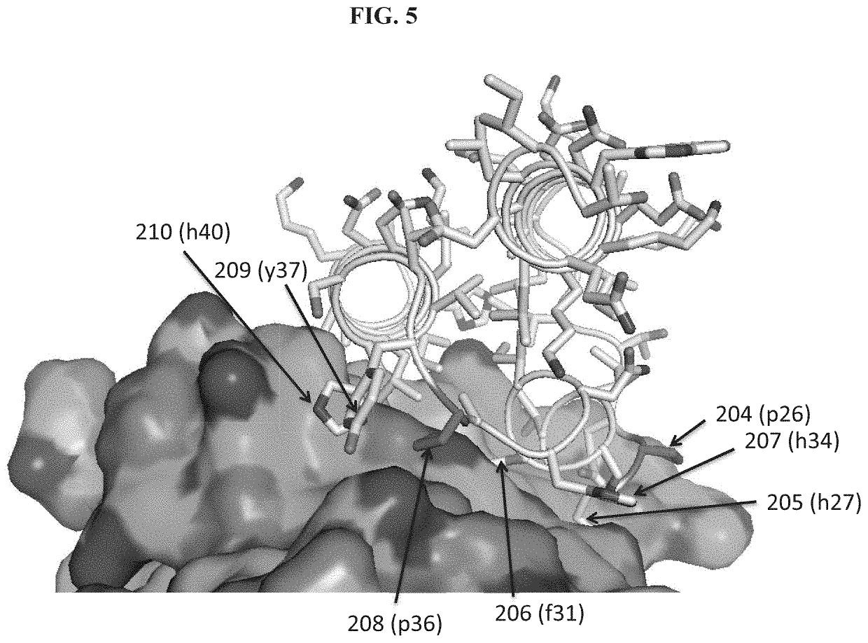

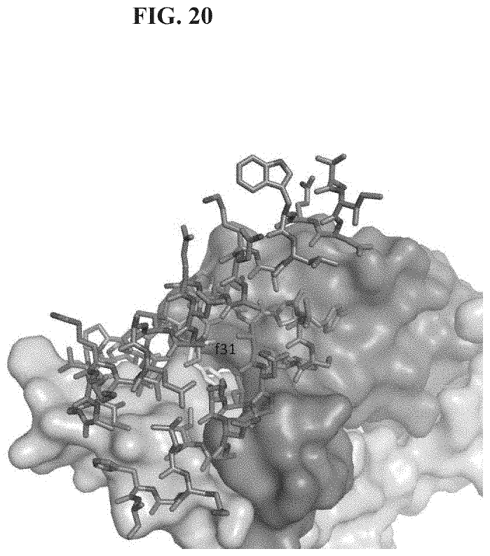

FIG. 6C depicts Degrado's model of an antiparallel three-stranded helix structure. Degrado's model of antiparallel three-stranded helices based on repeating heptad units is adapted herein to provide a structural model that relates the subject compound sequence motifs to the subject compounds' modified three-helix bundle structure including a VEGF-A binding surface. This structural model for the three helix bundle is consistent with the X-ray crystal structures of a native GA domain (e.g., FIG. 3A) and an exemplary VEGF-A binding compound (FIG. 3B). FIGS. 9A and 9C shows the model applied to an exemplary compound 1.1.1(c21a) where the amino acid residues of the compound sequence (FIG. 9C) are associated and structurally aligned with the various positions of the heptad repeat model, consistent with the X-ray structure (FIG. 9B). A comparison of the model in FIG. 9A to the X-ray structure (see e.g., views of FIG. 5 and FIG. 20) of the compound in complex with VEGF-A shows that the VEGF-A binding surface of the exemplary compound is located at the g-g face (FIG. 9 A) that is defined by Helix 2 and Helix 3.

Selected amino acid residues can be located at the VEGF-A binding surface of the subject compounds and configured to interact with VEGF-A (e.g., located at the solvent exposed c and/or g positions of the g-g face defined by Helix 2 and Helix 3).

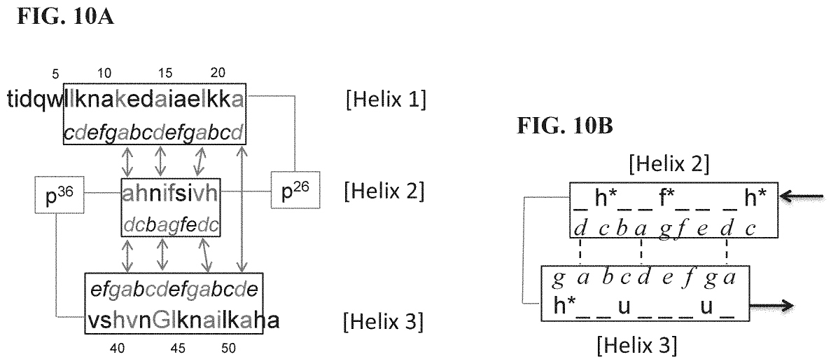

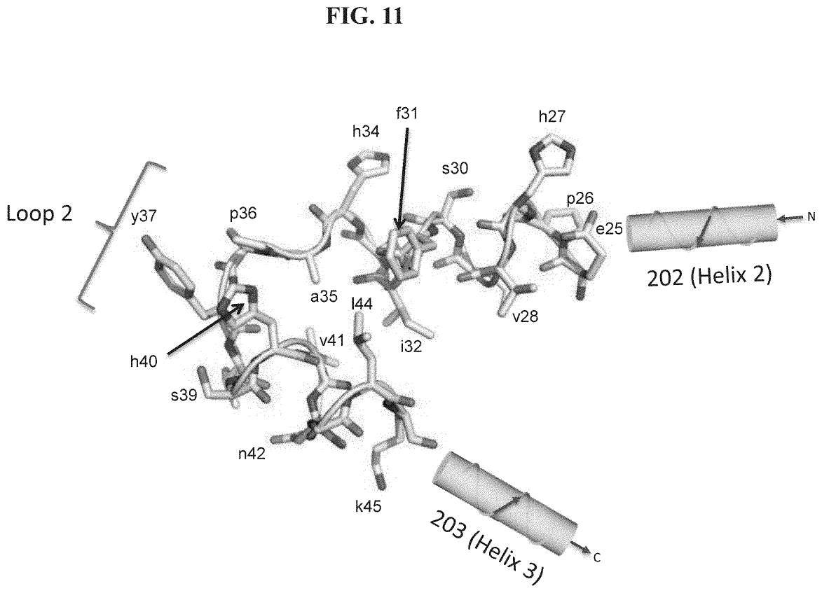

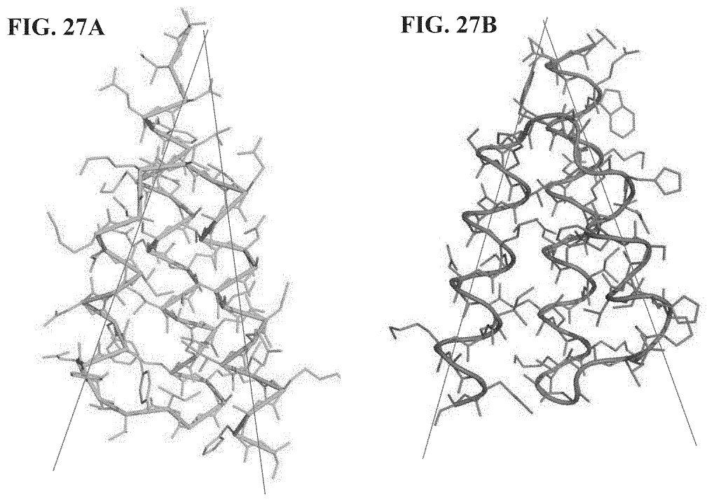

The hydrophobic core of the subject compounds can include a and d residues of [Helix 2] which contact corresponding d and a residues of [Helix 3]. FIG. 6B and FIG. 10A show alignments of exemplary compound 1.1.1 (c21a) with the heptad repeat model where hydrophobic contacts of core residues between the helices of the three-helix bundle are depicted. This is consistent with the partial structure shown in FIG. 11 of the [Helix 2]-[Linker 2]-[Helix 3] region including adjacent hydrophobic residues i32 (isoleucine, position 32) and a35 (alanine, position 35) of [Helix 2] and adjacent hydrophobic residues v41 (valine, position 41) and 144 (leucine, position 44) of [Helix 3] which provide desirable intramolecular hydrophobic contacts. It is understood that the model (e.g., as shown in FIG. 9 A) allows for an alignment of Helix 2 and 3 that is not exactly parallel (i.e., an interhelix angle of >0 degrees, e.g., as described herein and as depicted in FIG. 27).

As depicted in FIGS. 5 and 27, in some cases, although Helix 2 and 3 can have a substantially antiparallel configuration with respect to the direction of the helices and these helices do make several contacts with each other down the length of the helices, the axes of the helices can be aligned with an angle that is >0 degrees, such as about 10 degrees or more, about 15 degrees or more, about 20 degrees or more, about 25 degrees or more, or about 30 degrees or more. As such, in some instances of the subject compounds, Linker 2 is shorter than Linker 1, such that the angle between Helix 2 and 3 is measured from the Linker 2 connection of the helices. In some cases, the “a” and “d” residues that are furtherest away from the Linker 2 end of the helices are more likely to be partially solvent exposed and/or available to make contacts with VEGF-A.

In certain instances, the subject compound includes a helix terminating residue that provides for an increase in the angle between Helix 2 and 3, e.g., an increase of about 5 degrees or more, such as about 10 degrees or more, or about 15 degrees or more. See e.g., FIG. 27B versus FIG. 27A.

In some embodiments, [Helix 2] comprises the heptad repeat sequence [c¹d¹e¹g¹a²b²c²d²] and [Helix 3] comprises the heptad repeat sequence [e¹f¹g¹a²b²c²d²e²f²a³b³c³d³e³], where the individual heptad repeat residues can be numbered. In certain cases of this arrangement of [Helix 2] and [Helix 3], residues d², a² and d′ of [Helix 2] interact with residues a², d² and a³ of [Helix 3] to form a network of structure stabilizing interactions. In certain cases, residues c², g¹ and c¹ of [Helix 2] and residue g¹ of [Helix 3] are each independently an aromatic, heterocyclic or carbocyclic residue which are configured to contact VEGF-A.

The VEGF-A binding surface of the subject compounds can be defined by a configuration of aromatic amino acid residues located at the c and g positions of the heptad repeat model which residues are configured on the surface to interact with VEGF-A. In some cases, the VEGF-A binding surface includes 2 or more, 3 or more aromatic amino acid residues, such as 4 or more, or 5 or more aromatic amino acid residues located at the c and g positions of the heptad repeat sequences. FIG. 8D and FIG. 10B depict embodiments of variant domain motifs comprising a configuration of c and g residues of [Helix 2] and [Helix 3] capable of binding VEGF-A. In certain cases, the VEGF-A binding surface includes additional non-aromatic amino acid residue(s) that are non-polar amino acid residues at the c and g positions of the heptad repeat, e.g., at residues c and/or g of Helix 3 as shown in FIG. 10B. In certain cases, the VEGF-A binding surface includes additional non-aromatic amino acid residue(s) that are polar amino acid residues capable of hydrogen bonding interaction at the c and g positions of the heptad repeat, e.g., at c and/or g residues of Helix 3. Based on the present disclosure, it is understood that several of the amino acid residues of the GA domain motif which are not located at the VEGF-A binding surface of the structure can be modified without having a detrimental effect on the VEGF-A binding activity of the resulting modified compound.

In some embodiments of formula (I), [Helix 2] comprises a sequence of the formula:

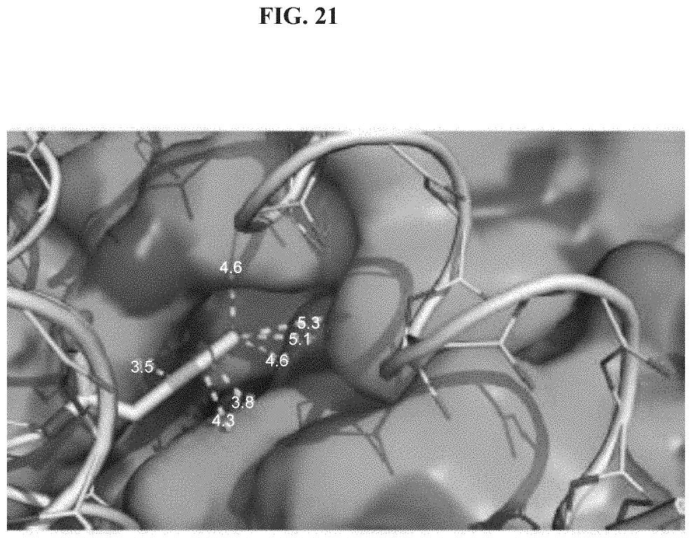

(II)
(SEQ ID NO: 143)
ΛjxxΛjxΛj

wherein: each “A” is independently a D-aromatic amino acid; each j is independently a hydrophobic residue; and each x is independently an amino acid residue. Aromatic amino acids of interest that find use in formula (II) include, but are not limited to, h, f, y and w, and substituted versions thereof. In some instances of formula (II), the first Λ is h, for y. The second A residue can be an aromatic residue comprising an aryl, heteroaryl, substituted aryl or substituted heteroaryl ring (e.g., a reside having a sidechain of formula —CH₂—Ar where Ar is aryl or substituted aryl). In some instances of formula (II), the second Λ is for y, or a substituted version thereof. The second A residue can be configured on the binding surface of the GA domain motif structure to interact with a VEGF-A protein, e.g., to project into the deep pocket on the surface of VEGF-A depicted in FIGS. 20 and 21. In some instances of formula (II), the second Λ is for a substituted version thereof. In some instances of formula (II), the third Λ is an aromatic residue comprising a heteroaryl or substituted heteroaryl ring (e.g., an aromatic residue comprising a sidechain group capable of hydrogen bonding to the VEGF-A). In some instances of formula (II), each j is independently selected from v, i, a and 1. In some instances of formula (II), the first j residue is valine. In some embodiments of formula (II), the [Helix 2] comprises a sequence of the formula: hv xxΛjxΛj.

In some embodiments of formula (I) and (II), [Helix 2] comprises a sequence of formula (III):

(III)
(SEQ ID NO: 151)
h*jxxf*jxh*j

wherein:

each h* is independently histidine or an analog thereof;

f* is phenylalanine or an analog thereof;

each j is independently a hydrophobic residue; and

each x is independently an amino acid residue.

In some embodiments of formula (III), the [Helix 2] comprises a sequence of the formula: hvxxf*jxh*j. The residue f* of formula (III) can be configured on the binding surface of the GA domain motif structure to interact with a VEGF-A protein, e.g., to project into the deep pocket on the surface of VEGF-A depicted in FIG. 21. FIG. 20 shows a wide view of the X-ray structure of the complex where residue f31 (phenylalanine, position 31) in Helix 2 of exemplary compound 1.1.1(c21a) is labelled and shown to project into the pocket on the surface of VEGF-A. FIG. 21 shows an expanded view of f31 which is configured to project into the pocket at the VEGF-A binding interface. Selected distances between atoms of the phenylalanine phenyl ring and adjacent residues of VEGF-A are shown in angstroms. An analysis of the crystal structure indicates that a variety of aromatic residues can be utilized at that location on the three helix bundle structure to project into the same deep pocket that f31 does and, in some cases, to increase desirable hydrophobic contacts with the VEGF-A pocket. In certain cases, the phenylalanine analog includes a substituent(s) on the phenyl ring. In some instances of formula (III), f* is phenylalanine In some instances of formula (III), f* is a substituted derivative of phenylalanine Phenylalanine derivatives of interest include, but are not limited to, 4-halogen substituted phenylalanine (e.g., 4-chloro, or 4-fluoro), 3-halogen substituted phenylalanine (e.g., chloro, bromo or fluoro), 3,5-halogen disubstituted phenylalanine (e.g., chloro or fluoro), 3,4-halogen disubstituted phenylalanine (e.g., chloro or fluoro), 4-methyl substituted phenylalanine, 4-trifluoromethyl-phenyl alanine and 4-ethyl substituted phenylalanine A variety of compounds including phenylalanine analogs at position 31 were prepared and shown to be active.

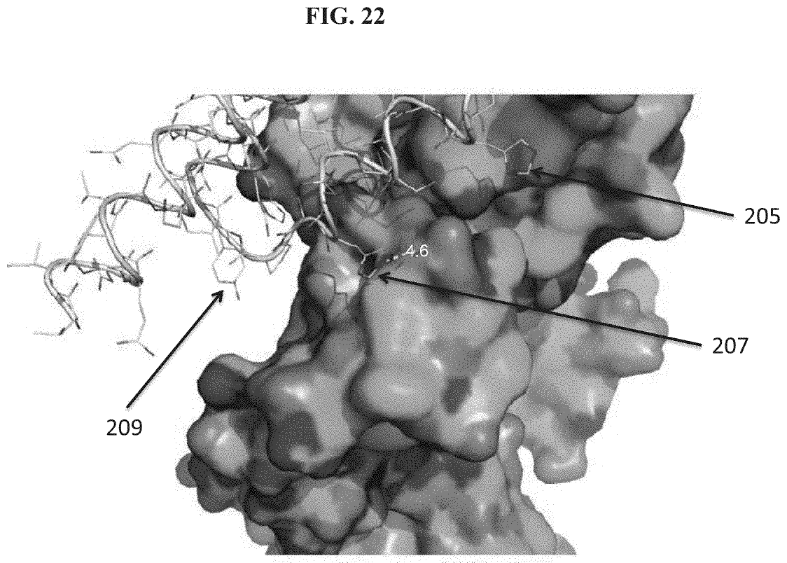

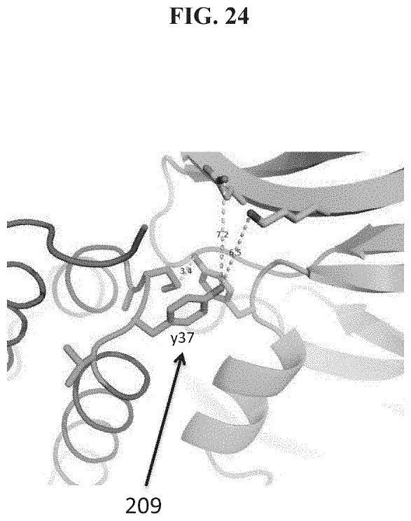

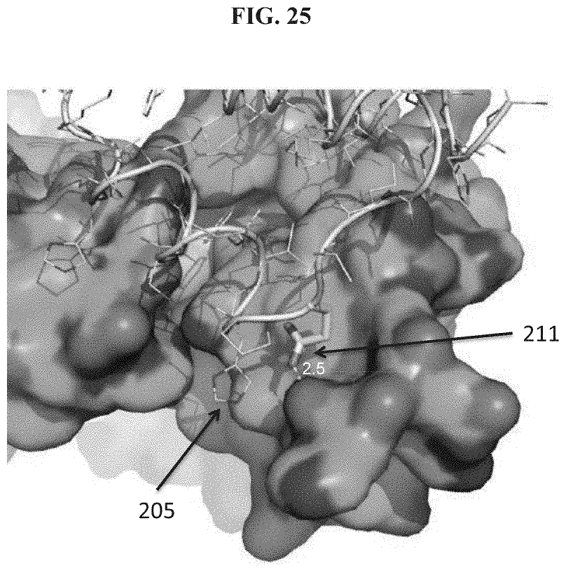

FIG. 22 and FIG. 25 show expanded views of residue h27 (205) of exemplary compound 1.1.1(c21a) in contact with the VEGF-A surface. An analysis of the crystal structure indicates that a variety of aromatic residues or histidine analogs can be utilized at location 27 on the three helix bundle structure to make contact with the same surface pocket that h27 does and in some cases to increase desirable contacts with the VEGF-A surface. In some instances of formula (III), the first h* is histidine, e.g., the residue at position 27. In some instances of formula (III), the first and/or second h* is a histidine analog (e.g., a residue having a sidechain including an alkyl-cycloalkyl group, such as a -alkyl-cyclopentyl or alkyl-cyclohexyl, or substituted version thereof). In some instances of formula (III), the first h* is an aromatic residue capable of primarily hydrophobic contacts with VEGF. In some instances of formula (III), the first h* is for y.

FIG. 22 shows an expanded view of residue h34 (207) of exemplary compound 1.1.1(c21a) in contact with the VEGF-A surface. Analysis of the complex structure indicates various histidine analogs are tolerated at position 34, e.g., an analog including a substituted or unsubstituted aryl or heterocyclic ring that can occupy the available space on the surface of VEGF-A and/or make a stronger hydrogen bond (e.g., of <4.6 angstrom in length) to adjacent residues of VEGF-A . In some instances of formula (III), the second h* is histidine, e.g., the residue at position 34. In some cases of formula (III), the second h* is an aromatic residue capable of hydrogen bonding with VEGF. In some embodiments of formula (III), the second h* is an aromatic residue comprising a heteroaryl or substituted heteroaryl ring (e.g., an aromatic residue comprising a sidechain group capable of hydrogen bonding to the VEGF-A).

In certain embodiments of formulae (II) and (III), h*²⁷, P³¹ and h*³⁴ are each variant residues. In certain embodiments of formulae (II) and (III), j²⁸ and x²⁹ are each variant residues. In certain embodiments of formulae (II) and (III), j^{28,} x²⁹ and x³⁰ are each variant residues. In some instances of formulae (II) and (III), each j is independently selected from a, i, l and v. In some instances of formula (II) and (III), the first j residue is valine. In some cases, the heptad repeat register of formulae (II) and (III) is b′a′gfedcba.

In some embodiments of formula (III), [Helix 2] is described by the following helical motif from positions 26 to 36 of the three-helix bundle:

(IV)
(SEQ ID NO: 144)
z²⁶h*jxxf*jxh*jz³⁶

wherein: each h*, f*, each j and each x are as defined above; and z²⁶ and z³⁶ are each independently a helix-terminating residue. It is understood that, in some cases, the helix-terminating residues are not considered to be helical residues of the structure but merely define the termination of the [Helix 2] region and the beginning of a turn or loop structure. The residue and each h* residue can be configured on the binding surface of the GA domain motif structure to make specific contact with a target VEGF-A protein, e.g., as described herein. In some embodiments of formula (IV), the [Helix 2] comprises a sequence of the formula:

(SEQ ID NO: 145)
z²⁶hvxxf*jxh*jp³⁶.

The term “helix-terminating residue” refers to an amino acid residue that has a high free energy penalty for forming a helix structure relative to an analogous alanine residue. In some cases, a high free energy helix penalty is referred to as a helix propensity value and is 0.5 kcal/mol or greater as defined by the method of Pace and Scholtz where higher values indicate increased penalty (“A Helix Propensity Scale Based on Experimental Studies of Peptides and Proteins”, Biophysical Journal Volume 75 July 1998 422-427). In some cases, a helix-terminating residue is a naturally occurring residue that has a helix propensity value of 0.5 or more (kcal/mol), such as 0.55 or more, 0.60 or more, 0.65 or more or 0.70 or more. For example, proline has a helix propensity value of 3.16 kcal/mol and glycine has a helix propensity value of 1.00 kcal/mol, as shown in Table 1. The helix propensity values of non-naturally occurring helix-terminating residues may be estimated by using the value of the closest naturally occurring residue having a sidechain group that is a structural analog. In some instances of formula (IV), the helix-terminating residues z²⁶ and z³⁶ are independently selected from from d, n, G and p. In some instances of formula (IV), the helix-terminating residues are independently selected from d, G and p. In some instances of formula (IV), the helix-terminating residues are independently selected from G and p. In some instances of formula (IV), the helix-terminating residues z²⁶ and z³⁶ are each p. In some instances of formula (IV), z³⁶ is p

TABLE 1
Naturally occurring amino acid alpha-helical propensities

| 3-Letter | 1-Letter | Helix propensity value (kcal/mol)* |
|---|---|---|
| Ala | A | 0 |
| Arg | R | 0.21 |
| Asn | N | 0.65 |
| Asp | D | 0.69 |
| Cys | C | 0.68 |
| Glu | E | 0.40 |
| Gln | Q | 0.39 |
| Gly | G | 1.00 |
| His | H | 0.61 |
| Ile | I | 0.41 |
| Leu | L | 0.21 |
| Lys | K | 0.26 |
| Met | M | 0.24 |
| Phe | F | 0.54 |
| Pro | P | 3.16 |
| Ser | S | 0.50 |
| Thr | T | 0.66 |
| Trp | W | 0.49 |
| Tyr | Y | 0.53 |
| Val | V | 0.61 |

*Estimated differences in free energy, estimated in kcal/mol per residue in an alpha-helical configuration, relative to Alanine arbitrarily set as zero. Higher numbers (more positive free energies) are less favored. In some cases, deviations from these average numbers are possible, depending on the identities of the neighboring residues.

In certain embodiments of formula (IV), z²⁶ is a framework residue, e.g., a residue corresponding to a residue of a scaffold domain motif. In certain cases of formula (IV), z²⁶ is a variant residue, e.g., a residue that differs from the corresponding residue of a scaffold domain motif such as one or more of SEQ ID NOs: 1-21. In certain instances of formula (IV), z³⁶ is a variant residue. In certain embodiments of formula (IV), h*²⁷, f*31 and h*³⁴ are each variant residues. In some embodiments of formula (IV), j²⁸ and x²⁹ are each variant residues. In some instances of formula (IV), j²⁸, x²⁹ and x³⁰ are each variant residues. In certain embodiments of formula (IV), h*²⁷ is selected from h, y and f. In certain embodiments of formula (IV), h*³⁴ is selected from h, y and f.

In some embodiments of the compound, [Helix 2] is defined by a sequence of the formula:

(V)
(SEQ ID NO: 93)
p²⁶hjjxfjxhjp³⁷

wherein: each j is independently a hydrophobic residue; and each x is an amino acid residue. In certain instances, each j is a residue independently selected from a, i, f, l and v. In certain cases, each j is a residue independently selected from a, i, l and v. In certain cases, each j is a residue independently selected from a, i and v. In certain cases of formula (V), j²⁸ is V. In certain instances of formula (V), j²⁹ is a, l or v. In some embodiments of formula (V), j²⁹ is i. In some instances of formula (V), j³² is i. In certain cases of formula (V), j³⁶ is a. In certain instances of formula (V), x³⁰ is a polar residue. In some cases of formula (V), x³³ is a polar residue. In certain embodiments of formula (V), x³⁰ and x³³ are independently selected from d, e, k, n, r, s, t and q. In certain instances of formula (V), x³⁰ and x³³ are independently selected from s and n. In certain cases of formula (V), x³⁰ is s. In some cases of formula (V), x³³ is n. In some embodiments of formula (V), the [Helix 2] comprises a sequence of the formula: p²⁶hvjxfjxhjp³⁷ (SEQ ID NO: 137).

In some embodiments of the compound, [Helix 2] in defined by a sequence of the formula (VI):

(VI)
(SEQ ID NO: 94)
z²⁶hvj²⁹x³⁰fix³³haz³⁷

wherein:

Z²⁶ is selected from d, p and G;

j²⁹ is selected from f and i;

x³⁰ is selected from n and s;

x³³ is selected from n and s; and

z³⁷ is selected from p and G.

In some cases of formula (VI), z²⁶ is p. In some instances of formula (VI), j²⁹ is i. In certain cases of formula (VI), x³⁰ is s. In some embodiments of formula (VI), x³³ is n. In some instances of formula (VI), z³⁷ is p.

In some instances of the compound, [Helix 2] is defined by a sequence selected from:

a) phvj²⁹x³⁰fix³³hap (VII) (SEQ ID NO: 95) wherein: j²⁹ is selected from f and i; and x³⁰ and x³³ are independently a polar amino acid residue; and

b) an amino acid sequence which has 80% or greater identity to the sequence of formula (VII) defined in a), such as 90% or greater identity to the sequence defined in a).

In some instances of the sequence of formula (VII) defined in a), x³⁰ and x³³ are independently selected from n, s, d, e and k. In some instances of the sequence of formula (VII) defined in a), j²⁹ is i. In some instances the sequence of formula (VII) defined in a), x³⁰ is s or n. In some instances the sequence of formula (VII) defined in a), x³³ is n. In some instances the sequence of formula (VII) defined in a), j²⁹ is i; x³⁰ is s or n; and x³³ is n.

In some embodiments of the compound, [Helix 2] has 66% identity or greater to the sequence of SEQ ID NO: 74, such as 77% identity or greater or 88% identity or greater to the sequence of SEQ ID NO: 74.

In some embodiments of formula (I), [Helix 3] comprises a sequence of the formula:

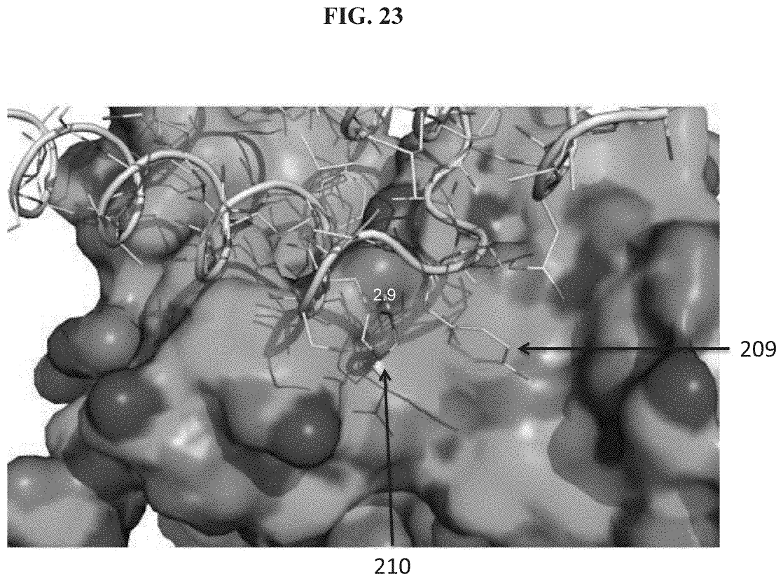

(VIII)
(SEQ ID NO: 146)
Λjxujxxuj

wherein: each “A” is independently an D-aromatic amino acid; each j is independently a hydrophobic residue; each u is independently a non-polar amino acid residue; and each x is independently an amino acid residue. In some cases, the heptad repeat register of formula (VIII) is edcbag′f′e′d′. In some instances of formula (VIII), the Λ is an aromatic residue comprising a heteroaryl or substituted heteroaryl ring (e.g., an aromatic residue comprising a sidechain group capable of hydrogen bonding to the VEGF-A). In certain instances, Λ is histidine or a substituted version thereof. FIG. 23 shows a medium strength hydrogen bond (2.9 angstrom) between a nitrogen atom of h40 (210) of an exemplary compound and adjacent Tyr48 of VEGF-A. Analysis of the complex structure indicates various histidine analogs are tolerated at position 40, including analogs that can occupy the available space and retain or strengthen the hydrogen bond to VEGF-A. In some instances of formula (VIII), each u is independently a non-polar residue having a sidechain selected from H, a lower alkyl and a substituted lower alkyl. In some instances of formula (VIII), each u is independently selected from G and a. In some instances of formula (VIII), the first u is G. In some instances of formula (VIII), the second u is a. In certain instances, each j is a residue independently selected from a, i, f, l and v. In certain cases, each j is a residue independently selected from a, i, 1 and v. In certain embodiments of formula (VIII), j²⁸ is V. In certain embodiments of formula (VIII), j²⁹ is a, l or v.

In some embodiments of formulae (I) or (VIII), [Helix 3] comprises a sequence of the formula (IX):

(IX)
(SEQ ID NO: 96)
x³⁸xh*jxujxxujx⁴⁹

wherein j, x, u are as defined above and h* is histidine or an analog thereof. In some cases, the heptad repeat register of formula (IX) is gfedcbag′f′e′d′c′. In some instances of formula (IX), h* is histidine. In some instances of formula (IX), h* is a histidine analog (e.g., a residue having a sidechain including an alkyl-cycloalkyl group, such as a -alkyl-cyclopentyl or alkyl-cyclohexyl, or substituted version thereof). In some instances of formula (IX), h* is a substituted histidine. In some instances of formula (XI), u⁴³ is G. In some instances of formula (IX), u⁴⁷ is a. In some instances of formula (IX), x³⁸ is v. In some instances of formula (IX), x³⁹ is s. In certain instances of formula (IX), each j is a residue independently selected from a, i, f, l and v. In certain embodiments of formula (IX), iⁿ is v. In some instances of formula (IX), j⁴⁴ is l. In some instances of formula (IX), j⁴⁸ is i. In some instances of formula (IX), x⁵¹ is a hydrophobic residue. In some instances of formula (IX), x⁵¹ is a. In some instances of formula (IX), x⁴² is n. In some instances of formula (IX), x⁴⁵ is k or r. In some instances of formula (IX), x⁴⁵ is k. In some instances of formula (IX), x⁴⁶ is n. In some instances of formula (IX), x⁴⁹ is l. In some instances of formula (IX), Helix 3 is capped with a C-terminal sequence of residues. In certain instances, Helix 3 of formula (IX) includes additional residues x⁵⁰x⁵¹, where x is an amino acid residue. In some cases, x⁵⁰ is k or r. In some instances of formula (IX), x⁵⁰ is k and x⁵¹ is a. In some instances of formula (IX), x⁵⁰ is e and x⁵¹ is d. In some instances of formula (IX), x⁵⁰ is G and x⁵¹ is r. In certain instances, Helix 3 of formula (IX) includes a C-terminal region selected from one of SEQ ID NO: 85-87. In some cases, [Helix 3] includes the heptad repeat register of gfedcbag′f′e′d′c′b′a′. It is understood that a variety of truncations (e.g., truncations of 1, 2 or 3 residues) and extensions (e.g., extensions of 1, 2, 3 or more residues) can be utilized at the C-terminal of [Helix 3] without significantly disrupting the three helix bundle structure or the variant domain, e.g., as depicted in FIG. 9B.

In some instances of formulae (IX), [Helix 3] is defined by a sequence selected from:

a) x₃₈x₃₉hvx⁴²Glx⁴⁵x⁴⁶aix (X) (SEQ ID NO: 97) wherein: x³⁸ is selected from v, e, k, r; ₓ³⁹, x⁴² and x⁴⁶ are independently selected from a polar amino acid residue; and x⁴⁵ and x⁴⁹ are independently selected from l, k, r and e; and

b) an amino acid sequence which has 75% or greater identity to the sequence of formula (X) defined in a), such as 83% identity or greater or 91% identity or greater to the sequence defined in a).

In some instances of formulae (IX), [Helix 3] is defined by a sequence selected from:

a) x³⁸x³⁹hvx⁴²Glx⁴⁵x⁴⁶aix⁴⁹x⁵⁰a (XI) (SEQ ID NO: 98) wherein: x³⁸ is selected from v, e, k, r; ₓ³⁹, x⁴², x⁴⁶ and x⁵⁰ are independently selected from a polar amino acid residue; and x⁴⁵ and x⁴⁹ are independently selected from l, k, r and e; and

b) an amino acid sequence which has 78% or greater identity to the sequence of formula (XI) defined in a), such as 85% identity or greater or 92% identity or greater to the sequence defined in a).

In some instances of formulae (X)-(XI), x³⁹, x⁴², x⁴⁶ and x⁵⁰ are independently selected from n, s, d, e and k. In some instances of formulae (X)-(XI), x³⁸ is V. In some instances of formulae (X)-(XI), x⁴⁵ is k. In some instances of formulae (X)-(XI), x⁴⁹ is l. In some instances of formulae (X)-(XI), x³⁹ is s. In some instances of formulae (X)-(XI), x⁴² is n. In some instances of formulae (X)-(XI), x⁴⁶ is n. In some instances of formula (XI), x⁵⁰ is k.

In some embodiments of the compound, [Helix 3] has 65% identity or greater to the sequence of SEQ ID NO: 79, such as 75% identity or greater, 83% identity or greater or 91% identity or greater to the sequence of SEQ ID NO: 79. In some embodiments of the compound, [Helix 3] has 70% identity or greater to the sequence of SEQ ID NO: 82, such as 78% identity or greater, 85% identity or greater or 92% identity or greater to the sequence of SEQ ID NO: 82.

In formula (I), [Linker 2] is a peptidic linker that connects [Helix 2] and [Helix 3] and which can make optional additional contacts with the surface of VEGF-A. [Linker 2] can be any convenient length. In some cases, [Linker 2] is a shorter linker than [Linker 1]. The N-terminal residue of [Linker 2] that is adjacent to [Helix 2] can be considered to be a helix-terminating residue, e.g., as described herein. In some cases, the C-terminal residue of [Linker 2] that is adjacent to [Helix 3] can be considered to be a helix-terminating residue, e.g., as described herein. In some cases, [Linker 2] can include 4 amino acid residues or less, such as 3 or less or 2 or less. In some instances, [Linker 2] has the same number of residues as the corresponding helices-connecting loop region of a native GA scaffold domain. In certain embodiments of formula (I), [Linker 2] is zx where z is a helix 2-terminating residue and x is an amino acid residue. In some instances of [Linker 2], z is p or G. In some instances of [Linker 2], z is p. In some instances of [Linker 2], x is a VEGF-A contacting residue. In some instances of [Linker 2], x is an aromatic residue. In some instances of [Linker 2], x is a w or h residue, or a substituted version thereof. In some instances of [Linker 2], x is tyrosine or an analog thereof. In certain instances, [Linker 2] includes a helix terminating proline residue that provides for a modified Helix 2 to Helix 3 interhelix angle (i.e., angle between axes of the helices), e.g., as described herein. See FIG. 27.

A tyrosine analog can be incorporated at position 37 in Linker 2, e.g., an analog including an substituted or unsubstituted, alkyl-aryl or alkyl-heteroaryl extended sidechain group that can make closer contacts (e.g., hydrophobic contacts and/or a hydrogen bond) with adjacent residues of VEGF-A. FIG. 23 depicts the binding interface between compound (1.1.1 (c21a)) and VEGF-A showing the phenolic oxygen of residue y37 (209) that projects towards the VEGF-A surface is 6.5 to 7.2 angstrom distant from adjacent VEGF-A residues. In some cases, x is a tyrosine analog having a sidechain of formula: —(CH₂)ₙ—Ar where n is 1, 2, 3 or 4; and Ar is an aryl, substituted aryl, heteroaryl or substituted heteroaryl. In certain instances of x, Ar is a substituted phenyl. In certain instances of x, Ar is a substituted phenyl and n is 2 or 3. In certain instances of x, Ar is a phenyl substituted with a hydrogen bond donor or acceptor-containing group configured to hydrogen bind to the adjacent residues of VEGF-A.

In some embodiments of formula (I), [Helix 2]-[Linker 2]-[Helix 3] comprises a sequence of the formula (XII) that defines a VEGF-A binding surface:

(XII)
(SEQ ID NO: 99)
z²⁶h*jxxf*jxh*jzy*xxh*jxujxxujx⁴⁹

wherein:

each z is a helix-terminating residue;

y* is tyrosine or an analog thereof;

each h* is independently histidine or an analog thereof;

f* is phenylalanine or an analog thereof;

each u is independently a non-polar residue.

each j is independently a hydrophobic residue; and

each x is independently an amino acid residue.

In certain instances, Helix 3 of formula (XII) includes additional residues x⁵⁰x⁵¹, where x is an amino acid residue. In some cases, x⁵⁰ is k or r. In some instances of extended formula (XII), x⁵⁰ is k and x⁵¹ is a. In some cases of extended formula (XII), x⁵⁰ is e and x⁵¹ is d. In certain instances of formula (XII), x⁵⁰ is G and x⁵¹ is r. In certain instances, Helix 3 of formula (XII) includes a C-terminal region selected from one of SEQ ID NO: 85-87. In some embodiments of extended formula (XII), x⁵¹ is framework residue. In some embodiments of extended formula (XII), x⁵¹ is a non-polar residue (u). In some embodiments of extended formula (XII), x⁵¹ is a hydrophobic residue.

In some embodiments of the compound, [Helix 2]-[Linker 2]-[Helix 3] has 70% identity or greater to the sequence of SEQ ID NO: 80, such as 75% identity or greater, 83% identity or greater, 87% identity or greater, 91% identity or greater or 95% identity or greater to the sequence of SEQ ID NO: 80. In some embodiments of the compound, [Helix 2]-[Linker 2]-[Helix 3] has 70% identity or greater to the sequence of SEQ ID NO: 83, such as 80% identity or greater, 84% identity or greater, 88% identity or greater, 92% identity or greater or 96% identity or greater to the sequence of SEQ ID NO: 83.

In certain instances of formula (I), [Linker 1] has a sequence of the formula:

(XIII)
(SEQ ID NO: 147)
z(x)ₙx′z

wherein: x′ is a polar residue; each x is an amino acid and n is an integer from 1-6; and each z is independently a helix-terminating residue, e.g., the first z is a Helix 1-terminating resdiue and the second z is a Helix 2-terminating residue. In certain instances, x′ is a polar residue capable of hydrogen bonding to VEGF-A. In some cases, x′ is selected from d, e, n, q, ornithine, 2-amino-3-guanidinopropionic acid and citrulline. In certain cases, n is 1, 2 or 3. In certain instances of formula (XIII), [Linker 1] has a sequence of the formula (XIV):

(XIV)
(SEQ ID NO: 148)
z(x)ₙe*z

wherein: each x is an amino acid and n is 1, 2 or 3; each z is independently a helix-terminating residue; and e* is glutamic acid or an analog thereof In some instances of formulae (XIII) and (XIV), each z is selected from G and p. In some instances of formulae (XIII) and (XIV), n is 2.

In certain instances of formula (I), [Linker 1]-[Helix 2]-[Linker 2]-[Helix 3] comprises a sequence of the formula:

(XV)
(SEQ ID NO: 100)
z²²xxe*zh*jxxf*jxh*jzy*xxh*jxujxxujxxx⁵¹

wherein:

e* is glutamic acid or an analog thereof;

each z is independently a helix-terminating residue;

y* is tyrosine or an analog thereof;

each j is independently a hydrophobic residue;

each u is independently a non-polar amino acid residue; and

each x is independently an amino acid residue.

In some instances of formulae (I), (XII) and (XV), [Helix 2] is defined by a sequence of the formula (XVI):

(XVI)
(SEQ ID NO: 101)
z²⁶hj²⁸xxfj³²xj³⁵z³⁶

wherein:

z²⁶ is selected from d, p and G;
Z³⁶ is selected from p and G;
j²⁸, j³² and j³⁵ are each independently a hydrophobic residue; and
each x is independently an amino acid residue.

In certain instances, j²⁸, j³² and j³⁵ are corresponding residues of a GA scaffold domain selected from SEQ ID NO: 1-21. In some cases, j²⁸, j³² and j³⁵ are independently selected from a, i, l and v.

In some instances of formulae (I), (XII), (XV) and (XVI), [Helix 2] is defined by a sequence selected from: a) phvx²⁹x³⁰fix³³hap (XVII) (SEQ ID NO: 102) wherein: x²⁹ is selected from f and i; and x³⁰ and x³³ are independently selected from a polar amino acid residue; and

b) an amino acid sequence which has 80% or greater identity to the sequence of formula (XVII) defined in a) (e.g., 90% or greater identity).

In some instances of formulae (XVI)-(XVII), x³⁰ and x³³ are independently selected from n, s, d, e and k. In some instances of formulae (XVI)-(XVII), x²⁹ is i. In some instances of formulae (XVI)-(XVII), x³⁰ is s or n. In some instances of formulae (XVI)-(XVII), x³³ is n. In some instances of formulae (XVI)-(XVII), x²⁹ is i; x³⁰ is s or n; and x³³ is n.

In some instances of formulae (I), (XII) and (XV), [Helix 3] is defined by a sequence of the formula (XVIII):

(XVIII)
(SEQ ID NO: 103)
xxhj⁴¹xuj⁴⁴xxuj⁴⁸xxx⁵¹

wherein:

j⁴¹, j⁴⁴ and j⁴⁸ and are each independently a hydrophobic residue;

each u is independently a non-polar amino acid residue; and

each x is independently an amino acid residue.

In some cases, x⁵⁰ is k or r. In some instances of formula (XVIII), x⁵⁰ is k and x⁵¹ is a. In some instances of formula (XVIII), x⁵⁰ is e and x⁵¹ is d. In some instances of formula (XVIII), x⁵⁰ is G and x⁵¹ is r. In certain instances, Helix 3 of formula (XVIII) includes a C-terminal region selected from one of SEQ ID NO: 85-87. In some embodiments of formula (XVIII), x⁵¹ is framework residue. In some embodiments of formula (XVIII), x⁵¹ is a non-polar residue (u). In some embodiments of formula (XVIII), x⁵¹ is a hydrophobic residue. In some embodiments of formula (XVIII), j⁴¹, j⁴⁴ and j⁴⁸ are independently selected from a, i, l and v. In some embodiments of formula (XVIII), j⁴¹, j⁴⁴ and j⁴⁸ are corresponding residues of a GA scaffold domain selected from SEQ ID NO: 1-21.

In some instances of formulae (I), (XII) and (XV), [Helix 3] is defined by a sequence selected from : a) x³⁸x³⁹hvx⁴²Glx⁴⁵x⁴⁶aix⁴⁹x⁵⁰a (XIX) (SEQ ID NO: 104) wherein:
x³⁸ is selected from v, e, k, r;
x³⁹, x⁴², x⁴⁶ and x⁵⁰ are independently selected from a polar amino acid residue; and
x⁴⁵ and x⁴⁹ are independently selected from l, k, r and e; and

b) an amino acid sequence which has 80% or greater identity to the sequence of formula (XIX) defined in a) (e.g., 90% or greater identity).

In some instances of formula (XIX), x³⁹, x⁴², x⁴⁶ and x⁵⁰ are independently selected from n, s, d, e and k. In some instances of formula (XIX), x³⁸ is V. In some instances of formula (XIX), x⁴⁵ is k. In some instances of formula (XIX), x⁴⁹ is 1. In some instances of formula (XIX), x³⁹ is s. In some instances of formula (XIX), x⁴² is n. In some instances of formula (XIX), x⁴⁶ is n. In some instances of formula (XIX), x⁵⁰ is k.

In certain cases, [Helix 1] comprises the following consensus sequence: l⁷..a¹⁰ke.ai.elk..²¹, where the residues at positions 8, 9, 13, 16, 20 and 21 are defined by any one of the corresponding residues of the sequences of the GA domains of Table 3. In certain cases, [Helix 1] comprises a sequence of 15 residues having 66% or more % identity, such as 73% or more, 80% or more, 86% or more, or 93% or more % identity, to the following sequence. l⁶lknakedaiaelkk²⁰.

In some embodiments of the compound, [Linker 1]-[Helix 2]-[Linker 2]-[Helix 3] has 70% identity or greater to the sequence of SEQ ID NO: 81, such as 78% identity or greater, 82% identity or greater, 85% identity or greater, 89% identity or greater, 92% identity or greater or 96% identity or greater to the sequence of SEQ ID NO: 81. In some embodiments of the compound, [Linker 1]-[Helix 2]-[Linker 2]-[Helix 3] has 70% identity or greater to the sequence of SEQ ID NO: 84, such as 80% identity or greater, 83% identity or greater, 86% identity or greater, 90% identity or greater, 93% identity or greater or 96% identity or greater to the sequence of SEQ ID NO: 84.

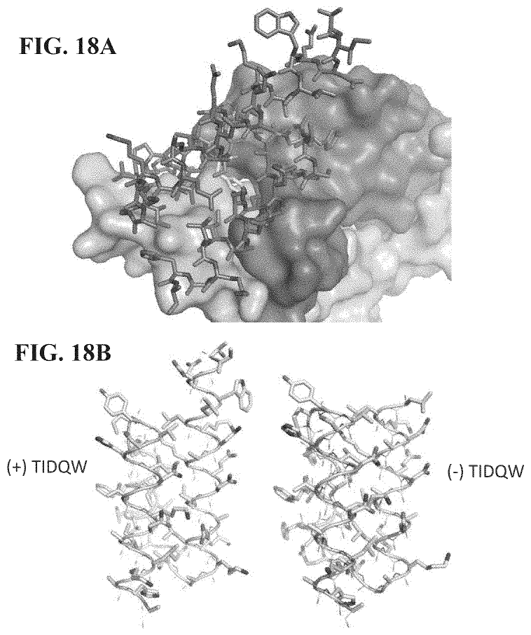

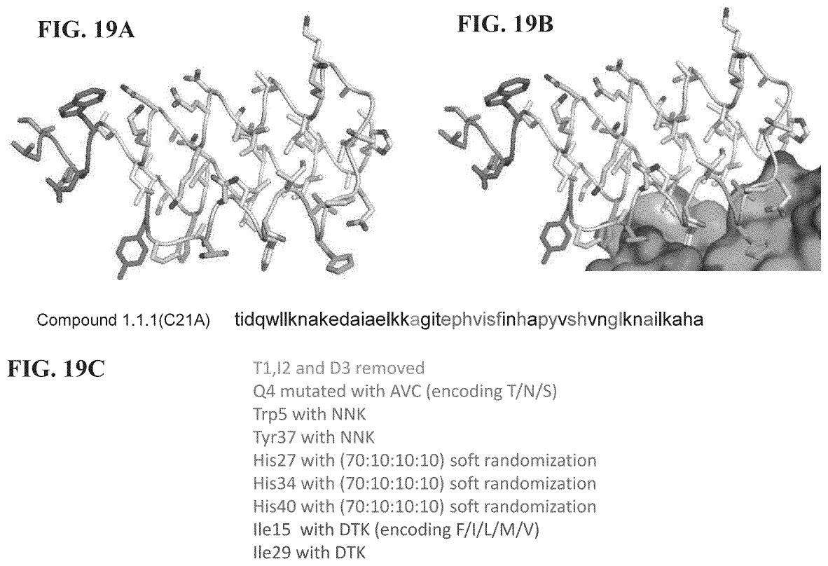

Any convenient N-terminal alpha-helical segments of GA domains of interest can be adapted for use in the subject compounds. In some cases, [Helix 1] includes a sequence of N-terminal residues from about position 6 up to about position 20. FIG. 18B shows a N-terminal truncated derivative of an exemplary compound where residues 1-5 can be removed from the compound, without significantly adversely affecting the intramolecular hydrophobic contacts of the compound that stabilize the three-helix bundle. In certain instance, the subject compound is truncated at the N-terminal by 6 or less residues, such as 5 or less, 4 or less, 3 or less, 2 or less or 1 residue relative to the numbering system 1-53 depicted described herein. In certain instances, one or more of the residues in positions 1-5 of the subject compound are either deleted or modified, e.g., to impart a desirable property on the resulting compound such as helix capping, increased water solubility, or a linkage to a molecule of interest (e.g., as described herein).

In certain cases, [Helix 1] comprises the following consensus sequence: l⁷..a¹⁰ke.ai.elk..²¹ (SEQ ID NO: 105), where the residues at positions 8, 9, 13, 16, 20 and 21 are defined by any one of the corresponding residues of the sequences of SEQ ID NO: 2-21. In certain cases, [Helix 1] comprises a sequence of 15 residues having 66% or more % identity, such as 73% or more, 80% or more, 86% or more, or 93% or more % identity, to the following sequence l⁶lknakedaiaelkk²⁰ (SEQ ID NO: 74).

Described herein are D-peptidic GA domains having VEGF specificity-determining motifs (SDM) defined by a configuration of variant amino acid residues comprised in an underlying sequence of peptidic framework residues. Based on the present disclosure, it is understood that variations of any of the SDMs and peptidic framework residues/sequences are also encompassed by the present disclosure. In some embodiments, the GA domain includes a VEGF SDM having 50% or more, 60% or more, 65% or more, 70% or more, such as 75% or more, 80% or more, 85% or more, 90% or more, or 95% or more identity with any one of embodiments of SDM residues and/or peptidic framework residues defined herein. In some embodiments, the GA domain includes a VEGF SDM having 1 to 5, e.g., 1 to 4, or 1 to 3 amino acid residue substitutions (e.g., 1, 2, 3, 4 or 5 substitutions) relative to any one of embodiments of SDM residues and/or peptidic framework residues defined herein. In certain embodiments, the 1 to 3 amino acid residue substitutions are selected from similar, conservative or highly conserved amino acid residue substitutions according to Table 6.

In some embodiments of the D-peptidic compound that specifically binds VEGF, the D-peptidic GA domain comprises a VEGF specificity-determining motif (SDM) defined by the following amino acid residues:

(SEQ ID NO: 149)
e²⁵phvisf--h³⁴-p³⁶x³⁷-s³⁹h--G⁴³---a⁴⁷

wherein x³⁷ is selected from s, n, and y. In some embodiments of the VEGF SDM, x³⁷ is s. In some embodiments of the VEGF SDM, x³⁷ is n. In some embodiments of the VEGF SDM, x³⁷ is y.

In some embodiments, the VEGF SDM is further defined by the following residues:

(SEQ ID NO: 150)
c⁷-----------------e²⁵phvisf--h³⁴-p³⁶x³⁷c³⁸sh--G⁴³---a⁴⁷

wherein x³⁷ is selected from s and n. In some embodiments of the VEGF SDM, x³⁷ is s. In some embodiments of the VEGF SDM, x³⁷ is n.

In some embodiments of the GA domain, Helix 1^{(#6-21)} comprises a peptidic framework sequence: x⁶x⁷knakedaiaelkka²⁰ (SEQ ID NO: 138)

wherein: x⁶ is selected from l, v, and i; and x⁷ is selected from l and c.

In some embodiments of Helix 1, x⁶ is l. In some embodiments of Helix 1, x⁶ is v. In some embodiments of Helix 1, x⁶ is i.

In some embodiments, the GA domain comprises an N-terminal peptidic framework sequence:

(SEQ ID NO: 139)
x¹x²x³qwx⁶x⁷knakedaiaelkkaGit²⁴

wherein:

x¹ is selected from t, y, f, i, p and r;

x² is selected from i, h, n, p, and s;

x³ is selected from d, i, and v;

x⁶ is selected from l, v, and i; and

x⁷ is selected from l and c.

In some embodiments of the peptidic framework sequence, x¹ is t. In some embodiments of the peptidic framework sequence, x¹ is y. In some embodiments of the peptidic framework sequence, x¹ is f. In some embodiments of the peptidic framework sequence, x¹ is i. In some embodiments of the peptidic framework sequence, x¹ is p. In some embodiments of the peptidic framework sequence, x¹ is r.

In some embodiments of the peptidic framework sequence, x² is i. In some embodiments of the peptidic framework sequence, x² is h. In some embodiments of the peptidic framework sequence, x² is n. In some embodiments of the peptidic framework sequence, x² is p. In some embodiments of the peptidic framework sequence, x² is s.

In some embodiments of the peptidic framework sequence, x³ is d. In some embodiments of the peptidic framework sequence, x³ is i. In some embodiments of the peptidic framework sequence, x³ is v.

In some embodiments of the peptidic framework sequence, x⁶ is 1. In some embodiments of the peptidic framework sequence, x⁶ is v. In some embodiments of the peptidic framework sequence, x⁶ is i.

In some embodiments of the peptidic framework sequence, x⁷ is 1. In some embodiments of the peptidic framework sequence, x⁷ is c.

In some embodiments, the D-peptidic GA domain comprises a C-terminal peptidic framework sequence: ilkaha (SEQ ID NO: 140).

In some embodiments, the D-peptidic GA domain comprises a sequence:

(SEQ ID NO: 141)
x¹x²x³qwx⁶x⁷knakedaiaelkkagitephvisfinhapx³⁷x³⁸shvn
Glknailkaha⁵³

wherein:

x¹is selected from t, y, f, i, p and r;

x² is selected from i, h, n, p, and s;

x³ is selected from d, i, and v;

x⁶ is selected from l, v, and i;

x⁷ is selected from l and c;

x³⁷ is selected from t, y, n, and s;

x³⁸ is selected from v and c;

x³⁹ is selected from e and s;

x⁴⁰ is selected from h and e;

x⁴³ is selected from g and a; and

x⁴⁷ selected from is a and e.

In some embodiments, x¹ is t. In some embodiments, x¹ is y. In some embodiments, x¹ is f. In some embodiments, x¹ is i. In some embodiments, x¹ is p. In some embodiments, x¹ is r. In some embodiments, x² is i. In some embodiments, x² is h. In some embodiments, x² is n. In some embodiments, x² is p. In some embodiments, x² is s. In some embodiments, x³ is d. In some embodiments, x³ is i. In some embodiments, x³ is v. In some embodiments, x⁶ is l. In some embodiments, x⁶ is v. In some embodiments, x⁶ is i. In some embodiments, x⁷ is l. In some embodiments, x⁷ is c. In some embodiments, x³⁷ is t. In some embodiments, x³⁷ is y. In some embodiments, x³⁷ is n. In some embodiments, x³⁷ is s. In some embodiments, x³⁸ is v. In some embodiments, x³⁸ is c. In some embodiments, x³⁹ is e. In some embodiments, x³⁹ is s. In some embodiments, x⁴⁰ is h. In some embodiments, x⁴⁰ is e. In some embodiments, x⁴³ is g. In some embodiments, x⁴³ is a. In some embodiments, x⁴⁷ is a. In some embodiments, x⁴⁷ is e.

In some embodiments, D-peptidic compound comprises a sequence selected from one of compounds 11055, 979102 and 979107-979110 (SEQ ID NOs: 108-113).

In some embodiments, D-peptidic compound comprises a sequence having 80% or more (e.g., 90% or more) identity with one of compounds 11055, 979102 and 979107-979110 (SEQ ID NOs: 108-113).

In some embodiments, D-peptidic compound comprises a sequence having 1 to 10 amino acid residue substitutions (e.g., 1 to 9, 1 to 8, 1 to 7, 1 to 6, 1 to 5, 1 to 4, 1 to 3, such as 1 or 2 amino acid residue substitutions), relative to one of compounds 11055, 979102 and 979107-979110 (SEQ ID NOs: 108-113). In certain embodiments, the 1 to 10 amino acid residue substitutions are selected from similar, conservative and highly conserved amino acid residue substitutions, e.g., according to Table 6.

### GA Scaffold Domain

Based on the present disclosure, it is understood that several of the amino acid residues of the GA domain motif which are not located at the VEGF-A binding surface of the structure can be modified without having a detrimental effect on the VEGF-A binding activity of the resulting modified compound. As such, any convenient amino acids can be incorporated into the subject compounds to impart a desirable property, including but not limited to, increased water solubility, ease of chemical synthesis, cost, bioconjugation site, stability, pI, aggregation, reduced non-specific binding and/or specific binding to a second target protein. The positions of the mutations may selected so as to minimize any disruption to the structure of the VEGF-A binding GA domain motif or specific binding to the target VEGF-A protein, e.g., by selecting positions on opposite sides of the structure from the VEGF-A binding surface. In some instances, the compound includes two or more, such as 3 or more, 4 or more, 5 or more, 6 or more, 7 or more, 8 or more, 9 or more, or 10 or more surface mutations at positions that are not part of the binding surface to the target VEGF-A protein.

For example, in some cases, one or more of the c, f and b residues of Helix 1 and the c andfresidues of Helices 2 and 3 can be modified since those residues are not directly involved in VEGF-A binding and solvent exposed (see heptad model of FIG. 3B). In certain cases, a variant amino acid residue can be selected for incorporation into a subject compound at a particular heptad repeat position according to the percentage occurrences of known amino acid at analogous positions, e.g., in known naturally occurring proteins. Table 2 provides a list of the amino acid percentage occurrences for three-stranded coiled-coil heptad positions which may be utilized to select variant amino acid residues, e.g., amino acid residues having percentage occurrences of 2% or more, such as 5% or more, 10% or more or even more. In some cases, surface mutations include mutating the residue to a polar residue, e.g., that imparts a desirable solubility on the compound. In some cases, surface mutations include mutating the residue to a charged residue e.g., that imparts a desirable solubility on the compound. In some cases, surface mutations include mutating the residue to a basic residue (e.g., k or h). In some cases, surface mutations include mutating the residue to an acidic residue (e.g., d or e), e.g., that imparts a desirable pI on the compound.

TABLE 2
Amino acid percentage occurrences for
three-stranded coiled-coil heptad positions*

| Amino | | | | | | | | |
|---|---|---|---|---|---|---|---|---|
| acid | a | b | c | d | e | f | g | M |
| Ala | 19.9 | 7.4 | 8.2 | 18.8 | 5.1 | 9.4 | 5.3 | 192 |
| Cys | 0 | 0.8 | 0.6 | 0 | 0 | 0 | 0.8 | 6 |
| Asp | 1.5 | 7.8 | 10.2 | 1.5 | 3.9 | [symbol] | 9.0 | 112 |
| Glu | 0.9 | 17.6 | 19.9 | 1.5 | 17.6 | [symbol] | 13.3 | 203 |
| Phe | 0 | 0.9 | 1.6 | 1.5 | 2.7 | 0.4 | 1.6 | 22 |
| Gly | 0.8 | 2.7 | 2.7 | 0 | 1. [symbol] | 1.8 | 0.8 | 26 |
| His | 0.8 | 1.2 | 2.0 | 1.2 | 1. [symbol] | 2.3 | 2.7 | 30 |
| Ile | 16.0 | 1.6 | 1.2 | 12.5 | 1.2 | 1.8 | 3.9 | 97 |
| Lys | 0.8 | 9.6 | 7.0 | 3.1 | 12.5 | 10. [symbol] | 12.5 | 144 |
| Leu | 25.0 | 3.1 | 2.3 | 30.1 | 9.4 | 5.1 | 12.9 | 225 |
| Met | 2.3 | 1.2 | 2.0 | 3.9 | 2.7 | 2.7 | 0.8 | 40 |
| Asn | 1.6 | 5.5 | 12.1 | 1.6 | 7.8 | 9.0 | 7.0 | 114 |
| Pro | 0.4 | 0.8 | 0.4 | 0.4 | 0 | 0 | 0 | 5 |
| Gln | 3.9 | 10.9 | 8.6 | 1.6 | 7.8 | 8.8 | 4.7 | 11 [symbol] |
| Arg | 0.4 | 6.3 | 6.6 | 0.4 | 7.8 | 10.2 | 3.9 | 91 |
| Ser | 5.1 | 8.6 | 9.4 | 2.3 | 8.2 | 8.2 | 4.7 | 119 |
| Thr | 3.9 | 8.2 | 3.9 | 4.7 | 5.1 | 5.9 | 7.4 | 100 |
| Val | 14.8 | 3.1 | 3.1 | 13.7 | 2.7 | 4.3 | 5.1 | 120 |
| Trp | 0.4 | 0.4 | 0 | 0 | 0.8 | 0 | 0.8 | 6 |
| Tyr | 1.5 | 2.3 | 1.2 | 1.2 | 1. [symbol] | 0.8 | 2.0 | 27 |
| Sum | 100 | 100 | 100 | 100 | 100 | 100 | 100 | 1792 |
| N | 256 | 256 | 256 | 256 | 256 | 256 | 256 | |

*M is total number of times a particular amino acid is found at a heptad position.
N is the total number of residue counted at that heptad position.
See Table 3 of DeGrado et al.. [symbol]
indicates data missing or illegible when filed

In some cases, the subject peptidic compounds were selected from a phage display library based on a GA scaffold domain and further developed (e.g., via additional affinity maturation and/or point mutations), to include several variant amino acids integrated with a GA scaffold domain. The variant motif comprises the variant amino acids and can define a VEGF-A binding surface of the subject compounds. SEQ ID NO: 25 shows a variant motif of exemplary compound 1.1.1(c21a). Aspects of the VEGF-A binding surface of the subject compounds are described above. It is understood that a variety of underlying GA scaffold domain sequences can be utilized in the subject compounds to provide a three-helix bundle scaffold structure in which the variant domain is incorporated. The structure of a subject compound can be defined by a combination of variant and framework domains. The sequence of a subject compound can be defined by a combination of variant and framework residues. As such, in some instances, the framework residues of a structural or sequence motif can be defined by the corresponding residues of a scaffold domain structure or sequence.

For example, a comparison of scaffold SCF32 (SEQ ID NO:2) and compound 1.1.1(c21a) (SEQ ID NO:24) gives a variant motif (SEQ ID NO:25) and a framework domain (SEQ ID NO:26). Aspects of the variant motif are described herein. It is understood that a variety of modifications can be incorporated into the framework domain without having a significant adverse effect on the three helix bundle structure or VEGF-A binding surface. FIGS. 3 and 4 show alignments of exemplary sequences and motifs onto the heptad repeat structural model of the subject compounds. Residues of Helix 1 that are solvent exposed and not involved in the hydrophobic core interactions can be any convenient amino acid residue, including but not limited to, polar residues. In some cases, the b, c and/or f residues (see e.g., FIG. 6B) of Helix 1 of the subject compounds can be varied without adversely affecting the VEGF-A binding activity of the compound and in certain cases provide for a desirable property. In some cases, the e and g residues of Helix 1 can also be varied. In certain embodiments, the fresidues of Helix 2 and/or Helix 3 can be varied without adversely affecting the VEGF-A binding activity of the compound and in certain cases provide for a desirable property. In certain instances, C-terminal modifications such as truncations or extensions may be included in Helix 3 (e.g., residues located at positions 50 -53 of Helix 3, see FIG. 10A) The subject compounds can have a framework domain motif as defined by one of SEQ ID NO: 2-21. In some cases, the framework domain motif of the compound is defined by SEQ ID NO: 1.

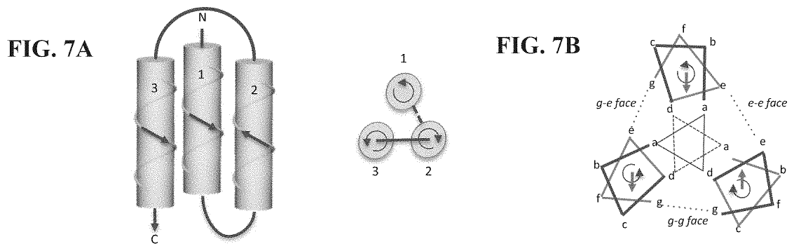

In some cases, modifications to residues that make contact with the hydrophobic core of a GA scaffold domain (e.g., a and d residues of the heptad repeat model as depicted in FIG. 7B) are less desirable as these residues are involved with helix to helix hydrophobic contacts that stabilize the three-helix bundle. However, a variety of non-polar or hydrophobic residues can find use in the hydrophobic core of the three-helix bundle of the subject compounds. FIG. 9A-9C shows the sequence and structure of an exemplary compound where the configuration of a and d residues of the heptad repeat model that can form inter-helix hydrophobic interactions are indicated in red. In certain instances, the C-terminal e residue of the Helix 3 heptad repeat which is located at the terminal of the helical region can be modified, e.g., to provide for a helix capping, helix truncation, or extension to a linking group. In certain instances, one, two or more of the N-terminal residue(s) of the Helix 1 heptad repeat (e.g., N-terminal residues of FIG. 10A) which is located at the terminal of the helical region can be modified, e.g., to provide for a helix capping, helix truncation, or extension to a linking group. In certain embodiments, the a and d residues of a subject compound can be selected from the corresponding hydrophobic core residues of any of SEQ ID NO: 1-21.

In certain instances, each a and d residue of [Helix 2] is a residue capable of imparting stability on the modified three-helix bundle structure of the subject compound. In certain cases, one or more of the a and d residues of the subject compound, e.g., at positions 28, 32 and 35 of [Helix 2] provide intramolecular contacts, that define in part the hydrophobic core of the compound. In certain embodiments of [Helix 2], each a and d residue is independently a hydrophobic residue. In certain cases of [Helix 2], each a and d residue is selected from a, i, f, m, l and v. In some embodiments of [Helix 2], each a and d residue is selected from a, i, f, l and v. In certain instances of [Helix 2], each a and d residue is selected from a, i, 1 and v. In some instances of [Helix 2], the a and d residues at positions 32 and 35 are part of a scaffold domain (e.g., framework residues that have the same identity as corresponding residues of a scaffold domain motif).

In certain instances, the “d” residues of [Helix 2] and [Helix 3] that are closest to the g-g face of the structure which contacts the VEGF-A can make contact with the protein. In such cases, the VEGF-A contacting “d” residues can be revered to as boundary residues. It is understood that the

Table 3 sets forth a list of sequences of exemplary scaffold domains, exemplary compounds, and exemplary compound regions of interest. In some embodiments of formula (I)-(XIX), the residues correspond to the residues located at the same positions of one of SEQ ID NOs: 22-71 set forth in Table 3. In certain embodiments of formula (I), the compound comprises a sequence of residues having 85% or more % identity, such as 88% or more, 90% or more, 92% or more, 94% or more, 96% or more, or 98% or more % identity, to one of SEQ ID NOs: 22-71. In some cases, the sequence identity comparison is based on sequence regions having the same length, e.g., 48 residue, 49 residues, 50 residues, 51 residues, 52 residues or 53 residues in length. These subject compounds can be further mutated to incorporate residues at surface positions of the GA domain motif not involved in contacting the target VEGF-A protein. The residues can be selected to confer on the resulting modified compound a desirable property (e.g., as described herein).

TABLE 3
Sequences of Scaffolds and Compounds of interest

| Scaffold Name | Sequence | SEQ ID NO: |
|---|---|---|
| GA domain consensus | ......l⁷..a¹⁰ke.ai.elk²⁰.Gi.sd.y..³⁰.inkaktve.⁴⁰v.alk.eil⁴⁹.... | 1 |
| SCF32 | t¹idqwllknakedaiaelkkaGitsdfyfnainkaktveevnalkneilkaha⁵³ | 2 |
| SCF32 fixed domain 1 | tidqwllknakedaiaelkkaGit.d..fn.in.a..v..vn..kn.ilkaha | 3 |
| SCF32 fixed domain 2 | tidqwllknakedaiaelkk.Git.......in.a..v..vn..kn.ilkaha | 4 |
| SCF32 fixed domain 3 | tidqwlkna¹⁰kedaiaelkk²⁰aGit......³⁰.in.a..v..⁴⁰vn.lkn.ilkaha | 5 |
| ALB8-GA | t¹idqwll⁷knakedaiaelkkaGitsdfyfnainkaktveevnalkneilkaka⁵³ | 6 |
| ALB1-GA | l⁷knakedaiaelkkaGitsdfyfnainkaktveGanalkneilka⁵¹ | 7 |
| ALB8-uGA | l⁷kltkeeaekalkklGitsefilnqidkatsreGleslvqtikqs⁵¹ | 8 |
| ALB1B-uGA | l⁷qeakdkaiqeakanGltsklllknienaktpesaksfaeeliks⁵¹ | 9 |
| L3316-GA1 | l⁷knakeeaikelkeaGitsdlyfslinkaktveGvealkneilka⁵¹ | 10 |
| L3316-GA2 | l⁷knakedaikelkeaGissdiyfdainkaktveGvealkneilka⁵¹ | 11 |
| L3316-GA3 | l⁷knakeaaikelkeaGitaeylfnlinkaktveGveslkneilka⁵¹ | 12 |
| L3316-GA4 | l⁷knakedaikelkeaGitsdiyfdainkaktieGvealkneilka⁵¹ | 13 |
| G148-GA1 | l⁷akakadalkefnkyGv-sdyyknlinnaktveGvkdlqaqvves⁵¹ | 14 |
| G148-GA2 | l⁷aeakvlanreldkyGv-sdyhknlinnaktveGvkdlqaqvves⁵¹ | 15 |
| G148-GA3 | l⁷aeakvlanreldkyGv-sdyyknlinnaktveGvkalideilaalp⁵³ | 16 |
| DG12-GA1 | l⁷dnaknaalkefdryGv-sdyyknlinkaktveGimelqaqvves⁵¹ | 17 |
| DG12-GA2 | l⁷seakemaireldanGv-sdfykdkiddaktveGvvalkdlilns⁵¹ | 18 |
| MAG-GA1 | l⁷aklaadtdldldvakiind-yttkvenaktaedvkkifee--sq⁵¹ | 19 |
| MAG-GA2 | l⁷akakadaieilkkyGi-GdyyiklinnGktaeGvtalkdeil--⁵¹ | 20 |
| ZAG-GA | l⁷leakeaainelkqyGi-sdyyvtlinkaktveGvnalkaeilsa⁵¹ | 21 |
| Compound Name | Sequence | SEQ ID NO: |
| 1 | tidqwllknakedaiaelkkaGitsdhvfnfinyapyvsdvnalkneilkaha | 107 |
| 1.1 | tidqwllknakedaiaelkkaGitedhvfnfinhapyvshvnGlknailkaha | 22 |
| 1.1.1 | tidqwllknakedaiaelkkcGitephvisfinhapyvshvnGlkanilkaha | 23 |
| 1.1.1(c21a) | tidqwllkna¹⁰kedaiaelkkk²⁰aGitephvis³⁰finhapyvsh⁴⁰vnGlknailk⁵⁰ | 24 |
| | aha | |
| 1.1.1(c21a) variant motif | ---------¹⁰----------²⁰----ephvis³⁰f--h-py-sh⁴⁰--G---a---⁵⁰ | 25 |
| | --- | |
| 1.1.1(c21a) framework | tidqwllkna¹⁰kedaiaelkk²⁰aGit......³⁰.in.a..v..⁴⁰vn.lkn.ilk⁵⁰ | 26 |
| domain | aha | |
| 1.1.1(c21a) framework | llkna¹⁰kedaiaelkk²⁰aGit......³⁰.in.a..v..⁴⁰vn.lkn.ilk⁵⁰a | 27 |
| domain N/C truncated | | |
| 1.1.1(c21a) variant | ------l⁷--a¹⁰ke-ai-elk-²⁰-Gi-ephvis³⁰finhapyvsh⁴⁰v-Glk-ail⁴⁹- | 28 |
| motif + GA domian | --- | |
| consensus | | |
| 1.1.1(c21a) truncated | llkna¹⁰kedaiaelkk²⁰aGitephvis³⁰finhapyvsh⁴⁰vnGlknailk⁵⁰aha | 29 |
| (-)TIDQW | | |
| 1.1.1(c21a): Ile15 to | llkna¹⁰deda-aelkk²⁰aGitpehvis³⁰finhapyvsh40vnGlknailk⁵⁰aha | 30 |
| hydrophobic (f, i, l, m | | |
| or v) | | |
| 1.1.1(c21a): Ile29 to | llkna¹⁰kedaiaelkk²⁰aGitephv-s³⁰finhapyvsh⁴⁰vnGlknailk⁵⁰aha | 31 |
| hydrophobic (f, i, l, m | | |
| or v) | | |
| 1.1.1(c21a): Ile15/29 to | llkna¹⁰keda-aelkk²⁰aGitephv-s³⁰finhapyvsh⁴⁰vnGlkanilk⁵⁰aha | 32 |
| hydrophob (f, i, l, m or | | |
| v) | | |
| 1.1.1(c21a): Trp5 | tidq-llkna¹⁰dedaiaelkk²⁰aGitephvis³⁰finhapyvsh⁴⁰vnGlknailk⁵⁰ | 33 |
| mutation (NNK) | aha | |
| 1.1.1(c21a): Tyr37 soft | tidqwllkna¹⁰kedaiaelkk²⁰aGitephvis³⁰finhap-vsh⁴⁰vnGlknailk⁵⁰ | 34 |
| randomization (NNK) | aha | |
| 1.1.1(c21a): Trp5 and | tidq-llkna¹⁰kedaiaelkk²⁰aGitephvis³⁰finhap-vsh⁴⁰vnGlknailk⁵⁰ | 35 |
| Tyr mutations | aha | |
| 1.1.1(c21a): truncated | qwllkna¹⁰kedaiaelkk²⁰aGitephvis³⁰finhapyvsh⁴⁰vnGlknailk⁵⁰aha | 36 |
| (-) TID | | |
| 1.1.1(c21a): Gln4 | -wllkna¹⁰kedaiaelkk²⁰aGitephvis³⁰finhapyvsh⁴⁰vnGlknailk⁵⁰aha | 37 |
| mutated with AVC to | | |
| (t, n or s) | | |
| 1.1.1(c21a): Trp5 | --llkna¹⁰kedaiaelkk²⁰aGitephvis³⁰finhapyvsh⁴⁰vnGlknailk⁵⁰aha | 38 |
| mutation (NNK) | | |
| 1.1.1(c21a): Ile15 to | --llkna¹⁰keda-aelkk²⁰aGitephvis³⁰finhapyvsh⁴⁰vnGlknailk⁵⁰aha | 39 |
| hydrophobic (f, i, l, m | | |
| or v) | | |
| 1.1.1(c21a): Ile29 to | --lkna¹⁰kedaiaelkk²⁰aGitephv-s³⁰finhapyvsh⁴⁰vnGlknailk⁵⁰aha | 40 |
| hydrophobic (f, i, l, m | | |
| or v) | | |
| 1.1.1(c21a): His27 | llkna¹⁰kedaiaelkk²⁰aGitep-vis³⁰finhapyvsh⁴⁰vnGlknailk⁵⁰aha | 41 |
| mutation | | |
| 1.1.1(c21a): His34 | llkna¹⁰kedaiaelkk²⁰aGitephvis-³⁰fin-apyvsh⁴⁰vnGlknailk⁵⁰aha | 42 |
| mutation | | |
| 1.1.1(c21a): His40 | llkna¹⁰kedaiaelkk²⁰aGitephvis-³⁰finhapyvs-⁴⁰vnGlknailk⁵⁰aha | 43 |
| mutation | | |
| 1.1.1(c21a): His27, | llkna¹⁰kedaiaelkk²⁰aGitep-vis³⁰fin-apyvs-⁴⁰vnGlknailk⁵⁰aha | 44 |
| His 34 and/or His 40 | | |
| mutate | | |
| 1.1.1(c21a): Phe31 to | llkna¹⁰kedaiaelkk²⁰aGitephvis³⁰f*inhapyvsh⁴⁰vnGlknailk⁵⁰aha | 45 |
| Phe analog (*) | | |
| 1.1.1(c21a): Tyr37 to | llkna¹⁰kedaiaelkk²⁰aGitephvis³⁰finhapy*vsh⁴⁰vnGlknailk⁵⁰aha | 46 |
| Tyr analog (*) | | |
| 1.1.1(c21a): Phe31 + | llkna¹⁰kedaiaelkk²⁰aGitephvis³⁰f*inhapy*vsh⁴⁰vnGlknailk⁵⁰aha | 47 |
| Tyr37 to analogs (*) | | |
| 1.1.1.2 | tidqwllknakedaiaelkkaGitedhvfnfinhapyvshvnGlknailGrtvp | 48 |
| 1.1.1.2 (pis) | tidqwllknakedaiaelkkaGitephvisfinhapyvshvnGlknailGrtvp | 49 |
| 1.1.1.2 (pis, asc) | tidqwllknakedaiaelkkaGitephvisfinhapyvshvnGlknailGrtvpasc | 50 |
| 1.1.1.2 (pa, pis) | pallknakedaiaelkkaGitephvisfinhapyvshvnGlknailGrtvp | 51 |
| 1.1.1.2 (pa, pis, asc) | pallknakedaiaelkkaGitephvisfinhapyvshvnGlknailGrtvpasc | 52 |
| 1.1.1.3 | tidqwllknakedaiaelkkaGitedhvfnfinhapyvshvnGlknailedwyl | 53 |
| 1.1.1.3 (pis) | tidqwllknakedaiaelkkaGitephvisfinhapyvshvnGlknailedwyl | 54 |
| 1.1.1.3 (pis, asc) | tidqwllknakedaiaelkkagitephvisfinhapyvshvnglknailedwylasc | 55 |
| 1.1.1.3 (-tidqw/pa, pis) | pallknakedaiaelkkagitephvisfinhapyvshvnglknailedwyl | 56 |
| 1.1 (-kaha, adfl) | tidqwllknakedaiaelkkaGitedhvfnfinhapyvshvnGlknailadfl | 57 |
| 1.1 (-kaha, edyl) | tidqwllknakedaiaelkkaGitedhvfnfinhapyvshvnGlkaniledyl | 58 |
| 1.1 (kaha, Grtvp) | tidqwllknakedaiaelkkaGitedhvfnfinhapyvshvnGlkanilGrtvp | 59 |
| 1.1 (kaha, edwyl) | tidqwllknakedaiaelkkaGitedhvfnfinhapyvshvnGlkaniledwyl | 60 |
| 1.1 (-kaha, GehGsp) | tidqwllknakedaiaelkkaGitedhvfnfinhapyvshvnGlkanilGehGsp | 61 |
| 1.1.1(c21a) (-kaha, | tidqwllknakedaiaelkkaGitephvisfinhapyvshvnGlnailGrtvp | 62 |
| Grtvp) | | |
| 1.1.1(c21a) (-tidqw, | llknakedaiaelkkaGitephvisfinhapyvshvnGlknailGrtvp | 63 |
| -kaha, Grtvp) | | |
| 1.1.1(c21a) (-tidqw, | pallknakedaiaelkkaGitephvisfinhapyvshvnGlknailGrtvp | 64 |
| pa, -kaha, Grtvp) | | |
| 1.1.1(c21a) (-tidqw, pa, | pallknakedaiaelkkaGitephvisfinhapyvshvnGlknailGrtvpasc | 65 |
| -kaha, Grtvpasc) | | |
| 1.1.1(c21a) (-kaha, | tidqwllknakedaiaelkkaGitephvisfinhapyvshvnGlknailedwyl | 66 |
| edwyl) | | |
| 1.1.1(c21a) (-tidqw, | llknakedaiaelkkaGitephvisfinhapyvshvnGlknailedwyl | 67 |
| -kaha, edwyl) | | |
| 1.1.1(c21a) (-tidqw, pa, | pallknakedaiaelkkaGitephvisfinhapyvshvnGlknailedwyl | 68 |
| -kaha,edwyl) | | |
| 1.1.1(c21a) (-kaha, | tidqwllknakedaiaelkkaGitephvisfinhapyvshvnGlknailedwylasc | 69 |
| edwylasc) | | |
| 1.1.1(c21a) (p26d) | tidqwllknakedaiaelkkaGitedhvisfinhapyvshvnGlknailkaha | 70 |
| 1.1.1(c21a) (c(Ac)54) | tidqwllknakedaiaelkkaGitephvisfinhapyvshvnGlknailkahac | 71 |
| | (acetyl) | |
| Compound regions of | | |
| Formula (I) | Sequence | SEQ ID NO: |
| N-terminal | t¹idqw⁵ | 72 |
| N-terminal | q⁴w⁵ | 73 |
| Helix 1 | l⁶lknakedaiaelkka²¹ | 74 |
| Linker 1 | G²²itep²⁶ | 75 |
| Helix 2 | h²⁷visfinha³⁵ | 76 |
| Capped Helix 2 | p²⁶hvisfinhap³⁶ | 77 |
| Linker 2 | p³⁶y³⁷ | 78 |
| Helix 3 | v³⁸shvnGlnail⁴⁹ | 79 |
| [Helix 2]-[Linker 2]- | h²⁷visfinhapyvshvnGlknail⁴⁹ | 80 |
| [Helix 3] | | |
| [Linker 1]-[Helix 2]- | G²²itephvisfinhapyvshvnGlknail⁴⁹ | 81 |
| [Linker 2]-[Helix 3] | | |
| Helix 3 | v³⁸shvnGlknailka⁵¹ | 82 |
| [Helix 2]-[Linker 2]- | h²⁷visfinhapyvshvnGlknailka⁵¹ | 83 |
| [Helix 3] | | |
| [Linker 1]-[Helix 2]- | G²²itephvisfinhapyvshvnGlknailka⁵¹ | 84 |
| [Linke 2]-[Helix 3] | | |
| C-terminal | k⁵⁰aha⁵³ | 85 |
| C-terminal | e⁵⁰dwyl⁵⁴ | 86 |
| C-terminal | G⁵⁰rtvp⁵⁴ | 87 |

TABLE 4
Exemplary D-Peptidic Z and GA Domains that bind VEGF

| | | VEGF | |
|---|---|---|---|
| | | Binding | |
| Compound | | Affinity | SEQ |
| # | Sequence | (K_{D}, nM) | ID NO: |
| GAdomain | tidqwllknakedaiaelkkaGitsdfyfnainkaktveevnalkneilkaha | No | 2 |
| wt | | binding | |
| 11055 | tidqwllknakedaiaelkkaGitephvisfinhapyvshvnGlknailkaha | 43 | 108 |
| 979102 | fniqwicknakedaiaelkkaGitephvisfinhapscshvnGlknailkaha | 5.2 | 109 |
| 979107 | ipiqwvcknakedaiaelkkaGitephvisfinhapscshvnGlknailkaha | 5.2 | 110 |
| 979108 | psvqwicknakedaiaelkkaGitephvisfinhapscshvnGlknailkaha | 5.8 | 111 |
| 979109 | rniqwvcknakedaiaelkkaGitephvisfinhapncshvnGlknailkaha | 3.7 | 112 |
| 979110 | yhiqwvcknakedaiaelkkaGitephvisfinhapncshvnGlknailkaha | 2.3 | 113 |
| 978333 | vdnkfnkewdnawleirhlpnlnheqkrafisslyddpsqsanllaeakklndaqapk | 2430 | 114 |
| 978334 | vdnkfnkewdnawreirhlpnlnheqkrafisslyddpsqsanllaeakklndaqapk | 1050 | 115 |
| 978335 | vdnkfnkewdnawreirhlpnlnleqkGafiaslyddpsqsanllaeakklndaqapk | 3010 | 116 |
| 978336 | vdnkfnkewdnawreirhlpnlnleqkrafisslyddpsqsanllaeakklndaqapk | 168 | 117 |
| 978337 | vdnkfnkewdnawteirhlpnlnreqkvafitslyddpsqsanllaeakklndaqapk | 1360 | 118 |
| 980174 | vdnkfnkewdnawkeirhlpnlnveqkrafihslyddpsqsanllaeakklndaqapk | 138 | 120 |
| 980175 | vdnkfnkewdnawreirhlpnlnieqkrafihslyddpsqsanllaeakklndaqapk | 110 | 121 |
| 980176 | vdnkfnkewdnawreirhlpnlnieqkrafirslyddpsqsanllaeakklndaqapk | 86 | 122 |
| 980177 | vdnkfnkewdnawreirhlpnlnieqkrafiyslyddpsqsanllaeakklndaqapk | 118 | 123 |
| 980178 | vdnkfnkewdnawreirhlpnlnleqkrafirslyddpsqsanllaeakklndaqapk | 102 | 124 |
| 980179 | vdnkfnkewdnawreirhlpnlnreqklafihslyddpsqsanllaeakklndaqapk | 87 | 125 |
| 980180 | vdnkfnkewdnawreirhlpnlnveqkrafikslyddpsqsanllaeakklndaqapk | 120 | 126 |
| 980181 | vdnkfnkewdnawreirhlpnlnveqkrafirslyddpsqsanllaeakklndaqapk | 17.6 | 119 |
| 981188 | vdnkfdkewdnawreirrlpnlnleqkrafisslyddpsqsanllaeakklndaqapk | 61 | 127 |
| 981189 | vdnkfnkewdnawreirrlpnlnleqkrafisslyddpsqsanllaeakklndaqapk | 50 | 128 |
| 981190 | vdnkfnkewdnawreirrlpnlnveqkrafisslyddpsqsanllaeakklndaqapk | 59 | 129 |

TABLE 5
Exemplary Multivalent VEGF-Binding D-Peptidic Compounds

| | | | | VEGF |
|---|---|---|---|---|
| | | | | Binding |
| Compd | | | | Affinity |
| # | Domain 1 | Linking Component | Domain 2 | (K_{D}, nM) |
| 979111 | 11055 | Maleimide-PEG8-Maleimide | 978336 | 0.47 |
| | N-terminal | N-terminal to N-terminal via cysteine- | N-terminal | |
| | cysteine | maleimide conjugations | cysteine | |
| 980870 | 979110 | {[yhiqwvcknakedaiaelk¹⁹(Azidoacetyl- | 980181 with | 0.31 |
| | With k19 | PEG2)kaGitephvisfinapncshvnGlknailkaha- | k7 linkage | |
| | linkage to | NH₂ (c to c disulfide bridge)]-interdomain | to 979110. | |
| | 980181 | click-[vdnfnk⁷(D-Pra- | Dimerized via | |
| | | PEG2)ewdnawreirhlpnlveqkrafirslyddpsqsanlla | -ak(-)NH₂ | |
| | | eakklndaqapk]}₂-ak(-)NH₂ | terminal | |
| | | | residues. | |
| 980871 | 979110 | {[yhiqwvcknakedaiaelk¹⁹(Azidoacetyl- | 980181 with | 0.42 |
| | With k19 | PEG3)kaGitephvisfinapncshvnGlknailkaha- | k7 linkage | |
| | linkage to | NH₂ (c to c disulfide bridge)]-interdomain- | to 979110. | |
| | 980181 | [vdnfnk⁷(D-Pra- | Dimerized via | |
| | | PEG2)ewdnawreirhlpnlveqkrafirslyddpsqsanlla | -ak(-)NH₂ | |
| | | eakklndaqapk]}₂-ak(-)NH₂ | terminal | |
| | | | residues. | |
| 980868 | 979110 | [yhiqwvcknakedaiael-k¹⁹(Azidoacetyl- | 980181 with | 0.1 |
| | With k19 | PEG2)kaGitephvisfinhapncshvnGlknailkaha- | k7 linkage | |
| | linkage to | NH2 (c to c disulfide bridge)]-interdomain- | to 979110 | |
| | 980181 | [vdnkfn-k⁷(DPra-PEG2)- | | |
| | | ewdnawreirhlpnlvdeqkrafirslyddpsqsanllaeakk | | |
| | | lndaqapk-NH₂] | | |
| 980869 | 979110 | [yhiqwvcknakedaiael-k¹⁹(Azidoacetyl- | 980181 with | 0.07 |
| | With k19 | PEG3)kaGitephvisfinhapncshvnGlknailkaha- | k7 linkage | |
| | linkage to | NH2 (c to c disulfide bridge)]-interdomain | to 979110 | |
| | 980181 | [vdnkfn-k⁷(DPra-PEG2)- | | |
| | | ewdnawreirhlpnlnveqkrafirslyddpsqsanllaeakk | | |
| | | lndaqapk-NH₂] | | |

Aspects of the present disclosure include compounds (e.g., as described herein), salts thereof (e.g., pharmaceutically acceptable salts), and/or solvate or hydrate forms thereof. It will be appreciated that all permutations of salts, solvates and hydrates are meant to be encompassed by the present disclosure. In some embodiments, the subject compounds are provided in the form of pharmaceutically acceptable salts. Compounds containing amine and/or nitrogen containing heteraryl groups may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

### Compound Properties

The variant D-peptidic domains of the subject multivalent compounds may define a binding surface area of a suitable size for forming protein-protein interactions of high functional affinity (e.g., equilibrium dissociation constant (K_{D})) and and specificity (e.g., 300 nM or less, such as 100 nM or less, 30 nM or less, 10 nM or less, 3 nM or less, 1 nM or less, 300 pM or less, or even less). The variant D-peptidic domains may each include a surface area of between 600 and 1800 Å², such as between 800 and 1600 Å², between 1000 and 1400 Å², between 1100 and 1300 Å², or about 1200 Å².

In some cases, the multivalent D-peptidic compound specifically binds a target protein with a binding affinity (K_{D}) 10-fold or more stronger, such as 30-fold or more, 100-fold or more, 300-fold or more, 1000-fold or more, or even more, than each of the binding affinities of the first and second D-peptidic domains alone for the target protein. A peptidic compound's affinity of a target protein can be determined by any convenient methods, such as using an SPR binding assay or an ELISA binding assay (e.g., as described herein). In certain cases, the multivalent D-peptidic compound has a binding affinity (K_{D}) for the target protein of 3 nM or less, such as 1 nM or less, 300 pM or less, 100 pM or less, and the binding affinities of the first and second D-peptidic domains alone for the target protein are each independently 100 nM or more, such as 200 nM or more, 300 nM or more, 400 nM or more, 500 nM or more, or 1 uM or more. The effective binding affinity of the multivalent D-peptidic compound as a whole may be optimized to provide for a desirable biological potency and/or other property such as in vivo half-life. By selecting individual D-peptidic domains having a particular individual affinities for their target binding site, the overall functional affinity of the multivalent D-peptidic compound can be optimized, as needed.

Potency of the compounds can be assessed using any convenient assays, such as via an ELISA assay measuring IC50 as described in the experimental section herein. In some instances, the subject multivalent compound has in vitro antagonist activity against the target protein that is at least 10-fold more potent, such as at least 30-fold, at least 100-fold, at least 300-fold, at least 1000-fold more potent, than the potency of each of the first and second D-peptidic domains alone.

In certain embodiments, the subject peptidic compounds specifically bind to VEGF-A target protein with high affinity, e.g., as determined by an SPR binding assay or an ELISA assay. The subject compounds may exhibit an affinity for VEGF-A of 1 uM or less, such as 300 nM or less, 100 nM or less, 30 nM or less, 10 nM or less, 5 nM or less, 2 nM or less, 1 nM or less, 600 pM or less, 300 pM or less, or even less.

The subject D-peptidic compounds may exhibit a specificity for VEGF-A, e.g., as determined by comparing the affinity of the compound for VEGF-A protein with that for a reference protein (e.g., an albumin protein), that is 5:1 or more 10:1 or more, such as 30:1 or more, 100: 1 or more, 300:1 or more, 1000:1 or more, or even more. In some cases, specificity can be a difference in binding affinities by a factor of 10³ or more, such as 10⁴ or more, 10⁵ or more, 10⁶ or more, or even more. In some cases, the peptidic compounds may be optimized for any desirable property, such as protein folding, protease stability, thermostability, compatibility with a pharmaceutical formulation, etc. Any convenient methods may be used to select the D-peptidic compounds, e.g., structure-activity relationship (SAR) analysis, affinity maturation methods, or phage display methods.

Also provided are D-peptidic compounds that have high thermal stability. In some cases, the compounds having high thermal stability have a melting temperature of 50° C. or more, such as 60° C. or more, 70° C. or more, 80° C. or more, or even 90° C. or more. Also provided are D-peptidic compounds that have high protease stability. The subject D-peptidic compounds are resistant to proteases and can have long serum and/or saliva half-lives. Also provided are D-peptidic compounds that have a long in vivo half-life. As used herein, “half-life” refers to the time required for a measured parameter, such the potency, activity and effective concentration of a compound to fall to half of its original level, such as half of its original potency, activity, or effective concentration at time zero. Thus, the parameter, such as potency, activity, or effective concentration of a polypeptide molecule is generally measured over time. For purposes herein, half-life can be measured in vitro or in vivo. In some cases, the peptidic compound has a half-life of 1 hour or longer, such as 2 hours or longer, 6 hours or longer, 12 hours or longer, 1 day or longer, 2 days or longer, 7 days or longer, or even longer. Stability in human blood may be measured by any convenient method, e.g., by incubating the compound in human EDTA blood or serum for a designated time, quenching a sample of the mixture and analyzing the sample for the amount and/or activity of the compound, e.g., by HPLC-MS, by an activity assay, e.g., as described herein.

Also provided are D-peptidic compounds that have low immunogenicity, e.g., are non-immunogenic. In certain embodiments, the D-peptidic compounds have low immunogenicity compared to an L-peptidic compound. In certain embodiments, the D-peptidic compounds are 10% or less, 20% or less, 30% or less, 40% or less, 50% or less, 70% or less, or 90% or less immunogenic compared to an L-peptidic compound, in an immunogenicity assay such as that described by Dintzis et al., “A Comparison of the Immunogenicity of a Pair of Enantiomeric Proteins” Proteins: Structure, Function, and Genetics 16:306-308 (1993).

Also provided are D-peptidic compounds that have been optimized for binding affinity and specificity to VEGF-A by affinity maturation, e.g., second generation D-peptidic compounds based on a parent compound that binds to VEGF-A. In some embodiments, the affinity maturation of a subject compound may include holding a fraction of the variant amino acid positions as fixed positions while the remaining variant amino acid positions are varied to select optimal amino acids at each position. A parent D-peptidic compound may be selected as a scaffold for an affinity maturation compound. In some cases, a number of affinity maturation compounds are prepared that include mutations at limited subsets of the variant amino acid positions of the parent, while the rest of the variant positions are held as fixed positions. The positions of the mutations may be tiled through the scaffold sequence to produce a series of compounds such that mutations at every variant position are represented and a diverse range of amino acids are substituted at every position (e.g., all 20 naturally occurring amino acids). Mutations that include deletion or insertion of one or more amino acids may also be included at variant positions of the affinity maturation compounds. An affinity maturation compound may be prepared and screened using any convenient method, e.g., phage display library screening, to identify second generation compounds having an improved property, e.g., increased binding affinity for a target molecule, protein folding, protease stability, thermostability, compatibility with a pharmaceutical formulation, etc.

In some embodiments, the affinity maturation of a subject compound may include holding most or all of the variant amino acid positions in the variable regions of the parent compound as fixed positions, and introducing contiguous mutations at positions adjacent to these variable regions. Such mutations may be introduced at positions in the parent compound that were previously considered fixed positions in the original GA scaffold domain. Such mutations may be used to optimize the compound variants for any desirable property, such as protein folding, protease stability, thermostability, compatibility with a pharmaceutical formulation, etc.

Aspects of the present disclosure include compounds (e.g., as described herein), salts thereof (e.g., pharmaceutically acceptable salts), and/or solvate, hydrate and/or prodrug forms thereof. It will be appreciated that all permutations of salts, solvates, hydrates, and prodrugs are meant to be encompassed by the present disclosure.

In some embodiments, the subject compounds, or a prodrug form thereof, are provided in the form of pharmaceutically acceptable salts. Compounds containing an amine or nitrogen containing heteraryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

### Multimeric Compounds

Any convenient D-peptidic compound (e.g., as described herein) may be multimerized, to provide a multimer of D-peptidic compounds. In certain embodiments, the multimer includes two or more D-peptidic compounds, such as 2 (e.g., a dimer), 3 (e.g., a trimer) or 4 or more compounds (e.g., a tetramer or a dendrimer, etc). In some cases, the multimer is described by the formula:

Y-(GA)ₙ

where: Y is a multivalent linking group; n is an integer greater than one; and GA is a D-peptidic compound comprising a GA domain motif (e.g., as described herein). In certain cases, n is 2. In certain cases, n is 3.

In certain cases, the multimer is a dimer of one of the formulae:

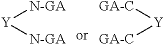

where each GA is independently a D-peptidic compound (e.g., as described herein); and Y is a linker connected to the N-terminal (N-GA) or the C-terminal (GA-C) of the compounds. In certain cases, the dimer is a homodimer of two identical GA domain motifs that each specifically bind VEGF-A. In certain instances, the dimer is a heterodimer. The heterodimer can be a dimer of two distinct GA domain motifs that each specifically bind VEGF-A, or a dimer of a subject D-peptidic compound and a second D-peptidic binding domain.

Any convenient linking groups can be utilized in the subject multimers. The terms “linker”, “linkage” and “linking group” are used interchangeably and refer to a linking moiety that covalently connects two or more compounds. In some cases, the linker is divalent. In certain cases, the linker is a branched or trivalent linking group. In some cases, the linker has a linear or branched backbone of 200 atoms or less (such as 100 atoms or less, 80 atoms or less, 60 atoms or less, 50 atoms or less, 40 atoms or less, 30 atoms or less, or even 20 atoms or less) in length. A linking moiety may be a covalent bond that connects two groups or a linear or branched chain of between 1 and 200 atoms in length, for example of about 1, 2, 3, 4, 5, 6, 8, 10, 12, 14, 16, 18, 20, 30, 40, 50, 100, 150 or 200 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, disulfide, amides, carbonates, carbamates, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. A linker may be peptidic, e.g., a linking sequence of residues.

Y can include any convenient group(s) or linker units, including but not limited to, amino acid residue(s), PEG, modified PEG (e.g., —NH(CH₂)ₘO[(CH₂)₂O]ₙ(CH₂)ₚCO— linking groups where m is 2-6, p is 1-6 and n is 1-50, such as 1-12 or 1-6), C2-C12 alkyl linkers, —CO—CH2CH2CO— units, and combinations thereof (e.g., linked via functional groups such as amide bonds, sulfonamide bonds, carbamates, ether bonds, ester bonds, or —NH—). In some instances, Y is peptidic. In some embodiments, Y is a linker comprising -(L1)a-(L2)b-(L3)c-(L4)d-(L5)e-, wherein L1, L2, L3, L4 and L5 are each a linker unit, and a, b, c, d and e are each independently 0 or 1, wherein the sum of a, b, c, d and e is 1 to 5. Other linkers are also possible, as shown in the multimeric compounds described herein.

In some instances, Y comprises a modified PEG linker that is connected to the D-peptidic compounds using any convenient linking chemistry. PEG is a polyethylene glycol or a modified polyethylene glycol. By modified PEG is meant that a polyethylene glycol or any convenient length where one or both of the terminals are modified to include a chemoselective functional group suitable for conjugation, e.g., to another linking group moiety or to the terminal or sidechain of a peptidic compound. Table 9 and and Examples section describe several exemplary homodimers of compound 1.1.1 (c21a) connected via either the N-terminals or C-terminals of the compounds. The D-peptidic compounds can be modified at the N- and/or C-terminals of the GA domain motifs to include one or more additional amino acid residues that can provide for a particular linkage or linking chemistry to connect to the Y group, such as a cysteine or a lysine.

Chemoselective reactive functional groups that may be utilized in linking the subject peptidic compounds via a linking group, include, but are not limited to: an amino group (e.g., a N-terminal amino or a lysine sidechain group), an azido group, an alkynyl group, a phosphine group, a thiol (e.g., a cysteine residue), a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

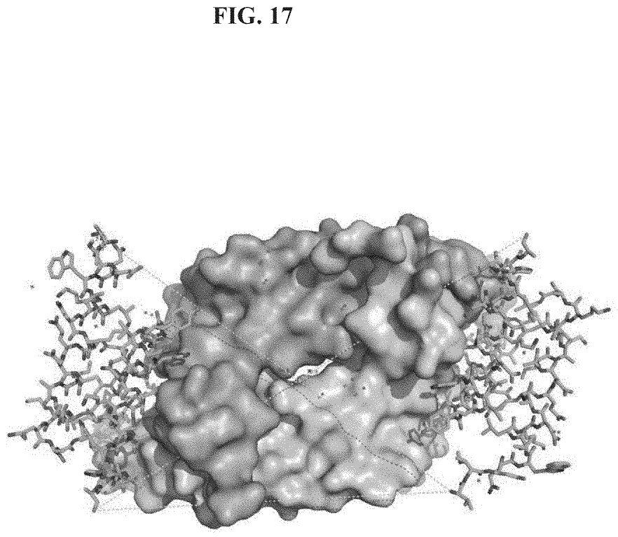

Any convenient multivalent linker may be utilized in the subject multimers. By multivalent is meant that the linker includes two or more terminal groups suitable for attachment to a subject compound, e.g., as described herein. In some cases, the multivalent linker is divalent or trivalent. In some instances, the multivalent linker Y is a dendrimer scaffold. Any convenient dendrimer scaffold may be adapted for use in the subject multimers. The dendrimer scaffold is a branched molecule that includes at least one branching point and two or more terminals suitable for connecting to the N-terminal or C-terminal of a GA domain motif via optional linkers. The dendrimer scaffold may be selected to provide a desired spatial arrangement of two or more GA domain motifs. In some cases, the spatial arrangement of the two or more GA domain motifs is selected to provide for a desired binding affinity and avidity for the target protein. FIG. 17 shows the X-ray crystal structure of compound 1.1.1 (c2 la) which includes a complex including two VEGF-A molecules and two compounds. In the view of the structure depicted the distances between the N-terminals (about 60 angstrom) and the C-terminals (about 70 angstrom) are marked by dotted lines. In some cases, the dimer includes a N-N linked Y group that is about 60 angstrom or more in length. In some cases, the dimer includes a C-C linked Y group that is about 70 angstrom or more in length.

In some cases, the D-peptidic compounds each independently include a specific binding moiety (e.g., a biotin or a peptide tag) where the D-peptidic compounds can be bound to each other via a multivalent binding moiety (e.g., a streptavidin, an avidin or an antibody) that specifically binds the specific binding moiety. In some embodiments, the two or more D-peptidic compounds, e.g., as described above, each include a specific binding moiety that is a biotin moiety. In certain embodiments, the specific binding moiety is a terminal biotin moiety, connected via an optional linker, to either the N-terminal or C-terminal of the compound. In certain cases, the terminal biotin moiety is Biotin-(Gly)ₙ- where n is 1 to 6 or Biotin-Ahx- (Ahx=6-aminohexanoic acid residue).

### Modified Compounds

Any convenient molecules or moieties of interest may be attached to the subject D-peptidic compounds. The molecule of interest may be peptidic or non-peptidic, naturally occurring or synthetic. Molecules of interest suitable for use in conjunction with the subject compounds include, but are not limited to, an additional protein domain, a polypeptide or amino acid residue, a peptide tag, a specific binding moiety, a polymeric moiety such as a polyethylene glycol (PEG), a carbohydrate, a dextran or a polyacrylate, a linker, a half-life extending moiety, a drug, a toxin, a detectable label and a solid support. In some cases, the molecule of interest may confer on the resulting peptidic compounds enhanced and/or modified properties and functions including, but not limited to, increased water solubility, ease of chemical synthesis, cost, bioconjugation site, stability, isoelectric point (pI), aggregation, reduced non-specific binding and/or specific binding to a second target protein, e.g., as described herein.

In some embodiments of any one of the VEGF-A binding GA domain motif sequences described herein, the motif may be extended to include one or more additional residues at the N-terminal and/or C-terminal of the sequence, such as two or more, three or more, four or more, five or more, 6 or more, or even more additional residues. Such additional residues may be considered part of the GA domain motif even though they do not provide a VEGF-A binding interaction. Any convenient residues may be included at the N-terminal and/or C-terminal of the VEGF-A binding GA domain motif to provide for a desirable property or group, such as increased solubility via a water soluble group, a linkage for dimerization or multimerization, a linkage for connecting to a label or a specific binding moiety.

In some cases, the subject modified compound is described by formula:

X-L-Z

where X is a VEGF-A binding GA domain motif (e.g., as described herein); L is an optional linking group; and Z is a molecule of interest, where L is attached to X at any convenient location (e.g., the N-terminal, C-terminal or via the sidechain of a surface residue not involved in binding to the target).

The D-peptidic compounds may include one or more molecules of interest, e.g., a N-terminal moiety and/or a C-terminal moiety. In some instances, the molecule of interest is covalently attached via the alpha-amino group of the N-terminal residue, or is covalently attached to the alpha-carboxyl acid group of the C-terminal residue. In other instances, an molecules of interest is attached to the motif via a sidechain group of a residue (e.g., via a c, k, d ore residue).

The molecules of interest may include a polypeptide or a protein domain. Polypeptides and protein domains of interest include, but are not limited to: gD tags, c-Myc epitopes, FLAG tags, His tags, fluorescence proteins (e.g., GFP), beta-galactosidase protein, GST, albumins, immunoglobulins, Fc domains, or similar antibody-like fragments, leucine zipper motifs, a coiled coil domain, a hydrophobic region, a hydrophilic region, a polypeptide comprising a free thiol which forms an intermolecular disulfide bond between two or more multimerization domains, a “protuberance-into-cavity” domain, beta-lactoglobulin, or fragments thereof.

The molecules of interest may include a half-life extending moiety. The term “half-life extending moiety” refers to a pharmaceutically acceptable moiety, domain, or “vehicle” covalently linked or conjugated to the subject compound, that prevents or mitigates in vivo proteolytic degradation or other activity-diminishing chemical modification of the subject compound, increases half-life or other pharmacokinetic properties (e.g., rate of absorption), reduces toxicity, improves solubility, increases biological activity and/or target selectivity of the subject compound with respect to a target of interest, increases manufacturability, and/or reduces immunogenicity of the subject compound, compared to an unconjugated form of the subject compound.

In certain embodiments, the half-life extending moiety is a polypeptide that binds a serum protein, such as an immunoglobulin (e.g., IgG) or a serum albumin (e.g., human serum albumin (HSA)). Polyethylene glycol is an example of a useful half-life extending moiety. Exemplary half-life extending moieties include a polyalkylene glycol moiety (e.g., PEG), a serum albumin or a fragment thereof, a transferrin receptor or a transferrin-binding portion thereof, and a moiety comprising a binding site for a polypeptide that enhances half-life in vivo, a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polysialic acid, a polyacetal, a long chain fatty acid, a long chain hydrophobic aliphatic group, an immunoglobulin Fc domain (see, e.g., U.S. Pat. No. 6,660,843), an albumin (e.g., human serum albumin; see, e.g., U.S. Pat. No. 6,926,898 and US 2005/0054051; U.S. Pat. No. 6,887,470), a transthyretin (TTR; see, e.g., US 2003/0195154; 2003/0191056), or a thyroxine-binding globulin (TBG).

An extended half-life can also be achieved via a controlled or sustained release dosage form of the subject compounds, e.g., as described by Gilbert S. Banker and Christopher T. Rhodes, Sustained and controlled release drug delivery system. In Modern Pharmaceutics, Fourth Edition, Revised and Expanded, Marcel Dekker, New York, 2002, 11. This can be achieved through a variety of formulations, including liposomes and drug-polymer conjugates.

In certain embodiments, the half-life extending moiety is a fatty acid. Any convenient fatty acids may be used in the subject modified compounds. See e.g., Chae et al., “The fatty acid conjugated exendin-4 analogs for type 2 antidiabetic therapeutics”, J. Control Release. 2010 May 21; 144(1):10-6.

In certain embodiments, the compound is modified to include a specific binding moiety. The specific binding moiety is a moiety that is capable of specifically binding to a second moiety that is complementary to it. In some cases, the specific binding moiety binds to the complementary second moiety with an affinity of at least 10⁻⁷M (e.g., as measured by a K_{D} of 100 nM or less, such as 30 nM or less, 10 nM or less, 3 nM or less, 1 nM or less, 300 pM or less, or 100 pM or even less). Complementary binding moiety pairs of specific binding moieties include, but are not limited to, a ligand and a receptor, an antibody and an antigen, complementary polynucleotides, complementary protein homo- or heterodimers, an aptamer and a small molecule, a polyhistidine tag and nickel, and a chemoselective reactive group (e.g., a thiol) and an electrophilic group (e.g., with which the reactive thiol group can undergo a Michael addition). The specific binding pairs may include analogs, derivatives and fragments of the original specific binding member. For example, an antibody directed to a protein antigen may also recognize peptide fragments, chemically synthesized, labeled protein, derivatized protein, etc. so long as an epitope is present. Protein domains of interest that find use as specific binding moieties include, but are not limited to, Fc domains, or similar antibody-like fragments, leucine zipper motifs, a coiled coil domain, a hydrophobic region, a hydrophilic region, a polypeptide comprising a free thiol which forms an intermolecular disulfide bond between two or more multimerization domains, or a “protuberance-into-cavity” domain (see e.g., WO 94/10308; U.S. Pat. No. 5,731,168, Lovejoy et al. (1993), Science 259: 1288-1293; Harbury et al. (1993), Science 262: 1401-05; Harbury et al. (1994), Nature 371:80-83; Hakansson et al. (1999), Structure 7: 255-64.

In certain embodiments, the molecule of interest is a linked specific binding moiety that specifically binds a target protein. The linked specific binding moiety can be an antibody, an antibody fragment, an aptamer or a second D-peptidic binding domain. The linked specific binding moiety can specifically bind any convenient target protein, e.g., a target protein that is desirable to target in conjunction with VEGF-A in the subject methods of treatment. Target proteins of interest include, but are not limited to, PDGF (e.g., PDGF-B), VEGF-B, VEGF-C, VEGF-D, EGF, EGFR, Her2, PD-1, PD-L1, OX-40 and LAG3. In certain instances, the linked specific binding moiety is a second D-peptidic binding domain that targets PDGF-B.

In certain embodiments, the specific binding moiety is an affinity tag such as a biotin moiety. Exemplary biotin moieties include biotin, desthiobiotin, oxybiotin, 2′-iminobiotin, diaminobiotin, biotin sulfoxide, biocytin, etc. In some cases, the biotin moiety is capable of specifically binding with high affinity to a chromatography support that contains immobilized avidin, neutravidin or streptavidin. Biotin moieties can bind to streptavidin with an affinity of at least 10⁻⁸M. In some cases, a monomeric avidin support may be used to specifically bind a biotin-containing compound with moderate affinity thereby allowing bound compounds to be later eluted competitively from the support (e.g., with a 2 mM biotin solution) after non-biotinylated polypeptides have been washed away. In certain instances, the biotin moiety is capable of binding to an avidin, neutravidin or streptavidin in solution to form a multimeric compound, e.g., a dimeric, or tetrameric complex of D-peptidic compounds with the avidin, neutravidin or streptavidin. A biotin moiety may also include a linker, e.g., -LC-biotin, -LC-LC-Biotin, -SLC-Biotin or -PEGₙ-Biotin where n is 3-12 (commercially available from Pierce Biotechnology). In certain embodiments, the compound is modified to include a detectable label. Examples of detectable labels include labels that permit both the direct and indirect measurement of the presence of the subject peptidic compound. Examples of labels that permit direct measurement of the compound include radiolabels, fluorophores, dyes, beads, nanoparticles (e.g., quantum dots), chemiluminescers, colloidal particles, paramagnetic labels and the like. Radiolabels may include radioisotopes, such as ³⁵S, ¹⁴C, ¹²⁵I, ³H, ⁶⁴Cu and ¹³¹I. The subject compounds can be labeled with the radioisotope using any convenient techniques, such as those described in Current Protocols in Immunology, Volumes 1 and 2, Coligen et al., Ed. Wiley-Interscience, New York, N.Y., Pubs. (1991), and radioactivity can be measured using scintillation counting or positron emission. Examples of detectable labels which permit indirect measurement of the presence of the modified compound include enzymes where a substrate may provide for a colored or fluorescent product. For example, the compound may include a covalently bound enzyme capable of providing a detectable product signal after addition of suitable substrate. Instead of covalently binding the enzyme to the compound, the compound may include a first member of specific binding pair which specifically binds with a second member of the specific binding pair that is conjugated to the enzyme, e.g. the compound may be covalently bound to biotin and the enzyme conjugate to streptavidin. Examples of suitable enzymes for use in conjugates include horseradish peroxidase, alkaline phosphatase, malate dehydrogenase and the like. Where not commercially available, such enzyme conjugates may be readily produced by any convenient techniques.

In certain embodiments, the detectable label is a fluorophore. The term “fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, QUASAR™ dyes etc.; dansyl derivatives; rhodamine dyes e. g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

In some embodiments, the compound includes a detectable label, such as a radiolabel. In certain embodiments, the radiolabel suitable for use in PET, SPECT and/or MR imaging. In certain embodiments, the radiolabel is a PET imaging label. In certain cases, the compound is radiolabeled with ¹⁸F, ⁶⁴Cu, ⁶⁸Ga, ⁹⁹mTc or ⁸⁶Y.

The detectable label may be attached to the peptidic compound at any convenient position and via any convenient chemistry. Methods and materials of interest include, but are not limited to those described by U.S. Pat. No. 8,545,809; Meares et al., 1984, Acc Chem Res 17:202-209; Scheinberg et al., 1982, Science 215:1511-13; Miller et al., 2008, Angew Chem Int Ed 47:8998-9033; Shirrmacher et al., 2007, Bioconj Chem 18:2085-89; Hohne et al., 2008, Bioconj Chem 19:1871-79; Ting et al., 2008, Fluorine Chem 129:349-58, the labeling method of Poethko et al. (J. Nucl. Med. 2004; 45: 892-902) in which 4-[18F]fluorobenzaldehyde is first synthesized and purified (Wilson et al, J. Labeled Compounds and Radiopharm. 1990; XXVIII: 1189-1199) and then conjugated to a peptide, labeling with succinimidyl [18F]fluorobenzoate (SFB) (e.g., Vaidyanathan et al., 1992, Int. J. Rad. Appl. Instrum. B 19:275), other acyl compounds (Tada et al., 1989, Labeled Compd. Radiopharm. XXVII:1317; Wester et al., 1996, Nucl. Med. Biol. 23:365; Guhlke et al., 1994, Nucl. Med. Biol 21:819), or click chemistry adducts (Li et al., 2007, Bioconj Chem. 18:1987).

Any convenient synthetic methods or bioconjugation methods may be utilized in preparing the subject modified D-peptidic compounds. In certain cases, the detectable label is connected to the compound via an optional linker. In certain embodiments, the detectable label is connected to the N-terminal of the compound. In certain embodiments, the detectable label is connected to the C-terminal of the compound. In certain embodiments, the detectable label is connected to a non-terminal residue of the compound, e.g., via a side chain moiety. In certain embodiments, the detectable label is connected to the N-terminal peptidic extension moiety of the compound via an optional linker. In some cases, the N-terminal peptidic extension moiety is modified to include a reactive functional group which is capable of reacting with a compatible functional group of a radiolabel containing moiety. Any convenient reactive functional groups, chemistries and radiolabel containing moieties may be utilized to attach a detectable label to the compound, including but not limited to, click chemistry, an azide, an alkyne, a cyclooctyne, copper-free click chemistry, a nitrone, a chelating group (e.g., selected from DOTA, TETA, NOTA, NODA, (tert-Butyl)₂NODA, NETA, C-NETA, L-NETA, S-NETA, NODA-MPAA, and NODA-MPAEM), a propargyl-glycine residue, etc.

In certain instances, the molecule of interest is a second active agent, e.g., an active agent or drug that finds use in conjunction with targeting VEGF-A in the subject methods of treatment. In certain instances, the molecule of interest is a small molecule, a chemotherapeutic, an antibody, an antibody fragment, an aptamer, or a L-protein. In some embodiments, the compound is modified to include a moiety that is useful as a pharmaceutical (e.g., a protein, nucleic acid, organic small molecule, etc.). Exemplary pharmaceutical proteins include, e.g., cytokines, antibodies, chemokines, growth factors, interleukins, cell-surface proteins, extracellular domains, cell surface receptors, cytotoxins, etc. Exemplary small molecule pharmaceuticals include small molecule toxins or therapeutic agents.

Any convenient therapeutic or diagnostic agent (e.g., as described herein) can be conjugated to a D-peptidic compound. A variety of therapeutic agents including, but not limited to, anti-cancer agents, antiproliferative agents, cytotoxic agents and chemotherapeutic agents are described below in the section entitled Combination Therapies, any one of which can be adapted for use in the subject modified compounds. Exemplary chemotherapeutic agents of interest include, for example, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, ABT-199. Any exemplary cytotoxic agents that find use in ADC can be adapted for use in the subject modified D-peptidic compounds. Cytotoic agents of interest include, but are not limited to, auristatins (e.g., MMAE, MMAF), maytansines, dolastatins, calicheamicins, duocarmycins, pyrrolobenzodiazepines (PBDs), centanamycin (ML-970; indolecarboxamide), doxorubicin, α-Amanitin, and derivatives and analogs thereof.In certain embodiments, the compound may include a cell penetrating peptide (e.g., tat). The cell penetrating peptide may facilitate cellular uptake of the molecule. Any convenient tag polypeptides and their respective antibodies may be used. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

In certain embodiments, the compound may include a cell penetrating peptide (e.g., tat). The cell penetrating peptide may facilitate cellular uptake of the molecule. Any convenient tag polypeptides and their respective antibodies may be used. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

The molecules of interest may be attached to the subject modified compounds via any convenient method. In some cases, a molecules of interest is attached via covalent conjugation to a terminal amino acid residue, e.g., at the amino terminal or at the carboxylic acid terminal. The molecule of interest may be attached to the peptidic GA domain motif via a single bond or a suitable linker, e.g., a PEG linker, a peptidic linker including one or more amino acids, or a saturated hydrocarbon linker. A variety of linkers (e.g., as described herein) find use in the subject modified compounds. Any convenient reagents and methods may be used to include a molecule of interest in a subject GA domain motif, for example, conjugation methods as described in G. T. Hermanson, “Bioconjugate Techniques” Academic Press, 2nd Ed., 2008, solid phase peptide synthesis methods, or fusion protein expression methods. Functional groups that may be used in covalently bonding the domain, via an optional linker, to produce the modified compound include: hydroxyl, sulfhydryl, amino, and the like. Certain moieties on the molecules of interest and/or GA domain motif may be protected using convenient blocking groups, see, e.g. Green & Wuts, Protective Groups in Organic Synthesis (John Wiley & Sons) 3rd Ed. (1999). The particular molecule of interest and site of attachment to the GA domain motif may be chosen so as not to substantially adversely interfere with the desired binding activity, e.g. for the target VEGF-A protein.

The molecule of interest may be peptidic. It is understood that a molecule of interest may further include one or more non-peptidic groups including, but not limited to, a biotin moiety and/or a linker. Any convenient protein domains may be adapted and utilized as molecules of interest in the subject modified peptidic compounds. Protein domains of interest include, but are not limited to, any convenient serum protein, serum albumin (e.g., human serum albumin; see, e.g., U.S. Pat. No. 6,926,898 and US 2005/0054051; U.S. Pat. No. 6,887,470), a transferrin receptor or a transferrin-binding portion thereof, immunoglobulin (e.g., IgG), an immunoglobulin Fc domain (see, e.g., U.S. Pat. No. 6,660,843), a transthyretin (TTR; see, e.g., US 2003/0195154; 2003/0191056), a thyroxine-binding globulin (TBG), or a fragment thereof.

A multimerizing group is any convenient group that is capable of forming a multimer (e.g., a dimer, a trimer, or a dendrimer), e.g., by mediating binding between two or more compounds (e.g., directly or indirectly via a multivalent binding moiety), or by connecting two or more compounds via a covalent linkage. In some cases, the multimerizing group Z is a chemoselective reactive functional group that conjugates to a compatible function group on a second D-peptidic compound. In other cases, the multimerizing group is a specific binding moiety (e.g., biotin or a peptide tag) that specifically binds to a multivalent binding moiety (e.g., a streptavidin or an antibody). In some cases, the compound includes a multimerizing group and is a monomer that has not yet been multimerized.

Chemoselective reactive functional groups for inclusion in the subject peptidic compounds, include, but are not limited to: an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

### Polynucleotides

Also provided are polynucleotides that encode a sequence corresponding to the subject peptidic compounds as described herein. The polynucleotide can encode a L-peptidic compound that specifically binds to a D-VEGF-A target protein.

In some embodiments, the polynucleotide encodes a peptidic compound that includes between 30 and 80 residues, between 40 and 70 residues, between 45 and 60 residues, between 45 and 60 residues, or between 45 and 55 residues. In certain instances, the polynucleotide encodes a peptidic compound sequence of between 35 and 55 residues, such as between 40 and 55 residues, or between 45 and 55 residues. In certain embodiments, the polynucleotide encodes a peptidic compound sequence of 45, 46, 47, 48, 49, 50, 51, 52 or 53 residues.

In certain embodiments, the polynucleotide is a replicable expression vector that includes a nucleic acid sequence encoding a L-peptidic compound that may be expressed in a protein expression system. In certain embodiments, the polynucleotide is a replicable expression vector that includes a nucleic acid sequence encoding a gene fusion, where the gene fusion encodes a fusion protein including the L-peptidic compound fused to all or a portion of a viral coat protein.

In certain embodiments, the subject polynucleotides are capable of being expressed and displayed in a cell-based or cell-free display system. Any convenient display methods may be used to display L-peptidic compounds encoded by the subject polynucleotides, such as cell-based display techniques and cell-free display techniques. In certain embodiments, cell-based display techniques include phage display, bacterial display, yeast display and mammalian cell display. In certain embodiments, cell-free display techniques include mRNA display and ribosome display.

### Methods

The herein-described compounds may be employed in a variety of methods. One such method includes contacting a subject compound with a VEGF-A target protein under conditions suitable for binding of VEGF-A to produce a complex. In some embodiments, the method includes administering a D-peptidic compound to a subject, where the compound binds to VEGF-A in the subject.

A subject compound may inhibit at least one activity of its VEGF-A target in the range of 10% to 100%, e.g., by 10% or more, 20% or more, 30% or more, 40% or more, 50% or more, 60% or more, 70% or more, 80% or more, or 90% or more. In certain assays, a subject compound may inhibit its VEGF-A target with an IC₅₀ of 1×10⁻⁵ M or less (e.g., 1×10⁻⁶ M or less, 1×10⁻⁷M or less, 1×10⁻⁸M or less, 1×10⁻⁹ M or less, 1×10⁻¹⁰ M or less, or 1×10⁻¹¹ M or less). In certain assays, a subject compound may inhibit its VEGF-A target with an IC₂₀ of 1×10⁻⁶ M or less (e.g., 500 nM or less, 200 nM or less, 100 nM or less, 30 nM or less, 10 nM or less, 3 nM or less, or 1 nM or less). In certain assays, a subject compound may inhibit its VEGF-A target with an IC₁₀ of 1×10⁻⁶ M or less (e.g., 500 nM or less, 200 nM or less, 100 nM or less, 30 nM or less, 10 nM or less, 3 nM or less, or 1 nM or less). In assays in which a mouse is employed, a subject compound may have an ED50 of less than 1 μg/mouse (e.g., 1 ng/mouse to about 1 μg/mouse).

In some embodiments, the subject method is an in vitro method that includes contacting a sample with a subject compound that specifically binds with high affinity to a target molecule. In certain embodiments, the sample is suspected of containing the target molecule and the subject method further comprises evaluating whether the compound specifically binds to the target molecule. In certain embodiments, the target molecule is a naturally occurring L-protein and the compound is D-peptidic. In certain embodiments, the subject compound is a modified compound that includes a label, e.g., a fluorescent label, and the subject method further includes detecting the label, if present, in the sample, e.g., using optical detection. In certain embodiments, the compound is modified with a support, such that any sample that does not bind to the compound may be removed (e.g., by washing). The specifically bound target protein, if present, may then be detected using any convenient means, such as, using the binding of a labeled target specific probe or using a fluorescent protein reactive reagent. In another embodiment of the subject method, the sample is known to contain the target protein. In certain embodiments, the target VEGF-A protein is a synthetic D-protein and the compound is L-peptidic. In certain embodiments, the target VEGF-A protein is a L-protein and the compound is D-peptidic.

In certain embodiments, a subject compound may be contacted with a cell in the presence of VEGF-A, and a VEGF-A response phenotype of the cell monitored. Exemplary VEGF-A assays include assays using isolated protein in cell free systems, in vitro using cultured cells or in vivo assays. Exemplary VEGF-A assays include, but are not limited to a receptor tyrosine kinase inhibition assay (see, e.g., Cancer Research Jun. 15, 2006; 66:6025-6032), an in vitro HUVEC proliferation assay (FASEB Journal 2006; 20: 2027-2035; Wells et al., Biochemistry 1998, 37, 17754-17764), an in vivo solid tumor disease assay (U.S. Pat. No. 6,811,779) and an in vivo angiogenesis assay (FASEB Journal 2006; 20: 2027-2035). The descriptions of these assays are hereby incorporated by reference. The protocols that may be employed in these methods are numerous and include, but are not limited to cell-free assays, e.g., binding assays; cellular assays in which a cellular phenotype is measured, e.g., gene expression assays; and in vivo assays that involve a particular animal (which, in certain embodiments may be an animal model for a condition related to the target). In certain cases, the assay may be a vascularization assay. In certain embodiments, the target protein is VEGF-A and the subject compound inhibits VEGF-A dependent angiogenesis. In certain embodiments, the target protein is VEGF-A and the subject compound inhibits VEGF-A dependent cellular proliferation. In certain instances, the target protein is VEGF-A and the compound inhibits VEGFR2 phosphorylation.

In some embodiments, the subject method is in vivo and includes administering to a subject a D-peptidic compound that specifically binds with high affinity to a target molecule. In certain embodiments, the compound is administered as a pharmaceutical preparation. A variety of subjects are treatable according to the subject methods. Generally such subjects are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some embodiments, the subject is human. The subject can be a subject in need of prevention of treatment of a disease or condition associated with angiogenesis in a subject (e.g., as described herein).

The subject compounds can bind to and inhibit VEGF-A and may thus be useful for treatment, in vivo diagnosis and imaging of diseases and conditions associated with angiogenesis. The term “diseases and conditions associated with angiogenesis” includes, but is not limited to, those diseases and conditions referred to herein. Reference is also made in this regard to WO 98/47541. Diseases and conditions associated with angiogenesis include different forms of cancer and metastasis, for example, breast, skin, colorectal, pancreatic, prostate, lung or ovarian cancer. Other diseases and conditions associated with angiogenesis are inflammation (for example, chronic inflammation), atherosclerosis, rheumatoid arthritis and gingivitis. Further diseases and conditions associated with angiogenesis are arteriovenous alformations, astrocytomas, choriocarcinomas, glioblastomas, gliomas, hemangiomas (childhood, capillary), hepatomas, hyperplastic endometrium, ischemic myocardium, endometriosis, Kaposi sarcoma, macular degeneration, melanoma, neuroblastomas, occluding peripheral artery disease, osteoarthritis, psoriasis, retinopathy (diabetic, proliferative), scleroderma, seminomas and ulcerative colitis. In some cases, the disease or condition associated with angiogenesis is cancer (e.g., breast, skin, colorectal, pancreatic, prostate, lung or ovarian cancer), an inflammatory disease, atherosclerosis, rheumatoid arthritis, macular degeneration and retinopathy. Of particular interest is treatment of diabetic macular edema (DME) or age-related macular degeneration (AMD).

The VEGF-A binding subject compounds are useful in the treatment of various neoplastic and non-neoplastic diseases and disorders. Neoplasms and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Non-neoplastic conditions that are amenable to treatment include rheumatoid arthritis, psoriasis, atherosclerosis, diabetic and other proliferative retinopathies including retinopathy of prematurity, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

The term “treating” or “treatment” as used herein means the treating or treatment of a disease or medical condition in a patient, such as a mammal (such as a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient. As such, treatment also includes situations where the pathological condition, or at least symptoms associated therewith, are completely inhibited, e.g., prevented from happening, or stopped, e.g., terminated, such that the subject no longer suffers from the pathological condition, or at least the symptoms that characterize the pathological condition. Treatment may also manifest in the form of a modulation of a surrogate marker of the disease condition, e.g., as described above.

Aspects of the present disclosure include methods of preventing or treating AMD, such as wet age-related macular degeneration (AMD). Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the subject VEGF-binding compounds find use in reducing the severity of AMD. In certain instances, the subject is a patient suffering from dry AMD and administration of a compound according to the subject methods prevents the occurrence, or reduces the severity, of wet AMD in the subject.

In certain embodiments, the subject methods include administering a compound, such as a VEGF-A binding compound, and then detecting the compound after it has bound to its target protein. In some methods, the same compound can serve as both a therapeutic and a diagnostic compound.

The VEGF-A binding compounds of the present disclosure are therapeutically useful for treating any disease or condition which is improved, ameliorated, inhibited or prevented by removal, inhibition, or reduction of a VEGF-A protein, or a fragment thereof.

In some embodiments, the subject method is a method of modulating angiogenesis in a subject, the method comprising administering to the subject an effective amount of a subject compound that specifically binds with high affinity to a VEGF-A protein. In certain embodiments, the method further comprises diagnosing the presence of a disease condition in the subject. In certain embodiments, the disease condition is a condition that may be treated by enhancing angiogenesis. In certain embodiments, the disease condition is a condition that may be treated by decreasing angiogenesis. In certain embodiments, the subject method is a method of inhibiting angiogenesis and the compound is a VEGF-A antagonist.

In some embodiments, the subject method is a method of treating a subject suffering from a cellular proliferative disease condition, the method including administering to the subject an effective amount of a subject compound that specifically binds with high affinity to a VEGF-A protein so that the subject is treated for the cellular proliferative disease condition.

In some embodiments, the subject method is a method of inhibiting tumor growth in a subject, the method comprising administering to a subject an effective amount of a subject compound that specifically binds with high affinity to the VEGF-A protein. In certain embodiments, the tumor is a solid tumor. In certain embodiments, the tumor is a non-solid tumor.

The amount of compound administered can be determined using any convenient methods to be an amount sufficient to produce the desired effect in association with a pharmaceutically acceptable diluent, carrier or vehicle. The specifications for the unit dosage forms of the present disclosure will depend on the particular compound employed and the effect to be achieved, and the pharmacodynamics associated with each compound in the subject.

In some embodiments, an effective amount of a subject compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a subject compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of the subject compound is administered. In other embodiments, multiple doses of the subject compound are administered. Where multiple doses are administered over a period of time, the D-peptidic compound is administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed for the presence and/or extent of angiogenesis. Assessment of the effectiveness of the methods of treatment on the subject can include assessment of the subject before, during and/or after treatment, using any convenient methods. Aspects of the subject methods further include a step of assessing the therapeutic response of the subject to the treatment.

In some embodiments, the method includes assessing the condition of the subject, including diagnosing or assessing one or more symptoms of the subject which are associated with the disease or condition of interest being treated (e.g., as described herein). In some embodiments, the method includes obtaining a biological sample from the subject and assaying the sample, e.g., for the presence of angiogenesis that is associated with the disease or condition of interest (e.g., as described herein). The sample can be a cellular sample. In some cases, the sample is a biopsy. The assessment step(s) of the subject method can be performed at one or more times before, during and/or after administration of the subject compounds, using any convenient methods.

In some cases, a subject compound or a salt thereof, e.g., as defined herein, finds use in medicine, particularly in the in vivo diagnosis or imaging, for example by PET, of a disease or condition associated with angiogenesis. In certain embodiments, the compound is a modified compound that includes a detectable label, and the method further includes detecting the label in the subject. The selection of the label depends on the means of detection. Any convenient labeling and detection systems may be used in the subject methods, see e.g., Baker, “The whole picture,” Nature, 463, 2010, p977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabelled and a secondary labeling agent is used for imaging. In certain embodiments, the subject methods include diagnosis of a disease condition in a subject by comparing the number, size, and/or intensity of labeled loci, to corresponding baseline values. The base line values can represent the mean levels in a population of undiseased subjects, or previous levels determined in the same subject.

In some cases, radiolabelled compounds may be administered to subjects for PET imaging in amounts sufficient to yield the desired signal. In certain instances, the radionuclide dosage is of 0.01 to 100 mCi, such as 0.1 to 50 mCi, or 1 to 20 mCi, which is sufficient per 70 kg bodyweight. The radiolabelled compounds may therefore be formulated for administration using any convenient physiologically acceptable carriers or excipients. For example, the compounds, optionally with the addition of pharmaceutically acceptable excipients, may be suspended or dissolved in an aqueous medium, with the resulting solution or suspension then being sterilized. Also provided is the use of a radiolabelled compound or a salt thereof as described herein for the manufacture of a radiopharmaceutical for use in a method of in vivo imaging, e.g., PET imaging, such as imaging of a disease or condition associated with angiogenesis; involving administration of the radiopharmaceutical to a human or animal body and generation of an image of at least part of said body.

In some embodiments, the method is a method for in vivo diagnosis or imaging of a disease or condition associated with angiogenesis involving administering a radiopharmaceutical to said body, e.g. into the vascular system and generating an image of at least a part of said body to which said radiopharmaceutical has distributed using PET, wherein said radiopharmaceutical comprises a radiolabelled compound or a salt thereof.

In some embodiments, the method is a method of monitoring the effect of treatment of a human or animal body with a drug, e.g., a cytotoxic agent, to combat a condition associated with angiogenesis e.g., cancer, said method comprising administering to said body a radiolabelled compound or a salt thereof and detecting the uptake of the compound by cell receptors, such as endothelial cell receptors, e.g., alpha.v.beta.3 receptors, the administration and detection optionally being effected repeatedly, e.g. before, during and after treatment with said drug.

In some embodiments, the method is a method for in vivo diagnosis or imaging of a disease or condition associated with angiogenesis comprising administering to a subject a D-peptidic compound and imaging at least a part of the subject. In certain embodiments, the imaging comprises PET imaging and the administering comprises administering the compound to the vascular system of the subject. In some instances, the method further comprising detecting uptake of the compound by cell receptors. In certain instances, the target is VEGF-A and the subject is human. In certain embodiments, the method includes administering a therapeutic antibody, e.g., avastin, to the subject, wherein the disease or condition is a condition associated with cancer.

The subject methods may be diagnostic methods for detecting the expression of a target protein in specific cells, tissues, or serum, in vitro or in vivo. In some cases, the subject method is a method for in vivo imaging of a target protein in a subject. The methods may include administering the compound to a subject presenting with symptoms of a disease condition related to a target protein. In some cases, the subject is asymptomatic. The subject methods may further include monitoring disease progression and/or response to treatment in subjects who have been previously diagnosed with the disease.

The subject VEGF-A binding compounds may be used as affinity purification agents. In this process, the compounds are immobilized on a solid phase such a Sephadex resin or filter paper, using any convenient methods. The subject VEGF-A binding compound is contacted with a sample containing the VEGF-A protein (or fragment thereof) to be purified, and thereafter the support is washed with a suitable solvent that will remove substantially all the material in the sample except the VEGF protein, which is bound to the immobilized compound. Finally, the support is washed with another suitable solvent, such as glycine buffer, pH 5.0 that will release the VEGF-A protein from the immobilized compound.

The subject VEGF-A binding compounds may also be useful in diagnostic assays for VEGF-A protein, e.g., detecting its expression in specific cells, tissues, or serum. Such diagnostic methods may be useful in cancer diagnosis. For diagnostic applications, the subject compound may be modified as described above.

### Combination Therapies

In some embodiments, the subject compounds may be administered in combination with one or more additional active agents or therapies. Any convenient agents may be utilized, including compounds useful for treating diseases that are targeted by the subject methods. The terms “agent,” “compound,” and “drug” are used interchangeably herein. Additional active agents or therapies include, but are not limited to, a small molecule, an antibody, an antibody fragment, an aptamer, a L-protein, a second target-binding molecule such as a second D-peptidic compound, a chemotherapeutic agent, surgery, catheter devices, and radiation. Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and a cytotoxic agent, a chemotherapeutic agent or a growth inhibitory agent can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

The terms “co-administration” and “in combination with” include the administration of two or more therapeutic agents (e.g., a D-peptidic compound and a second agent) either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent (e.g., a D-peptidic compound) can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the D-peptidic compound and second agent at such time that both the known drug and the composition of the present disclosure will have a therapeutic effect. Such concomitant administration may involve concurrent (i.e. at the same time), prior, or subsequent administration of the drug with respect to the administration of a subject D-peptidic compound. Routes of administration of the two agents may vary, where representative routes of administration are described in greater detail below. A person of ordinary skill in the art would have no difficulty determining the appropriate timing, sequence and dosages of administration for particular drugs and compounds of the present disclosure.

In some embodiments, the compounds (e.g., a subject D-peptidic compound and a second agent) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

Dosage levels of the order of from about 0.01 mg to about 140 mg/kg of body weight per day are useful in representative embodiments, or alternatively about 0.5 mg to about 7 g per patient per day. Those of skill will readily appreciate that dose levels can vary as a function of the specific compound, the severity of the symptoms and the susceptibility of the subject to side effects. Dosages for a given compound are readily determinable by those of skill in the art by a variety of means.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, such as 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Any convenient second agents can find use in the subject methods. In some cases, the second active agent specifically binds a target protein selected from platelet-derived growth factor (PDGF), VEGF-B, VEGF-C, VEGF-D, EGF, EGFR, Her2, PD-1, PD-L1, OX-40, LAG3, Ang2, IL-1, IL-6 and IL-17. Second active agents of interest include, but are not limited to, pegpleranib (Fovista), ranibizumab (Lucentis), trastuzumab (Herceptin), Bevacizumab (Avastin), aflibercept (Eylea), nivolumab, atezolizumab, Durvalumab, gefitinib, erlotinib and Pembrolizumab.

For the treatment of cancer, the subject compounds can be administered in combination with a chemotherapeutic agent selected from the group consisting of taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine) and other cell therapies. Specific chemotherapeutic agents include, for example, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, ABT-199.

For the treatment of cancer (e.g., melanoma, non-small cell lung cancer or a lymphoma such as Hodgkin's lymphoma), the subject compounds can be administered in combination with an immune checkpoint inhibitor. Any convenient checkpoint inhibitors can be utilized, including but not limited to, cytotoxic T-lymphocyte-associated antigen 4 (CTLA-4) inhibitors, programmed death 1 (PD-1) inhibitors and PD-L1 inhibitors. Exemplary checkpoint inhibitors of interest include, but are not limited to, ipilimumab, pembrolizumab and nivolumab. In certain embodiments, for treatment of cancer and/or inflammatory disease, the subject compounds can be administered in combination with a colony-stimulating factor-1 receptor (CSF1R) inhibitors. CSF1R inhibitors of interest include, but are not limited to, emactuzumab.

Any convenient cancer vaccine therapies and agents can be used in combination with the subject immunomodulatory polypeptide compositions and methods. For treatment of cancer, e.g., ovarian cancer, the subject compounds can be administered in combination with a vaccination therapy, e.g., a dendritic cell (DC) vaccination agent that promotes Th1/Th17 immunity. Th17 cell infiltration correlates with markedly prolonged overall survival among ovarian cancer patients. In some cases, the immunomodulatory polypeptide finds use as adjuvant treatment in combination with Th17-inducing vaccination.

Also of interest are agents that are CARP-1/CCAR1 (Cell division cycle and apoptosis regulator 1) inhibitors, including but not limited to those described by Rishi et al., Journal of Biomedical Nanotechnology, Volume 11, Number 9, September 2015, pp. 1608-1627(20), and CD47 inhibitors, including, but not limited to, anti-CD47 antibody agents such as Hu5F9-G4.

### Utility

The compounds of the invention, e.g., as described above, find use in a variety of applications. Applications of interest include, but are not limited to: therapeutic applications, research applications, and screening applications. Each of these different applications are now reviewed in greater details below.

### Therapeutic Applications

The subject compounds find use in a variety of therapeutic applications. Therapeutic applications of interest include those applications in which the activity of the target is the cause or a compounding factor in disease progression. As such, the subject compounds find use in the treatment of a variety of different conditions in which the modulation of target activity in the host is desired.

The subject compounds are useful for treating a disorder relating to its target, VEGF-A. Examples of disease conditions which may be treated with compounds of the invention are described above.

In certain embodiments, the disease conditions include, but are not limited to: cancer, inhibition of angiogenesis and metastasis, osteoarthritis pain, chronic lower back pain, cancer-related pain, age-related macular degeneration (AMD), diabetic macular edema (DME), idiopathic pulmonary fibrosis (IPF) and graft survival of transplanted corneas.

In one embodiment, the present disclosure provides a method of treating a subject for a VEGF-A-related condition. The method generally involves administering a subject compound to a subject having a VEGF-A related disorder in an amount effective to treat at least one symptom of the VEGF-A related disorder. VEGF-A related conditions are generally characterized by excessive vascular endothelial cell proliferation, vascular permeability, edema or inflammation such as brain edema associated with injury, stroke or tumor; edema associated with inflammatory disorders such as psoriasis or arthritis, including rheumatoid arthritis; asthma; generalized edema associated with burns; ascites and pleural effusion associated with tumors, inflammation or trauma; chronic airway inflammation; capillary leak syndrome; sepsis; kidney disease associated with increased leakage of protein; and eye disorders such as age related macular degeneration and diabetic retinopathy. Such conditions include breast, lung, colorectal and renal cancer.

### Research Applications

The subject compounds and methods find use in a variety of research applications. The subject compounds and methods may be used to analyze the roles of target proteins in modulating various biological processes, including but not limited to, angiogenesis, inflammation, cellular growth, metabolism, regulation of transcription and regulation of phosphorylation. Other target protein binding molecules such as antibodies have been similarly useful in similar areas of biological research. See e.g., Sidhu and Fellhouse, “Synthetic therapeutic antibodies,” Nature Chemical Biology, 2006, 2(12), 682-688. Such methods can be readily modified for use in a variety of research applications of the subject compounds and methods.

### Diagnostic Applications

The subject compounds and methods find use in a variety of diagnostic applications, including but not limited to, the development of clinical diagnostics, e.g., in vitro diagnostics or in vivo tumor imaging agents. Such applications are useful in diagnosing or confirming diagnosis of a disease condition, or susceptibility thereto. The methods are also useful for monitoring disease progression and/or response to treatment in patients who have been previously diagnosed with the disease.

Diagnostic applications of interest include diagnosis of disease conditions, such as those conditions described above, including but not limited to: cancer, inhibition of angiogenesis and metastasis, osteoarthritis pain, chronic lower back pain, cancer-related pain, age-related macular degeneration (AMD), diabetic macular edema (DME), ideopathic pulmonary fibrosis (IPF) and graft survival of transplanted corneas. In some methods, the same compound can serve as both a treatment and diagnostic reagent.

Other target protein binding molecules, such as aptamers and antibodies, have also found use in the development of clinical diagnostics. Such methods can be readily modified for use in a variety of diagnostics applications of the subject compounds and methods, see for example, Jayasena, “Aptamers: An Emerging Class of Molecules That Rival Antibodies in Diagnostics,” Clinical Chemistry, 1999, 45, 1628-1650.

### Pharmaceutical Preparations

Also provided are pharmaceutical preparations. Pharmaceutical preparations are compositions that include a compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention. Administration of compounds of the present disclosure may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compounds and compositions of the invention are administered orally. In certain embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The subject compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

A compound of the present disclosure may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. A compound of the invention may also be incorporated into existing nutraceutical formulations, such as are available conventionally, which may also include an herbal extract.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the compounds may be administered in the form of a free base, their pharmaceutically acceptable salts, or they may also be used alone or in appropriate association, as well as in combination, with other pharmaceutically active compounds.

In some embodiments, a pharmaceutical composition includes a subject compound that specifically binds with high affinity to a target protein, and a pharmaceutically acceptable vehicle. In certain embodiments, the target protein is a VEGF protein and the subject compound is a VEGF antagonist.

### Kits

Also provided are kits that include compounds of the present disclosure. Kits of the present disclosure may include one or more dosages of the compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat cellular conditions associated with pathogenic angiogenesis. The term kit refers to a packaged active agent or agents. In some embodiments, the subject system or kit includes a dose of a subject compound (e.g., as described herein) and a dose of a second active agent (e.g., as described herein) in amounts effective to treat a subject for a disease or condition associated with angiogenesis (e.g., as described herein).

In addition to the above-mentioned components, a subject kits may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, a kit includes a first dosage of a subject pharmaceutical composition and a second dosage of a subject pharmaceutical composition. In certain embodiments, the kit further includes a second angiogenesis modulatory agent.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. §112 are to be accorded full statutory equivalents under 35 U.S.C. § 112.

### Definitions

The term “peptidic” refers to a moiety that is composed primarily of amino acid residues linked together as a polypeptide. The term “peptidic” is meant to include compounds in which one, two or more residues of a conventional polypeptide sequence have been replaced with a peptidomimetic. A peptidomimetic is a small organic group designed to mimic a peptide or amino acid residue. A peptidomimetic group of a peptidic moiety can include a non-naturally occurring or synthetic backbone group linked to the conventional polypeptide backbone and an optional sidechain group that mimics the sidechain group of any convenient amino acid residue of interest. In some embodiments, a peptidic compound that is composed primarily of amino acid residues has 2 residues or less per 10 amino acid residues of a parent polypeptide sequence replaced with a peptidomimetic moiety. Any convenient peptidomimetic groups and chemistries can be utilized in the subject peptidic compounds. The term peptidic is also meant to include multimeric peptidic compounds where two or more peptidic compounds of interest are covalently linked. The term peptidic is also meant to include modified peptidic compounds where a non-proteinaceous moiety has been covalently linked to the compound.

The terms “polypeptide,” “peptide,” and “protein” are used interchangeably to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The terms include polypeptides in which one or more conventional amino acids have been replaced with non-naturally occurring or synthetic amino acids. A polypeptide may be of any length, e.g., 2 or more amino acids, 4 or more amino acids, 10 or more amino acids, 20 or more amino acids, 30 or more amino acids, 40 or more amino acids, 50 or more amino acids, 60 or more amino acids, 100 or more amino acids, 300 or more amino acids, 500 or more or 1000 or more amino acids.

For the polypeptide sequences and motifs depicted herein, unless noted otherwise, capital letter codes refer to L-amino acid residues and small letter codes refer to D-amino acid residues. The amino acid residue glycine is represented as G or Gly. “a” is alanine. “c” is cysteine. “d” is aspartic acid. “e” is glutamic acid. “f” is phenylalanine “h” is histidine. “i” is isoleucine. “k” is lysine. “1” is leucine. “m” is methionine. “n” is asparagine. “o” is ornithine. “p” is proline. “q” is glutamine. “r” is arginine “s” is serine. “t” is threonine. “v” is valine. “w” is tryptophan. “y” is tyrosine. It is understood that for any of the sequences and motifs described herein, e.g., sequences defining a peptidic compound that specifically binds VEGF-A, a mirror image compound is also encompassed which specifically binds to the mirror image of VEGF-A. The present disclosure is meant to encompass both versions of the subject compounds, e.g., L-peptidic compounds that specifically bind D-VEGF-A and D-peptidic compounds that specifically bind L-VEGF-A. It is understood that D-VEGF-A protein may be targeted primarily in a variety of in vitro applications, while L-VEGF-A protein may be targeted for a variety of in vitro and/or in vivo applications.

The term “analog” of an amino acid residue refers to a residue having a sidechain group that is a structural and/or functional analog of the sidechain group of the reference amino acid residue. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as F, Cl or Br, etc.), deletion of a group, substitution of a covalent bond (single bond for double bond, etc.), or combinations thereof. For example, amino acid analogs may include α-hydroxy acids, and α-amino acids, and the like. In some cases, an analog of an amino acid residue is a substituted version of the amino acid. The term “substituted version” of an amino acid residue refers to a residue having a sidechain group that includes one or more additional substituents on the sidechain group that are not present in the sidechain of the reference amino acid residue.

The terms “aromatic amino acid” and “aromatic residue” are used interchangeably to refer to an amino acid residue where the sidechain group includes an aryl, a substituted aryl, a heteroaryl or a substituted heteroaryl group. In some cases, the sidechain group is an aryl-alkyl, a substituted aryl-alkyl, a heteroaryl-alkyl or a substituted heteroaryl-alkyl group. The terms are meant to include naturally occurring and non-naturally occurring alpha-amino acids. Naturally occurring aromatic residues of interest include phenylalanine, tyrosine, tryptophan and histidine.

The terms “carbocyclic amino acid” and “carbocyclic residue” are used interchangeably to refer to an amino acid residue where the sidechain group includes an aryl or a saturated carbocyclic group. In some cases, the sidechain group is an cycloalkyl-alkyl or a substituted cycloalkyl-alkyl group. Non-naturally occurring sidechain groups of interest include, but are not limited to, cyclohexyl-CH₂—, cyclopentyl-CH₂, cyclohexyl-(CH₂)₂— and cyclopentyl-(CH₂)₂—.

The terms “heterocyclic amino acid” and “heterocyclic residue” are used interchangeably to refer to an amino acid residue where the sidechain group includes a heterocyclic group, such as a heteroaryl group or a saturated heterocyclic group. In some cases, the sidechain group is an heterocycle-alkyl or a substituted heterocycle-alkyl group. The terms are meant to include naturally occurring and non-naturally occurring alpha-amino acids. Naturally occurring heterocyclic residues of interest include tryptophan and histidine.

The terms “non-polar amino acid residue” and “non-polar residue” refer to an amino acid residue that includes a sidechain that is hydrogen (i.e., G) or a non-polar group. In some cases, a non-polar amino acid sidechain is a hydrophobic group. The terms are meant to include naturally occurring and non-naturally occurring alpha-amino acids. Naturally occurring non-polar amino acid residues of interest include naturally occurring hydrophobic residues.

The terms “hydrophobic amino acid” and “hydrophobic residue” are used interchangeably to refer to an amino acid residue where the sidechain group is a hydrophobic group. The terms are meant to include naturally occurring and non-naturally occurring alpha-amino acids. Naturally occurring hydrophobic residues of interest include alanine, isoleucine, leucine, phenylalanine, proline and valine.

The terms “polar amino acid” and “polar residue” are used interchangeably to refer to an amino acid residue where the sidechain group includes a polar group or charged group. In certain cases, the polar group is capable of being a hydrogen bond donor or acceptor. The terms are meant to include naturally occurring and non-naturally occurring alpha-amino acids. Naturally occurring polar residues of interest include arginine, asparagine, aspartic acid, histidine, lysine, serine, threonine, tyrosine, cysteine, methionine, glutamic acid, glutamine and tryptophan.

The terms “scaffold” and “scaffold domain” are used interchangeably and refer to a reference peptidic framework motif from which a subject peptidic compound arose, or against which the subject peptidic compound is able to be compared, e.g., via a sequence or structural alignment method. The structural motif of a scaffold domain can be based on a naturally occurring protein domain structure. For a particular protein domain structural motif, several related underlying sequences may be available, any one of which can provide for the particular three-dimensional structure of the scaffold domain. A scaffold domain can be defined in terms of a characteristic consensus sequence motif. FIG. 14 shows one possible consensus sequence for a GA scaffold domain based on an alignment and comparison of 16 related naturally occurring protein domain sequences which provide for the three-helix bundle structural motif of a GA scaffold domain.

The terms “parent amino acid sequence”, “parent sequence” and “parent polypeptide” refer to a polypeptide comprising an amino acid sequence from which a variant peptidic compound arose and against which the variant peptidic compound is being compared. The parent polypeptide lacks one or more of the modifications or variant amino acids disclosed herein and can differ in function compared to a variant peptidic compound as disclosed herein. The parent polypeptide may be a native domain sequence (e.g., SEQ ID NO: 2-21), a native domain scaffold sequence having pre-existing amino acid sequence modifications (such as any convenient point mutations or truncations known to confer a desirable physical property upon the domain, e.g., increased stability or solubility), or a non-naturally occurring consensus sequence (e.g., a sequence of a consensus motif based on several native domains of interest, see e.g., FIG. 14).

The terms “corresponding residue” and “residue corresponding to” are used to refer to an amino acid residue located at equivalent positions of variant and parent sequences, e.g., as defined by the GA domain numbering scheme shown in FIG. 13. It is understood that the numbering scheme of FIG. 13 is not meant to define a minimum or maximum number of residues that must be included in the sequence of the subject compounds. A subject compound based on a 53 residue numbering scheme can include any convenient number of residues sufficient to retain a three-helix bundle structural motif. In some cases, a subject compound includes less than 53 residues, including a N-terminal and/or C-terminal truncated sequence (e.g., as described herein).

The terms “variant amino acid” and “variant residue” are used interchangeably to refer to the particular residues of a subject compound which are modified or mutated by comparison to an underlying scaffold domain. The variant residues encompass those residues that were selected (e.g., via mirror image screening, affinity maturation and/or point mutation(s)) to provide for a desirable domain motif structure that specific binds to the target. When a compound includes amino acid mutations or modifications at particular positions by comparison to a scaffold domain, the amino acid residues of the peptidic compound located at those particular positions are referred to as “variant amino acids.” Such variant amino acids may confer on the resulting peptidic compounds different functions, such as specific binding to a target protein, increased water solubility, ease of chemical synthesis, metabolic stability, etc. Aspects of the present disclosure include peptidic compounds that were selected from a phage display library based on a GA scaffold domain and further developed (e.g., via additional affinity maturation and/or point mutations), and as such include several variant amino acids integrated with a GA scaffold domain.

The terms “variant domain” and “variant motif” refers to an arrangement of variant amino acids incorporated at particular locations of a scaffold domain. The variant motif can encompass a continuous and/or a discontinuous sequence of residues. The variant motif can encompass variant amino acids located at one face of the compound structure. The variant domain may be considered to be incorporated into, or integrated with, an underlying scaffold domain structure or sequence. In the subject compounds, the scaffold domain can provide a stable three-dimensional protein structural motif, e.g., of a naturally occurring protein domain, while the variant domain can be defined by an arrangement of characteristic minimum number of variant residues at a modified surface of the structure that is capable of specifically binding a target protein.

The term “framework residues” refers to residual amino acid residues of a scaffold domain of a peptidic compound that are not variant amino acids. As such, a structural or sequence motif composed of framework residues is defined by the corresponding arrangement of residues of an underlying scaffold domain structure or sequence. The sequence and structure of a subject compound can be defined by a combination of variant and framework residues.

The term “mutation” refers to a deletion, insertion, or substitution of an amino acid(s) residue or nucleotide(s) residue relative to a reference sequence, such as a scaffold sequence. The term “domain” refers to a continuous or discontinuous sequence of amino acid residues. A domain can include one or more regions or segments. The terms “region” and “segment” are used interchangeably to refer to a continuous sequence of amino acid residues that, in some cases, can define a particular secondary structural feature.

The term “non-core mutation” refers to an amino acid mutation of a peptidic compound that is located at a position in the structure that is not part of the hydrophobic core of the structure. Amino acid residues in the hydrophobic core of a peptidic compound are not significantly solvent exposed but rather tend to form intramolecular hydrophobic contacts. A methodology used to specify hydrophobic core residues is described by Dahiyat et al., (“Probing the role of packing specificity in protein design,” Proc. Natl. Acad. Sci. USA, 1997, 94, 10172-10177) where a PDB structure was used to calculate which side chains expose less than 10% of their surface area to solvent. In some cases, Degrado's heptad repeat model (DeGrado et al. “Analysis and design of three-stranded coiled coils and three-helix bundles”, Folding & Design 1998, 3: R29-R40) can be utilized to define “a” and “d” residues of a hydrophobic core, as depicted in FIG. 6. Such methods can be modified for use with the GA domain scaffold.

The term “surface mutation” refers to an amino acid mutation in a scaffold domain that is located at a position in the structure that is solvent exposed. Such variant amino acid residues at surface positions of a D-peptidic compound can be capable of interacting directly with a target molecule, whether or not such an interaction occurs. In some cases, Degrado's heptad repeat model can be utilized to define “c” and “g” residues that are highly solvent exposed, as depicted in FIG. 6.

The term “boundary mutation” refers to an amino acid mutation in a scaffold that is located at a position in the structure that is at the boundary between the hydrophobic core and the solvent exposed surface. Such variant amino acid residues at boundary positions of a peptidic compound may be in part contacting hydrophobic core residues and/or in part solvent exposed and capable of some interaction with a target molecule, whether or not such an interaction occurs. One criteria for describing core, surface and boundary residues of a structure is described by Mayo et al. Nature Structural Biology, 5(6), 1998, 470-475. In some cases, Degrado's heptad repeat model can be utilized to define “c” and “g” residues that are at least partially solvent exposed, as depicted in FIGS. 6 and 7B. Such methods and criteria can be modified for use with the subject compounds.

The term “linking sequence” refers to a continuous sequence of amino acid residues, or analogs thereof, that connect two peptidic motifs or regions. In certain cases, a linking sequence is a loop or turn region (e.g., as described herein) connecting two antiparallel helical regions.

The term “stable” refers to a compound that is able to maintain a folded state under physiological conditions at a certain temperature, such that it retains at least one of its normal functional activities, for example binding to a target protein. The stability of the compound can be determined using standard methods. For example, the “thermostability” of a compound can be determined by measuring the thermal melt (“Tm”) temperature. The Tm is the temperature in degrees Celsius at which half of the compound becomes unfolded. In some instances, the higher the Tm, the more stable the compound.

The terms “similar,” “conservative,” and “highly conservative” amino acid substitutions are defined as shown in Table 6, below. The determination of whether an amino acid residue substitution is similar, conservative, or highly conservative can be based on the side chain of the amino acid residue and not the polypeptide backbone.

TABLE 6
Classification of Amino Acid Substitutions

| Amino Acid | Similar | Conservative | Highly Conservative |
|---|---|---|---|
| in Subject | Amino Acid | Amino Acid | Amino Acid |
| Polypeptide | Substitutions | Substitutions | Substitutions |
| Glycine (G) | A, S, N | A | n/a |
| Alanine (A) | S, G, T, V, C, | S, G, T | S |
| | P, Q | | |
| Serine (S) | T, A, N, G, Q | T, A, N | T, A |
| Threonine (T) | S, A, V, N, M | S, A, V, N | S |
| Cysteine (C) | A, S, T, V, I | A | n/a |
| Proline (P) | A, S, T, K | A | n/a |
| Methionine (M) | L, I, V, F | L, I, V | L, I |
| Valine (V) | I, L, M, T, A | I, L, M | I |
| Leucine (L) | M, I, V, F, T, | M, I, V, F | M, I |
| | A | | |
| Isoleucine (I) | V, L, M, F, T, | V, L, M, F | V, L, M |
| | C | | |
| Phenylalanine (F) | W, Y, L, M, I, | W, L | n/a |
| | V | | |
| Tyrosine (Y) | F, W, H, L, I | F, W | F |
| Tryptophan (W) | F, L, V | F | n/a |
| Asparagine (N) | Q | Q | Q |
| Glutamine (Q) | N | N | N |
| Aspartic Acid (D) | E | E | E |
| Glutamic Acid (E) | D | D | D |
| Histidine (H) | R, K | R, K | R, K |
| Lysine (K) | R, H, O | R, H, O | R, O |
| Arginine (R) | K, H, O | K, H, O | K, O |
| Ornithine (O) | R, H, K | R, H, K | K, R |

A “specificity determining motif” refers to an arrangement of variant amino acids incorporated at particular locations of a variant scaffold domain that provides for specific binding of the variant domain to a target protein. The motif can encompass continuous and/or a discontinuous sequences of residues. The motif can encompass variant amino acids located at one face of the compound structure and which are capable of contacting the target protein, or can encompass variant residues which do not provide contacts with the target but rather provide for a modification to the natural domain structure that enhances binding to the target. The motif may be considered to be incorporated into, or integrated with, an underlying scaffold domain structure or sequence, e.g., a three helix bundle of a naturally occurring GA or Z domain.

A compound that “specifically binds” to an epitope or binding site of a target protein is a term well understood in the art, and methods to determine such specific or preferential binding are also well known in the art. A compound exhibits “specific binding” if it associates more frequently, more rapidly, with greater duration and/or with greater affinity with a particular cell or substance (target protein) than it does with alternative cells or substances. A D-peptidic compound “specifically binds” to a target if it binds with greater affinity, avidity, more readily, and/or with greater duration than it binds to other substances. For example, a compound that specifically or preferentially binds to a VEGF epitope or site is an antibody that binds this epitope or site with greater affinity, avidity, more readily, and/or with greater duration than it binds to other VEGF epitopes or non-VEGF epitopes. It is also understood by reading this definition that, for example, a compound that specifically or preferentially binds to a first target may or may not specifically or preferentially bind to a second target. As such, “specific binding” does not necessarily require (although it can include) exclusive binding. Generally, but not necessarily, reference to binding means specific binding.

The compounds may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids and polypeptides. The present disclosure is meant to include all such possible isomers, as well as, their racemic, diastereomeric, and optically pure forms. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “a target protein” refers to all members of the target family, and fragments and enantiomers thereof, and protein mimics thereof. The target proteins of interest that are described herein are intended to include all members of the target family, and fragments and enantiomers thereof, and protein mimics thereof, unless explicitly described otherwise. The target protein may be any protein of interest, such as a therapeutic or diagnostic target. The term “target protein” is intended to include recombinant and synthetic molecules, which can be prepared using any convenient recombinant expression methods or using any convenient synthetic methods, or purchased commercially, as well as fusion proteins containing a target molecule, as well as synthetic L- or D-proteins.

The term “VEGF” or its non-abbreviated form “vascular endothelial growth factor”, as used herein, refers to the protein products encoded by the VEGF gene. The term VEGF includes all members of the VEGF family, such as, VEGF-A, VEGF-B, VEGF-C, VEGF-D, VEGF-E, and fragments and enantiomers thereof. The term VEGF is intended to include recombinant and synthetic VEGF molecules, which can be prepared using any convenient recombinant expression methods or using any convenient synthetic methods, or purchased commercially (e.g. R & D Systems, Catalog No. 210-TA, Minneapolis, Minn.), as well as fusion proteins containing a VEGF molecule, as well as synthetic L- or D-proteins. VEGF is involved in both vasculogenesis (the de novo formation of the embryonic circulatory system) and angiogenesis (the growth of blood vessels from pre-existing vasculature) and can also be involved in the growth of lymphatic vessels in a process known as lymphangiogenesis. Members of the VEGF family stimulate cellular responses by binding to tyrosine kinase receptors (the VEGFRs) on the cell surface, causing them to dimerize and become activated through transphosphorylation. The VEGF receptors have an extracellular portion containing 7 immunoglobulin-like domains, a single transmembrane spanning region and an intracellular portion containing a split tyrosine-kinase domain. VEGF-A binds to VEGFR-1 (Flt-1) and VEGFR-2 (KDR/Flk-1). VEGFR-2 appears to mediate several of the cellular responses to VEGF. VEGF, its biological activities, and its receptors are well studied and are described in Matsumoto et al. (VEGF receptor signal transduction Sci STKE. 2001:RE21 and Marti et al (Angiogenesis in ischemic disease. Thromb Haemost. 1999 Suppl 1:44-52). Amino acid sequences of exemplary VEGFs are found in the NCBI's Genbank database and a full description of VEGF proteins and their roles in various diseases and conditions is found in NCBI's Online Mendelian Inheritance in Man database.

### Exemplary Embodiments

Aspects of the present disclosure are embodied in the clauses and exemplary embodiments set forth below.
Clause 1. A D-peptidic compound that specifically binds VEGF-A, with the proviso that the compound does not comprise a GB1 domain scaffold.
Clause 2. The D-peptidic compound of clause 1, comprising: a VEGF-A binding two-helix complex comprising at least two antiparallel helical regions [Helix A] and [Helix B] that together define a VEGF-A binding face comprising six or more VEGF-A contacting residues independently selected from non-polar, aromatic, heterocyclic and carbocyclic residues.
Clause 3. The D-peptidic compound of clause 2, wherein [Helix A] and [Helix B] each comprise a heptad repeat sequence (abcdefg)ₙ and wherein the six or more VEGF-A contacting residues are located at the c and g positions of the heptad repeat sequences.
Clause 4. The D-peptidic compound of clause 1, comprising:

a VEGF-A binding three-helix bundle comprising helical regions [Helix 1], [Helix 2] and [Helix 3] each comprising a heptad repeat sequence (abcdefg)ₙ and configured to define a hydrophobic core substantially comprising a and d residues;

wherein [Helix 2] and [Helix 3] are configured antiparallel to each other and together define a VEGF-A binding g-g face of the three-helix bundle comprising six or more VEGF-A contacting residues independently selected from non-polar, aromatic, heterocyclic and carbocyclic residues.
Clause 5. The D-peptidic compound of clause 4, wherein the three-helix bundle is a GA domain motif of formula (I):

[Helix 1]-[Linker 1]-[Helix 2]-[Linker 2]-[Helix 3] (I)

wherein [Linker 1] and [Linker 2] are independently peptidic linking sequences of between 1 and 10 residues.
Clause 6. The D-peptidic compound of any one of clauses 4-5, wherein the six or more VEGF-A contacting amino acid residues comprise four or more aromatic amino acid residues that are configured to contact VEGF-A and are located at c and g solvent exposed positions of the g-g face.
Clause 7. The D-peptidic compound of any one of clauses 4-6, wherein [Helix 2] comprises the heptad repeat sequence [c¹d¹e¹f¹g¹a²b²c²d²] and [Helix 3] comprises the heptad repeat sequence [e¹f¹g¹a² b² c²d²e²f²g²a³b³c³d³e³], wherein:

residues d², a² and d¹ of [Helix 2] interact with residues a², d² and a³ of [Helix 3]; and

residues c², g¹ and c¹ of [Helix 2] and residue g¹ of [Helix 3] are each independently an aromatic, heterocyclic or carbocyclic residue.
Clause 8. The D-peptidic compound of any one of clauses 2-7, wherein the VEGF-A binding surface comprises the following configuration of VEGF-A contacting residues located at the c and g positions of the heptad repeat sequences of Helix A and Helix B:

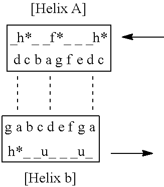

wherein:

each h* is independently histidine or an analog thereof;

f* is phenylalanine or an analog thereof; and

each u is independently a non-polar amino acid residue.
Clause 9. The D-peptidic compound of any one of clauses 4-5, wherein:

[Helix 2] comprises a sequence of the formula:

(SEQ ID NO: 151)
h*jxxf*jxh*j

[Helix 3] comprises a sequence of the formula:

(SEQ ID NO: 152)
h*jxujxxuj

wherein:
each h* is independently histidine or an analog thereof;
f* is phenylalanine or an analog thereof;
each u is independently a non-polar amino acid residue.
each j is independently a hydrophobic residue; and
each x is independently an amino acid residue.
Clause 10. The D-peptidic compound of clause 9, wherein [Helix 2] is defined by a sequence of the formula:

(SEQ ID NO: 153)
zh*jxxf*jxh*jz

wherein each z is independently a helix-terminating residue.
Clause 11. The D-peptidic compound of clause 10, wherein each helix-terminating residue (z) is independently selected from d, p and G.
Clause 12. The D-peptidic compound of any one of clauses 5-11, wherein [Linker 2] is 2 amino acid residues or less in length and comprises a tyrosine residue or an analog thereof.
Clause 13. The D-peptidic compound of any one of clauses 5-12, wherein [Helix 2]-[Linker 2]-[Helix 3] comprises a sequence of the formula:

(SEQ ID NO: 154)
zh*jxxf*jxh*jzy*xxh*jxujxxujx

wherein:
y* is tyrosine or an analog thereof;
each h* is independently histidine or an analog thereof;
f* is phenylalanine or an analog thereof;
each u is independently a non-polar amino acid residue.
each j is independently a hydrophobic residue; and
each x is independently an amino acid residue.
Clause 14. The D-peptidic compound of any one of clauses 5-13, wherein [Linker 1] has a sequence of the formula

(SEQ ID NO: 148)
z(x)ₙe*z

wherein:

each xis an amino acid and n is 1, 2 or 3;

each z is independently a helix-terminating residue (e.g., G or p); and

e* is glutamic acid or an analog thereof.
Clause 15. The D-peptidic compound of any one of clauses 5-14, wherein [Linker 1]-[Helix 2]-[Linker 2]-[Helix 3] comprises a sequence of the formula:

(SEQ ID NO: 155)
zxxe*zh*jxxf*jxh*jzy*xxh*jxujxxujx

wherein:
e* is glutamic acid or an analog thereof;
each z is independently a helix-terminating residue;
y* is tyrosine or an analog thereof;
each j is independently a hydrophobic residue;
each u is independently a non-polar amino acid residue; and
each x is independently an amino acid residue.
Clause 16. The D-peptidic compound of any one of clauses 4-15, wherein [Helix 2] is defined by a sequence of the formula:

(SEQ ID NO: 101)
z²⁶hj²⁸xxfj³²xhj³⁵z³⁶.

wherein:
z²⁶ is selected from d, p and G;
z³⁶ is selected from p and G;
j²⁸, j³² and j³⁵ are each independently a hydrophobic residue; and
each x is independently an amino acid residue.
Clause 17. The D-peptidic compound of clause 16, wherein j²⁸, j³² and j³⁵ are independently selected from a, i, l and v.
Clause 18. The D-peptidic compound of clause 17, wherein j²⁸, j³² and j³⁵ are corresponding residues of a GA scaffold domain selected from any one of SEQ ID NO: 1-21 of U.S. 62/865,469, filed Jun. 24, 2019.
Clause 19. The D-peptidic compound of any one of clauses 4-18, wherein [Helix 2] is defined by a sequence selected from:

a) phvx²⁹x³⁰fix³³hap (SEQ ID NO: 102)

wherein:
x²⁹ is selected from f and i;
x³⁰ and x³³ are independently selected from a polar amino acid residue; and

b) an amino acid sequence which has 80% or greater identity (e.g., 2 residue changes) to the sequence defined in a).
Clause 20. The D-peptidic compound of clause 19, wherein:
   x²⁹ is i;
   x³⁰ is s or n; and
   x³³ is n.
Clause 21. The D-peptidic compound of any one of clauses 4-20, wherein [Helix 3] is defined by a sequence of the formula:

(SEQ ID NO: 103)
xxhj⁴¹xuj⁴⁴xxuj⁴⁸xxx

wherein:
j⁴¹, j⁴⁴ and j⁴⁸ are each independently a hydrophobic residue;
each u is independently a non-polar amino acid residue; and
each x is independently an amino acid residue.
Clause 22. The D-peptidic compound of clause 21, wherein j⁴¹, j⁴⁴ and j⁴⁸ are independently selected from a, i, l and v.
Clause 23. The D-peptidic compound of clause 21, wherein j⁴¹, j⁴⁴ and j⁴⁸ are corresponding residues of a GA scaffold domain selected from SEQ ID NO: 1-21 of U.S. 62/865,469, filed Jun. 24, 2019.
Clause 24. The D-peptidic compound of clause 21, wherein [Helix 3] is defined by a sequence selected from:

a) x³⁸x³⁹hvx⁴²Glu⁴⁵x⁴⁶aix⁴⁹x⁵⁰a (SEQ ID NO: 98)

wherein:
x³⁸ is selected from v, e, k, r;
x³⁹, x⁴², x⁴⁶ and x⁵⁰ are independently selected from a hydrophilic amino acid residue (e.g., n, s, d, e and k); and
x⁴⁵ and x⁴⁹ are independently selected from l, k, r and e; and

b) an amino acid sequence which has 80% or greater identity (e.g., 2 residue changes) to the sequence defined in a).
Clause 25. The D-peptidic compound of clause 24, wherein: x³⁸ is V; X³⁹ is s; x⁴² is n; x⁴⁵ is k, x⁴⁶ is n; x⁴⁹ is l; and x⁵⁰ is k.
Clause 26. The D-peptidic compound of any one of clauses 4-25, wherein the VEGF-A binding domain of the compound comprises 6 or more variant amino acid residues relative to a reference GA scaffold sequence, wherein the 6 or more variant amino acids are selected from: e at position 25; p at position 26; h at position 27; vat position 28; i at position 29; s at position 30; f at position 31; h at position 34; p at position 36; y at position 37; s at position 39; h at position 40; G at position 43; and a at position 47.
Clause 27. The D-peptidic compound of clause 26, wherein the compound comprises p at position 26, fat position 31 and p at position 36.
Clause 28. The D-peptidic compound of clause 26, wherein the compound comprises the following variant amino acids: p at position 26, i at position 29 and s at position 30.
Clause 29. The D-peptidic compound of any one of clauses 26-28, wherein the compound comprises hat positions 27, 34 and 40.
Clause 30. The D-peptidic compound of any one of clauses 26-29, wherein the compound comprises G at position 43; and a at position 47.
Clause 31. The D-peptidic compound of any one of clauses 26-30, wherein the compound comprises v at position 28.
Clause 32. The D-peptidic compound of any one of clauses 1-31, wherein the compound comprises an amino acid sequence selected from:

a) llknakedaiaelkkcGitephvisfinhapyvshvnGlknailka; and

b) an amino acid sequence which has 85% or greater identity to the sequence defined in a).
Clause 33. The D-peptidic compound of any one of clauses 4-32, wherein [Helix 1] comprises a sequence selected from: a) l⁶lknakedaiaelkka²¹ (SEQ ID NO: 74); and b) an amino acid sequence which has 75% or greater identity to the sequence defined in a).
Clause 34. The D-peptidic compound of any one of clauses 1-33, wherein the compound comprises a sequence selected from: a) G²²itephvisfinhapyvshvnGlknailka⁵¹ (SEQ ID NO: 84); and b) an amino acid sequence which has 75% or greater identity to the sequence defined in a).
Clause 35. The D-peptidic compound of any one of clauses 1-34, wherein the compound comprises a peptidic framework sequence selected from: a)

l⁶lknakedaiaelkkaGit.......in.a..v..vn..kn.ilka⁵¹ (SEQ ID NO: 156); and b) an amino acid sequence which has 88% or greater identity to the sequence defined in a).
Clause 36. The D-peptidic compound of any one of clauses 1-35, wherein the compound comprises a peptidic framework sequence selected from: a)

t^{l}idqwllknakedaiaelkkaGit.......in.a..v..vn..kn.ilkaha⁵³ (SEQ ID NO: 157); and b)

an amino acid sequence which has 90% or greater identity to the sequence defined in a).
Clause 37. The D-peptidic compound of any one of clauses 1-36, wherein the compound comprises a sequence selected from SEQ ID NO:22-71 of U.S. 62/865,469, filed Jun. 24, 2019.
Clause 38. The D-peptidic compound of any one of clauses 1-37, further comprising a linked non-proteinaceous polymer moiety.
Clause 39. The D-peptidic compound of any one of clauses 1-37, further comprising a linked specific binding moiety.
Clause 40. The D-peptidic compound of clause 39, wherein the linked specific binding moiety is a second D-peptidic binding domain.
Clause 41. The D-peptidic compound of any one of clauses 39-40, wherein the compound comprises a multimeric configuration of a VEGF-binding GA domain.
Clause 42. The D-peptidic compound of any one of clauses 40-41, wherein compound is homodimeric and comprises two linked VEGF-A-binding GA domains.
Clause 43. The D-peptidic compound of clause 42, wherein the VEGF-A-binding GA domains are connected by N-terminal residues via a polymeric linker.
Clause 44. The D-peptidic compound of clause 42, wherein the VEGF-A-binding GA domain motifs are connected by N-terminal residues via a peptidic linker.
Clause 45. The D-peptidic compound of any one of clauses 40-41, wherein the compound is heterodimeric.
Clause 46. The D-peptidic compound of clause 45, wherein the second D-peptidic binding domain specifically binds a target protein selected from PDGF, VEGF-B, VEGF-C, VEGF-D, EGF, EGFR, Her2, Her3, PD-1, PD-L1, CTLA4, OX-40, DR3, Ang-2, LAG3, HSA and Ig.
Clause 47. The D-peptidic compound of any one of clauses 1-46, wherein the compound specifically binds to the VEGF-A protein with a K_{D} value of 100 nM or less (e.g., 30 nM or less, 10 nM or less, 3 nM or less, 1 nM or less etc.).
Clause 48. The D-peptidic compound of any one of clauses 1-47, wherein the VEGF-binding GA domain comprises between 45 and 60 residues (e.g., between 46 and 55 residues, between 50 and 54 residues, etc).
Clause 49. A pharmaceutical composition, comprising the D-peptidic compound of any one of clauses 1-48, or a pharmaceutically acceptable salt thereof, and a pharmaceutically acceptable excipient.
Clause 50. The pharmaceutical composition of clause 49, wherein the composition is formulated for the treatment of an eye disease or condition.
Clause 51. A method of treating or preventing a disease or condition associated with angiogenesis in a subject, the method comprising administering to a subject in need thereof an effective amount of a compound according to any one of clauses 1-48, or an effective amount of the pharmaceutical composition according to any one of clauses 49-50.
Clause 52. The method of clause 51, wherein the disease or condition associated with angiogenesis is cancer (e.g., breast, skin, colorectal, pancreatic, prostate, lung or ovarian cancer), an inflammatory disease, atherosclerosis, rheumatoid arthritis, macular degeneration, retinopathy and skin disease (e.g., rosacea).
Clause 53. The method of clause 51, wherein the disease or condition associated with angiogenesis is diabetic macular edema (DME).
Clause 54. The method of clause 51, wherein the disease or condition associated with angiogenesis is wet age-related macular degeneration (AMD).
Clause 55. The method of any one of clauses 51-54, further comprising administering an effective amount of a second active agent to the subject.
Clause 56. The method according to clause 55, wherein the second active agent is a D-peptidic compound.
Clause 57. The method according to clause 55, wherein the second active agent is a small molecule, a chemotherapeutic, an antibody, an antibody fragment, an aptamer, or a L-protein.
Clause 58. The method according to any one of clauses 55-57, wherein the second active agent specifically binds a target protein selected from platelet-derived growth factor (PDGF), VEGF-B, VEGF-C, VEGF-D, EGF, EGFR, Her2, Her3, PD-1, PD-L1, CTLA4, OX-40, DR3, LAG3, Ang2, IL-1, IL-6 and IL-17.
Clause 59. The method according to clause 55, wherein the second active agent specifically binds PDGF-B.
Clause 60. The method according to clause 55, wherein the second active agent is selected from: pegpleranib (Fovista), ranibizumab (Lucentis), trastuzumab (Herceptin), Bevacizumab (Avastin), aflibercept (Eylea), nivolumab, atezolizumab, Durvalumab, gefitinib, erlotinib and Pembrolizumab.
Clause 61. A method for in vivo diagnosis or imaging of a disease or condition associated with angiogenesis comprising administering to a subject a D-peptidic compound according to any one of clauses 1-49 and imaging at least a part of the subject.
Clause 62. The method according to clause 61, wherein the imaging comprises PET imaging and the administering comprises administering the compound to the vascular system of the subject.
Clause 63. The method according to clause 61, further comprising detecting uptake of the compound by cell receptors.
Clause 64. The method according to clause 61, further comprising administering avastin to the subject, wherein the disease or condition is a condition associated with cancer.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Selection of D-Peptidic Compounds

The subject compounds were identified via mirror image screening of a scaffolded GA domain phage display library for binding to a synthetic D-VEGF-A target protein using methods as described by Uppalapati et al. in WO2014/140882. FIG. 13 shows a depiction of the GA domain library including an underlying 53 residue scaffold sequence (SEQ ID NO: 2) and mutation positions in bold at positions 25, 27, 28, 31, 34, 36, 37, 39, 40, 43, 44 and 47 of the scaffold which positions define the variation in the phage display library.

Briefly, 5 ug/ml of D-VEGFA was coated in NUNC Maxisorp plates. After blocking, a pool of 8 scaffold libraries including the GA domain library, was added to the plate after depletion on an empty well. The bound phage was eluted and amplified overnight in OmniMax² T1R cells. For Rounds 3 and 4, a lower concentration of amplified phage pools (˜5×10¹¹ cfu/ml) was used compared to the standard concentration of 1 x 10¹³ cfu/ml, as the elute concentration was too high in Round 2. Several hits were obtained from various libraries including 17 different sequences from GA domain library. Based on sequence identity between the clones, 3 representative clones including Compound 1 (See FIG. 15) were selected for further optimization. Compound 1 retained binding to D-VEGFA upon cloning into a p3-fusion vector for affinity maturation.

For the first round of affinity maturation, a soft-randomization strategy was utilized (Fairbrother et.al, 1998) where the polynucleotides encoding each of the randomized positions 25, 27, 28, 31, 34, 36, 37, 39, 40, 43, 44 and 47 were doped with handmixed bases so that the native nucleotide is baised at 70% and the other three nucleotides occur at 10% frequency. This allows for a 40% chance of retention of amino acids found in the parent sequence of Compound 1 in each of these positions. The affinity maturation library was constructed by site-directed mutagenesis protocols (Fellouse et.al.) using the following oligonucleotide and ssDNA from GA domain original sequence as template.

(SEQ ID NO: 130)
AAGGCTGGTATCACC (N4)(N2)(N4) GAC (N2)(N1)(N4) (N3)
(N4)(N4) TTCAAC (N4)(N4)(N4) ATCAAT (N4)(N1)(N4)
GCG (N2)(N2)(N4) (N4)(N1)(N4) GTG (N4)(N2)(N4)
(N3)(N1)(N4) GTTAAC (N3)(N2)(N1) (N2)(N4)(N3)
AAGAAC (N3)(N1)(N3) ATCCTGAAAGCTCAC

### Where N1 is a mix of 70% A, 10% C, 10% G and 10% T

N2 is a mix of 10% A, 70% C, 10% G and 10% T

N3 is a mix of 10% A, 10% C, 70% G and 10% T

N4 is a mix of 10% A, 10% C, 10% G and 70% T

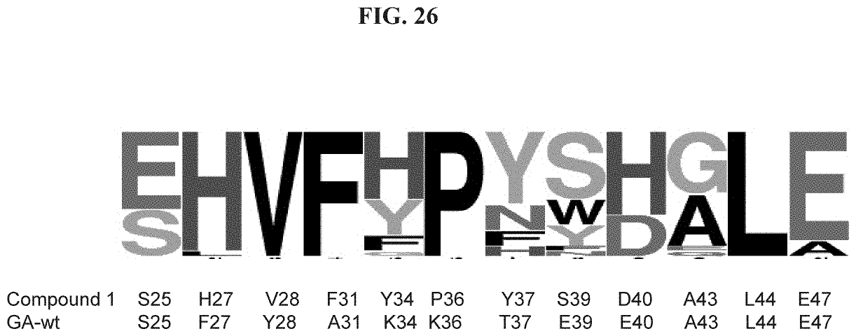

The affinity maturation library was panned against D-VEGFA using standard procedures (Fellouse et.al.) 24 clones from Round 3 were analyzed and a competitive ELISA was performed to rank them by affinity. Compound 1.1 was selected as a clone of interest from this list. A sequence logo of selected positions of all the clones is shown in FIG. 26 in comparison to Compound 1 and native GA domain (GA-wt). In this study, positions 27, 28, 31, 36 and 44 were highly conserved or retained in all clones as His27, Val28, Phe31, Pro36 and Leu44. Aromatic residues His, Tyr and Phe were predominant in position 34. His or Asp residues were predominant in position 40. Glu or Ala were predominant in position 47.

A second round of affinity maturation was performed to improve the affinity and stability of Compound 1.1. Given that a Pro residue was heavily conserved in Position 36, the change in backbone conformation may alter the orientation of Helix2 with respective to core residues and possibly affect the stability of selected Compound 1.1. Moreover, surface exposed residues near the C-terminus can form additional contacts. Therefore the following positions including core and surface exposed positions were selected for further optimization: Positions 15, 18, 19, 21, 23, 25, 26, 28, 29, 30, 47, 48, 49, 50, 51 and 52. Again a soft randomization strategy was used with the following oligonucleotides for site directed mutagenesis

(SEQ ID NO: 131)
GCGAAAGAAGATGCT (N1)(N4)(N4) GCAGAA (N2)(N4)(N2)
(N1)(N1)(N1) AAG (N2)(N2)(N4) GGT (N1)(N4)(N2)
ACC (N2)(N1)(N1) (N2)(N1)(N2) CAT (N2)(N4)(N4)
(N4)(N4)(N2) (N1)(N1)(N2) TTTATCAATCACGCGC
(N2) (N4)(N2) (N1) (N1)(N1)
(SEQ ID NO: 132)
GTTAACGGGCTGAAGAAC (N2)(N2)(N2) (N1)(N4)(N2)
(N2)(N2)(N4) (N2)(N1)(N2) GCCGGGAGCTCTGGAG

The library was constructed and panned against D-VEGFA with a modified protocol. Given that D-VEGF-A is highly stable and retains fold even at 3M guanidine hydrochloride (GuHCl), it was hypothesized that selection of binders in the presence of a low-medium concentration of denaturant can select for clones with improved affinity and stability at the same time. In this procedure, the library or the amplified phage pool was resuspended in PBT buffer (PBS, 0.2% BSA, 0.05% Tween20) with varying concentrations of denaturant guanidine hydrochloride (GuHCl) for each round of selection. The phage was incubated at 37° C. for 2 hours for equilibration. The selections were also carries out at 37° C. The following conditions were used for each round.

| | D-VEGFA coating | GuHCl conc in buffer | Washes |
|---|---|---|---|
| Round 1 | 5 ug/ml | 0.5M | 8 |
| Round 2 | 5 ug/ml | 1M | 8 |
| Round 3 | 5 ug/ml | 1M | 8 |
| Round 4 | 5 ug/ml | 1.5M | 8 |

After four rounds of affinity maturation several clones were sequenced and Compound 1.1.1 was selected as a clone of interest via assessment with a competitive ELISA assay. Cys21 was identified as a bystander mutation and reverted back to Ala (e.g., to eliminate possibility of disulfide dimerization) to give a lead compound of interest, compound 1.1.1(C21A).

In addition, a variety of the scaffolded phage display libraries that are described by Uppalapati et al. in WO2014/140882 were screened for binding to synthetic D-VEGF-A target protein. Several of the scaffolded domain libraries produced hit clones during phage display screening studies indicating that the subject D-peptidic compounds that specifically bind VEGF-A can have one of a variety of underlying scaffold domains. Initially, the hit clones from the GA domain scaffolded library were selected for further investigation.

TABLE 7
List of scaffolds that generated hits to D-VEGFA

SCF2-DGCR8 dimerization domain-56aa
SCF3-Get5 C-terminal domain-41aa
SCF7- KorB C-terminal domain-58aa
SCF8- Lsr2 dimerization domain-55aa
SCF15-Symfoil 4P (designed beta-trefoil)-42aa
SCF24-GRIP domain of Golgin245-51aa
SCF28- C-terminal domain of Ku-51aa
SCF 32-GA domain of Protein G-53aa
SCF29-Cue domain of Cue2-49aa
SCF37-PEM1 like protein-44aa
SCF40- Nucleotide exchange factor C-terminal domain-60aa
SCF42-Transcription factor anti-termination protein-59aa
SCF44- This protein-65aa
SCF53-Rhodnin kazal inhibitor -51aa
SCF55-Anti-TRAP-48aa
SCF56- TNF receptor 17 (BCMA) -39aa
SCF63- Fyn SH3 -61aa
SCF64- E3 ubiquitin-protein ligase UBR5-65aa
SCF65- DNA repair endonuclease XPF-63cta
SCF66- rad23 hom.B, xpcb domain -61aa
SCF70- LEM domain of Emerin-47aa
SCF75- GspC-68aa
SCF95- Protein Z -58aa
SCF96- B1 domain of protein G (GB1)-55aa

### Example 2: Synthesis and Folding of D-Peptidic Compounds

Selected compounds were synthesized and purified using conventional Fmoc solid phase peptide synthesis methods. In some cases, additional point mutations were included, e.g., as described herein. Compounds were folded in buffer and assessed for VEGF-A inhibition activity as described herein.

### Example 3: X-Ray Crystal Structure of VEGF-A Complex

An X-ray crystal structure of Compound 1.1.1(C A) in complex with L-VEGF-A was obtained. FIG. 1 shows a view of the X-ray crystal structure of exemplary compound 1.1.1(c21a) (white stick representation) in complex with VEGF-A (space filling representation). The complex is dimeric. In FIGS. 1 and 2, the binding site residues of VEGF-A which contact the compound are depicted in pink. VEGF-A (8-109) binding site residues are indicated in bold:

(SEQ ID NO: 88)
GQNHHEVVKFMDVYQRSYCHPIETLVDIFQEYPDEIEYIFKPSCVPLMR
CGGCCNDEGLECVPTEESNITMQIMRIKPHQGQHIGEMSFLQHNKCECR
PKKD;

where a single binding site in the dimer is defined by the following residues of:

Chain A:
(SEQ ID NO: 89)
KFMDVYQRSY
and
(SEQ ID NO: 90)
NDEGL;
and
Chain B:
(SEQ ID NO: 91)
YIFKP
and
(SEQ ID NO: 92)
IMRIKPHQGQHI.

### Example 4: Assessing Potency of Selected Compounds

The binding affinity of compounds of interest for VEGF-A was measured using a surface plasmon resonance (SPR) assay.

TABLE 8
D-VEGF-A binding affinity of exemplary L-peptidic compounds

| Compound | K_{d} (M) |
|---|---|
| 1.1 | 1.4 × 10⁻⁷ |
| 1.1 (-tidqw) | 4.5 × 10⁻⁷ |

Compounds of interest were assessed for VEGF-A binding in a competitive phage ELISA assay.

TABLE 9
D-VEGF-A binding activity of exemplary L-peptidic compounds

| Compound | IC₅₀ (nM) |
|---|---|
| 1.1 | 100-105 |
| 1.1.1 | 20-34 |
| 1.1 (-kaha, adf1) | 12 |
| 1.1 (-kaha, edy1) | 5 |
| 1.1 (-kaha, Grtvp) | 1-1.1 |
| 1.1 (-kaha, edwy1) | 5-5.4 |
| 1.1 (-kaha, GehGsp) | 14 |
| 1.1.1 (c21a) | 7 |
| 1.1.1(c21a) (-kaha, Grtvp) | 0.27-0.30 |
| 1.1.1(c21a) (-tidqw, -kaha, Grtvp) | 0.42-0.66 |
| 1.1.1(c21a) (-kaha, edwy1) | 0.31-0.66 |
| 1.1.1(c21a) (-tidqw, -kaha, edwy1) | 0.86-1.1 |

Compounds of interest were assessed for inhibition of VEGF-A:VEGFR1 in an Octet assay. Exemplary conditions: VEGF-A at 10 nM, inhibitor at nM conc.; VEGF-A:VEGFR1 K_{d}=25 pM.

TABLE 10
VEGF-A: VEGFR1 inhibition activity
of exemplary D-peptidic compounds

| Compound | Potency in Octet Assay IC₅₀ (nM) |
|---|---|
| 1.1 | 105 |
| 1.1.1 | 9 |

TABLE 11
VEGF-A: VEGFR1 inhibition activity
of exemplary D-peptidic compounds

| Compound | % inhibition at 80 nM compound |
|---|---|
| 1.1.1(c21a) (c(Ac)54) | 68 |
| 1.1.1(c21a) (-kaha, Grtvp) | 27 |
| 1.1.1.2 (pis) | 27 |
| 1.1.1.2 (pa, pis) | 18 |
| 1.1.1.3 (pis) | 23 |

### Example 5: Preparation and Evaluation of Dimeric Compounds

A series of dimers of modified compound 1.1.1 (c21a) were prepared having linkers of various lengths by conjugation of a variety of PEG-based linkers to either the N- or C-terminals of the compounds using cysteine maleimide or disulfide conjugation chemistry. A cysteine residue was incorporated either at the C-terminal or N-terminal of the compound and dimerization was achieved via cysteine-maleimide conjugation chemistry with a bifunctional modified PEG linker. Structures of exemplary dimeric compounds are shown below:

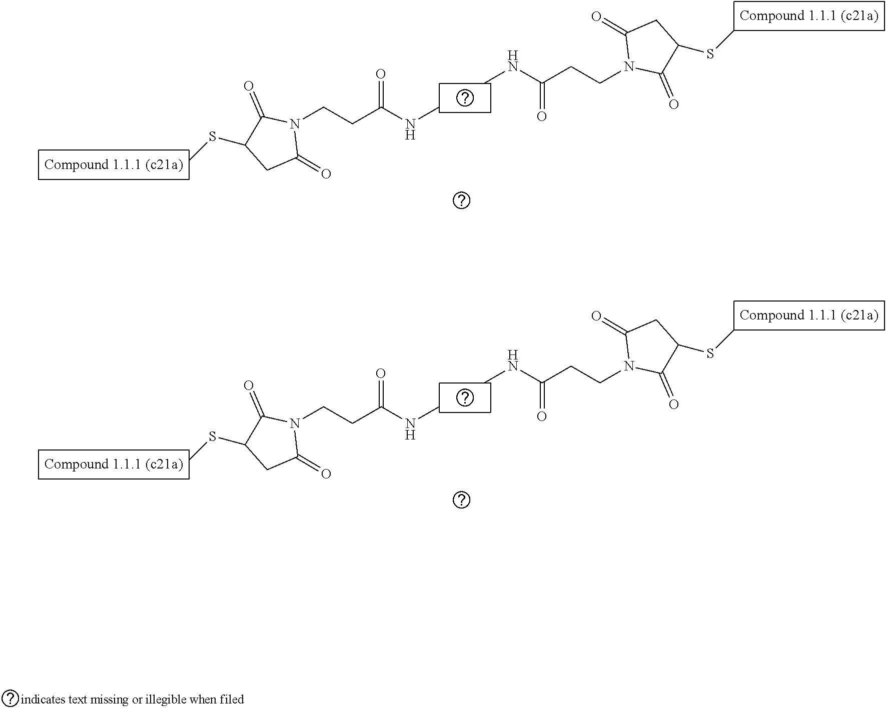

The resulting dimeric compounds were assayed for VEGF-A inhibition activity in an octet assay.

TABLE 12
Inhibition of VEGF-A binding to VEGFR1 receptor by Octet assay

| Linker | N-N dimerization | C-C dimerization |
|---|---|---|
| Disulfide | | 35.3 |
| PEG (3 units) | 93.4 | 102.7 |
| PEG (6 units) | 95.7 | 101.7 |
| PEG (11 units) (approx. 60 Å | 95.6* | 95.4* |
| length) | | |
| PEG (1000K MW) (approx. 100 Å | 94.3* | 67.8* |
| length) | | |
| PEG (2000K MW) (approx. 180 Å | 95.7* | 98.9* |
| length) | | |

Conditions: VEGF-A at 10 nM, inhibitor at 20 nM (or 25 nM*); VEGF-A:VEGFR1 K_{d}=25 pM. 100%=100 nM of (1.1.1 (c21a)) dimer C-C-linked with PEG11 linker

### Example 6: Preparation and Evaluation of Synthetic Point Mutations including Phenylalanine 31 and/or Tyrosine 37 Amino Acid Analogs

Based on an analysis of the X-ray crystal structure as shown in FIGS. 21 and 24, a variety of non-naturally occurring amino acid analogs of phenylalanine 31 and tyrosine 37 were selected for incorporation into exemplary compound 1.1.1(c21a). A series of analogs of the compound 1.1.1(c21a)-PEG6 N to N linked dimer were prepared according to the methods described herein. The activity of the compounds is assessed in an inhibition assay under the following conditions. Table 13 shows % inhibition at 20 nM compound, 10 nM VEGF-A relative to reference compound 1.1.1(c21a)-PEG6 N to N linked dimer at 20 nM.

TABLE 13
Activity of 1.1.1(c21a)-PEG6 N to N dimer analogs compounds
having synthetic point mutations

| 1.1.1(c21a)-PEG6 N to N | Position 31 | Position 37 | |
|---|---|---|---|
| dimer | sidechain | sidechain | % inhibition |
| Control compound | 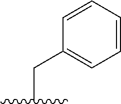 [SMILES: *Cc1ccccc1] | 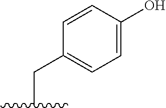 [SMILES: *Cc1ccc(O)cc1] | 58 |
| f31[(4-fluoro)f] | 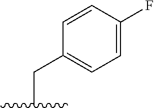 [SMILES: *Cc1ccc(F)cc1] | 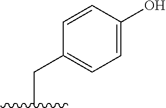 [SMILES: *Cc1ccc(O)cc1] | 22 |
| f31[(3-fluoro)f] | 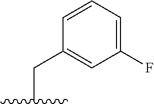 [SMILES: *Cc1cccc(F)c1] | 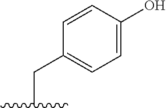 [SMILES: *Cc1ccc(O)cc1] | 95 |
| f31[(4-chloro)f] | 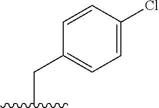 [SMILES: *Cc1ccc(Cl)cc1] | 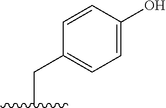 [SMILES: *Cc1ccc(O)cc1] | 66 |
| f31[(3-chloro)f] | 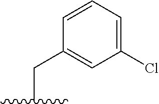 [SMILES: *Cc1cccc(Cl)c1] | 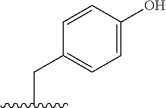 [SMILES: *Cc1ccc(O)cc1] | 40 |
| f31[(4-methyl)f] | 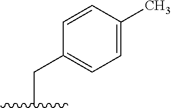 [SMILES: *Cc1ccc(C)cc1] | 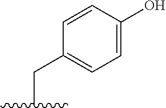 [SMILES: *Cc1ccc(O)cc1] | 8 |
| f31[(3-methyl)f] | 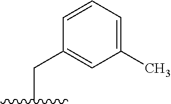 [SMILES: *Cc1cccc(C)c1] | 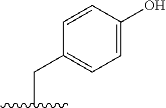 [SMILES: *Cc1ccc(O)cc1] | 48 |
| f31[(4-CF₃)f] | 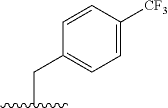 [SMILES: *Cc1ccc(C(F)(F)F)cc1] | 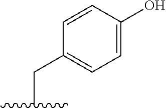 [SMILES: *Cc1ccc(O)cc1] | 0 |
| f31[(3-CF₃)f] | 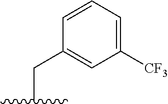 [SMILES: *Cc1cccc(C(F)(F)F)c1] | 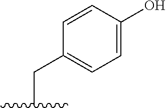 [SMILES: *Cc1ccc(O)cc1] | 2 |
| f31[(4-aminomethyl)f] | 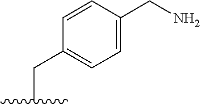 [SMILES: *Cc1ccc(CN)cc1] | 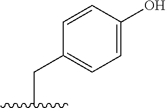 [SMILES: *Cc1ccc(O)cc1] | 0 |
| y37[(4-aminomethyl)f] | 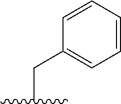 [SMILES: *Cc1ccccc1] | 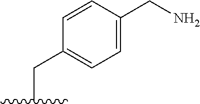 [SMILES: *Cc1ccc(CN)cc1] | 87 |
| f31[(3-fluoro)f] and y37[(4-aminomethyl)f] | 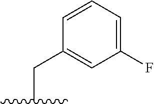 [SMILES: *Cc1cccc(F)c1] | 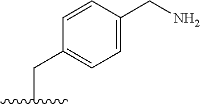 [SMILES: *Cc1ccc(CN)cc1] | not determined |

### Example 7: Affinity Optimization of a D-Peptidic antagonist of VEGF-A

A D-peptidic VEGF-A antagonist was identified using mirror image phage display screening of a GA-domain library. See U.S. 62/688,272, filed Jun. 21, 2018, by Uppalapati et al. and entitled “D-Peptidic VEGF-A Binding Compounds and Methods for Using the Same”. Exemplary compound 11055 (FIG. 3B), exhibited a VEGF-A binding affinity of 31 nM as determined by surface plasmon resonance (SPR). This is significantly weaker than bevacizumab (Avastin), a clinically approved VEGF-A antagonist, which exhibits sub-nanamolar binding to VEGF-A and is able to block its biological activity in vivo. The present disclosure describes use of phage display based affinity maturation to provide high affinity variants of 11055 that are potent antagonists of VEGF-A.

In order to engineer a high affinity variant of 11055, a pIII-fused phage display library was designed based on an analysis of the X-ray crystal structure of compound 11055 bound to VEGF-A. A 2.3 angstrom resolution structure was solved of 11055 in complex with VEGF-A using hanging drop method. Diffraction quality crystals were grown in 0.1 M Tris pH 8.5, 0.2 M calcium chloride, 18% w/v PEG 4000. The structure was solved by molecular replacement. The crystal structure shows two molecules of 11055 bound to a VEGF-A homodimer where they occupy identical binding sites on the VEGF-A monomers, these sites overlap with the VEGFR2 receptor binding site of VEGF-A (FIGS. 28A and 28B).

Based on this structure a library was designed to further stabilize the variant GA domain three helix structure. A total of 7 amino acid residues were selected for randomization at the packing interface between helix 1 (H1) and the loop connecting helix 2 (H2) and helix 3 (H3) (FIG. 29A). Kunkel mutagenesis was used to prepare a library and simultaneously randomize each selected residue with the NNC degenerate codon representing 15 possible AA substitutions (FIG. 29B). The resulting phage library contained >1×10¹⁰ individual variants and was screened for binding to refolded D-VEGF-A target using mirror image phage display methods. See, e.g., Mandal et al., PNAS (2012), 109(37), 14779-14784. Briefly, 4 rounds of panning against biotinylated D-VEGF-A target were carried out. For each round, phage library was incubated with target in phosphate buffered saline (PBS), and the target-bound phage captured on streptavidin coated beads, washed, and eluted for the next round of infection and phage amplification. During each round bound phage clones were exposed to increasingly stringent temperature and wash conditions to increase selective pressure for generating high affinity binders to the target. After the fourth round of selection individual phage clones were sequenced and a preferred consensus motif was identified, containing two fixed cysteine residues at positions 7 and 38 of the variant GA domain and preferred amino acid residues at positions 1, 2, 3, 6 and 37 (FIG. 30A).

Based on the X-ray crystal structure described above (FIG. 29A), cysteine mutations at positions 7 and 38 appear to place the sidechain sulfhydryl groups within close enough proximity to form an intra-molecular disulfide bond (FIG. 30C). This analysis of the three dimensional structure is consistent with the fixed conservation of paired cysteines at positions 7 and 38 shown in the consensus motif results (FIG. 30A). Five representative variants (SEQ ID NOs: 21-25) were synthesized as D-enantiomers and their binding affinities to natural L-VEGF-A were measured using SPR (FIG. 30B). Variant 979110 had the highest VEGF-A affinity with a measured equilibrium dissociation constant (K_{D}) of 3.6 nM. Thus, affinity optimization improved VEGF binding nearly 10-fold over 11055.

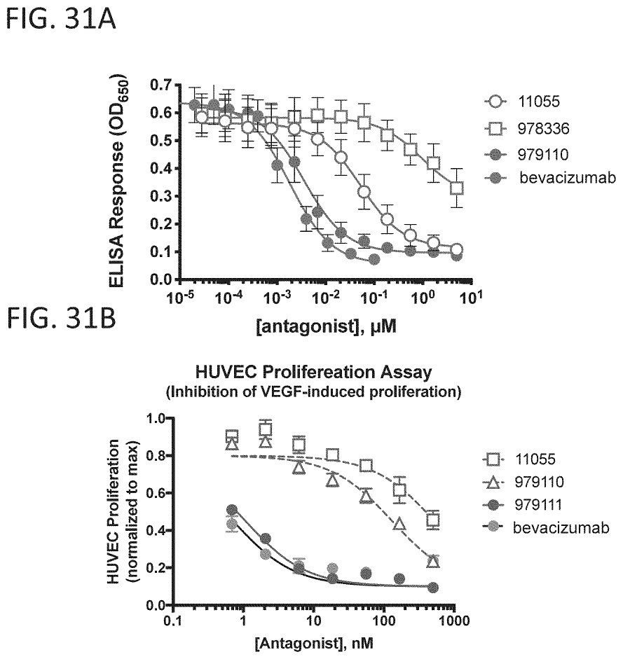

The affinity matured D-peptidic compounds were characterized in a VEGF-A blocking ELISA in order to measure their antagonistic activity. Here, a VEGFR1-Fc fusion was coated overnight on Maxisorp plates at 1 μg/mL in PBS. 1 nM biotinylated-VEGF-A was mixed with antagonist titrations and binding of biotinylated-VEGF-A to VEGFR1-Fc was detected with streptavidin-HRP. Variant compound 979110 blocks VEGF-A binding to VEGFR1 and exhibited an inhibition constant (IC50) in this assay of 3.5 nM, 14.8-fold better than 11055 (52 nM), consistent with the improved binding affinity (FIG. 31A).

A HUVEC cell proliferation assay was used to assess the ability of the D-peptidic compounds to block VEGF-A signaling. Here, HUVEC cell proliferation is increased in the presence of recombinant VEGF-A and antagonist compounds that block VEGF-A signaling reduce HUVEC cell proliferation. The apparent IC50 of compound 979110 in the HUVEC assay was 131 nM, which is 4-fold more potent than parent compound 11055, but remains 185-fold weaker than bevacizumab (Avastin), (FIG. 31B). These data suggest that the improvement in binding affinity of 979110 relative to 11055 may not be sufficient to block VEGF-A biological activity in vivo with a potency comparable to bevacizumab (Avastin).

### Example 8A: Engineering D-Peptidic Antagonists to Non-Overlapping Epitopes on VEGF-A

The structures of VEGF-A in complex with VEGF receptors, VEGFR1 and VEGFR2 are available and reveal multivalent interactions between Ig-like domains of VEGFR1 or VEGFR2 and two identical binding sites on the VEGF-A homodimer (Markovic-Mueller et al., Structure (2017), 25, 341-352)(Brozzo et al., Blood (2012), 119(7), 1781-1788.). An overlay of the compound 11055/VEGF-A complex structure with VEGFR2 highlights significant overlap between the 11055 binding epitope and one of the Ig-like domains of VEGFR2 (Domain 2, D2) (See FIG. 28B), consistent with the antagonistic activity of 11055 (FIG. 31A). However, a second Ig-like domain of VEGFR2 (Domain 3, D3) binds to an additional binding site on VEGF-A that is separate from the 11055 binding site (FIG. 28B). We sought to engineer a second D-peptidic antagonist that would bind to the VEGFR2 D3 binding site on VEGF-A, thereby blocking an additional receptor binding site independent of 11055.

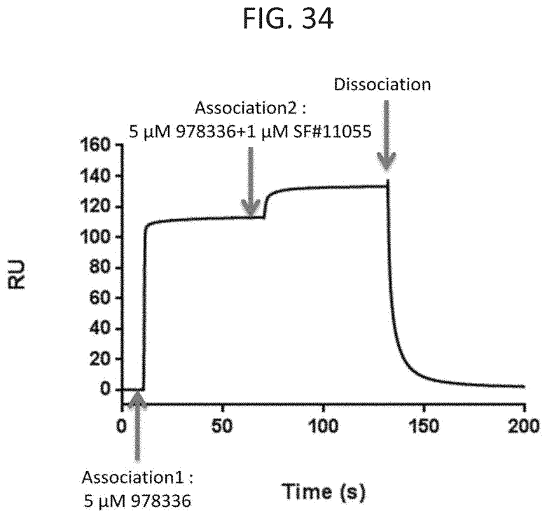

A new phage display library based on the Z-domain scaffold was generated as a pVIII-fusion to M13 phage. Ten positions were selected within the Z-domain for randomization using kunkel mutagenesis with trinucleotide codons representing all amino acids except cysteine (FIGS. 32A and 32B). The resulting library was screened for binding to refolded D-VEGF-A target using mirror image phage display methods. Briefly, 3 rounds of panning against biotinylated D-VEGF-A were carried out under increasingly stringent wash conditions. After the 3^{rd} round, the phage pools were transferred to a p111-fusion to reduce the copy number on phage particles and the transferred phage were put through 2 additional rounds of panning After the last round of selection on P3, individual phage clones were sequenced and a preferred consensus motif was identified containing the fixed amino acids W, D, W, R, K and Y at positions 9, 10, 13, 17, 27 and 35, respectively (FIG. 33A). Five representative variant D-peptidic compounds were synthesized (SEQ ID NOs: 26-31) and their binding affinities to native L-VEGF-A were measured using SPR (FIG. 33B). Variant 978336 had the highest VEGF-A affinity with a measured K_{D} of 500 nM. Epitope mapping using SPR was carried out to determine whether compound 978336 and compound 11055 bound non-overlapping binding sites on VEGF-A. Here, biotinylated VEGF-A was captured on the SPR chip and 5 μM of compound 978336 was bound in the first association step in order to saturate its binding site. In a second association step, 5 μM compound 978336 was mixed with 1 μM 11055 and the change in steady state binding was measured. The sensorgram data displayed an increase in response units due to compound 11055 binding, which was above the saturating response level of compound 978336, indicating additive binding of compounds 978336 and 11055 (FIG. 34). Finally, in a VEGF-A blocking ELISA, compound 978336 could antagonize the interaction between VEGF-A and VEGFR1 with a measured IC₅₀ of 935 nM (FIG. 31A). These data indicate that 978336 binds to a non-overlapping epitope independent of the 11055 site and is a VEGF-A antagonist.

To further characterize the VEGF-A binding site for compound 978336 a 2.9 angstrom resolution crystal structure was solved of L-VEGF-A in complex with 978336. Diffraction quality crystals were grown in 0.1M Bis-Tris, pH 5.5, 0.15 M magnesium chloride, 25% w/v PEG 3350 using the hanging drop method. The structure was solved by molecular replacement. Two molecules of 978336 were bound to identical binding sites on a single VEGF-A homodimer (FIG. 35A). The structure reveals that compound 978336 directly overlaps with the D3 binding site on VEGF-A (FIG. 35B) and confirms that 11055 and 978336 have non-overlapping epitopes directly blocking both D2 and D3 sites on VEGF-A, respectively (FIGS. 28B and 35B).

### Example 8B: Affinity Maturation Screening of Compound 978336

Structure-based affinity maturation methods were used to improve upon the VEGF-A binding affinity of compound 978336. Based on the consensus sequence of VEGF-A binding polypeptides defined in FIG. 6A, four residue positions (14, 24, 28 and 32) lacked strong consensus and displayed significant variation (i.e., r14, 124, r28, and s32). In the crystal structure of 978336 bound to VEGF-A (FIG. 35E) these four residues were not buried interfacial contacts, but in general appear to make weaker unoptimized interactions. Specifically, residues r14 and s28 do not make direct contact with VEGF, 124 is a hydrophobic sidechain positioned near an acidic patch, and r28 is too distant from any acidic sidechains to form an optimal salt-bridge (less than 4 angstroms). These sites were selected for soft-randomization using kunkel mutagenesis (see x positions in FIG. 35G). The resulting pIII phage library (SEQ ID NO: 158) was panned using similar high-stringency conditions as described above to identify improved binders to D-VEGF-A. After the third round of selection a preferred consensus motif was identified, containing two predominant mutations, L24V and S32R as compared to parent compound 978336 (SEQ ID NO: 117) (FIG. 35F). A representative clone, variant Z domain 980181 (FIG. 35G; SEQ ID NO: 119) was synthesized as a new D-protein binder and exhibited a VEGF-A affinity of 66 nM as measured by SPR (FIG. 35G). Thus, affinity optimization improved VEGF binding affinity by approximately 8-fold over parent compound 978336.

### Example 9: Bivalent D-Peptidic Antagonists of VEGF-A

Given the D-peptidic antagonist compounds 11055 and 978336 bind to non-overlapping epitopes on VEGF-A and directly block both the D2 and D3 binding sites, we engineered a chemically linked conjugate of compounds 11055 and 978336 in order to assess the overall effect on binding to target and antagonistic activity. Both compounds 11055 and 978336 were chemically synthesized with additional N-terminal cysteine residues, which were conjugated with a bis-maleimide PEG8 linker using conventional methods to provide for an N-terminal to N-terminal linkage (FIG. 36A).

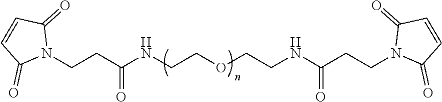

Bis-Mal-PEG(n) bifunctional linker, where n is 3, 6 or 8

The new heterodimer, compound 979111, exhibited a VEGF-A binding affinity of 1.7 nM as measured by SPR (FIG. 9B). This is consistent with an avidity effect whereby linking the two independent binders into single heterodimer results in a molecule with higher affinity than either binder alone. Importantly, in the HUVEC cell proliferation assay the heterodimer 979111 exhibited similar VEGF-A blocking activity to Avastin. The IC50 for inhibition of cell proliferation in response to VEGF signaling was 1.1 nM for 979111 and 0.7 nM for Avastin, representing >500-fold improvement over 11055 (FIG. 31B). Together these results show that heterodimeric D-peptidic antagonists of VEGF-A can effectively block signaling activity in a cell-based assay and have therapeutic potential as VEGF antagonists.

### Example 10: Tetradomain D-Peptidic antagonists of VEGF-A

To further improve both the affinity and potency of the D-peptidic compounds, a scheme was devised for the chemical linkage of the monomeric D-protein antagonists into a dimeric bivalent antagonist. Conceptually, two 980181 polypeptides are tethered to each other through their carbon-termini and then a polypeptide 979110 is site-specifically conjugated to each of the 980181 polypeptides in the dimer to provide a tetradomain D-protein that would mimic VEGF receptor engagement. FIG. 38A shows an overlay of structures of both compounds 11055 and 978336 bound to VEGF-A dimer where exemplary sites for chemical linkage of the domains is indicated using PEG-derivatives (FIG. 38A). Specifically, the 978336 carbon-termini are within ˜15 angstroms from one other and two lysine sidechains, k19 in 11055 and k7 in 978336, are within ˜23 angstroms.

A synthesis strategy was developed whereby two components would be synthesized in parallel using solid-phase peptide synthesis methods and a single click conjugation step would assemble the full tetradomain compound for final purification (FIG. 38B). D-protein 979110 was synthesized as a monomer containing either a PEG2-azide or PEG3-azide derivative extending from Lysine 19, and an oxidized intramolecular disulfide bond between c7-c38. 980181 was synthesized from aa carbon-terminal coupled linker resin, creating a homodimer during synthesis. In addition, a PEG2-alkyne derivative was incorporated at lysine 7 to facilitate conjugation to 979110. In the final conjugation step, two copies of 979110 were linked to the 980181 homodimer using click chemistry to yield tetradomain D-protein derivatives with either a PEG2/PEG2 (980870) or a PEG3/PEG2 (980871) combination of linker lengths (FIG. 38C) . SPR titrations of the tetrameric D-proteins exhibited ultra-high binding affinity with K_{D} measurements of 0.32 nM for 980870 and 0.42 nM for 980871.

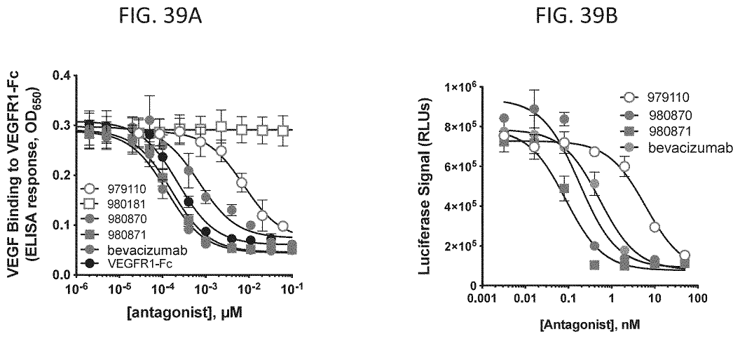

Since the D-protein tetrdomain compound is capable of sub-nanomolar binding to VEGF-A, a more accurate characterization of its antagonistic activity could be obtained in the VEGF-A/VEGFR1 blocking ELISA using a sub-nanomolar concentration of VEGF-A and long-incubation equilibrium binding conditions. Specifically, 150 pM of VEGF-A was incubated overnight with the antagonist titrations, then incubated on plate-coated VEGFR1-Fc for 5 hr to allow any free VEGF-A to bind the receptor. Under these conditions, the affinity matured monomer 979110 had an IC50 of 7 nM while the D-protein tetadomain compounds exhibited potent IC₅₀s of 128 pM (980870) and 163 pM (980871), in agreement with their sub-nanomolar binding affinities (FIG. 39A). Importantly, the D-protein tetradomain compounds were ˜4-fold more potent than bevacizumab which had an IC50 of 701 pM, and also slightly better than the soluble decoy VEGFR1-Fc (IC50 of 220 pM).

To translate these findings to VEGF signaling blockade, we used a cell-based assay for VEGFR2 signaling in a 293 luciferase reporter cell line. Here VEGF-A activates VEGFR2 signaling in 293 cells resulting in luciferase expression as a functional readout. Inhibition of VEGF-A signaling in this system results in a loss of luciferase signal. In an effort to mimic the ELISA conditions, 150 pM of VEGF-A was used to elicit a measurable luciferase signal and the antagonist were titrated to block this activity. Here, 979110 showed an IC50 of 6.1 nM while the tetradomain D-proteins showed sub-nanomolar IC₅₀s of 180 pM (980870) and 90 pM (980871), in very good agreement with the in vitro ELISA results (FIG. 39B). Furthermore, in this setting the D-protein tetradomain compounds were 3-6-fold more potent than bevacizumab (IC₅₀ of 530 pM) in blocking the activity of VEGF, demonstrating the potential of synthetic D-proteins to achieve antibody-like activity.

### Example 11: A Potent, Non-immunogenic D-Protein Antagonist of Vascular Endothelial Growth Factor Prevents Retinal Vascular Leakage and Inhibits Tumor Growth

A chemically synthesized D-protein blocks VEGF signaling with antibody-like potency, exhibits efficacy in ophthalmic and oncology disease models, and evades the humoral anti-drug antibody response.

Mirror-image phage display and structure-guided optimization were used to engineer a fully synthetic D-protein that antagonizes VEGF-A using a receptor mimicry mechanism. Phage panning against mirror-image D-VEGF-A yielded independent proteins that bound canonical receptor interaction sites. Crystal structures guided affinity maturation and the design of a chemical linkage to create a heterodimeric D-protein that tightly bound natural VEGF-A, inhibiting signaling activity at picomolar concentrations. The D-protein VEGF antagonist described herein, prepared by total chemical synthesis, prevented vascular leakage in a rabbit eye model of wet age-related macular degeneration, slowed tumor growth in the MC38 syngeneic mouse tumor model and was non-immunogenic during treatment or following subcutaneous immunization.

### Main Text:

D-Proteins are mirror-image molecules composed entirely of D-amino acids and the achiral amino acid glycine. D-proteins resist digestion by endogenous proteases, avoiding fragmentation into peptides required for immunologic presentation (1, 4, 8), and are reported to not stimulate an immune response, even when emulsified in a strong adjuvant and repeatedly administered by subcutaneous injection (1, 2).

The antagonist of VEGF of as described herein was able to completely block vascular leakage induced by VEGF-A in a rabbit eye model of wet AMD. Furthermore, cross-species activity against human and murine VEGF-A enabled demonstration of tumor growth inhibition in the MC38 syngeneic mouse model and lack of immunogenicity following treatment. In addition, there was complete absence of a humoral antibody response following repeated subcutaneous immunization with our D-protein antagonist emulsified in an adjuvant.

Mirror-Image Protein Phage Display

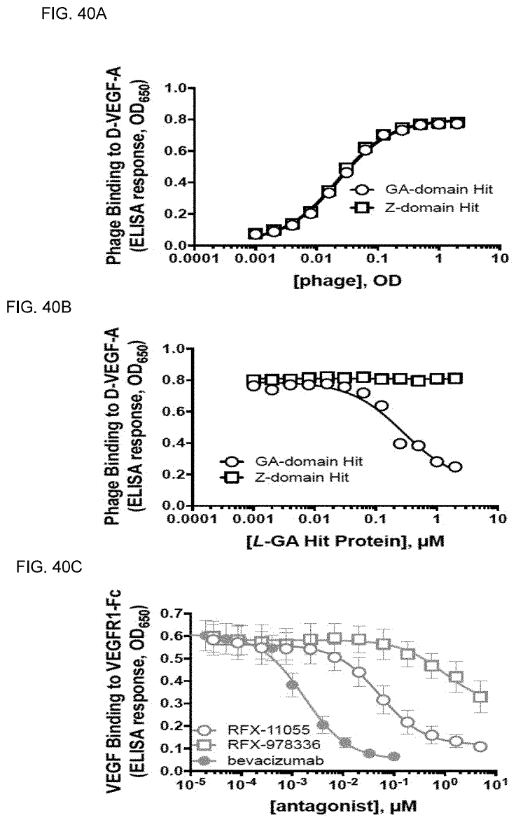

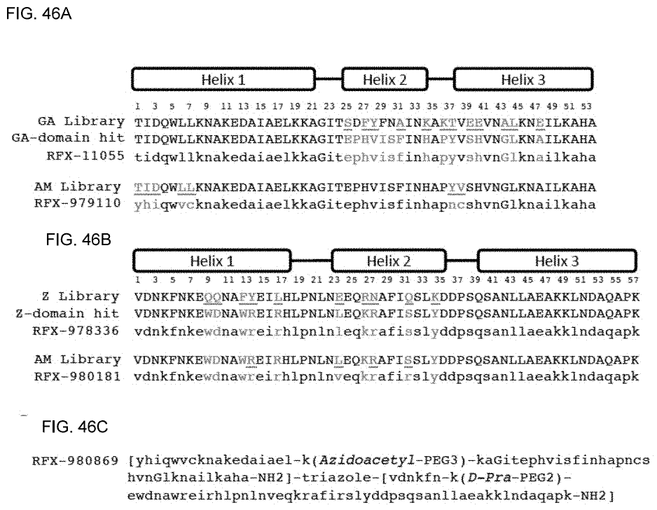

To develop a multivalent D-protein antagonist, protein binders to non-overlapping epitopes on VEGF-A were identified. The 53-residue GA domain and 58-residue Z domain proteins derived from bacterial protein G and protein A respectively (22, 23), were selected as two different 3-helix bundle scaffolds for phage display because of their high stability, small size, and ease of chemical synthesis. M13 phage display libraries were generated for the Z and GA-domain scaffolds containing 10 and 12 hard-randomized library positions, respectively (FIG. 46A-46C). A biotinylated form of the target D-VEGF-A(8-109) was prepared by total chemical synthesis, and each phage library was panned separately against D-VEGF-A-biotin under increasingly stringent target concentrations and wash conditions (Sup methods). In a qualitative binding ELISA, phage clones representing the consensus hits for both the GA and Z domains bound to D-VEGF-A in a concentration-dependent manner (FIG. 40A). The GA binder was synthesized as an L-protein and utilized as a competitor in phage competition ELISAs to confirm reversible binding prior to synthesizing hits as D-proteins. The GA binder directly blocked its parent phage clone from binding to D-VEGF-A with an IC50 of 280 nM, but had no effect on the binding of the Z-domain phage clone (FIG. 40B), suggesting the two proteins targeted independent epitopes on VEGF-A.

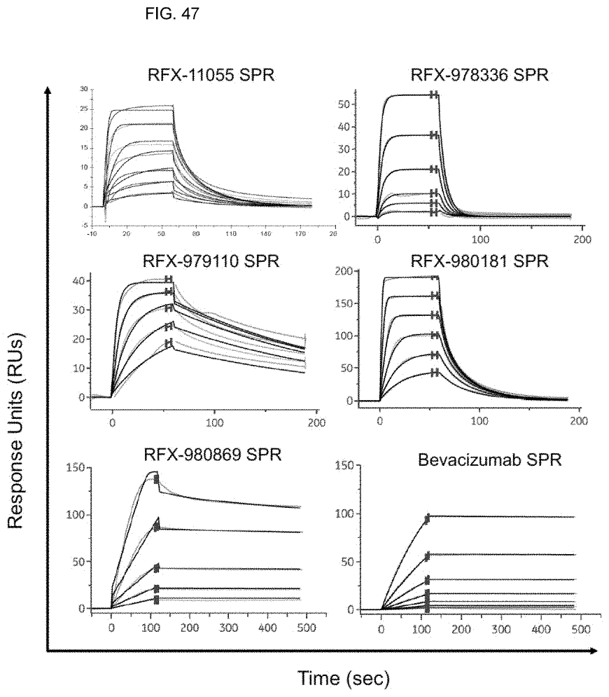

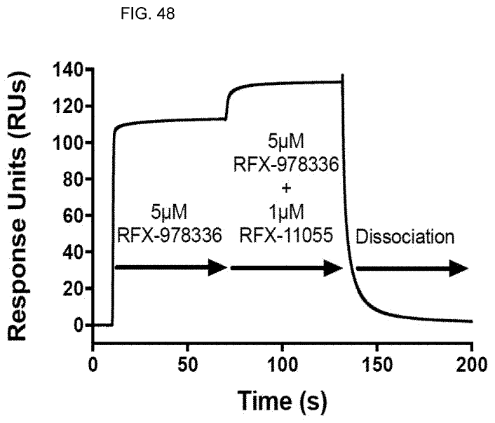

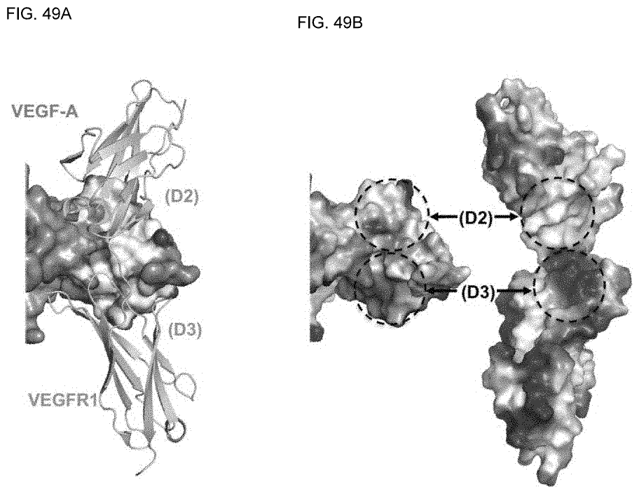

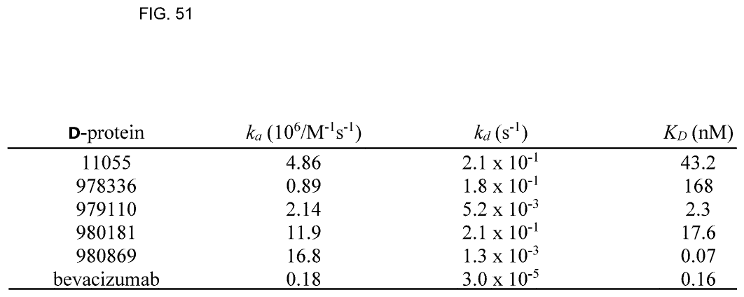

Both GA and Z-domain hits were synthesized as D-proteins (RFX-11055 and RFX-978336, respectively) for further characterization as binders to the natural L-protein form of VEGF-A. Titrations of the D-protein binders performed against L-VEGF-A using surface plasmon resonance (SPR) revealed binding affinities of 43 nM for the GA-domain binder RFX-11055 and 168 nM for the Z-domain binder RFX-978336 (FIG. 47 and FIG. 51), demostrating the D-enantiomers retained specific binding activity. Furthermore, SPR-based epitope mapping studies showed that RFX-11055 and RFX-978336 were capable of simultaneous and additive binding to VEGF-A (FIG. 48), confirming they bound to independent and non-overlapping epitopes.

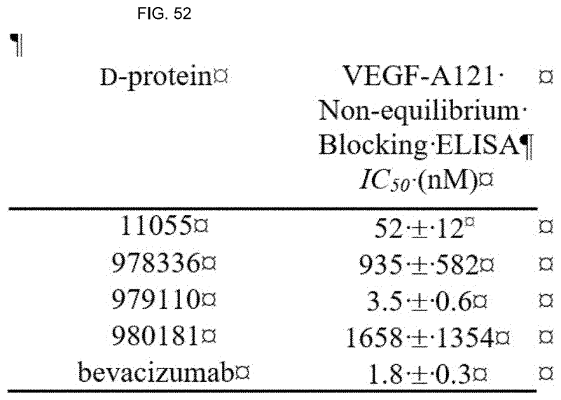

Antagonists of VEGF-A signaling need to block the VEGF receptors from interacting with two binding sites formed at the interface of the symmetrical VEGF-A homodimer (16, 24). To assess VEGF antagonism, a non-equilibrium VEGF-A121 blocking ELISA was employed that measures binding of biotinylated VEGF-A isoform 121 (VEGF-A121-biot) to VEGFR1-Fc coated on a plate (Sup methods). Both RFX-11055 and RFX-978336 exhibited inhibition of VEGF-A121 binding to VEGFR1 with apparent IC50 values of 52 nM and 935 nM, respectively (FIG. 40C and FIG. 52). These D-proteins showed clear inhibitory activity.

Structure-Guided Affinity Maturation of D-Protein VEGF-A Antagonists

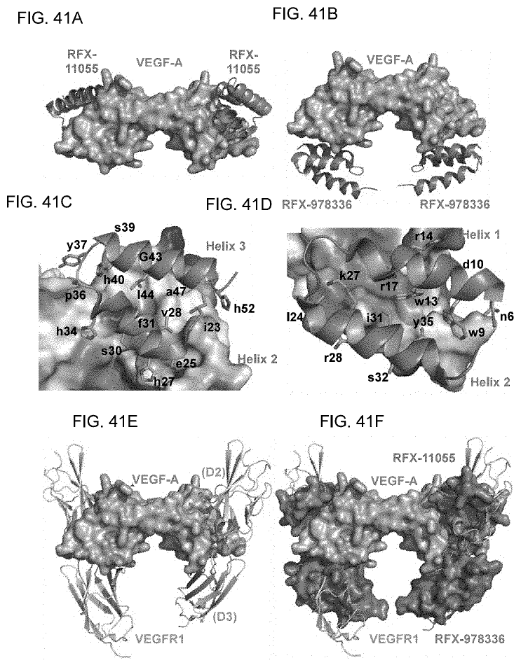

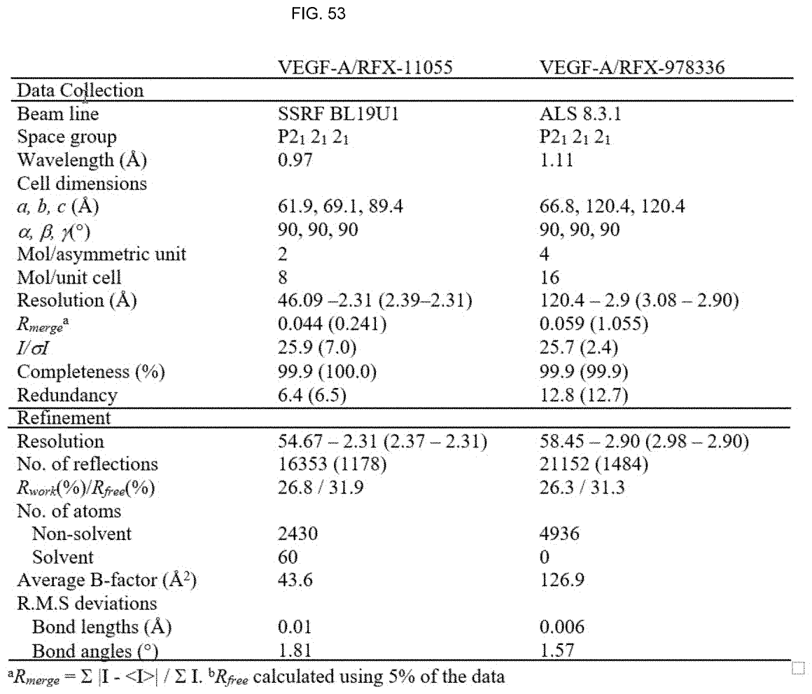

In order to guide further optimization of the D-protein antagonists, two independent crystal structures of VEGF-A in complex with RFX-11055 and RFX-978336 at 2.3 Å and 2.9 Å, respectively were solved (FIG. 53). In both cases, the D-proteins interact symmetrically with the binding sites at distal ends of VEGF-A (FIG. 41A and FIG. 41B). RFX-11055 utilizes predominantly hydrophobic and polar residues selected through panning (h27, v28, f31, h34, p36, y37, h40, l144 and a47) to interact with ˜800 A2 surface area on VEGF-A (FIG. 41C). In contrast, the D-protein RFX-978336 employs highly basic contacts (r14, r17, k27 and r28) to interact with acidic patches on VEGF-A, in addition to a few polar contacts (w9, w13 and y35), ultimately comprising a smaller surface area of ˜450 A2 (FIG. 41D). The structures of VEGF-A in complex with VEGFR1 and VEGFR2 and the details of the interactions formed between the homodimeric multi-Ig domain receptor and VEGF-A have been described (16, 24). Specifically, the receptor Ig-like domains 2 and 3 (D2 and D3) bind two identical sites on the distal ends of homodimeric VEGF-A protein molecule, which has C2 symmetry (FIG. 41E). An overlay of the VEGF-A/VEGFR1 structure with bound RFX-11055 and RFX-978336 highlights the direct overlap between D2 and D3 of VEGFR1 and the D-proteins, revealing a competitive mechanism of receptor binding inhibition (FIG. 41F). Interestingly, the predominant use of hydrophobic contacts by RFX-11055 and polar contacts by RFX-978336 closely mimics the nature of the specific interactions made by D2 and D3 with VEGF-A (FIG. 49A-49B).

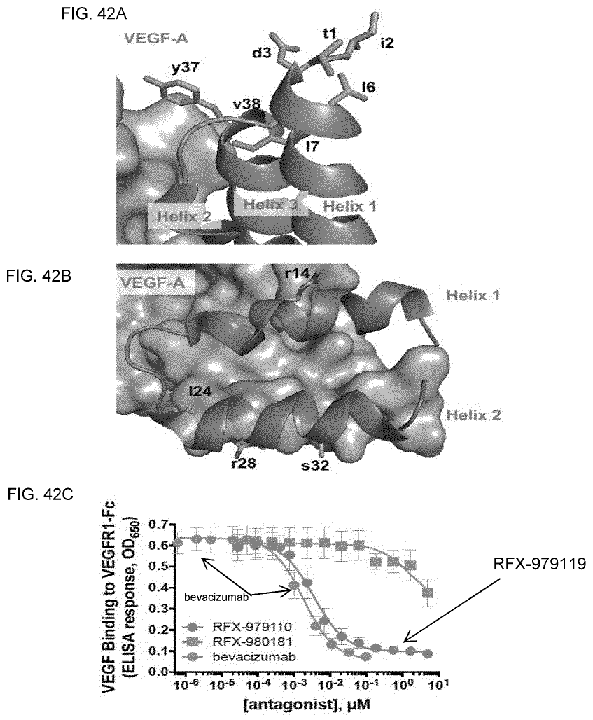

Based on the 3-helix bundle structure of RFX-11055, a seven-residue soft randomization library was designed to stabilize the packing between the N-terminal helix 1 and the helix 2-3 loop (FIG. 42A). Kunkel mutagenesis was used to simultaneously randomize each selected residue with the NNC degenerate codon representing 15 possible substitutions including cysteine. After four rounds of high-stringency panning using L-RFX-11055 as a competitor protein against D-VEGF-A, a consensus motif was identified containing two fixed cysteine residues at positions L7 and V38 (FIGS. 46A-46C). The conserved cysteine mutations at positions 7 and 38 would appear to place sidechain sulfhydryl groups in proximity to form an intra-molecular disulfide bond. The consensus variant, RFX-979110 synthesized as a D-protein with an oxidized disulfide bond, had a binding affinity of 2.3 nM by SPR, representing a 19-fold affinity improvement over RFX-11055 (FIG. 47 and FIG. 51).

Affinity maturation of RFX-978336 involved selecting VEGF-A contact residues showing minimal conservation from the initial panning for further interrogation using soft-randomization. A total of 4 residues were selected and Kunkel mutagenesis was used to soft-randomize each residue (FIG. 42B and FIGS. 46A-46C). A similar high-stringency panning approach was employed using synthesized L-RFX-978336 as a competitor. After 3 rounds of selection, a preferred consensus motif was identified containing L24V and S32R mutations (FIG. 42B). The Z-domain consensus variant, RFX-980181, was synthesized as a D-protein and exhibited a measured binding affinity of 18 nM, representing a 9-fold affinity improvement over RFX-978336 (FIG. 47 and FIG. 51).

The affinity-matured D-proteins were evaluated in a non-equilibrium VEGF-A121 blocking ELISA to measure their antagonistic activity. RFX-979110 blocked VEGF-A121 binding to VEGFR1-Fc with an IC50 of 3.5 nM, a 15-fold improvement over RFX-11055 and approaching the potency of bevacizumab, which had an IC50 of 1.8 nM in this assay (FIG. 42C and FIG. 52). In contrast to RFX-979110, the improved binding affinity for RFX-980181 showed no effect on its antagonistic activity (IC50 of 1,658 nM, within experimental uncertainty of the original lead RFX-978336 measured in the same assay (FIG. 52)). Given that previous studies showed VEGFR1 binding of VEGF-A is mainly driven by the high-affinity D2 domain (15), a likely explanation is that blocking of the D3 domain site has an ancillary effect on overall receptor engagement.

Total Chemical Synthesis of a Heterodimeric D-Protein Antagonist of VEGF-A Signaling\

The affinity and potency of the monomeric D-proteins were enhanced by chemically linking them together, recapitulating the interactions between the VEGF receptor D2 and D3 domains and VEGF-A. Based on the structures of RFX-11055 and RFX-978336 bound to VEGF-A, and their similarity to RFX-979110 and RFX-980181, site-specifically linkage was carried out between them through chemically modified lysine side chains K19 and K7, respectively, using a Click reaction to create a heterodimeric D-protein construct designed to mimic natural receptor engagement (FIG. 50A and FIG. 50B). By employing total chemical synthesis, RFX-979110 was synthesized as a monomer containing a PEG3-azide derivative extending from the side chain of Lys19, with an intramolecular disulfide bond between Cys7-Cys38. The D-protein RFX-980181 was synthesized with a PEG2-alkyne derivative incorporated within RFX-980181 on the side chain of Lys7 to facilitate conjugation to the PEG-azide equipped RFX-979110. In the final linkage step, RFX-979110 was reacted with RFX-980181 using Click chemistry, yielding a 13 kDa heterodimeric D-protein (RFX-980869) with a PEG3/PEG2 linker (Supplemental methods, FIGS. 46C and 50B). RFX-980869 was characterized by LC/MS spectra for following chemical synthesis and purification

SPR titrations of the heterodimeric D-protein RFX-980869 demonstrated ultra-high binding affinities with KD measurement of 0.07 nM, similar to that of bevacizumab at 0.16 nM (FIG. 47 and FIG. 51). The bevacizumab antibody was titrated under similar conditions and it was concluded that the limit of measurement was reached for accurately determining affinities in the sub-nanomolar concentration range. The extraordinarily high binding affinities observed are consistent with a multivalent interaction enabled by the chemical linkage of the individual D-proteins into a heterodimer.

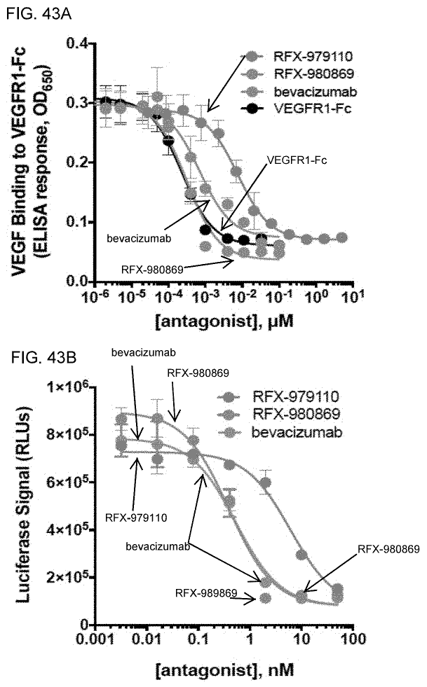

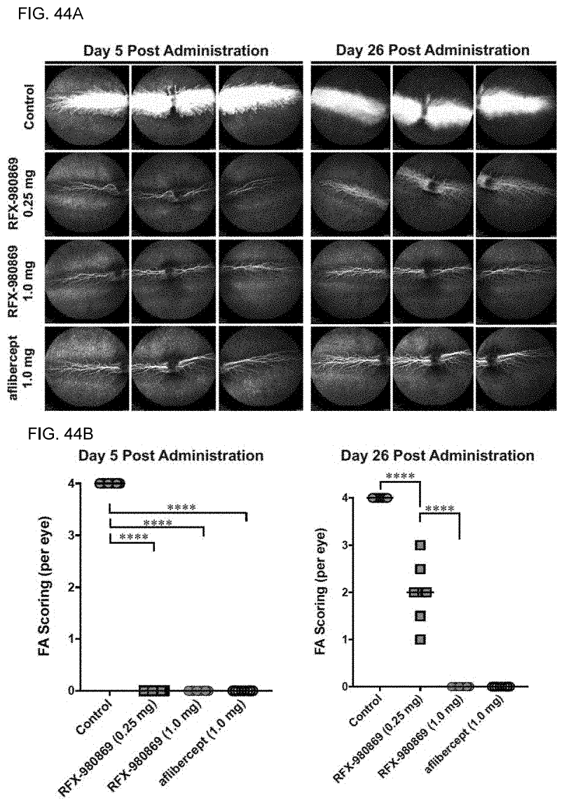

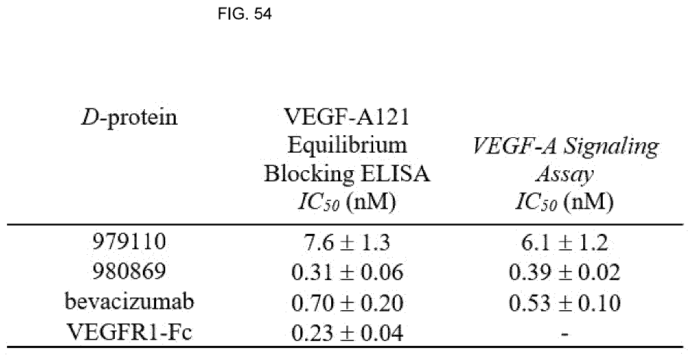

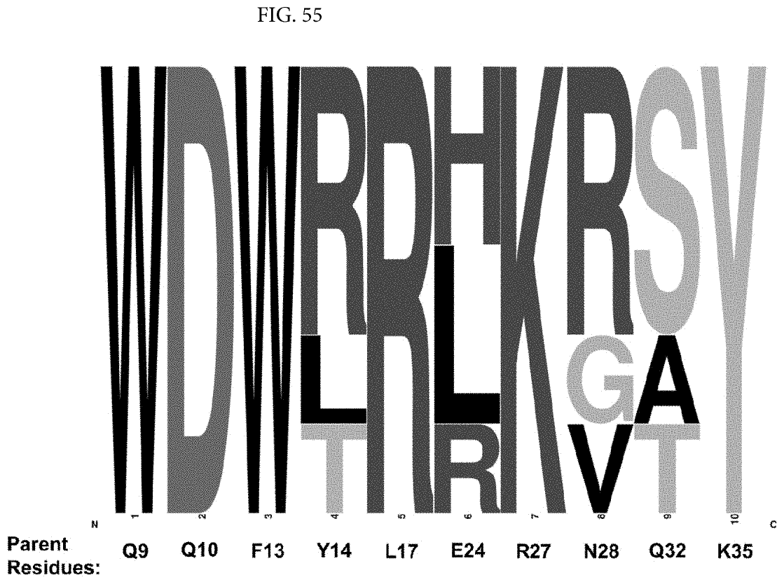

To further characterize its antagonistic activity a VEGF-A121/VEGFR1 blocking ELISA was employed using a sub-nanomolar concentration of VEGF-A121 under long-incubation equilibrium binding conditions (Supplementary methods). Under these conditions, the affinity-matured monomer RFX-979110 showed an IC50 of 7.6 nM, while the D-protein heterodimer exhibited an IC50 value of 0.31 nM, in reasonable agreement with the affinity measured by SPR (FIG. 43A and FIG. 54). Notably, the IC50 value of the D-protein heterodimer was lower than bevacizumab (IC50 of 0.70 nM) and similar to a soluble decoy receptor VEGFR1-Fc, which had an IC50 value of 0.23 nM. The measured IC50 values for the synthetic heterodimer and the soluble decoy receptor were approaching the concentration of VEGF-A121 in the assay, indicating that their potency may be higher than what can be measured in this assay.

To demonstrate the effects of these D-protein antagonists on VEGF signaling a cell-based luciferase reporter assay was used driven by VEGFR2 receptor activation. In this assay, 150 pM of VEGF-A activates VEGFR2 signaling causing an increase in luciferase expression, while inhibition of VEGF-A results in a decrease in luciferase expression. The monomeric D-protein RFX-979110 had an IC50 of 6.1 nM while the heterodimeric D-protein RFX-980869 exhibited a sub-nanomolar IC50 value of 0.49 nM, equivalent to bevacizumab (IC50 of 0.53 nM) in blocking VEGFR2 signaling (FIG. 43B and FIG. 54). In summary, these data demonstrate that chemical linkage of monomeric D-proteins, using total chemical synthesis, resulted in a heterodimer capable of disrupting the very high-affinity interaction between VEGF-A and its receptor.

RFX-980869 Exhibits Potent Activity In Vivo and is Non-Immunogenic

The activity of RFX-980869 was explored in a rabbit eye model for wet AMD and a syngeneic mouse tumor model in order to demonstrate applications in both ophthalmic and oncology diseases, respectively. In the rabbit eye model for wet AMD, intravitreal challenge with exogenous VEGF-A165 induces vascular leakage of the retina that can be monitored using fluorescein angiography (FA). VEGF-A blockade can prevent the diffuse leakage of fluorescein into the eye, which serves as a measure of efficacy. Here, we tested RFX-980869 for dose-dependent efficacy and durability in comparison to aflibercept. After a single intravitreal administration of RFX-980869 at 0.25 mg or 1.0 mg per eye, rabbits were challenged with exogenous VEGF-A twice over a 1-month period (Day 2 and Day 23) and their eyes were examined three days later (Day 5 and Day 26). Notably, a single dose of RFX-980869 at either 0.25 mg or 1 mg was able to significantly block the vascular leakage observed in control eyes following both VEGF challenges (FIG. 43C). Furthermore, at Day 26 the 1.0 mg dose of RFX-980869 completely blocked vascular leakage on par with 1.0 mg of aflibercept while the 0.25 mg dose showed a reduction in efficacy that was characterized by fluorescein leakage and increased vessel tortuosity (FIG. 43C). These results were confirmed by detailed examination and scoring of FA images from all eyes involved in the study at Day 26 (FIG. 44A-44B) and demonstrate clear dose-dependent durability of treatment with RFX-980869.

To assess the tumor growth inhibition potential of RFX-980869, the cross-reactivity of RFX-980869 with mouse VEGF-A (data not shown) was studied and used the syngeneic MC38 mouse tumor model. MC38 colon cancer tumors were established in C57BL6 mice transgenic for human PD-1, and reached 82 mm3 prior to initiation of treatment. Nivolumab was used as a positive control since we could not find published precedence for the efficacy of VEGF-A antagonists in a syngeneic MC38 tumor model. RFX-980869 dosed daily at 6 mg/kg for 2 weeks exhibited inhibition of tumor growth similar to nivolumab dosed biweekly at 3 mg/kg (FIG. 44A). Both RFX-980869 at 2 mg/kg and nivolumab at 1 mg/kg failed to show tumor growth inhibition with respect to the vehicle control group, confirming there was dose-dependent efficacy of the two treatments in this setting. At day 15, following the termination of daily RFX-980869 dosing, tumor growth inhibition was 31% for RFX-980869 at 6 mg/kg and 48% for nivolumab at 3 mg/kg (FIG. 44B).

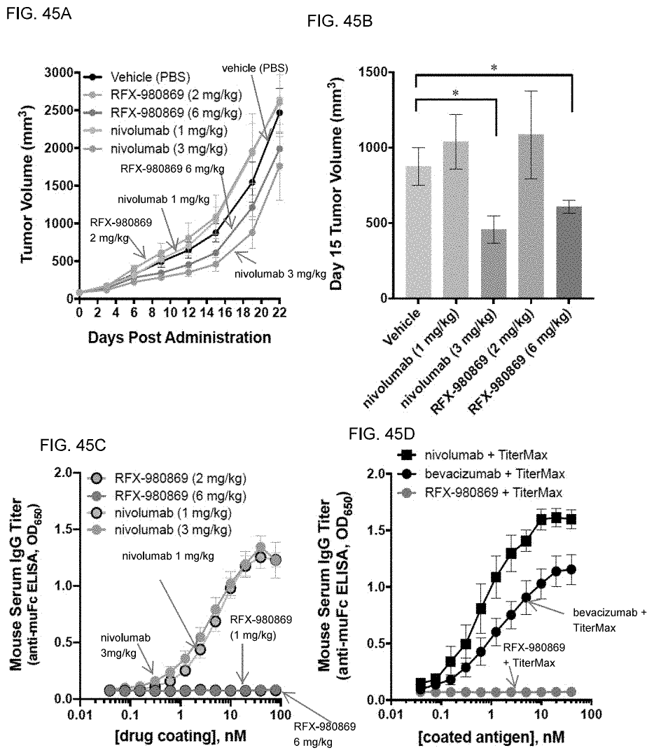

To highlight the non-immunogenic potential of our heterodimeric D-protein antagonist, mouse serum was analyzed for anti-drug-antibodies (ADAs) at the termination of the tumor study. In this fully immuno-competent mouse tumor model, plasma from both the low and high dose RFX-980869 treatment groups exhibited a complete lack of an IgG titer response against RFX-980869, while the nivolumab treatment groups had saturating levels of IgG titer (FIG. 45C). Thus, despite both agents being completely foreign antigens, only nivolumab elicited a strong ADA response. Given their different mechanisms of tumor growth inhibition, a separate study was performed to directly immunize mice with repeated subcutaneous injections of either RFX-980869, nivolumab, or bevacizumab emulsified in an adjuvant to determine if non-immunogenicity is an inherent property of RFX-980869 Immunization with the monoclonal antibodies generated strong IgG titers after Day 42, while RFX-980869 completely evaded the humoral antibody response (FIG. 45D). Taken together, the in vivo results not only demonstrate the potent activity of our synthetic VEGF-A antagonist in both ophthalmic and oncology settings, but also show a clear differentiation over monoclonal antibodies with respect to its lack of immunogenicity.

Discussion

Mirror-image protein phage display and structure-guided optimization was used to independently mature two different 3-helix bundles into D-protein antagonists that occupied the D2 and D3 binding sites on VEGF-A (FIG. 41F). Through side chain-selective chemical linkage of the monomers, the resulting 13 kDa D-protein was able to bind approximately 1,250 A2 of

VEGF-A surface area, achieving picomolar affinity while replicating a mechanism that closely resembles VEGF receptor binding. By blocking all four receptor interaction sites on VEGF-A, the resulting neutralization of VEGF-A is likely to be irreversible on the timescale of turnover and clearance in vivo. Like aflibercept, which utilizes a receptor decoy mechanism to block VEGF-A (25, 26), the heterodimeric D-protein VEGF antagonist described herein also uses receptor mimicry, blocking all of the VEGF receptor binding sites on VEGF-A albeit in a much smaller, chemically synthesized D-protein format.

The heterodimeric D-protein VEGF antagonist described herein is half the size of brolucizumab, is readily soluble in PBS (pH 7.4), and is amenable to high-dose formulations. Its small size enables better retinal penetration and rapid systemic clearance after leaving the eye. Moreover, its properties including increased proteolytic stability and lack of immunogenicity provide further advantages in the durability of a therapeutic response, lower inflammation, and an absence of ADA from long-term chronic treatment.

### REFERENCES

1. H. M. Dintzis, D. E. Symer, R. Z. Dintzis, L. E. Zawadzke, J. M. Berg, A comparison of the immunogenicity of a pair of enantiomeric proteins. Proteins Struct. Bioinforma. 16, 306-308 (1993).
2. M. Uppalapati et al., A Potent D-Protein Antagonist of VEGF-A is Nonimmunogenic, Metabolically Stable, and Longer-Circulating in Vivo. ACS Chem. Biol. 11, 1058-1065 (2016).
3. L. Zhao, W. Lu, Mirror-image proteins. Curr. Opin. Chem. Biol. 22, 56-61 (2014).
4. M. Sauerborn, V. Brinks, W. Jiskoot, H. Schellekens, Immunological mechanism underlying the immune response to recombinant human protein therapeutics. Trends Pharmacol. Sci. 31, 53-59 (2010).
5. M. Krishna, S. G. Nadler, Immunogenicity to biotherapeutics—The role of anti-drug immune complexes. Front. Immunol. 7 (2016), doi:10.3389/fimmu.2016.00021.
6. F. A. Harding, M. M. Stickler, J. Razo, R. DuBridge, The immunogenicity of humanized and fully human antibodies. MAbs. 2, 256-265 (2010).
7 R. Dingman, S. V. Balu-Iyer, Immunogenicity of Protein Pharmaceuticals. J. Pharm. Sci., 1-18 (2019).
8. R. C. Milton, S. C. Milton, S. B. Kent, Total chemical synthesis of a D-enzyme: the enantiomers of HIV-1 protease show reciprocal chiral substrate specificity [corrected]. Science. 256, 1445-8 (1992).
9. T. Katoh, K. Tajima, H. Suga, Consecutive Elongation of D-Amino Acids in Translation. Cell Chem. Biol. 24, 46-54 (2017).
10. T. N. Schumacher et al., Identification of D-peptide ligands through mirror-image phage display. Science. 271, 1854-7 (1996).
11. D. M. Eckert, V. N. Malashkevich, L. H. Hong, P. A. Carr, P. S. Kim, Inhibiting HIV-1 entry: Discovery of D-peptide inhibitors that target the gp41 coiled-coil pocket. Cell. 99, 103-115 (1999).
12. T. Van Groen et al., Reduction of Alzheimer's disease amyloid plaque load in transgenic mice by D3, a D-enantiomeric peptide identified by minor-image phage display. ChemMedChem. 3, 1848-1852 (2008).
13. M. Liu et al., D-peptide inhibitors of the p53-MDM2 interaction for targeted molecular therapy of malignant neoplasms. Proc. Natl. Acad. Sci. 107, 14321-14326 (2010).
14. B. D. Welch, A. P. VanDemark, A. Heroux, C. P. Hill, M. S. Kay, Potent D-peptide inhibitors of HIV-1 entry. Proc. Natl. Acad. Sci. 104, 16828-16833 2007).
15. C. Wiesmann et al., Crystal structure at 1.7 A resolution of VEGF in complex with domain 2 of the Flt-1 receptor. Cell. 91, 695-704 (1997).
16. M. S. Brozzo et al., Thermodynamic and structural description of allosterically regulated VEGFR-2 dimerization. Blood. 119, 1781-1788 (2012).
17. L. G. Presta et al., Humanization of an anti-vascular endothelial growth factor monoclonal antibody for the therapy of solid tumors and other disorders. Cancer Res. 57, 4593-9 (1997).
18. Y. Chen et al., Selection and analysis of an optimized Anti-VEGF antibody: Crystal structure of an affinity-matured Fab in complex with antigen. J. Mol. Biol. 293, 865-881 (1999).
19. P. E. Dawson, T. W. Muir, I. Clark-Lewis, S. B. Kent, Synthesis of proteins by native chemical ligation. Science. 266, 776-9 (1994).
20. K. Mandal, S. B. H. Kent, Total chemical synthesis of biologically active vascular endothelial growth factor. Angew. Chemie—Int. Ed. 50, 8029-8033 (2011).
21. K. Mandal et al., Chemical synthesis and X-ray structure of a heterochiral {D-protein antagonist plus vascular endothelial growth factor} protein complex by racemic crystallography. Proc. Natl. Acad. Sci. 109, 14779-14784 (2012).
22. S. Lejon, I. M. Frick, L. Björck, M. Wikström, S. Svensson, Crystal structure and biological implications of a bacterial albumin binding module in complex with human serum albumin. J. Biol. Chem. 279, 42924-42928 (2004).
23. M. Tashiro et al., High-resolution solution NMR structure of the Z domain of staphlococcal protein A. J. Mol. Biol. 272, 573-590 (1997).
24. S. Markovic-Mueller et al., Structure of the Full-length VEGFR-1 Extracellular Domain in Complex with VEGF-A. Structure. 25, 341-352 (2017).
25. J. Holash et al., VEGF-Trap: A VEGF blocker with potent antitumor effects. Proc. Natl. Acad. Sci. 99, 11393-11398 (2002).
26. E. S. Kim et al., Potent VEGF blockade causes regression of coopted vessels in a model of neuroblastoma. Proc. Natl. Acad. Sci. 99, 11399-11404 (2002).
27. T.W. Olsen et al., Retina/Vitreous Preferred Practice Pattern Development Process and Participants (2015).
28. M. Van Lookeren Campagne, J. Lecouter, B. L. Yaspan, W. Ye, Mechanisms of age-related macular degeneration and therapeutic opportunities. J. Pathol. 232, 151-164 (2014).
29. J. Tietz et al., Affinity and Potency of RTH258 (ESBA1008), a Novel Inhibitor of Vascular Endothelial Growth Factor A for the Treatment of Retinal Disorders. IOVS. 56, 1501 (2015).
30. F. G. Holz et al., Single-Chain Antibody Fragment VEGF Inhibitor RTH258 for Neovascular Age-Related Macular Degeneration: A Randomized Controlled Study. Ophthalmology. 123, 1080-1089 (2016).
31. Efficacy and Safety of RTH258 Versus Aflibercept. www(dot)clinicaltrials(dot)gov/ct2/show/NCTO2 307682 (2014)
32. Efficacy and Safety of RTH258 Versus Aflibercept-Study 2. www(dot) clinicaltrials(dot)gov/ct2/show/NCT02434328 (2015)
33. G. Gasparini, R. Longo, M. Toi, N. Ferrara, Angiogenic inhibitors: a new therapeutic strategy in oncology. Nature Clinical Practice Oncology. 2, 562-577 (2005).
34. J. J. Wallin et al., Atezolizumab in combination with bevacizumab enhances antigen-specific T-cell migration in metastatic renal cell carcinoma. Nature Communications. 7,1-8 (2016).
35. M. Reck et al., Articles Atezolizumab plus bevacizumab and chemotherapy in non-small-cell lung cancer (IMpower150): key subgroup analyses of patients with EGFR mutations or baseline liver metastases in a randomised, open-label phase 3 trial. Lancet Respiratory Medicine. 19,1-15 (2019).
36. S. S. Sidhu, B. K. Feld, G. A. Weiss, M13 Bacteriophage Coat Proteins Engineered for Improved Phage Display. Protein Eng. Protoc., 205-220 (2006).
37. T. A. Kunkel, Rapid and efficient site-specific mutagenesis without phenotypic selection. Proc. Natl. Acad. Sci. 82, 488-492 (1985).

### Materials and Methods

Protein Synthesis Reagents

Fmoc-D-amino acids were purchased from Chengdu Zhengyuan Company, Ltd and Chengdu Chengnuo New-Tech Company, Ltd. Fmoc-D-Ile-OH was purchased from Chemimpex International, Inc. Fmoc-D-propargylglycine (Fmoc-D-Pra-OH) was purchased from Haiyu Biochem. MBHA Resin was purchased from Sunresin New Materials Co. Ltd., Xian Rink Amide linker was purchased from Chengdu Tachem Company, Ltd. Chloro-(2-Cl)-trityl-resin was purchased from Tianjin Nankai Hecheng Science and Technology Company, Ltd. Fmoc-NH2(PEG)n-COOH and other PEG linkers were purchased from Biomatrik Inc. 2-Azidoacetic acid was purchased from Amatek Scientific Company Ltd. Sodium ascorbate was purchased from TCI (Shanghai) Ltd. Copper sulfate pentahydrate (CuSO4.5H2O) was purchased from Energy Chemical.

D-VEGF-A Synthesis and Refolding

The D-VEGF-A polypeptide chain (COOH acid, residues 8-109 (33)) was chemically synthesized using solid phase peptide synthesis (SPPS) and native chemical ligation, and folded to form the protein covalent homodimer, using methods adapted from our previous work (21). Individual peptide fragments corresponding to 1: Gly¹-to-D-Tyr¹⁸, 2: D-Cys¹⁹-to-D-Arg⁴⁹, 3: D-Cys⁵⁰-to-D-Asp^{102,} were synthesized using standard Fmoc chemistry protocols for stepwise SPPS. Fragments 1 and 2 were synthesized on NH₂NH-(2-Cl)trityl-resin and fragment 3 was synthesized from pre-loaded Wang Resin. Briefly, preloaded Fmoc-aminoacyl-Wang Resin was initially swelled with DMF (10 mL/g) for 1 hour, then treated with 20% piperidine/DMF (30 min) to remove the Fmoc group and washed again with DMF (5 times). Fmoc-D-amino acid residues were coupled by addition of a pre-activated solution of 3 equivalents each of protected amino acid (0.4 M in DMF), diisopropylcarbodiimide (DIC), and hydroxybenzotriazole (HOBt) to the resin. After 1-2 h, the ninhydrin test showed the reaction was completed and the resin was washed with DMF (3 times). To remove the Fmoc group, piperidine (20% in DMF) was added to the resin for 30 min. After removal of the final Fmoc group, the resin was rinsed with DMF (3 times) and MeOH (2 times), dried under vacuum, then taken up in 85% TFA, 5% thioanisole, 5% EDT, 2.5% phenol and 2.5% water for cleavage. After 2 h, the resin was washed with TFA and the eluted peptide was concentrated by bubbling nitrogen gas. The crude peptides were precipitated with cold ether, pelleted by centrifugation, and washed with cold ether 2 times before drying under vacuum. Peptide residue was dissolved in water, purified by preparative reverse phase HPLC and analyzed by HPLC and MS.

Ligations between D-peptide-hydrazide fragments and D-Cys-peptide fragments were performed as follows: D-Peptide-hydrazide was dissolved in Buffer A (0.2M sodium phosphate containing 6 M GnHCl, pH 3.0), cooled to −15° C. in an ice-salt bath, and gently stirred by magnetic stirrer. NaNO₂ (7 equivalents) was added and the solution stirred for 20 min to oxidize the D-peptide-hydrazide to the peptide-azide. A solution of 4-mercaptophenyl acetic acid (MPAA) (50 eq) dissolved in Buffer B (0.2M sodium phosphate containing 6 M GnHCl, pH 7.0) was quickly added to the solution containing the newly-formed D-Peptide-azide (equal volume) to eliminate excess NaNO₂ and to convert the peptide-azide to the peptide-MPAA thioester. Then a solution of D-Cys-peptide in Buffer B (equal volume) was added to the solution containing the newly formed peptide-MPAA thioester. And the reaction mixture was adjusted to pH 7 with NaOH to initiate overnight native chemical ligation. Reaction progress was monitored by analytic RP-HPLC until completion, then treated by TCEP before HPLC purification.

Purification of the ligated peptide product was performed on a CXTHLC6000/Hanbon NU3000 prep system on Phenomenex C18/YMC C4 silica with columns of dimension 21.2×250 mm/20.0×250 mm. Crude peptides were loaded onto the prep column and eluted at a flow rate of 5 mL per minute with a shallow gradient of increasing concentrations of solvent B (0.1% TFA in 80% acetonitrile) in solvent A (0.1% TFA in water). Fractions containing the purified target peptide were identified by analytical LC-MS, combined, and lyophilized.

Final linear D-VEGF-A peptide was folded at pH 8.4 in aqueous Gu.HCl (0.15 M) containing a glutathione-reduced (2 mM)/glutathione-oxidized (0.4 mM) redox couple and stirred for 5 days to reach completion (21). Folded D-VEGF-A was purified by RP-HPLC.

Phage Display Libraries and Panning

Naïve GA- and Z-domain scaffold libraries were constructed as fusions to the N-terminal gene 8 major coat protein by previously described methods (34). Randomization of desired library positions (FIG. 46A-46C) was performed using Kunkel mutagenesis (35) with trinucleotide oligos allowing incorporation of all natural amino acids except cysteine. The resulting libraries contained >1010 unique members. For affinity maturation libraries, Kunkel mutagenesis was performed on RFX-11055 or RFX-978336 parent sequences using targeted NNC or soft-randomization oligos, respectively. Positions targeted for affinity maturation are highlighted in FIG. 51.

All phage selections were executed according to previously established protocols (34). Briefly, selections with the peptide libraries were performed using biotinylated D-VEGF captured with streptavidin-coated magnetic beads (Promega). Initially, three rounds of selection were completed with decreasing amounts of D-VEGF (2.0 mM, 1.0 mM, and 0.5 mM). The phage pools were then transferred to a N-terminal gene 3 minor coat protein display vector and subjected to an additional three rounds of panning with decreasing amounts of D-VEGF (200 nM, 100 nM, and 50 nM) and increased wash times. Individual phage clones were then sent in for sequencing analysis.

Synthesis of D-Protein Binders

The polypeptide chains of the affinity matured D-proteins RFX-979110 and RFX-98018 (FIG. 46A-46C) were prepared manually by Fmoc chemistry stepwise SPPS on Rink Amide MBHA Resin. Side-chain protection for amino acids was as follows: D-Arg(Pbf), D-Asp(OtBu), D-Glu(OtBu), D-Asn(Trt), D-Gln(Trt), D-Ser(tBu), D-Thr(tBu), D-Tyr(tBu), D-His(Trt), D-Lys(Boc), D-Trp(Boc). After chain assembly of the D-polypeptides was complete and the final Fmoc group removed, the resulting D-peptides had their side-chains deprotected and were simultaneously cleaved from the resin support by treatment with TFA containing 2.5% triisopropylsilane and 2.5% H₂O for 2.5 h at room temperature. Crude D-polypeptide products were recovered from resin by filtration, precipitated, and triturated with chilled diethyl ether then dried under vacuum. D-polypeptide chains folded spontaneously upon dissolution in appropriate buffer to yield the functional D-protein binder molecules.

Synthesis of the D-Protein Heterodimer

Step 1: Preparation of Azido-PEG3-D-979110 Resin.

Fmoc-aminoacyl-Rink Amide MBHA Resin was swelled in DMF (10-15 mL/g resin) for 1 hour. The suspension was filtered, exchanged into DMF containing 20% piperidine, and kept at room temperature for 0.5 hr under continuous nitrogen gas perfusion. The resin was then washed 5 times with DMF. Fmoc-D-amino acid-OH, DIC, HOBt and DMF were added to the resin. The suspension was kept at room temperature for 1 hr while a stream of nitrogen was bubbled through it. The ninhydrin test was used to monitor the coupling reaction until completion. The remaining D-amino acids corresponding to the affinity matured D-protein RFX-979110 were coupled to the peptidyl resin sequentially. Azido-PEG3-COOH was coupled to the primary amine of Lys¹⁹. After assembly of the amino acid sequence of the protected RFX-979110 polypeptide chain was complete, the final Fmoc group was removed by treatment with DMF containing 20% piperidine. The peptidyl-resin was washed with DMF (5 times), MeOH (2 times), DCM (2 times) and MeOH (2 times), then dried under vacuum overnight.

Step 2: Cleavage and Deprotection of Azido-PEG3-D-979110-Resin.

Cleavage solution (TFA/Thioanisole/EDT/Phenol/H₂O=87.5/5/2.5/2.5/2.5 v/v, 10 mL/g peptide Resin) was added to the dried Azido-PEGS-D-979110-resin. The suspension was shaken for 3 h and was filtered and the filtrate collected. Cold ether was added to the filtrate to precipitate the peptide which was recovered by centrifugation. The white precipitate was washed with ether twice, then dried under vacuum overnight to give crude Azido-PEG3-D-979110 as a white solid.

Step 3: Oxidation and Purification. Crude Azido-PEG3-D-979110 was oxidized using I₂.

Briefly, peptide (23.5 mg) was dissolved in 11 mL of 30% ACN and mixed with 330 μL of CH₃COOH. An I₂/MeOH solution was added dropwise until the mixture was pale yellow then aqueous sodium ascorbate was added dropwise to quench excess I₂. Purification of oxidized Azido-PEG3-D-979110 was performed on a CXTH LC6000/Hanbon NU3000 prep system on Phenomenex C18 silica with columns of dimension 21.2×250 mm. Crude peptides were loaded onto the prep column and eluted at a flow rate of 5 mL/min with a shallow gradient of increasing concentrations of solvent B (0.1% TFA in 80% acetonitrile in water) in solvent A (0.1% TFA in water). Fractions containing the pure target peptide were identified by analytical LC-MS, and were combined and lyophilized to give purified Azido-PEG3-D-979110 for subsequent click reaction with (Alkynyl-PEG2)-D-980181.

Step 4: Preparation ofAlkynyl-PEG2-D-980181 Resin.

Fmoc-aminoacyl-Rink Amide MBHA Resin was swelled in DMF (10-15 mL/g resin) for 1 hour. The suspension was filtered, exchanged into DMF containing 20% piperidine, and kept at room temperature for 0.5 hr under continuous nitrogen gas perfusion. The resin was then washed 5 times with DMF. Fmoc-D-amino acid-OH, DIC, HOBt and DMF were added to the resin. The suspension was kept at room temperature for 1 hr while a stream of nitrogen was bubbled through it. The ninhydrin test was used to monitor the coupling reaction until completion. The remaining D-amino acids corresponding to the affinity matured D-protein 980181 polypeptide chain were added to the sequentially, in order. Alkynyl-PEG2-COOH was coupled to the primary amine of Lys⁷. After assembly of the amino acid sequence of the protected RFX-979181 polypeptide chain was complete, the final Fmoc group was removed by treatment with DMF containing 20% piperidine.

The peptidyl-resin was washed with DMF (5 times), MeOH (2 times), DCM (2 times) and MeOH (2 times), then dried under vacuum overnight.

Step 5: Cleavage and Deprotection of Alkynyl-PEG2-D-980181.

Cleavage solution (TFA/TIS/H₂O 95/2.5/2.5v/v, 10 mL/g peptide Resin) was added into the alkynyl-PEG2-D-980181 homodimer resin. The mixture was shaken for 3 h and the filtrate was collected. Cold ether was added to the filtrate to precipitate the peptide which was collected by centrifugation. The white precipitate was washed with ether twice and dried under vacuum overnight to give crude alkynyl-PEG2-D-980181 homodimer as a white solid.

Step 6: Purification.

Purification of crude alkynyl-PEG2-D-980181 homodimer was performed on a CXTH LC6000/Hanbon NU3000 prep system on YMC C4 silica with columns of dimension 21.2×250 mm. Crude peptides were loaded onto the prep column and eluted at a flow rate of 10 mL per minute with a shallow gradient of increasing concentrations of solvent B (0.1% TFA in 80% acetonitrile in water) in solvent A (0.1% TFA in water). Fractions containing the pure target peptide were identified by analytical LCMS, combined, and lyophilized to give purified alkynyl-PEG2-D-980181 homodimer used for the click reaction with azido-PEGn-D-979110.

Step 7: Click Reaction and Purification.

Azido-PEG3-D-979110 and the alkynyl-PEG2-D-980181 were dissolved in ethanol:H₂O (v/v, 1:1), then 0.2 M CuSO₄ was added to the reaction mixture, followed by the addition of 0.2M of sodium ascorbate, and the reaction mixture was stirred at 30° C. for 2 h. The reaction mixture was loaded onto RP-HPLC without further workup and purified by gradient elution as described above. Fractions containing the desired product were identified by LCMS, combined, and lyophilized. Observed mass (LC-MS): 13174.0 Da; Calculated masses (average isotope composition): 13176.8 Da.

LC-MS Analysis of D-Proteins

Analytical RP-HPLC was performed on a HP 1090 system with Waters C4/Phenomenex C18 silica columns (4.6×150 mm, 3.5 μm/4.6×150 mm, 5.0 μm particle size) at a flow rate of 1.0 mL/min (50° C. column temperature). Peptides were eluted from the column using a 1.0% B/min gradient of water/0.1% TFA (solvent A) versus 80% acetonitrile in water/0.1% TFA (solvent B). Peptide masses were obtained by in-line electrospray MS detection using an Agilent 6120 LC/MSD ion trap.

Surface Plasmon Resonance Affinity Measurements

Surface plasmon resonance (SPR) binding measurements were carried out on a Biacore S200 (GE). Biotinylated VEGF-A(8-109) was immobilized on a biotin CAPture chip (GE) and serial dilutions of D-proteins were flowed over the chip at 30 μL/min in running buffer (10 mM Hepes, pH 7.4, 150 mM NaCl, 0.05% P20). Association reactions were 60 seconds for RFX-11055, -978336, -979110 and -980181 and 120 seconds for RFX-980869. Dissociation reactions were carried out in running buffer for either 120 seconds (RFX-11055, -978336, -979110, -980181) or 360 seconds (RFX-980869). All measurements were carried out at 25° C. SPR data are representative of multiple independent titrations. Kinetic fits were performed using Biacore software using a global single site binding model.

Expression and Purification of VEGF-A for Crystallography

The gene sequence for the VEGF-A (8-109) polypeptide chain was cloned into the expression vector pET21b with a His₆-tag and TEV cleavage site sequence added at the N-terminus. The recombinant plasmid was transformed into E. coli BL21-Gold, grown in LB medium supplemented with Ampicillin (100 μg/ml) and expression of the His-tagged protein was induced by 0.3 mM isopropyl-b-D-thiogalactoside (IPTG) at 16° C. overnight. Cells were harvested by centrifugation and then stored at −80° C.

Pelleted cells from 30 L of culture were resuspended in 1 L buffer A (20 mM Tris, pH 8.0, 400 mM NaCl) and then passed through high-pressure homogenization (3 cycles). His-tagged protein from supernatant was captured on a Ni-NTA resin column (30 ml). The column was washed with 20 CV of Buffer A containing 20 mM imidazole, 5 CV of Buffer C (20 mM Tris, pH 8.0, 1M NaCl) and 10 CV of buffer A containing 50 mM imidazole. The His₆-tagged-TEV site-VEGF-A protein was eluted with a high concentration of imidazole (0.25 M) in buffer A (5 CV). The eluted protein was digested with TEV protease at a 1:20 ratio (TEV: Protein) and dialyzed against 5 L buffer (20 mM Tris, pH 8.0, 50 mM NaCl.) at 4° C. overnight. Cleaved sample was loaded onto a 2^{nd} Ni-NTA column to remove free His-tag. Eluted VEGF-A protein was further purified by ion exchange chromatography on a Resource Q column (6 ml). A final SEC polishing step was performed using HiLoad 16/60 Superdex 75 pg column equilibrated with buffer A. Monodisperse VEGF-A peak fractions were identified by absorbance at 280 nm and were combined and concentrated to 10-15 mg/mL in the buffer A. Final purified VEGF-A(8-109) protein was 95% pure as assessed by SDS-PAGE analysis and the molecular weight was confirmed by direct injection MS.

Crystallography of VEGF-A/D-Protein Complexes

VEGF-A/RFX-11055 complex. Crystals for VEGF-A/RFX-11055 were grown by hanging drop vapor diffusion at 18° C. The drop was composed of 0.8 μL of VEGF-A/D-protein complex (2.72 mg/ml VEGF-A and 0.5 mM RFX-11055) mixed 1:1 with 0.8 μl of the crystallization solution containing 0.2 M Calcium Chloride, 0.1 M Tris pH 8.5, 18% w/v PEG 4000. Crystals were soaked in a cryo-protectant solution containing crystallization solution plus 20% (v/v) glycerol and were flash-frozen in liquid nitrogen. The diffraction data were collected at the Shanghai Synchrotron Radiation Facility beam line BL19U1 to 2.31 Angstroms resolution and processed in space group P2₁2₁2₁ using XDS. The structure was solved by molecular replacement using Phaser with VEGF structure (PDB ID: 3QTK) as the search model. Structure refinement and model building on the initial model were iteratively performed between Refmac5 and Coot. There are two copies of the {VEGF-A plus RFX-11055} complexes in an asymmetric unit. The detailed data processing and structure refinement statistics are listed in FIG. 53.

VEGF-A/RFX-978336 Complex.

Crystals for VEGF-A/RFX-978336 were grown by hanging drop vapor diffusion at 18° C. The drop was composed of 0.8 μL of VEGF-A/D-protein complex (5.44 mg/ml VEGF-A and 0.46 mM RFX-978336) mixed 1:1 with 0.80 of the crystallization solution containing 0.15 M Magnesium Chloride, 0.1 M Bis-Tris pH 5.5, 25% w/v PEG 3350. Crystals were soaked in a cryo-protectant solution containing crystallization solution plus 10% (v/v) glycerol and were flash-frozen in liquid nitrogen. The diffraction data were collected at ALS beam line 8.3.1 to 2.9 Angstroms resolution and indexed in space group P2₁2₁2₁ using XDS. The structure was solved by molecular replacement using Phaser with VEGF structure (PDB ID: 3QTK) as the search model. Structure refinement and model building on the initial model were iteratively performed between Refmac5 and Coot. There are four copies of the {VEGF_A plus RFX-978336} complexes in an asymmetric unit. The detailed data processing and structure refinement statistics are listed in FIG. 53. All structural images were rendered using Pymol (Schrodinger).

VEGF-A121/VEGFR1-Fc Binding ELISAs

Biotinylated human VEGF-A121 (isoform 121) was purchased from Acro Biosystems (cat #VE1-H82E7). VEGFR-1-Fc was purchased from R&D Systems (cat #3516-FL-050).

Bevacizumab was manufactured by Genentech Inc. (Lot #3067997). In all cases, 1 μg/mL of VEGFR1-Fc was coated on MaxiSorp plates overnight at 4° C. The following day, coated wells were blocked with Super Block (Rockland) for 2 hr with shaking at room temp. For non-equilibrium ELISAs, titrations of D-proteins and Bevacizumab were incubated with 1.0 nM of biotinylated VEGF-A121 for 30 min before addition to blocked VEGFR1-Fc coated wells.

Antagonist/VEGF-A121 mixture was incubated on VEGFR1-Fc wells for 1 hr with shaking at room temp, washed 3 times with wash buffer (PBS, 0.05% Tween 20), and bound biotinylated VEGF-A121 was detected with streptavidin-HRP (ThermoFisher). For equilibrium binding ELISAs, titrations of D-proteins, bevacizumab, and soluble VEGFR1-Fc were incubated with 0.15 nM of biotinylated VEGF-A121 overnight at 4° C. before addition to blocked VEGFR1-Fc coated wells. Antagonist/VEGF-A121 mixture was incubated on VEGFR1-Fc wells for 5 hr with shaking at room temp and developed as above. Data plotted are mean±standard deviation of triplicate measurements. IC50 values were derived from 3-parameter fits using Prism (GraphPad) and the error reported is derived from fits.

VEGF Cell Signaling Assay

Measurement of VEGF cellular signaling was performed using the VEGF Bioassay (Promega). Briefly, HEK293 cells are engineered to express VEGFR-2 coupled to a luciferase response element (KDR/NFAT-RE HEK293). VEGF signaling through VEGFR-2 mediates expression of luciferase which can be quantified using bioluminescence. Plated cells are incubated in the presence of 0.15 nM VEGF-A165 plus D-protein or Bevacizumab titrations and incubated at 37° C., 5% CO₂ for 6 hours. Following incubation Bio-Glo is added to wells according to the manufacturer's protocol and relative luminescence units (RLUs) were measured on a PerkinElmer 2300 Enspire Multimode plate reader. Data plotted are mean±standard deviation of triplicate measurements. IC₅₀ values were derived from 3-parameter fits using Prism (GraphPad) and error reported are derived from fits.

Rabbit Wet AMD Model

Dutch Belted rabbits (1.5-2.5 kg) were purchased from Western Oregon Rabbit Company. aflibercept was purchased from Regeneron Pharmaceuticals. On Day 0 Rabbits were randomized into treatment groups (N=5 per group) and baseline ophthalmic exams were done prior to a single intravitreal injection (25 μL per eye) of RFX-980869 (0.25 mg or 1.0 mg) or Eylea (1.0 mg). Rabbits were challenged with in 1 μg VEGF-A165 in both eyes on Days 2 and 23. On Days 5 and 26 fluorescein angiography was performed on both eyes and images were taken to assess vascular leakage. Scoring of vascular leakage based on FA images was carried out at Day 5 and 26 (FIG. 44B)

MC38 Syngeneic Tumor Model in C57BL6 Mice

Female C57BL6 mice transgenic for human PD-1 (12-13 weeks) were purchased from Beijing Biocytogen Co.). Nivolumab was purchased from Bristol Myers Squibb, lot #AAY1999. MC38 tumor cells (1×10⁶) were implanted subcutaneously in the right front flank and tumors were allowed to establish until the mean volume was 82 mm³. Mice were randomized into treatment groups (N=6 per group) on Day 0 when treatment initiation began. RFX-980869 at 2 mg/kg or 6 mg/kg was injected i.p. daily for 2 weeks (14 doses) and nivolumab at 1 mg/kg or 3 mg/kg was injected i.p. biweekly for 6 doses. All data is plotted as mean ±SEM.

Subcutaneous Immunization in BALB/c Mice

Adjuvant was purchased from TiterMax. Bevacizumab was purchased from Genentech/Roche. Female BALB/c mice (6-8 weeks) were randomized into immunization groups on Day 0 (n=5 per group) Immunizations were performed on Days 0, 21, 35 by subcutaneous injection of 25 μg of antigen. Antigens were emulsified in adjuvant (TiterMax) for injection on Day 0 and administered in PBS for Days 21 and 35. Serum pre-bleeds were performed on Days 0, 21, 35 prior to immunizations. Final bleeds for max titer response were taken on Day 42.

Although the particular embodiments have been described in some detail by way of illustration and example for purposes of clarity of understanding, it is readily apparent in light of the teachings of this invention that certain changes and modifications may be made thereto without departing from the spirit or scope of the appended claims.

Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A multivalent D-peptidic compound that specifically binds VEGF, comprising:
a D-peptidic Z domain capable of specifically binding a first binding site of VEGF;
a D-peptidic GA domain capable of specifically binding a second binding site of VEGF; and
a linking component that covalently links the D-peptidic Z and GA domains.

2. (canceled)

3. The D-peptidic compound of claim 1, wherein:
the D-peptidic Z domain comprises a VEGF specificity-determining motif (SDM) comprising 5 or more variant amino acid residues at positions selected from 9, 10, 13, 14, 17, 24, 27, 28, 32 and 35; and
the D-peptidic GA domain comprises a VEGF specificity-determining motif (SDM) comprising 5 or more variant amino acid residues at positions selected from 25, 27, 30, 31, 34, 36, 37, 39, 40 and 42-48.

4. The D-peptidic compound of claim 3, wherein the D-peptidic Z domain comprises:
a) a VEGF specificity-determining motif (SDM) defined by the following amino acid residues:
(SEQ ID NO: 160)
w⁹d¹⁰--w¹³x¹⁴--r¹⁷------x²⁴--k²⁷x²⁸---x³²--y³⁵
wherein:
x¹⁴ is selected from l, r and t;
x²⁴ is selected from h, i, l, r and v;
x²⁸ is selected from G, r and v;
x³² is selected from a, r, h, s and t; and
x³⁵ is selected from k or y;
b) a VEGF SDM having 80% or more identity with the SDM residues defined in (a); or c) a VEGF SDM having 1 to 3 amino acid residue substitutions relative to the SDM residues defined in (a), wherein the 1 to 3 amino acid residue substitutions are selected from:
i) a similar amino acid residue substitution according to Table 6;
ii) a conservative amino acid residue substitution according to Table 6;
iii) a highly conserved amino acid residue substitution according to Table 6; and
iv) an amino acid residue substitution according to the motif defined in FIG. 33A.

5. (canceled)

6. The D-peptidic compound of claim 3, wherein the D-peptidic GA domain comprises:
a) a VEGF specificity-determining motif (SDM) defined by the following amino acid residues:
(SEQ ID NO: 149)
e²⁵phvisf--h³⁴-p³⁶x³⁷-s³⁹h--G⁴³---a⁴⁷
wherein X³⁷ is selected from s, n, and y;
b) a VEGF SDM having 80% or more identity with the SDM residues defined in (a); or
c) a VEGF SDM having 1 to 3 amino acid residue substitutions relative to the SDM residues defined in (a), wherein the 1 to 3 amino acid residue substitutions are selected from:
i) a similar amino acid residue substitution according to Table 6;
ii) a conservative amino acid residue substitution according to Table 6;
iii) a highly conserved amino acid residue substitution according to Table 6; and
iv) an amino acid residue substitution according to the motif defined in FIG. 26.

7-20. (canceled)

21. The D-peptidic compound of claim 1, wherein the compound is bivalent.

22-25. (canceled)

26. The D-peptidic compound of claim 1, wherein the compound comprises four D-peptidic domains configured as a dimer of two bivalent D-peptidic compounds each comprising the D-peptidic Z and GA domains.

27-30. (canceled)

31. A D-peptidic compound that specifically binds VEGF, comprising:
a D-peptidic Z domain comprising:
a) a VEGF specificity-determining motif (SDM) defined by the following amino acid residues:
(SEQ ID NO: 160)
w⁹d¹⁰--w¹³x¹⁴--r¹⁷------x²⁴--k²⁷x²⁸---x³²--y³⁵
wherein:
x¹⁴ is selected from l, r and t;
x²⁴ is selected from h, i, l, r and v;
x²⁸ is selected from G, r and v;
x³² is selected from a, r, h, s and t; and
x³⁵ is selected from k or y;
b) a VEGF SDM having 80% or more identity with the SDM residues defined in (a); or
c) a VEGF SDM having 1 to 3 amino acid residue substitutions relative to the SDM residues defined in (a), wherein the 1 to 3 amino acid residue substitutions are selected from:
i) a similar amino acid residue substitution according to Table 6;
ii) a conservative amino acid residue substitution according to Table 6;
iii) a highly conserved amino acid residue substitution according to Table 6; and
iv) an amino acid residue substitution according to the motif defined in FIG. 33A.

32. The D-peptidic compound of claim 31, wherein the SDM residues defined in (a) are:
(SEQ ID NO: 161)
w⁹d¹⁰--w¹³r¹⁴--r¹⁷------l²⁴--k²⁷r²⁸---s³²--y³⁵
or
(SEQ ID NO: 162)
w⁹d¹⁰--w¹³r¹⁴--r¹⁷------v²⁴--k²⁷r²⁸---r³²--y³⁵.

33. (canceled)

34. The D-peptidic compound of claim 31, wherein the SDM residues are comprised in a peptidic framework sequence comprising:
a) peptidic framework residues defined by the following amino acid residues:
--n¹¹a--e¹⁵i-h¹⁸lpnln-e²⁵q--a²⁹fi-s³³l-;
b) peptidic framework residues having 80% or more (e.g., 90% or more) identity with the residues defined in (a); or
c) peptidic framework residues having 1 to 3 amino acid residue substitutions relative to the residues defined in (a), wherein the 1 to 3 amino acid residue substitutions are selected from:
i) a similar amino acid residue substitution according to Table 6;
ii) a conservative amino acid residue substitution according to Table 6; and
iii) a highly conserved amino acid residue substitution according to Table 6.

35. The D-peptidic compound of claim 31, comprising a SDM-containing sequence having 80% or more identity to the amino acid sequence:
(SEQ ID NO: 133)
w⁹d¹⁰naw¹³x¹⁴eir¹⁷hlpnlnx²⁴eqk²⁷x²⁸afix³²sly³⁵
wherein:
x¹⁴ is selected from l, r and t;
x²⁴ is selected from h, i, 1, r and v;
x²⁸ is selected from G, r and v;
x³² is selected from a, r, h, s and t; and
x³⁵ is selected from k or y.

36. The D-peptidic compound of claim 31, wherein the D-peptidic Z domain is a three-helix bundle of the structural formula:
[Helix 1^{(#8-18)}]-[Linker 1^{(#19-24)}]-[Helix 2^{(#25-36)}]-[Linker 2^{(#37-40)}]-[Helix 3^{(#41-54)}]
wherein:
# denotes reference positions of amino acid residues comprised in the D-peptidic GA domain; and
Helix 3^{(#41-54)} comprises a peptidic framework sequence selected from:
a) s⁴¹anllaeakklnda⁵⁴ (SEQ ID NO: 134);
b) a sequence having 70% or more identity to the sequence set forth in (a); or
c) a sequence having 1 to 5 amino acid residue substitutions relative to the sequence set forth in (a), wherein the 1 to 5 amino acid residue substitutions are selected from:
i) a similar amino acid residue substitution according to Table 6;
ii) a conservative amino acid residue substitution according to Table 6; and
iii) a highly conserved amino acid residue substitution according to Table 6.

37-38. (canceled)

39. The D-peptidic compound of claim 31, comprising:
(a) a sequence selected from one of compounds 978333 to 978337 (SEQ ID NOs: 114-118), 980181 (SEQ ID NO: 119), 980174 to 980180 (SEQ ID NOs: 120-126), and 981188 to 981190 (SEQ ID NOs: 127-129);
(b) a sequence having 80% or more sequence identity with the sequence defined in (a); or (c) a sequence having 1 to 10 amino acid substitutions relative to the sequence defined in (a), wherein the 1 to 10 amino acid substitutions are:
i) a similar amino acid substitution according to Table 6;
ii) a conservative amino acid substitution according to Table 6; or
iii) a highly conservative amino acid substitution according to Table 6.

40. The D-peptidic compound of claim 39, comprising an amino acid sequence of one of compounds 978333 to 978337 and 980181 (SEQ ID NOs:114-119).

41-42. (canceled)

43. A D-peptidic compound that specifically binds VEGF, comprising:
a D-peptidic GA domain comprising:
a) a VEGF specificity-determining motif (SDM) defined by the following amino acid residues:
(SEQ ID NO: 149)
e²⁵phvisf--h³⁴-p³⁶x³⁷-s³⁹h--G⁴³---a⁴⁷
wherein x³⁷ is selected from s, n, and y;
b) a VEGF SDM having 80% or more identity with the SDM residues defined in (a); or
c) a VEGF SDM having 1 to 3 amino acid residue substitutions relative to the SDM residues defined in (a), wherein the 1 to 3 amino acid residue substitutions are selected from:
i) a similar amino acid residue substitution according to Table 6;
ii) a conservative amino acid residue substitution according to Table 6;
iii) a highly conserved amino acid residue substitution according to Table 6; and
iv) an amino acid residue substitution according to the motif defined in FIG. 26.

44. The D-peptidic compound of claim 43, wherein the VEGF SDM defined in (a) is further defined by the following residues:
(SEQ ID NO: 150)
c⁷-----------------e²⁵phvisf--h³⁴-p³⁶x³⁷c³⁸sh--G⁴³-a⁴⁷
wherein x³⁷ is selected from s and n.

45. The D-peptidic compound of claim 43 or 11, further comprising the following segments (I)-(II):
x¹x²x³qwx⁶x⁷ (I)
x³⁷x³⁸ (II)
wherein:
x¹ to x³ are independently selected from any D-amino acid residue;
x⁶ is selected from i and v;
x³⁷ is selected from s and n; and
x⁷ and x³⁸ are amino acid residues connected via an intradomain linker having a backbone of 3 to 7 atoms in length as measured between the alpha-carbons of amino acid residues x⁷ and x³⁸.

46-49. (canceled)

50. The D-peptidic compound of claim 45, wherein x⁷ and x³⁸ are each cysteine and the intradomain linker comprises a disulfide linkage between the c⁷ and c³⁸ amino acid residues.

51-53. (canceled)

54. The D-peptidic compound of claim 43, wherein the D-peptidic GA domain comprises a three-helix bundle of the structural formula:
[Helix 1^{(#6-21)}]-[Linker 1^{(#22-26)}]-[Helix 2^{(#27-35)}]-[Linker 2^{(#36-37)}]-[Helix 3^{(#38-51)}]
wherein:
# denotes reference positions of amino acid residues comprised in the D-peptidic GA domain; and
Helix 1⁽⁴⁶⁻²¹⁾ comprises a peptidic framework sequence selected from:
a) x⁶x⁷knakedaiaelkka²¹ (SEQ ID NO: 138)
wherein:
x⁶ is selected from l, v, and i; and
x⁷ is selected from l and c; and
b) a sequence having 70% or more identity relative to the sequence defined in (a).

55-56. (canceled)

57. The D-peptidic compound of claim 56, wherein the D-peptidic GA domain comprises a sequence:
(SEQ ID NO: 141)
x¹x²x³qwx⁶x⁷knakedaiaelkkagitephvisfinhapx³⁷x³⁸shvnGl
knailkaha⁵³
wherein:
x¹is selected from t, y, f, i, p and r;
x² is selected from i, h, n, p, and s;
x³ is selected from d, i, and v;
x⁶ is selected from l, v, and i;
x⁷ is selected from l and c;
x³⁷ is selected from t, y, n, and s;
x³⁸ is selected from v and c;
x³⁹ is selected from e and s;
x⁴⁰ is selected from h and e;
x⁴³ is selected from g and a; and
x⁴⁷ selected from is a and e.

58. The D-peptidic compound of claim 43, comprising:
(a) a sequence selected from one of compounds 11055, 979102 and 979107-979110 (SEQ ID NOs: 108-113);
b) a sequence having 80% or more identity with the sequence defined in (a); or
c) a sequence having 1 to 10 amino acid residue substitutions relative to the sequence defined in (a), wherein the 1 to 10 amino acid residue substitutions are selected from:
i) a similar amino acid residue substitution according to Table 6;
ii) a conservative amino acid residue substitution according to Table 6; and
iii) a highly conserved amino acid residue substitution according to Table 6.

59. The D-peptidic compound of claim 58, comprising one of compounds 11055, 979102 and 979107-979110 (SEQ ID NOs: 108-113).

60-61. (canceled)

62. A pharmaceutical composition, comprising:
the D-peptidic compound according to claim 1, or a pharmaceutically acceptable salt thereof; and
a pharmaceutically acceptable excipient.

63. (canceled)

64. A method of treating or preventing a disease or condition associated with angiogenesis in a subject, the method comprising administering to a subject in need thereof an effective amount of a D-peptidic compound that specifically binds VEGF, or a pharmaceutically acceptable salt thereof according to claim 1.

65-73. (canceled)

74. A method for in vivo diagnosis or imaging of a disease or condition associated with angiogenesis comprising:
administering to a subject a D-peptidic compound that specifically binds VEGF according to claim 1; and
imaging at least a part of the subject.

75-77. (canceled)


SEQUENCE LISTING (169 sequences)

SEQ ID NO: 1 - protein, 53 aa, Artificial Sequence
  Synthetic sequence
  misc_feature (1)..(6): Xaa can be any amino acid
  misc_feature (8)..(9): Xaa can be any amino acid
  misc_feature (13)..(13): Xaa can be any amino acid
  misc_feature (16)..(16): Xaa is any amino acid
  misc_feature (20)..(21): Xaa is any amino acid
  misc_feature (24)..(24): Xaa is any amino acid
  misc_feature (27)..(27): Xaa is any amino acid
  misc_feature (29)..(31): Xaa is any amino acid
  misc_feature (40)..(40): Xaa is any amino acid
  misc_feature (42)..(42): Xaa is any amino acid
  misc_feature (46)..(46): Xaa is any amino acid
  misc_feature (50)..(53): Xaa is any amino acid
XXXXXXLXXAKEXAIXELKXXGIXSDXYXXXINKAKTVEXVXALKXEILXXXX

SEQ ID NO: 2 - protein, 53 aa, Artificial Sequence
  Synthetic sequence
TIDQWLLKNAKEDAIAELKKAGITSDFYFNAINKAKTVEEVNALKNEILKAHA

SEQ ID NO: 3 - protein, 53 aa, Artificial Sequence
  Synthetic sequence
  misc_feature (25)..(25): Xaa is any amino acid
  misc_feature (27)..(28): Xaa is any amino acid
  misc_feature (31)..(31): Xaa is any amino acid
  misc_feature (34)..(34): Xaa is any amino acid
  misc_feature (36)..(37): Xaa is any amino acid
  misc_feature (39)..(40): Xaa is any amino acid
  misc_feature (43)..(44): Xaa is any amino acid
  misc_feature (47)..(47): Xaa is any amino acid
TIDQWLLKNAKEDAIAELKKAGITXDXXFNXINXAXXVXXVNXXKNXILKAHA

SEQ ID NO: 4 - protein, 53 aa, Artificial Sequence
  Synthetic sequence
  misc_feature (21)..(21): Xaa is any amino acid
  misc_feature (25)..(31): Xaa is any amino acid
  misc_feature (34)..(34): Xaa is any amino acid
  misc_feature (36)..(37): Xaa is any amino acid
  misc_feature (39)..(40): Xaa is any amino acid
  misc_feature (43)..(44): Xaa is any amino acid
  misc_feature (47)..(47): Xaa is any amino acid
TIDQWLLKNAKEDAIAELKKXGITXXXXXXXINXAXXVXXVNXXKNXILKAHA

SEQ ID NO: 5 - protein, 53 aa, Artificial Sequence
  Synthetic sequence
  misc_feature (25)..(31): Xaa is any amino acid
  misc_feature (34)..(34): Xaa is any amino acid
  misc_feature (36)..(37): Xaa is any amino acid
  misc_feature (39)..(40): Xaa is any amino acid
  misc_feature (43)..(43): Xaa is any amino acid
  misc_feature (47)..(47): Xaa is any amino acid
TIDQWLLKNAKEDAIAELKKAGITXXXXXXXINXAXXVXXVNXLKNXILKAHA

SEQ ID NO: 6 - protein, 53 aa, Artificial Sequence; identical to SEQ ID NO: 2
  Synthetic sequence

SEQ ID NO: 7 - protein, 45 aa, Artificial Sequence
  Synthetic sequence
LKNAKEDAIAELKKAGITSDFYFNAINKAKTVEGANALKNEILKA

SEQ ID NO: 8 - protein, 45 aa, Artificial Sequence
  Synthetic sequence
LKLTKEEAEKALKKLGITSEFILNQIDKATSREGLESLVQTIKQS

SEQ ID NO: 9 - protein, 45 aa, Artificial Sequence
  Synthetic sequence
LQEAKDKAIQEAKANGLTSKLLLKNIENAKTPESAKSFAEELIKS

SEQ ID NO: 10 - protein, 45 aa, Artificial Sequence
  Synthetic sequence
LKNAKEEAIKELKEAGITSDLYFSLINKAKTVEGVEALKNEILKA

SEQ ID NO: 11 - protein, 45 aa, Artificial Sequence
  Synthetic sequence
LKNAKEDAIKELKEAGISSDIYFDAINKAKTVEGVEALKNEILKA

SEQ ID NO: 12 - protein, 45 aa, Artificial Sequence
  Synthetic sequence
LKNAKEAAIKELKEAGITAEYLFNLINKAKTVEGVESLKNEILKA

SEQ ID NO: 13 - protein, 45 aa, Artificial Sequence
  Synthetic sequence
LKNAKEDAIKELKEAGITSDIYFDAINKAKTIEGVEALKNEILKA

SEQ ID NO: 14 - protein, 45 aa, Artificial Sequence
  Synthetic sequence
  misc_feature (18)..(18): Xaa is any amino acid
LAKAKADALKEFNKYGVXSDYYKNLINNAKTVEGVKDLQAQVVES

SEQ ID NO: 15 - protein, 45 aa, Artificial Sequence
  Synthetic sequence
  misc_feature (18)..(18): Xaa is any amino acid
LAEAKVLANRELDKYGVXSDYHKNLINNAKTVEGVKDLQAQVVES

SEQ ID NO: 16 - protein, 47 aa, Artificial Sequence
  Synthetic sequence
  misc_feature (18)..(18): Xaa is any amino acid
LAEAKVLANRELDKYGVXSDYYKNLINNAKTVEGVKALIDEILAALP

SEQ ID NO: 17 - protein, 45 aa, Artificial Sequence
  Synthetic sequence
  misc_feature (18)..(18): Xaa is any amino acid
LDNAKNAALKEFDRYGVXSDYYKNLINKAKTVEGIMELQAQVVES

SEQ ID NO: 18 - protein, 45 aa, Artificial Sequence
  Synthetic sequence
  misc_feature (18)..(18): Xaa is any amino acid
LSEAKEMAIRELDANGVXSDFYKDKIDDAKTVEGVVALKDLILNS

SEQ ID NO: 19 - protein, 45 aa, Artificial Sequence
  Synthetic sequence
  misc_feature (21)..(21): Xaa is any amino acid
  misc_feature (42)..(43): Xaa is any amino acid
LAKLAADTDLDLDVAKIINDXYTTKVENAKTAEDVKKIFEEXXSQ

SEQ ID NO: 20 - protein, 45 aa, Artificial Sequence
  Synthetic sequence
  misc_feature (18)..(18): Xaa is any amino acid
  misc_feature (44)..(45): Xaa is any amino acid
LAKAKADAIEILKKYGIXGDYYIKLINNGKTAEGVTALKDEILXX

SEQ ID NO: 21 - protein, 45 aa, Artificial Sequence
  Synthetic sequence
  misc_feature (18)..(18): Xaa is any amino acid
LLEAKEAAINELKQYGIXSDYYVTLINKAKTVEGVNALKAEILSA

SEQ ID NO: 22 - protein, 53 aa, Artificial Sequence
  Synthetic sequence
TIDQWLLKNAKEDAIAELKKAGITEDHVFNFINHAPYVSHVNGLKNAILKAHA

SEQ ID NO: 23 - protein, 53 aa, Artificial Sequence
  Synthetic sequence
TIDQWLLKNAKEDAIAELKKCGITEPHVISFINHAPYVSHVNGLKNAILKAHA

SEQ ID NO: 24 - protein, 53 aa, Artificial Sequence
  Synthetic sequence
TIDQWLLKNAKEDAIAELKKAGITEPHVISFINHAPYVSHVNGLKNAILKAHA

SEQ ID NO: 25 - protein, 52 aa, Artificial Sequence
  Synthetic sequence
  misc_feature (1)..(23): Xaa is any amino acid
  misc_feature (31)..(32): Xaa is any amino acid
  misc_feature (34)..(34): Xaa is any amino acid
  misc_feature (37)..(37): Xaa is any amino acid
  misc_feature (40)..(41): Xaa is any amino acid
  misc_feature (43)..(45): Xaa is any amino acid
  misc_feature (47)..(52): Xaa is any amino acid
XXXXXXXXXXXXXXXXXXXXXXXEPHVISFXXHXPYXSHXXGXXXAXXXXXX

SEQ ID NO: 26 - protein, 54 aa, Artificial Sequence
  Synthetic sequence
  misc_feature (21)..(21): Xaa is any amino acid
  misc_feature (26)..(32): Xaa is any amino acid
  misc_feature (35)..(35): Xaa is any amino acid
  misc_feature (37)..(38): Xaa is any amino acid
  misc_feature (40)..(41): Xaa is any amino acid
  misc_feature (44)..(44): Xaa is any amino acid
  misc_feature (48)..(48): Xaa is any amino acid
TIDQWLLKNAKEDAIAELKKXAGITXXXXXXXINXAXXVXXVNXLKNXILKAHA

SEQ ID NO: 27 - protein, 46 aa, Artificial Sequence
  Synthetic sequence
  misc_feature (20)..(26): Xaa is any amino acid
  misc_feature (29)..(29): Xaa is any amino acid
  misc_feature (31)..(32): Xaa is any amino acid
  misc_feature (34)..(35): Xaa is any amino acid
  misc_feature (38)..(38): Xaa is any amino acid
  misc_feature (42)..(42): Xaa is any amino acid
LLKNAKEDAIAELKKAGITXXXXXXXINXAXXVXXVNXLKNXILKA

SEQ ID NO: 28 - protein, 53 aa, Artificial Sequence
  Synthetic sequence
  misc_feature (1)..(6): Xaa is any amino acid
  misc_feature (8)..(9): Xaa is any amino acid
  misc_feature (13)..(13): Xaa is any amino acid
  misc_feature (16)..(16): Xaa is any amino acid
  misc_feature (20)..(21): Xaa is any amino acid
  misc_feature (24)..(24): Xaa is any amino acid
  misc_feature (42)..(42): Xaa is any amino acid
  misc_feature (46)..(46): Xaa is any amino acid
  misc_feature (50)..(53): Xaa is any amino acid
XXXXXXLXXAKEXAIXELKXXGIXEPHVISFINHAPYVSHVXGLKXAILXXXX

SEQ ID NO: 29 - protein, 48 aa, Artificial Sequence
  Synthetic sequence
LLKNAKEDAIAELKKAGITEPHVISFINHAPYVSHVNGLKNAILKAHA

SEQ ID NO: 30 - protein, 48 aa, Artificial Sequence
  Synthetic sequence
  misc_feature (10)..(10): Xaa is any amino acid
LLKNAKEDAXAELKKAGITEPHVISFINHAPYVSHVNGLKNAILKAHA

SEQ ID NO: 31 - protein, 48 aa, Artificial Sequence
  Synthetic sequence
  misc_feature (24)..(24): Xaa is any amino acid
LLKNAKEDAIAELKKAGITEPHVXSFINHAPYVSHVNGLKNAILKAHA

SEQ ID NO: 32 - protein, 48 aa, Artificial Sequence
  Synthetic sequence
  misc_feature (10)..(10): Xaa is any amino acid
  misc_feature (24)..(24): Xaa is any amino acid
LLKNAKEDAXAELKKAGITEPHVXSFINHAPYVSHVNGLKNAILKAHA

SEQ ID NO: 33 - protein, 53 aa, Artificial Sequence
  Synthetic sequence
  misc_feature (5)..(5): Xaa is any amino acid
TIDQXLLKNAKEDAIAELKKAGITEPHVISFINHAPYVSHVNGLKNAILKAHA

SEQ ID NO: 34 - protein, 53 aa, Artificial Sequence
  Synthetic sequence
  misc_feature (37)..(37): Xaa is any amino acid
TIDQWLLKNAKEDAIAELKKAGITEPHVISFINHAPXVSHVNGLKNAILKAHA

SEQ ID NO: 35 - protein, 53 aa, Artificial Sequence
  Synthetic sequence
  misc_feature (5)..(5): Xaa is any amino acid
  misc_feature (37)..(37): Xaa is any amino acid
TIDQXLLKNAKEDAIAELKKAGITEPHVISFINHAPXVSHVNGLKNAILKAHA

SEQ ID NO: 36 - protein, 50 aa, Artificial Sequence
  Synthetic sequence
QWLLKNAKEDAIAELKKAGITEPHVISFINHAPYVSHVNGLKNAILKAHA

SEQ ID NO: 37 - protein, 50 aa, Artificial Sequence
  Synthetic sequence
  misc_feature (1)..(1): Xaa is any amino acid
XWLLKNAKEDAIAELKKAGITEPHVISFINHAPYVSHVNGLKNAILKAHA

SEQ ID NO: 38 - protein, 50 aa, Artificial Sequence
  Synthetic sequence
  misc_feature (1)..(2): Xaa is any amino acid
XXLLKNAKEDAIAELKKAGITEPHVISFINHAPYVSHVNGLKNAILKAHA

SEQ ID NO: 39 - protein, 50 aa, Artificial Sequence
  Synthetic sequence
  misc_feature (1)..(2): Xaa is any amino acid
  misc_feature (12)..(12): Xaa is any amino acid
XXLLKNAKEDAXAELKKAGITEPHVISFINHAPYVSHVNGLKNAILKAHA

SEQ ID NO: 40 - protein, 50 aa, Artificial Sequence
  Synthetic sequence
  misc_feature (1)..(2): Xaa is any amino acid
  misc_feature (26)..(26): Xaa is any amino acid
XXLLKNAKEDAIAELKKAGITEPHVXSFINHAPYVSHVNGLKNAILKAHA

SEQ ID NO: 41 - protein, 48 aa, Artificial Sequence
  Synthetic sequence
  misc_feature (22)..(22): Xaa is any amino acid
LLKNAKEDAIAELKKAGITEPXVISFINHAPYVSHVNGLKNAILKAHA

SEQ ID NO: 42 - protein, 48 aa, Artificial Sequence
  Synthetic sequence
  misc_feature (29)..(29): Xaa is any amino acid
LLKNAKEDAIAELKKAGITEPHVISFINXAPYVSHVNGLKNAILKAHA

SEQ ID NO: 43 - protein, 48 aa, Artificial Sequence
  Synthetic sequence
  misc_feature (35)..(35): Xaa is any amino acid
LLKNAKEDAIAELKKAGITEPHVISFINHAPYVSXVNGLKNAILKAHA

SEQ ID NO: 44 - protein, 48 aa, Artificial Sequence
  Synthetic sequence
  misc_feature (22)..(22): Xaa is any amino acid
  misc_feature (29)..(29): Xaa is any amino acid
  misc_feature (35)..(35): Xaa is any amino acid
LLKNAKEDAIAELKKAGITEPXVISFINXAPYVSXVNGLKNAILKAHA

SEQ ID NO: 45 - protein, 48 aa, Artificial Sequence; identical to SEQ ID NO: 29
  Synthetic sequence
  misc_feature (26)..(26): F is phenylalanine or an analog thereof

SEQ ID NO: 46 - protein, 48 aa, Artificial Sequence; identical to SEQ ID NO: 29
  Synthetic sequence
  misc_feature (32)..(32): Y is tyrosine or an analog thereof

SEQ ID NO: 47 - protein, 48 aa, Artificial Sequence; identical to SEQ ID NO: 29
  Synthetic sequence
  misc_feature (26)..(26): F is phenylalanine or an analog thereof
  misc_feature (32)..(32): Y is tyrosine or an analog thereof

SEQ ID NO: 48 - protein, 54 aa, Artificial Sequence
  Synthetic sequence
TIDQWLLKNAKEDAIAELKKAGITEDHVFNFINHAPYVSHVNGLKNAILGRTVP

SEQ ID NO: 49 - protein, 54 aa, Artificial Sequence
  Synthetic sequence
TIDQWLLKNAKEDAIAELKKAGITEPHVISFINHAPYVSHVNGLKNAILGRTVP

SEQ ID NO: 50 - protein, 57 aa, Artificial Sequence
  Synthetic sequence
TIDQWLLKNAKEDAIAELKKAGITEPHVISFINHAPYVSHVNGLKNAILGRTVPASC

SEQ ID NO: 51 - protein, 51 aa, Artificial Sequence
  Synthetic sequence
PALLKNAKEDAIAELKKAGITEPHVISFINHAPYVSHVNGLKNAILGRTVP

SEQ ID NO: 52 - protein, 54 aa, Artificial Sequence
  Synthetic sequence
PALLKNAKEDAIAELKKAGITEPHVISFINHAPYVSHVNGLKNAILGRTVPASC

SEQ ID NO: 53 - protein, 54 aa, Artificial Sequence
  Synthetic sequence
TIDQWLLKNAKEDAIAELKKAGITEDHVFNFINHAPYVSHVNGLKNAILEDWYL

SEQ ID NO: 54 - protein, 54 aa, Artificial Sequence
  Synthetic sequence
TIDQWLLKNAKEDAIAELKKAGITEPHVISFINHAPYVSHVNGLKNAILEDWYL

SEQ ID NO: 55 - protein, 57 aa, Artificial Sequence
  Synthetic sequence
TIDQWLLKNAKEDAIAELKKAGITEPHVISFINHAPYVSHVNGLKNAILEDWYLASC

SEQ ID NO: 56 - protein, 51 aa, Artificial Sequence
  Synthetic sequence
PALLKNAKEDAIAELKKAGITEPHVISFINHAPYVSHVNGLKNAILEDWYL

SEQ ID NO: 57 - protein, 53 aa, Artificial Sequence
  Synthetic sequence
TIDQWLLKNAKEDAIAELKKAGITEDHVFNFINHAPYVSHVNGLKNAILADFL

SEQ ID NO: 58 - protein, 53 aa, Artificial Sequence
  Synthetic sequence
TIDQWLLKNAKEDAIAELKKAGITEDHVFNFINHAPYVSHVNGLKNAILEDYL

SEQ ID NO: 59 - protein, 54 aa, Artificial Sequence; identical to SEQ ID NO: 48
  Synthetic sequence

SEQ ID NO: 60 - protein, 54 aa, Artificial Sequence; identical to SEQ ID NO: 53
  Synthetic sequence

SEQ ID NO: 61 - protein, 55 aa, Artificial Sequence
  Synthetic sequence
TIDQWLLKNAKEDAIAELKKAGITEDHVFNFINHAPYVSHVNGLKNAILGEHGSP

SEQ ID NO: 62 - protein, 54 aa, Artificial Sequence; identical to SEQ ID NO: 49
  Synthetic sequence

SEQ ID NO: 63 - protein, 49 aa, Artificial Sequence
  Synthetic sequence
LLKNAKEDAIAELKKAGITEPHVISFINHAPYVSHVNGLKNAILGRTVP

SEQ ID NO: 64 - protein, 51 aa, Artificial Sequence; identical to SEQ ID NO: 51
  Synthetic sequence

SEQ ID NO: 65 - protein, 54 aa, Artificial Sequence; identical to SEQ ID NO: 52
  Synthetic sequence

SEQ ID NO: 66 - protein, 54 aa, Artificial Sequence; identical to SEQ ID NO: 54
  Synthetic sequence

SEQ ID NO: 67 - protein, 49 aa, Artificial Sequence
  Synthetic sequence
LLKNAKEDAIAELKKAGITEPHVISFINHAPYVSHVNGLKNAILEDWYL

SEQ ID NO: 68 - protein, 51 aa, Artificial Sequence; identical to SEQ ID NO: 56
  Synthetic sequence

SEQ ID NO: 69 - protein, 57 aa, Artificial Sequence; identical to SEQ ID NO: 55
  Synthetic sequence

SEQ ID NO: 70 - protein, 53 aa, Artificial Sequence
  Synthetic sequence
TIDQWLLKNAKEDAIAELKKAGITEDHVISFINHAPYVSHVNGLKNAILKAHA

SEQ ID NO: 71 - protein, 54 aa, Artificial Sequence
  Synthetic sequence
TIDQWLLKNAKEDAIAELKKAGITEPHVISFINHAPYVSHVNGLKNAILKAHAC

SEQ ID NO: 72 - protein, 5 aa, Artificial Sequence
  Synthetic sequence
TIDQW

SEQ ID NO: 73 - protein, 2 aa, Artificial Sequence
  Synthetic sequence
QW

SEQ ID NO: 74 - protein, 16 aa, Artificial Sequence
  Synthetic sequence
LLKNAKEDAIAELKKA

SEQ ID NO: 75 - protein, 5 aa, Artificial Sequence
  Synthetic sequence
GITEP

SEQ ID NO: 76 - protein, 9 aa, Artificial Sequence
  Synthetic sequence
HVISFINHA

SEQ ID NO: 77 - protein, 11 aa, Artificial Sequence
  Synthetic sequence
PHVISFINHAP

SEQ ID NO: 78 - protein, 2 aa, Artificial Sequence
  Synthetic sequence
PY

SEQ ID NO: 79 - protein, 12 aa, Artificial Sequence
  Synthetic sequence
VSHVNGLKNAIL

SEQ ID NO: 80 - protein, 23 aa, Artificial Sequence
  Synthetic sequence
HVISFINHAPYVSHVNGLKNAIL

SEQ ID NO: 81 - protein, 28 aa, Artificial Sequence
  Synthetic sequence
GITEPHVISFINHAPYVSHVNGLKNAIL

SEQ ID NO: 82 - protein, 14 aa, Artificial Sequence
  Synthetic sequence
VSHVNGLKNAILKA

SEQ ID NO: 83 - protein, 25 aa, Artificial Sequence
  Synthetic sequence
HVISFINHAPYVSHVNGLKNAILKA

SEQ ID NO: 84 - protein, 30 aa, Artificial Sequence
  Synthetic sequence
GITEPHVISFINHAPYVSHVNGLKNAILKA

SEQ ID NO: 85 - protein, 4 aa, Artificial Sequence
  Synthetic sequence
KAHA

SEQ ID NO: 86 - protein, 5 aa, Artificial Sequence
  Synthetic sequence
EDWYL

SEQ ID NO: 87 - protein, 5 aa, Artificial Sequence
  Synthetic sequence
GRTVP

SEQ ID NO: 88 - protein, 102 aa, Artificial Sequence [sequence omitted]

SEQ ID NO: 89 - protein, 10 aa, Artificial Sequence
  Synthetic sequence
KFMDVYQRSY

SEQ ID NO: 90 - protein, 5 aa, Artificial Sequence
  Synthetic sequence
NDEGL

SEQ ID NO: 91 - protein, 5 aa, Artificial Sequence
  Synthetic sequence
YIFKP

SEQ ID NO: 92 - protein, 12 aa, Artificial Sequence
  Synthetic sequence
IMRIKPHQGQHI

SEQ ID NO: 93 - protein, 11 aa, Artificial Sequence
  Synthetic sequence
  misc_feature (3)..(4): Xaa is a hydrophobic amino acid residue
  misc_feature (5)..(5): Xaa is any amino acid
  misc_feature (7)..(7): Xaa is a hydrophobic amino acid residue
  misc_feature (8)..(8): Xaa is any amino acid
  misc_feature (10)..(10): Xaa is a hydrophobic amino acid residue
PHXXXFXXHXP

SEQ ID NO: 94 - protein, 11 aa, Artificial Sequence
  Synthetic sequence
  misc_feature (1)..(1): Xaa is d, p or G
  misc_feature (4)..(4): Xaa is f or i
  misc_feature (5)..(5): Xaa is n or s
  misc_feature (8)..(8): Xaa is n or s
  misc_feature (11)..(11): Xaa is p or G
XHVXXFIXHAX

SEQ ID NO: 95 - protein, 11 aa, Artificial Sequence
  Synthetic sequence
  misc_feature (4)..(4): Xaa is f or i
  misc_feature (5)..(5): Xaa is a polar amino acid residue
  misc_feature (8)..(8): Xaa is a polar amino acid residue
PHVXXFIXHAP

SEQ ID NO: 96 - protein, 10 aa, Artificial Sequence
  Synthetic sequence
  misc_feature (1)..(1): Xaa is any amino acid
  misc_feature (2)..(2): Xaa is any amino acid
  misc_feature (3)..(3): H is a histidine or a histidine analog
  misc_feature (4)..(4): Xaa is a, i, l, or v
  misc_feature (4)..(5): Xaa is any amino acid
  misc_feature (6)..(6): Xaa is a non-polar residue having a sidechain selected from H, a lower alkyl and a substituted lower alkyl
  misc_feature (7)..(7): Xaa is a, i, l, or v
  misc_feature (8)..(9): Xaa is any amino acid
  misc_feature (10)..(10): Xaa is a non-polar residue having a sidechain selected from H, a lower alkyl and a substituted lower alkyl
  misc_feature (11)..(11): Xaa is a, i, l, or v
XXHXXXXXXX

SEQ ID NO: 97 - protein, 12 aa, Artificial Sequence
  Synthetic sequence
  misc_feature (1)..(1): Xaa is v, e, k, or r
  misc_feature (2)..(2): Xaa is a polar amino acid residue
  misc_feature (5)..(5): Xaa is a polar amino acid residue
  misc_feature (8)..(8): Xaa is l, k, r, or e
  misc_feature (9)..(9): Xaa is a polar amino acid residue
  misc_feature (12)..(12): Xaa is l, k, r, or e
XXHVXGLXXAIX

SEQ ID NO: 98 - protein, 14 aa, Artificial Sequence
  Synthetic sequence
  misc_feature (1)..(1): Xaa is v, e, k, or r
  misc_feature (2)..(2): Xaa is a polar amino acid residue
  misc_feature (5)..(5): Xaa is a polar amino acid residue
  misc_feature (8)..(8): Xaa is l, k, r, or e
  misc_feature (9)..(9): Xaa is a polar amino acid residue
  misc_feature (12)..(12): Xaa is l, k, r, or e
  misc_feature (13)..(13): Xaa is a polar amino acid residue
XXHVXGLXXAIXXA

SEQ ID NO: 99 - protein, 24 aa, Artificial Sequence
  Synthetic sequence
  misc_feature (1)..(1): Xaa is a helix-terminating residue
  misc_feature (2)..(2): H is a histidine or histidine analog
  misc_feature (3)..(3): Xaa is a hydrophobic residue
  misc_feature (4)..(4): Xaa is any amino acid
  misc_feature (5)..(5): Xaa is any amino acid
  misc_feature (7)..(7): Xaa is a hydrophobic residue
  misc_feature (8)..(8): f is phenylalanine or an phenylalanine analog
  misc_feature (8)..(8): Xaa is any amino acid
  misc_feature (9)..(9): H is a histidine or histidine analog
  misc_feature (10)..(10): Xaa is a hydrophobic residue
  misc_feature (11)..(11): Xaa is any amino acid
  misc_feature (12)..(12): y is a tyrosine or a tyrosine analog
  misc_feature (13)..(13): Xaa is any amino acid
  misc_feature (14)..(14): Xaa is any amino acid
  misc_feature (15)..(15): H is a histidine or histidine analog
  misc_feature (16)..(16): Xaa is a hydrophobic residue
  misc_feature (17)..(17): Xaa is any amino acid
  misc_feature (18)..(18): Xaa is a non-polar residue
  misc_feature (19)..(19): Xaa is a hydrophobic residue
  misc_feature (20)..(20): Xaa is any amino acid
  misc_feature (21)..(21): Xaa is any amino acid
  misc_feature (22)..(22): Xaa is a non-polar residue
  misc_feature (23)..(23): Xaa is a hydrophobic residue
  misc_feature (24)..(24): Xaa is any amino acid
XHXXXFXXHXXYXXHXXXXXXXXX

SEQ ID NO: 100 - protein, 30 aa, Artificial Sequence
  Synthetic sequence
  misc_feature (1)..(3): Xaa is any amino acid
  misc_feature (4)..(4): e is glutamic acid or glutamic acid analog
  misc_feature (5)..(5): Xaa is a helix-terminating residue
  misc_feature (7)..(7): Xaa is a hydrophobic residue
  misc_feature (8)..(9): Xaa is any amino acid
  misc_feature (11)..(11): Xaa is a hydrophobic residue
  misc_feature (12)..(12): Xaa is any amino acid
  misc_feature (14)..(14): Xaa is a hydrophobic residue
  misc_feature (15)..(15): Xaa is a helix-terminating residue
  misc_feature (16)..(16): y is a tyrosine or tyrosine analog
  misc_feature (17)..(18): Xaa is any amino acid
  misc_feature (20)..(20): Xaa is a helix-terminating residue
  misc_feature (21)..(21): Xaa is any amino acid
  misc_feature (22)..(22): Xaa is a non-polar amino acid residue
  misc_feature (23)..(23): Xaa is a helix-terminating residue
  misc_feature (24)..(24): Xaa is any amino acid
  misc_feature (25)..(25): Xaa is any amino acid
  misc_feature (26)..(26): Xaa is a non-polar amino acid residue
  misc_feature (27)..(27): Xaa is a helix-terminating residue
  misc_feature (28)..(28): Xaa is any amino acid
  misc_feature (29)..(29): Xaa is any amino acid
  misc_feature (30)..(30): Xaa is any amino acid
XXXEXHXXXFXXHXXYXXHXXXXXXXXXXX

SEQ ID NO: 101 - protein, 11 aa, Artificial Sequence
  Synthetic sequence
  misc_feature (1)..(1): Xaa is d, p, or G
  misc_feature (3)..(3): Xaa is a hydrophobic residue
  misc_feature (4)..(4): Xaa is any amino acid
  misc_feature (5)..(5): Xaa is any amino acid
  misc_feature (7)..(7): Xaa is a hydrophobic residue
  misc_feature (8)..(8): Xaa is any amino acid
  misc_feature (10)..(10): Xaa is a hydrophobic residue
  misc_feature (11)..(11): Xaa is p or G
XHXXXFXXHXX

SEQ ID NO: 102 - protein, 11 aa, Artificial Sequence
  Synthetic sequence
  misc_feature (4)..(4): Xaa is f or i
  misc_feature (5)..(5): Xaa is a polar amino acid
  misc_feature (8)..(8): Xaa is a polar amino acid
PHVXXFIXHAP

SEQ ID NO: 103 - protein, 14 aa, Artificial Sequence
  Synthetic sequence
  misc_feature (1)..(2): Xaa is any amino acid
  misc_feature (4)..(4): Xaa is a hydrophobic residue
  misc_feature (5)..(5): Xaa is any amino acid
  misc_feature (6)..(6): Xaa is a non-polar amino acid residue
  misc_feature (7)..(7): Xaa is a hydrophobic residue
  misc_feature (8)..(8): Xaa is any amino acid
  misc_feature (9)..(9): Xaa is any amino acid
  misc_feature (10)..(10): Xaa is a non-polar amino acid residue
  misc_feature (11)..(11): Xaa is a hydrophobic residue
  misc_feature (12)..(12): Xaa is any amino acid
  misc_feature (13)..(13): Xaa is any amino acid
  misc_feature (14)..(14): Xaa is any amino acid
XXHXXXXXXXXXXX

SEQ ID NO: 104 - protein, 14 aa, Artificial Sequence
  Synthetic sequence
  misc_feature (1)..(1): Xaa is v, e,, k, or r
  misc_feature (2)..(2): Xaa is a polar amino acid residue
  misc_feature (5)..(5): Xaa is a polar amino acid residue
  misc_feature (8)..(8): Xaa is l, k, r, or e
  misc_feature (9)..(9): Xaa is a polar amino acid residue
  misc_feature (12)..(12): Xaa is l, k, r, or e
  misc_feature (13)..(13): Xaa is a polar amino acid residue
XXHVXGLXXAIXXA

SEQ ID NO: 105 - protein, 15 aa, Artificial Sequence
  Synthetic sequence
  misc_feature (2)..(3): Xaa is any amino acid
  misc_feature (7)..(7): Xaa is any amino acid
  misc_feature (10)..(10): Xaa is any amino acid
  misc_feature (14)..(15): Xaa is any amino acid
LXXAKEXAIXELKXX

SEQ ID NO: 106 - protein, 53 aa, Artificial Sequence
  Synthetic sequence
TIDQWLLKNAKEDAIAELKKAGITSDHVFNFINYAPYVSDVDALKNEILKAHA

SEQ ID NO: 107 - protein, 53 aa, Artificial Sequence
  Synthetic sequence
TIDQWLLKNAKEDAIAELKKAGITSDHVFNFINYAPYVSDVNALKNEILKAHA

SEQ ID NO: 108 - protein, 53 aa, Artificial Sequence; identical to SEQ ID NO: 24
  Synthetic sequence

SEQ ID NO: 109 - protein, 53 aa, Artificial Sequence
  Synthetic sequence
FNIQWICKNAKEDAIAELKKAGITEPHVISFINHAPSCSHVNGLKNAILKAHA

SEQ ID NO: 110 - protein, 53 aa, Artificial Sequence
  Synthetic sequence
IPIQWVCKNAKEDAIAELKKAGITEPHVISFINHAPSCSHVNGLKNAILKAHA

SEQ ID NO: 111 - protein, 53 aa, Artificial Sequence
  Synthetic sequence
PSVQWICKNAKEDAIAELKKAGITEPHVISFINHAPSCSHVNGLKNAILKAHA

SEQ ID NO: 112 - protein, 53 aa, Artificial Sequence
  Synthetic sequence
RNIQWVCKNAKEDAIAELKKAGITEPHVISFINHAPNCSHVNGLKNAILKAHA

SEQ ID NO: 113 - protein, 53 aa, Artificial Sequence
  Synthetic sequence
YHIQWVCKNAKEDAIAELKKAGITEPHVISFINHAPNCSHVNGLKNAILKAHA

SEQ ID NO: 114 - protein, 58 aa, Artificial Sequence
  Synthetic sequence
VDNKFNKEWDNAWLEIRHLPNLNHEQKRAFISSLYDDPSQSANLLAEAKKLNDAQAPK

SEQ ID NO: 115 - protein, 58 aa, Artificial Sequence
  Synthetic sequence
VDNKFNKEWDNAWREIRHLPNLNHEQKRAFISSLYDDPSQSANLLAEAKKLNDAQAPK

SEQ ID NO: 116 - protein, 58 aa, Artificial Sequence
  Synthetic sequence
VDNKFNKEWDNAWREIRHLPNLNLEQKGAFIASLYDDPSQSANLLAEAKKLNDAQAPK

SEQ ID NO: 117 - protein, 58 aa, Artificial Sequence
  Synthetic sequence
VDNKFNKEWDNAWREIRHLPNLNLEQKRAFISSLYDDPSQSANLLAEAKKLNDAQAPK

SEQ ID NO: 118 - protein, 58 aa, Artificial Sequence
  Synthetic sequence
VDNKFNKEWDNAWTEIRHLPNLNREQKVAFITSLYDDPSQSANLLAEAKKLNDAQAPK

SEQ ID NO: 119 - protein, 58 aa, Artificial Sequence
  Synthetic sequence
VDNKFNKEWDNAWKEIRHLPNLNVEQKRAFIHSLYDDPSQSANLLAEAKKLNDAQAPK

SEQ ID NO: 120 - protein, 58 aa, Artificial Sequence
  Synthetic sequence
VDNKFNKEWDNAWREIRHLPNLNIEQKRAFIHSLYDDPSQSANLLAEAKKLNDAQAPK

SEQ ID NO: 121 - protein, 58 aa, Artificial Sequence
  Synthetic sequence
VDNKFNKEWDNAWREIRHLPNLNIEQKRAFIRSLYDDPSQSANLLAEAKKLNDAQAPK

SEQ ID NO: 122 - protein, 58 aa, Artificial Sequence
  Synthetic sequence
VDNKFNKEWDNAWREIRHLPNLNIEQKRAFIYSLYDDPSQSANLLAEAKKLNDAQAPK

SEQ ID NO: 123 - protein, 58 aa, Artificial Sequence
  Synthetic sequence
VDNKFNKEWDNAWREIRHLPNLNLEQKRAFIRSLYDDPSQSANLLAEAKKLNDAQAPK

SEQ ID NO: 124 - protein, 58 aa, Artificial Sequence
  Synthetic sequence
VDNKFNKEWDNAWREIRHLPNLNREQKLAFIHSLYDDPSQSANLLAEAKKLNDAQAPK

SEQ ID NO: 125 - protein, 58 aa, Artificial Sequence
  Synthetic sequence
VDNKFNKEWDNAWREIRHLPNLNVEQKRAFIKSLYDDPSQSANLLAEAKKLNDAQAPK

SEQ ID NO: 126 - protein, 58 aa, Artificial Sequence
  Synthetic sequence
VDNKFNKEWDNAWREIRHLPNLNVEQKRAFIRSLYDDPSQSANLLAEAKKLNDAQAPK

SEQ ID NO: 127 - protein, 58 aa, Artificial Sequence
  Synthetic sequence
VDNKFDKEWDNAWREIRRLPNLNLEQKRAFISSLYDDPSQSANLLAEAKKLNDAQAPK

SEQ ID NO: 128 - protein, 58 aa, Artificial Sequence
  Synthetic sequence
VDNKFNKEWDNAWREIRRLPNLNLEQKRAFISSLYDDPSQSANLLAEAKKLNDAQAPK

SEQ ID NO: 129 - protein, 58 aa, Artificial Sequence
  Synthetic sequence
VDNKFNKEWDNAWREIRRLPNLNVEQKRAFISSLYDDPSQSANLLAEAKKLNDAQAPK

SEQ ID NO: 130 - DNA, 99 bp, Artificial Sequence
  Synthetic sequence
  misc_feature (16)..(16): n is a mix of  10% A, 10% C, 10% G and 70% T
  misc_feature (17)..(17): n is mix of  10% A, 70% C, 10% G and 10% T
  misc_feature (18)..(18): n is a mix of  10% A, 10% C, 10% G and 70% T
  misc_feature (22)..(22): n is mix of  10% A, 70% C, 10% G and 10% T
  misc_feature (23)..(23): n is a mix of  70% A, 10% C, 10% G and 10% T
  misc_feature (24)..(24): n is a mix of  10% A, 10% C, 10% G and 70% T
  misc_feature (25)..(25): n is a mix of  10% A, 10% C, 70% G and 10% T
  misc_feature (26)..(26): n is a mix of  10% A, 10% C, 10% G and 70% T
  misc_feature (27)..(27): n is a mix of  10% A, 10% C, 10% G and 70% T
  misc_feature (34)..(34): n is a mix of  10% A, 10% C, 10% G and 70% T
  misc_feature (35)..(35): n is a mix of  10% A, 10% C, 10% G and 70% T
  misc_feature (36)..(36): n is a mix of  10% A, 10% C, 10% G and 70% T
  misc_feature (43)..(43): n is a mix of  10% A, 10% C, 10% G and 70% T
  misc_feature (44)..(44): n is a mix of  70% A, 10% C, 10% G and 10% T
  misc_feature (45)..(45): n is a mix of  10% A, 10% C, 10% G and 70% T
  misc_feature (49)..(49): n is a mix of  10% A, 70% C, 10% G and 10% T
  misc_feature (50)..(50): n is a mix of  10% A, 70% C, 10% G and 10% T
  misc_feature (51)..(51): n is a mix of  10% A, 10% C, 10% G and 70% T
  misc_feature (52)..(52): n is a mix of  10% A, 10% C, 10% G and 70% T
  misc_feature (53)..(53): n is a mix of  70% A, 10% C, 10% G and 10% T
  misc_feature (54)..(54): n is a mix of  10% A, 10% C, 10% G and 70% T
  misc_feature (58)..(58): n is a mix of  10% A, 10% C, 10% G and 70% T
  misc_feature (59)..(59): n is mix of  10% A, 70% C, 10% G and 10% T
  misc_feature (60)..(60): n is a mix of  10% A, 10% C, 10% G and 70% T
  misc_feature (61)..(61): n is a mix of  10% A, 10% C, 70% G and 10% T
  misc_feature (62)..(62): n is a mix of 70% A, 10% C, 10% G and 10% T
  misc_feature (63)..(63): n is a mix of  10% A, 10% C, 10% G and 70% T
  misc_feature (70)..(70): n is a mix of  10% A, 10% C, 70% G and 10% T
  misc_feature (71)..(71): n is a mix of  10% A, 70% C, 10% G and 10% T
  misc_feature (72)..(72): n is a mix of  70% A, 10% C, 10% G and 10% T
  misc_feature (73)..(73): n is a mix of  10% A, 70% C, 10% G and 10% T
  misc_feature (74)..(74): n is a mix of  10% A, 10% C, 10% G and 70% T
  misc_feature (75)..(75): n is a mix of  10% A, 10% C, 70% G and 10% T
  misc_feature (82)..(82): n is a mix of  10% A, 10% C, 70% G and 10% T
  misc_feature (83)..(83): n is a mix of  70% A, 10% C, 10% G and 10% T
  misc_feature (84)..(84): n is a mix of  10% A, 10% C, 70% G and 10% T
aaggctggtatcaccnnngacnnnnnnttcaacnnnatcaatnnngcgnnnnnngtgnnnnnngttaacnnnnnnaagaacnnnatcctgaaagctcac

SEQ ID NO: 131 - DNA, 79 bp, Artificial Sequence
  Synthetic sequence
  misc_feature (16)..(16): n is a mix of  70% A, 10% C, 10% G and 10% T
  misc_feature (17)..(17): n is a mix of  10% A, 10% C, 10% G and 70% T
  misc_feature (18)..(18): n is a mix of  10% A, 10% C, 10% G and 70% T
  misc_feature (25)..(25): n is a mix of  10% A, 70% C, 10% G and 10% T
  misc_feature (26)..(26): n is a mix of  10% A, 10% C, 10% G and 70% T
  misc_feature (27)..(27): n is a mix of  10% A, 70% C, 10% G and 10% T
  misc_feature (28)..(28): n is a mix of  70% A, 10% C, 10% G and 10% T
  misc_feature (29)..(29): n is a mix of  70% A, 10% C, 10% G and 10% T
  misc_feature (30)..(30): n is a mix of  70% A, 10% C, 10% G and 10% T
  misc_feature (34)..(34): n is a mix of  10% A, 70% C, 10% G and 10% T
  misc_feature (35)..(35): n is a mix of  10% A, 70% C, 10% G and 10% T
  misc_feature (36)..(36): n is a mix of  10% A, 10% C, 10% G and 70% T
  misc_feature (40)..(40): n is a mix of  70% A, 10% C, 10% G and 10% T
  misc_feature (41)..(41): n is a mix of  10% A, 10% C, 10% G and 70% T
  misc_feature (42)..(42): n is a mix of  10% A, 70% C, 10% G and 10% T
  misc_feature (46)..(46): n is a mix of  10% A, 70% C, 10% G and 10% T
  misc_feature (47)..(47): n is a mix of  70% A, 10% C, 10% G and 10% T
  misc_feature (48)..(48): n is a mix of  70% A, 10% C, 10% G and 10% T
  misc_feature (49)..(49): n is a mix of  10% A, 70% C, 10% G and 10% T
  misc_feature (50)..(50): n is a mix of  70% A, 10% C, 10% G and 10% T
  misc_feature (51)..(51): n is a mix of  10% A, 70% C, 10% G and 10% T
  misc_feature (55)..(55): n is a mix of  10% A, 70% C, 10% G and 10% T
  misc_feature (56)..(56): n is a mix of  10% A, 10% C, 10% G and 70% T
  misc_feature (57)..(57): n is a mix of  10% A, 10% C, 10% G and 70% T
  misc_feature (58)..(58): n is a mix of  10% A, 10% C, 10% G and 70% T
  misc_feature (59)..(59): n is a mix of  10% A, 10% C, 10% G and 70% T
  misc_feature (60)..(60): n is a mix of  10% A, 70% C, 10% G and 10% T
  misc_feature (61)..(61): n is a mix of  70% A, 10% C, 10% G and 10% T
  misc_feature (62)..(62): n is a mix of  70% A, 10% C, 10% G and 10% T
  misc_feature (63)..(63): n is a mix of  10% A, 70% C, 10% G and 10% T
gcgaaagaagatgctnnngcagaannnnnnaagnnnggtnnnaccnnnnnncatnnnnnnnnntttatcaatcacgcgc

SEQ ID NO: 132 - DNA, 52 bp, Artificial Sequence
  Synthetic sequence
  misc_feature (19)..(19): n is a mix of  10% A, 70% C, 10% G and 10% T
  misc_feature (20)..(20): n is a mix of  10% A, 70% C, 10% G and 10% T
  misc_feature (21)..(21): n is a mix of  10% A, 70% C, 10% G and 10% T
  misc_feature (22)..(22): n is a mix of  70% A, 10% C, 10% G and 10% T
  misc_feature (23)..(23): n is a mix of  10% A, 10% C, 10% G and 70% T
  misc_feature (24)..(24): n is a mix of  10% A, 70% C, 10% G and 10% T
  misc_feature (25)..(25): n is a mix of  10% A, 70% C, 10% G and 10% T
  misc_feature (26)..(26): n is a mix of  10% A, 10% C, 10% G and 70% T
  misc_feature (27)..(27): n is a mix of  10% A, 70% C, 10% G and 10% T
  misc_feature (28)..(28): n is a mix of  70% A, 10% C, 10% G and 10% T
  misc_feature (29)..(29): n is a mix of  70% A, 10% C, 10% G and 10% T
  misc_feature (30)..(30): n is a mix of  70% A, 10% C, 10% G and 10% T
  misc_feature (31)..(31): n is a mix of  10% A, 70% C, 10% G and 10% T
  misc_feature (32)..(32): n is a mix of  10% A, 70% C, 10% G and 10% T
  misc_feature (33)..(33): n is a mix of  10% A, 10% C, 10% G and 70% T
  misc_feature (34)..(34): n is a mix of  10% A, 70% C, 10% G and 10% T
  misc_feature (35)..(35): n is a mix of  70% A, 10% C, 10% G and 10% T
  misc_feature (36)..(36): n is a mix of  10% A, 70% C, 10% G and 10% T
gttaacgggctgaagaacnnnnnnnnnnnnnnnnnngccgggagctctggag

SEQ ID NO: 133 - protein, 27 aa, Artificial Sequence
  Synthetic sequence
  misc_feature (6)..(6): Xaa is l, r, or t
  misc_feature (16)..(16): Xaa is h, i, l, r, or v
  misc_feature (20)..(20): Xaa is G, r, or V
  misc_feature (24)..(24): Xaa is a, r, h, s, or t
WDNAWXEIRHLPNLNXEQKXAFIXSLY

SEQ ID NO: 134 - protein, 14 aa, Artificial Sequence
  Synthetic sequence
SANLLAEAKKLNDA

SEQ ID NO: 135 - protein, 23 aa, Artificial Sequence
  Synthetic sequence
DDPSQSANLLAEAKKLNDAQAPK

SEQ ID NO: 136 - protein, 8 aa, Artificial Sequence
  Synthetic sequence
VDNKFNKE

SEQ ID NO: 137 - protein, 11 aa, Artificial Sequence
  Synthetic sequence
  misc_feature (4)..(5): Xaa is any amino acid
  misc_feature (7)..(8): Xaa is any amino acid
  misc_feature (10)..(10): Xaa is any amino acid
PHVXXFXXHXP

SEQ ID NO: 138 - protein, 16 aa, Artificial Sequence
  Synthetic sequence
  misc_feature (1)..(1): Xaa is l, v, or i
  misc_feature (2)..(2): Xaa is l or c
XXKNAKEDAIAELKKA

SEQ ID NO: 139 - protein, 24 aa, Artificial Sequence
  Synthetic sequence
  misc_feature (1)..(1): Xaa is t, y, f, i, p, or r
  misc_feature (2)..(2): Xaa is i, h, n, p, or s
  misc_feature (3)..(3): Xaa is d, i, or v
  misc_feature (6)..(6): Xaa is l, v, or i
  misc_feature (7)..(7): Xaa is l or c
XXXQWXXKNAKEDAIAELKKAGIT

SEQ ID NO: 140 - protein, 6 aa, Artificial Sequence
  Synthetic sequence
ILKAHA

SEQ ID NO: 141 - protein, 53 aa, Artificial Sequence
  Synthetic sequence
  misc_feature (1)..(1): Xaa is t, y, f, i, p and r
  misc_feature (2)..(2): Xaa is i, h, n, p, and s
  misc_feature (3)..(3): Xaa is d, i, and v
  misc_feature (6)..(6): Xaa is l, v, and i
  misc_feature (7)..(7): Xaa is l and c
  misc_feature (37)..(37): Xaa is t, y, n, and s
  misc_feature (38)..(38): Xaa is v or c
XXXQWXXKNAKEDAIAELKKAGITEPHVISFINHAPXXSHVNGLKNAILKAHA

SEQ ID NO: 142 - protein, 7 aa, Artificial Sequence
  Synthetic sequence
  misc_feature (1)..(1): Xaa is any D-amino acid residue
  misc_feature (2)..(2): Xaa is any D-amino acid residue
  misc_feature (3)..(3): Xaa is any D-amino acid residue
  misc_feature (6)..(6): Xaa is i or v
  misc_feature (7)..(7): Xaa is s or n
XXXQWXX

SEQ ID NO: 143 - protein, 9 aa, Artificial Sequence
  Synthetic sequence
  misc_feature (1)..(1): Xaa is any a D-aromatic amino acid
  misc_feature (2)..(2): Xaa is a hydrophobic residue
  misc_feature (3)..(3): Xaa is any amino acid
  misc_feature (4)..(4): Xaa is any amino acid
  misc_feature (5)..(5): Xaa is any D-aromatic amino acid
  misc_feature (6)..(6): Xaa is a hydrophobic residue
  misc_feature (7)..(7): Xaa is any amino acid
  misc_feature (8)..(8): Xaa is any D-aromatic amino acid
  misc_feature (9)..(9): Xaa is a hydrophobic residue
XXXXXXXXX

SEQ ID NO: 144 - protein, 11 aa, Artificial Sequence
  Synthetic sequence
  misc_feature (1)..(1): Xaa is a helix-terminating residue
  misc_feature (3)..(3): Xaa is a, i, l, or v
  misc_feature (4)..(4): Xaa is any amino acid
  misc_feature (5)..(5): Xaa is any amino acid
  misc_feature (7)..(7): Xaa is a, i, l, or v
  misc_feature (8)..(8): Xaa is any amino acid
  misc_feature (10)..(10): Xaa is a, i, l, or v
  misc_feature (11)..(11): Xaa is a helix-terminating residue
XHXXXFXXHXX

SEQ ID NO: 145 - protein, 11 aa, Artificial Sequence
  Synthetic sequence
  misc_feature (1)..(1): Xaa is a helix-terminating residue
  misc_feature (4)..(4): Xaa is any amino acid
  misc_feature (5)..(5): Xaa is any amino acid
  misc_feature (7)..(7): Xaa is a, i, l, or v
  misc_feature (8)..(8): Xaa is any amino acid
  misc_feature (10)..(10): Xaa is a, i, l, or v
XHVXXFXXHXP

SEQ ID NO: 146 - protein, 9 aa, Artificial Sequence
  Synthetic sequence
  misc_feature (1)..(1): Xaa is a D-aromatic amino acid
  misc_feature (2)..(2): Xaa is a hydrophobic residue
  misc_feature (3)..(3): Xaa is any amino acid
  misc_feature (4)..(4): Xaa is a non-polar amino acid residue
  misc_feature (5)..(5): Xaa is a hydrophobic residue
  misc_feature (6)..(6): Xaa is any amino acid
  misc_feature (7)..(7): Xaa is any amino acid
  misc_feature (8)..(8): Xaa is a non-polar amino acid residue
  misc_feature (9)..(9): Xaa is a hydrophobic residue
XXXXXXXXX

SEQ ID NO: 147 - protein, 4 aa, Artificial Sequence
  Synthetic sequence
  misc_feature (1)..(1): Xaa is a helix-terminating residue
  misc_feature (2)..(2): Xaa is any amino acid
  misc_feature (3)..(3): Xaa is a polar amino acid residue
  misc_feature (4)..(4): Xaa is a helix-terminating residue
XXXX

SEQ ID NO: 148 - protein, 4 aa, Artificial Sequence
  Synthetic sequence
  misc_feature (1)..(1): Xaa is a helix-terminating residue
  misc_feature (2)..(2): Xaa is any amino acid
  misc_feature (4)..(4): Xaa is a helix-terminating residue
XXEX

SEQ ID NO: 149 - protein, 23 aa, Artificial Sequence
  Synthetic sequence
  misc_feature (8)..(8): Xaa is any amino acid
  misc_feature (9)..(9): Xaa is any amino acid
  misc_feature (11)..(11): Xaa is any amino acid
  misc_feature (13)..(13): Xaa is s, n, or y
  misc_feature (14)..(14): Xaa is any amino acid
  misc_feature (17)..(17): Xaa is any amino acid
  misc_feature (18)..(18): Xaa is any amino acid
  misc_feature (20)..(20): Xaa is any amino acid
  misc_feature (21)..(21): Xaa is any amino acid
  misc_feature (22)..(22): Xaa is any amino acid
EPHVISFXXHXPXXSHXXGXXXA

SEQ ID NO: 150 - protein, 41 aa, Artificial Sequence
  Synthetic sequence
  misc_feature (2)..(2): Xaa is any amino acid
  misc_feature (3)..(3): Xaa is any amino acid
  misc_feature (4)..(4): Xaa is any amino acid
  misc_feature (5)..(5): Xaa is any amino acid
  misc_feature (6)..(6): Xaa is any amino acid
  misc_feature (7)..(7): Xaa is any amino acid
  misc_feature (8)..(8): Xaa is any amino acid
  misc_feature (9)..(9): Xaa is any amino acid
  misc_feature (10)..(10): Xaa is any amino acid
  misc_feature (11)..(11): Xaa is any amino acid
  misc_feature (12)..(12): Xaa is any amino acid
  misc_feature (13)..(13): Xaa is any amino acid
  misc_feature (14)..(14): Xaa is any amino acid
  misc_feature (15)..(15): Xaa is any amino acid
  misc_feature (16)..(16): Xaa is any amino acid
  misc_feature (17)..(17): Xaa is any amino acid
  misc_feature (18)..(18): Xaa is any amino acid
  misc_feature (26)..(26): Xaa is any amino acid
  misc_feature (27)..(27): Xaa is any amino acid
  misc_feature (29)..(29): Xaa is any amino acid
  misc_feature (31)..(31): Xaa is s or n
  misc_feature (35)..(35): Xaa is any amino acid
  misc_feature (36)..(36): Xaa is any amino acid
  misc_feature (38)..(38): Xaa is any amino acid
  misc_feature (39)..(39): Xaa is any amino acid
  misc_feature (40)..(40): Xaa is any amino acid
CXXXXXXXXXXXXXXXXXEPHVISFXXHXPXCSHXXGXXXA

SEQ ID NO: 151 - protein, 9 aa, Artificial Sequence
  Synthetic sequence
  misc_feature (2)..(2): Xaa is a hydrophobic residue
  misc_feature (3)..(3): Xaa is any amino acid
  misc_feature (4)..(4): Xaa is any amino acid
  misc_feature (6)..(6): Xaa is a hydrophobic residue
  misc_feature (7)..(7): Xaa is any amino acid
  misc_feature (9)..(9): Xaa is a hydrophobic residue
HXXXFXXHX

SEQ ID NO: 152 - protein, 9 aa, Artificial Sequence
  Synthetic sequence
  misc_feature (2)..(2): Xaa is a hydrophobic residue
  misc_feature (3)..(3): Xaa is any amino acid
  misc_feature (4)..(4): Xaa is a non-polar amino acid residue
  misc_feature (5)..(5): Xaa is a hydrophobic residue
  misc_feature (6)..(6): Xaa is any amino acid
  misc_feature (7)..(7): Xaa is any amino acid
  misc_feature (8)..(8): Xaa is a non-polar amino acid residue
  misc_feature (9)..(9): Xaa is a hydrophobic residue
HXXXXXXXX

SEQ ID NO: 153 - protein, 11 aa, Artificial Sequence
  Synthetic sequence
  misc_feature (1)..(1): Xaa is a helix-terminating residue
  misc_feature (3)..(3): Xaa is a hydrophobic residue
  misc_feature (4)..(4): Xaa is any amino acid
  misc_feature (5)..(5): Xaa is any amino acid
  misc_feature (7)..(7): Xaa is a hydrophobic residue
  misc_feature (8)..(8): Xaa is any amino acid
  misc_feature (10)..(10): Xaa is a hydrophobic residue
  misc_feature (11)..(11): Xaa is a helix-terminating residue
XHXXXFXXHXX

SEQ ID NO: 154 - protein, 24 aa, Artificial Sequence
  Synthetic sequence
  misc_feature (1)..(1): Xaa is a helix-terminating residue
  misc_feature (3)..(3): Xaa is a hydrophobic residue
  misc_feature (4)..(4): Xaa is any amino acid
  misc_feature (5)..(5): Xaa is any amino acid
  misc_feature (7)..(7): Xaa is a hydrophobic residue
  misc_feature (8)..(8): Xaa is any amino acid
  misc_feature (10)..(10): Xaa is a hydrophobic residue
  misc_feature (11)..(11): Xaa is a helix-terminating residue
  misc_feature (13)..(13): Xaa is any amino acid
  misc_feature (14)..(14): Xaa is any amino acid
  misc_feature (16)..(16): Xaa is a hydrophobic residue
  misc_feature (17)..(17): Xaa is any amino acid
  misc_feature (18)..(18): Xaa is a non-polar amino acid residue
  misc_feature (19)..(19): Xaa is a hydrophobic residue
  misc_feature (20)..(20): Xaa is any amino acid
  misc_feature (21)..(21): Xaa is any amino acid
  misc_feature (22)..(22): Xaa is a non-polar amino acid residue
  misc_feature (23)..(23): Xaa is a hydrophobic residue
  misc_feature (24)..(24): Xaa is any amino acid
XHXXXFXXHXXYXXHXXXXXXXXX

SEQ ID NO: 155 - protein, 28 aa, Artificial Sequence
  Synthetic sequence
  misc_feature (1)..(1): Xaa is a helix-terminating residue
  misc_feature (2)..(2): Xaa is any amino acid
  misc_feature (3)..(3): Xaa is any amino acid
  misc_feature (5)..(5): Xaa is a helix-terminating residue
  misc_feature (7)..(7): Xaa is a hydrophobic residue
  misc_feature (8)..(8): Xaa is any amino acid
  misc_feature (9)..(9): Xaa is any amino acid
  misc_feature (11)..(11): Xaa is a hydrophobic residue
  misc_feature (12)..(12): Xaa is any amino acid
  misc_feature (14)..(14): Xaa is a hydrophobic residue
  misc_feature (15)..(15): Xaa is a helix-terminating residue
  misc_feature (17)..(17): Xaa is any amino acid
  misc_feature (18)..(18): Xaa is any amino acid
  misc_feature (20)..(20): Xaa is a hydrophobic residue
  misc_feature (21)..(21): Xaa is any amino acid
  misc_feature (22)..(22): Xaa is a non-polar amino acid residue
  misc_feature (23)..(23): Xaa is a hydrophobic residue
  misc_feature (24)..(24): Xaa is any amino acid
  misc_feature (25)..(25): Xaa is any amino acid
  misc_feature (26)..(26): Xaa is a non-polar amino acid residue
  misc_feature (27)..(27): Xaa is a hydrophobic residue
  misc_feature (28)..(28): Xaa is any amino acid
XXXEXHXXXFXXHXXYXXHXXXXXXXXX

SEQ ID NO: 156 - protein, 46 aa, Artificial Sequence
  Synthetic sequence
  misc_feature (20)..(20): Xaa is any amino acid
  misc_feature (21)..(21): Xaa is any amino acid
  misc_feature (22)..(22): Xaa is any amino acid
  misc_feature (23)..(23): Xaa is any amino acid
  misc_feature (24)..(24): Xaa is any amino acid
  misc_feature (25)..(25): Xaa is any amino acid
  misc_feature (26)..(26): Xaa is any amino acid
  misc_feature (29)..(29): Xaa is any amino acid
  misc_feature (31)..(31): Xaa is any amino acid
  misc_feature (32)..(32): Xaa is any amino acid
  misc_feature (34)..(35): Xaa is any amino acid
  misc_feature (38)..(38): Xaa is any amino acid
  misc_feature (39)..(39): Xaa is any amino acid
  misc_feature (42)..(42): Xaa is any amino acid
LLKNAKEDAIAELKKAGITXXXXXXXINXAXXVXXVNXXKNXILKA

SEQ ID NO: 157 - protein, 53 aa, Artificial Sequence
  Synthetic sequence
  misc_feature (25)..(25): Xaa is any amino acid
  misc_feature (26)..(26): Xaa is any amino acid
  misc_feature (27)..(27): Xaa is any amino acid
  misc_feature (28)..(28): Xaa is any amino acid
  misc_feature (29)..(29): Xaa is any amino acid
  misc_feature (30)..(30): Xaa is any amino acid
  misc_feature (31)..(31): Xaa is any amino acid
  misc_feature (34)..(34): Xaa is any amino acid
  misc_feature (36)..(36): Xaa is any amino acid
  misc_feature (37)..(37): Xaa is any amino acid
  misc_feature (39)..(39): Xaa is any amino acid
  misc_feature (40)..(40): Xaa is any amino acid
  misc_feature (43)..(43): Xaa is any amino acid
  misc_feature (44)..(44): Xaa is any amino acid
  misc_feature (47)..(47): Xaa is any amino acid
TIDQWLLKNAKEDAIAELKKAGITXXXXXXXINXAXXVXXVNXXKNXILKAHA

SEQ ID NO: 158 - protein, 58 aa, Artificial Sequence
  Synthetic sequence
  misc_feature (14)..(14): Xaa is any amino acid
  misc_feature (24)..(24): Xaa is any amino acid
  misc_feature (28)..(28): Xaa is any amino acid
  misc_feature (32)..(32): Xaa is any amino acid
VDNKFNKEWDNAWXEIRHLPNLNXEQKXAFIXSLYDDPSQSANLLAEAKKLNDAQAPK

SEQ ID NO: 159 - protein, 53 aa, Artificial Sequence
  Synthetic sequence
  misc_feature (25)..(25): Xaa is any amino acid
  misc_feature (27)..(27): Xaa is any amino acid
  misc_feature (31)..(31): Xaa is any amino acid
  misc_feature (34)..(34): Xaa is any amino acid
  misc_feature (36)..(36): Xaa is any amino acid
  misc_feature (37)..(37): Xaa is any amino acid
  misc_feature (39)..(39): Xaa is any amino acid
  misc_feature (40)..(40): Xaa is any amino acid
  misc_feature (43)..(43): Xaa is any amino acid
  misc_feature (44)..(44): Xaa is any amino acid
  misc_feature (47)..(47): Xaa is any amino acid
TIDQWLLKNAKEDAIAELKKAGITXDXYFNXINXAXXVXXVNXXKNXILKAHA

SEQ ID NO: 160 - protein, 27 aa, Artificial Sequence
  Synthetic sequence
  misc_feature (3)..(3): Xaa is any amino acid
  misc_feature (4)..(4): Xaa is any amino acid
  misc_feature (6)..(6): Xaa is l, r, or t
  misc_feature (7)..(7): Xaa is any amino acid
  misc_feature (8)..(8): Xaa is any amino acid
  misc_feature (10)..(10): Xaa is any amino acid
  misc_feature (11)..(11): Xaa is any amino acid
  misc_feature (12)..(12): Xaa is any amino acid
  misc_feature (13)..(13): Xaa is any amino acid
  misc_feature (14)..(14): Xaa is any amino acid
  misc_feature (15)..(15): Xaa is any amino acid
  misc_feature (16)..(16): Xaa is h, i, l, r, or v
  misc_feature (17)..(17): Xaa is any amino acid
  misc_feature (18)..(18): Xaa is any amino acid
  misc_feature (20)..(20): Xaa is G, r, or v
  misc_feature (21)..(21): Xaa is any amino acid
  misc_feature (22)..(22): Xaa is any amino acid
  misc_feature (23)..(23): Xaa is any amino acid
  misc_feature (24)..(24): Xaa is a, r, h, s, or t
  misc_feature (25)..(25): Xaa is any amino acid
  misc_feature (26)..(26): Xaa is any amino acid
WDXXWXXXRXXXXXXXXXKXXXXXXXY

SEQ ID NO: 161 - protein, 27 aa, Artificial Sequence
  Synthetic sequence
  misc_feature (3)..(3): Xaa is any amino acid
  misc_feature (4)..(4): Xaa is any amino acid
  misc_feature (7)..(7): Xaa is any amino acid
  misc_feature (8)..(8): Xaa is any amino acid
  misc_feature (10)..(10): Xaa is any amino acid
  misc_feature (11)..(11): Xaa is any amino acid
  misc_feature (12)..(12): Xaa is any amino acid
  misc_feature (13)..(13): Xaa is any amino acid
  misc_feature (14)..(14): Xaa is any amino acid
  misc_feature (15)..(15): Xaa is any amino acid
  misc_feature (17)..(17): Xaa is any amino acid
  misc_feature (18)..(18): Xaa is any amino acid
  misc_feature (21)..(21): Xaa is any amino acid
  misc_feature (22)..(22): Xaa is any amino acid
  misc_feature (23)..(23): Xaa is any amino acid
  misc_feature (25)..(25): Xaa is any amino acid
  misc_feature (26)..(26): Xaa is any amino acid
WDXXWRXXRXXXXXXLXXKRXXXSXXY

SEQ ID NO: 162 - protein, 27 aa, Artificial Sequence
  Synthetic sequence
  misc_feature (3)..(3): Xaa is any amino acid
  misc_feature (4)..(4): Xaa is any amino acid
  misc_feature (7)..(7): Xaa is any amino acid
  misc_feature (8)..(8): Xaa is any amino acid
  misc_feature (10)..(10): Xaa is any amino acid
  misc_feature (11)..(11): Xaa is any amino acid
  misc_feature (12)..(12): Xaa is any amino acid
  misc_feature (13)..(13): Xaa is any amino acid
  misc_feature (14)..(14): Xaa is any amino acid
  misc_feature (15)..(15): Xaa is any amino acid
  misc_feature (17)..(17): Xaa is any amino acid
  misc_feature (18)..(18): Xaa is any amino acid
  misc_feature (21)..(21): Xaa is any amino acid
  misc_feature (22)..(22): Xaa is any amino acid
  misc_feature (23)..(23): Xaa is any amino acid
  misc_feature (25)..(25): Xaa is any amino acid
  misc_feature (26)..(26): Xaa is any amino acid
WDXXWRXXRXXXXXXVXXKRXXXRXXY

SEQ ID NO: 163 - protein, 57 aa, Artificial Sequence
  Synthetic sequence
DNKFNKEQQNAFYEILHLPNLNEEQRNAFIQSLKDDPSQSANLLAEAKKLNDAQAPK

SEQ ID NO: 164 - protein, 131 aa, Artificial Sequence
  Synthetic sequence
GCGAAAGAAGATGCTNNNGCAGAANNNNNNAAGNNNGGTNNNACCNNNNNNCATNNNNNNNNNTTTATCAATCACGCGCGTTAACGGGCTGAAGAACNNNNNNNNNNNNNNNNNNGCCGGGAGCTCTGGAG

SEQ ID NO: 165 - protein, 58 aa, Artificial Sequence [sequence omitted]

SEQ ID NO: 166 - protein, 58 aa, Artificial Sequence
  Synthetic sequence
  misc_feature (9)..(9): Xaa is any amino acid
  misc_feature (10)..(10): Xaa is any amino acid
  misc_feature (13)..(13): Xaa is any amino acid
  misc_feature (14)..(14): Xaa is any amino acid
  misc_feature (17)..(17): Xaa is any amino acid
  misc_feature (24)..(24): Xaa is any amino acid
  misc_feature (27)..(27): Xaa is any amino acid
  misc_feature (28)..(28): Xaa is any amino acid
  misc_feature (32)..(32): Xaa is any amino acid
  misc_feature (35)..(35): Xaa is any amino acid
VDNKFNKEXXNAXXEIXHLPNLNXEQXXAFIXSLXDDPSQSANLLAEAKKLNDAQAPK

SEQ ID NO: 167 - protein, 114 aa, Artificial Sequence
  Synthetic sequence
CAVDNKFNKEWDNAWREIRHLPNLNLEQKRAFISSLYDDPSQSANLLAEAKKLNDAQAPKCTIDQWLLKNAKEDAIAELKKAGITEPHVISFINHAPYVSHVNGLKNAILKAHA

SEQ ID NO: 168 - protein, 111 aa, Artificial Sequence
  Synthetic sequence
YHIQWVCKNAKEDAIAELKKAGITEPHVISFINHAPNCSHVNGLKNAILKAHAVDNKFNKEWDNAWREIRHLPNLNVEQKRAFIRSLYDDPSQSANLLAEAKKLNDAQAPK

SEQ ID NO: 169 - protein, 110 aa, Artificial Sequence
  Synthetic sequence
YHIQWVCKNAKEDAIAELKKAGITEPHVISFINHAPNCSHVNGLKAILKAHAVDNKFNKEWDNAWREIRHLPNLNVEQKRAFIRSLYDDPSQSANLLAEAKKLNDAQAPK